(19) United States
(12) Patent Application Publication
(10) Pub. No.: US 2025/0144236 A1
(43) Pub. Date: May 8, 2025

(54) NITROGEN-BRANCHED NON-LINEAR PEGYLATED LIPID AND APPLICATION THEREOF

(71) Applicant: XIAMEN SINOPEG BIOTECH CO., LTD., Xiamen, Fujian (CN)

(72) Inventors: Wengui WENG, Xiamen (CN); Chao LIU, Xiamen (CN); Ailan WANG, Xiamen (CN); Dandan CHEN, Xiamen (CN); Sheng LIN, Xiamen (CN); Guohua WEI, Xiamen (CN); Qi ZHU, Xiamen (CN); Congming LIN, Xiamen (CN)

(73) Assignee: XIAMEN SINOPEG BIOTECH CO., LTD., Xiamen, Fujian (CN)

(21) Appl. No.: 18/838,083

(22) PCT Filed: Apr. 11, 2023
(86) PCT No.: PCT/CN2023/087701

§ 371 Date: Aug. 13, 2024

(30) Foreign Application Priority Data
Apr. 12, 2022 (CN) 202210380598.6

(51) Int. Cl. A61K 48/00; A61K 31/7105; A61K 31/711; A61K 47/10; A61K 47/18; A61K 47/22; A61K 47/24

(52) U.S. Cl. CPC A61K 48/0041; A61K 31/7105; A61K 31/711; A61K 47/10; A61K 47/183; A61K 47/22; A61K 47/24

(57) ABSTRACT

A nitrogen-branched non-linear PEGylated lipid of Formula (1), wherein, X is —CRₐ< or

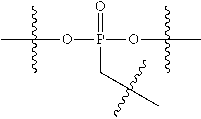 [SMILES: *CP(=O)(O*)O*]

(Rₐ is H or a C₁₋₁₂ alkyl group); B₁ and B₂ are linking bonds or C₁₋₂₀ alkylene groups; L₁ and L₂ are linking bonds or divalent linking groups; R₁ and R₂ are C₁₋₅₀ aliphatic hydrocarbon groups or C₁₋₅₀ residues of aliphatic hydrocarbon derivative, each containing 0-10 heteroatoms; L_{d} is a linking bond or a divalent linking group; N_{core} is a multivalent group of valence y+1, and contains a trivalent nitrogen-atom branching core connected to L_{d}; y is 2, 3, 4, 5, 6, 7, 8, 9, or ≥10; Lₓ is a linking bond or a divalent linking group; XPEG is a polyethylene glycol component. The non-linear PEGylated lipid herein can realize better surface modification of LNP. The lipid nanoparticle pharmaceutical composition and its formulation exhibit higher drug efficacy, especially for nucleic acid drugs.

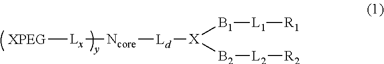

### CROSS-REFERENCE TO RELATED APPLICATIONS

This application is a U.S. National Phase Application of International Application No. PCT/CN2023/087701, filed Apr. 11, 2023 which claims priority to Chinese Application No. CN202210380598.6, filed Apr. 12, 2022. All of the aforementioned patent applications are hereby incorporated by reference in their entireties.

### TECHNICAL FIELD

The present invention belongs to the field of drug delivery, and overall relates to a PEGylated lipid; specifically, it relates to a nitrogen-branched non-linear PEGylated lipid that can be used as a component for drug carriers. The present invention also relates to a lipid composition and a lipid pharmaceutical composition, both containing the lipid, and formulation and application thereof.

### BACKGROUND OF THE INVENTION

Polyethylene glycol (PEG) has the advantages of hydrophilicity, flexibility, non-toxicity, low immunogenicity and biodegradability, and is widely used in the surface modification of drugs or drug carriers. Covalent binding of polyethylene glycol to a drug or a surface component (such as lipid) of a carrier, i.e., PEGylation, stands as a gold standard for reducing non-specific cellular uptake during drug delivery, enabling stealth effects of drugs or carriers thereof. The hydrated film formed by PEG under physiological conditions can effectively protect the modified drug or drug carrier, which not only makes the drug more stable, avoids rapid degradation or removal, but also strongly avoids the adsorption of plasma proteins on the surface of the drug carrier, thereby prolonging the blood circulation time of drugs or formulations of nanoparticle pharmaceutical compositions. PEG can effectively shield the immunogenicity of the modified substance, reduce systemic toxicity, and enhance the passive accumulation of the carrier in tumor or inflammatory tissue to improve the therapeutic effect.

PEG is commonly used in the modification of pharmaceutical proteins and drug carriers such as liposomes, wherein liposomes are widely used to deliver nucleic acid drugs, genetic vaccines, anti-tumor drugs, small molecule drugs, polypeptide drugs, protein drugs, etc. Lipid nanoparticles (LNPs), an improved form of traditional liposomes, have attracted much attention in the pharmaceutical industry as promising drug carriers. Especially with the application of lipid nanoparticles in mRNA vaccines against the novel coronavirus epidemic, lipid nanoparticle technology has entered a stage of rapid optimization and development. Unmodified LNP drug delivery systems usually have limitations such as short blood circulation time, poor stability, and lack of targeting, while the use of PEGylated lipids can significantly improve the efficacy of LNP drug formulations.

However, many studies have pointed out that PEGylated lipids will gradually detach from the surface of liposomes or LNPs, causing liposomes or LNPs to gradually lose their invisibility partially or completely during the systemic circulation, and such situation is exacerbated by plasma proteins and blood shear forces. Therefore, the stable presence of PEGylated lipids on the surface of liposomes or LNPs is required to maintain drug stability in blood circulation. On the other hand, the protective effect brought about by PEG modification will reduce the cellular uptake efficiency of drugs, wherein the modification with shorter-chain PEG has a reduced effect, which may be due to the poor shielding ability of short-chain PEG; whereas, when nonlinear PEG is used for the modification, the umbrella structure formed has a higher shielding ability (Veronese et al., BioDrugs, 2008, 22, 315-329). Therefore, compared with the modification with linear PEG one with branched PEG of similar molecular weight will lead to a relatively lower cellular uptake efficiency of the carrier modified.

In terms of pharmacokinetics, in some cases, compared with long-chain PEG modification, short-chain PEG modification can make liposomes or LNPs more durable in the systemic circulation, presumably because the longer the PEG chain, the easier it is for the corresponding PEGylated lipid to fall off from the liposome or LNP under shear force (Mastrotto et al., Mol. Pharmaceuticals, 2020, 17, 472-487). In the case of similar single-chain lengths, nonlinear PEGylated lipids are easier to fall off than linear PEGylated lipids, due to the higher molecular weight of PEG in a single lipid molecule and the greater shear force experienced.

Currently, PEGylated lipids that are widely used and studied are basically modified with linear PEG Typical examples include DMG-PEG2000 and ALC-0159, while non-linear PEGylated lipids are rarely used.

To solve the aforementioned problems, it is necessary to develop a novel nonlinear PEGylated lipid.

### SUMMARY OF THE INVENTION

The present invention provides a novel non-linear PEGylated lipid, and applies the lipid in the preparation of lipid nanoparticles and pharmaceutical formulations thereof, realizing the advantages of excellent protection, longer systemic circulation time, high biocompatibility, low toxicity, high encapsulation efficiency, high delivery efficiency, etc.

The above-mentioned purpose of the present invention is achieved by the following technical solutions.

An embodiment of the present invention is as follows:

A PEGylated lipid having the structure represented by the general formula (1):

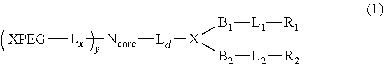

wherein, X is —CRₐ< or

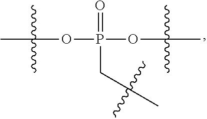 [SMILES: *CP(=O)(O*)O*]

and Rₐ is H or a C₁₋₁₂ alkyl group;
B₁ and B₂ are each independently a linking bond or a C₁₋₂₀ alkylene group;
L₁ and L₂ are each independently a linking bond or a divalent linking group;
R₁ and R₂ are each independently a C₁₋₅₀ aliphatic hydrocarbon group or a C₁₋₅₀ residue of aliphatic hydrocarbon derivative, containing 0-10 heteroatoms; the heteroatom is B, O, N, Si, P or S;
L_{d} is a linking bond or a divalent linking group;
N_{core} is a multivalent group having a valence of y+1, and contains a trivalent nitrogen-atom branching core connected to L_{d};
y is 2, 3, 4, 5, 6, 7, 8 or 9, or y≥10;
y instances of Lₓ are each independently a linking bond or a divalent linking group;
XPEG is a polyethylene glycol component; y instances of XPEG each independently contains one, two, three or four RPEG; RPEG is a single-chain polyethylene glycol component containing at least 4 EO units, and the EO unit is —CH₂CH₂O— or —OCH₂CH₂—; RPEG in the same XPEG have the same terminal group T; T is a hydrogen atom, an alkyl group or R₀₁-L₀₁-, wherein L₀₁ is a linking bond or a divalent linking group, and R₀₁ is a functional group that can interact with bio-related substances;
the alkyl group, alkylene group, aliphatic hydrocarbon group, and residue of aliphatic hydrocarbon derivative are each independently substituted or unsubstituted;
the PEGylated lipid is monodisperse or polydisperse;
or a salt, tautomer, stereoisomer or solvate thereof.

The present invention provides another embodiment:

A lipid composition containing the PEGylated lipid having the structure represented by the general formula (1).

The present invention provides another embodiment:

A lipid pharmaceutical composition containing the aforementioned lipid composition and drugs.

The present invention provides another embodiment:

A formulation of lipid pharmaceutical composition containing the aforementioned lipid pharmaceutical composition and pharmaceutically acceptable diluents or excipients.

Compared with the prior art, the present invention has the following beneficial effects:

The present invention provides a lipid compound modified by non-linear polyethylene glycol, which can enable the LNP to possess an excellent “stealth effect”. The non-linear PEGylated lipid of the present invention can, under the condition of smaller molar amounts and/or shorter polyethylene glycol chains, achieve an LNP surface modification which is comparable to or even better than one using the linear PEGylated lipid, and thus result in better protection, prolong the systemic circulation time of LNP, and reduce the biological toxicity of LNP to a greater extent. The non-linear PEGylated lipid nanoparticle of the present invention can be used as a carrier for targeted delivery of one or more bioactive agents, or used in an antigen display system. The invention provides a non-linear PEGylated lipid nanoparticle pharmaceutical composition and formulation thereof, which can contribute to better drug efficacy, and especially in some specific embodiments, lead to comparable or even better nucleic acid transfection efficiency than commercially available transfection reagents. When the polyethylene glycol component of the non-linear PEGylated lipid of the present invention has a targeting group at its end, better targeting properties of LNP can also be obtained.

### 1. DETAILED DESCRIPTION OF THE INVENTION

The present invention has provided a detailed description of the specific embodiments; however, it should be understood that, it is only provided in an illustrative manner rather than a restrictive manner. Any changes and modifications within the scope of the present invention will be obvious to those skilled in the art.

If a reference cited herein has a description different from the description in the present invention, the present invention shall prevail. This principle applies to all references cited throughout the specification.

### 1.1. Terminology

In the present invention, unless otherwise described, all technical and scientific terms used herein have the same meaning as is commonly understood by one of ordinary skill in the art. The disclosures of all patents and other publications cited herein are incorporated by reference in their entireties. In the event that any description or interpretation of a term herein conflicts with any document incorporated herein by reference, the following description or interpretation of the term shall prevail. Unless otherwise specified, each term has the following meaning.

In the present invention, two or more objects are “preferably, each independently selected from” their own scopes. When multiple levels of preferred selections are present, it is not mandatory that all are selected from the preferred groups at the same level; it is allowed to have one selected from a larger preferred group and one from a smaller preferred group; it is also allowed to have one selected from the largest preferred group and another from any preferred group; or, the selections can also be from preferred groups at the same level.

In the present invention, the terms “each independently”, “each independently selected from”, and “preferably, each independently selected from” not only mean that different objects can be each independently, or each independently selected from, or preferably, each independently selected from any option within the definitions, but also apply to the same object at different locations or occurrences. For example, provided that “a divalent group is selected from the group consisting of —NR_{c}C(═O)—, —C(═O)NR_{c}—, —NR_{c}C(═O)NR_{c}—, —OC(═O)NR_{c}—, —NR_{c}C(═O)O—, —SC(═O)NR_{c}—, —NR_{c}C(═O)S—, —C(R_{c})═N—NR_{c}—, and —NR_{c}—N═C(R_{c})—; wherein, R_{c} is, at each occurrence, independently H or a C₁₋₁₂ alkyl group”, such description indicates that the two R_{c} groups can be the same or different in “—NR_{c}—N═C(R_{c})—”, and that any R_{c} in “—NR_{c}C(═O)NR_{c}—” can be the same as or different from the R_{c} in “—NR_{c}C(═O)O—”.

In the present invention, when at least two items are listed, the “combination” of the listed items is composed of any two or more of the aforementioned listed items; wherein, the number of an item is not limited, and the number of any item can be one or greater than one; when the number of an item is greater than 1, any two of the items can appear in the same or different specific forms. For example, provided that “a divalent linking group is selected from the group consisting of —CH₂—, —O—, —S—, —C(═O)—, —NR_{c}—, and combinations thereof; wherein, R_{c} is, at each occurrence, independently a hydrogen atom or a C₁₋₅ alkyl group”, the divalent linking group can be —CH₂—, —O—, —S—, —C(═O)— or —NR_{c}—, or a combination, e.g., —CH₂—NH—CH₂— (wherein, the number of —CH₂— is 2, and the number of —NR_{c}— is 1), —(CH₂)₂—NH—(CH₂)₄—N(CH₃)— (wherein, the number of —CH₂— is 6, and the number of —NR_{c}— is 2 with unidentical specific forms), or —(CH₂)₂—NHC(═O)— (wherein, the number of —CH₂— is 2, the number of —NR_{c}— is 1, and the number of —C(═O)— is 1), etc. Particularly, a combination composed of a linking bond and any linking group is still the linking group itself. In the present invention, a “linking group” contains at least one atom by default.

In the present invention, unless otherwise specified, the terms “include”, “contain”, “cover” and similar expressions in the specification and claims herein shall be interpreted as “include(s) but is/are not limited to” or “include(s) without limitation” in an open and inclusive manner.

In the present invention, the term “include(s) but is/are not limited to” followed by a certain range means that the items within the range are selectable but the selections are not limited to the range. Not all structures within the range are applicable, and especially, the items explicitly excluded by the present invention are not for consideration. The basic principle is to use the successful implementation of the present invention as a screening criterion.

In the present invention, the interpretation of numerical intervals includes a numerical interval indicated by a dash (e.g., 1-6), a numerical interval indicated by a wavy line (e.g., 1˜6), and a numerical interval indicated by “to” (e.g., 1 to 6). Unless otherwise specified, an interval in the form of a range can represent a group consisting of all integers and non-integers within the range including two endpoints. For example, provided that an average number of EO units is selected from 22˜100, the range of selections is not limited to integers but also any non-integers within the interval. For example, the “integer in the range of 1-6” represents a group consisting of 1, 2, 3, 4, 5 and 6. For example, —(CH₂)₁₋₄— represents a group consisting of —CH₂—, —(CH₂)₂—, —(CH₂)₃—, and —(CH₂)₄—. For example,

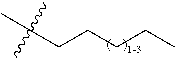

represents a group consisting of

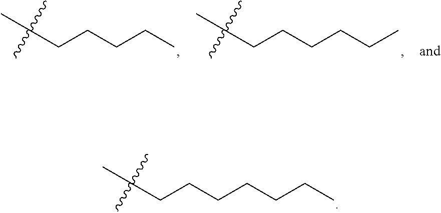

The numerical intervals in the present invention, including but not limited to the numerical intervals represented by integers, non-integers, percentages, and fractions, all include two endpoints unless otherwise specified.

In the present invention, with respect to the molecular weight of polymers, the terms “about” and “approximately” generally suggest a ±10% numerical range which can be amplified to ±15% in some cases but not exceeding ±20%, using the preset value as the base value. For example, the deviations of 11 kDa and 12 kDa relative to 10 kDa are 10% and 20%, respectively; therefore, “approximately 10 kDa” includes but is not limited to 11 kDa and 12 kDa. For example, provided that the molecular weight of a PEG component of a general formula is about 5 kDa, the corresponding molecular weight or number average molecular weight is allowed to be variable within the range of 5 kDa±10%, i.e., 4500-5500 Da.

In the present invention, with respect to percentages, the terms “about” and “approximately” generally suggest a numerical range of 0.5*N when the given value (without “%”) is accurate to the N (including 1, 0.1, 0.01, 0.001, etc.). For example, “approximately 1%” represents the range of 0.5%-1.5%, and “approximately 2.2%” represents the range of 2.15%-2.25%.

In the present invention, unless otherwise specified, the terms “multi-step” and “stepwise” have the same meaning with respect to preparation methods. Stepwise/multi-step reactions can be completed by a technical personnel in multiple practical operations, or in a single practical operation. For instance, in a reaction system of a practical operation, two reactive groups in one molecule of a low-molecular-weight lipid compound both undergo coupling reactions with a single-chain polyethylene glycol derivative reagent to obtain a non-linear PEGylated lipid compound with two single-chain PEG arms; considering that the two coupling reactions actually occur successively, the whole process can also be regarded as introducing polyethylene glycol components in a stepwise/multi-step way.

In the present invention, unless otherwise specified, a divalent linking group such as a hydrocarbylene group, an alkylene group, an arylene group, an amide group, etc., can be connected to another group using either one of its two connecting ends. For example, when an amide bond is used as a divalent linking group between GroupA and GroupB, both GroupA-C(═O)NH-GroupB and GroupB—NHC(═O)-GroupA are allowable.

With respect to the structural representation in the present invention, when it comes to where the atoms of connected groups belong, “ [symbol] ” is used to indicate the linking bond. For example,

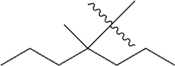 [SMILES: *C(C)(CCC)CCC]

represents the structure of a group; specifically,

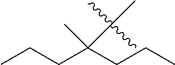 [SMILES: *C(C)(CCC)CCC]

represents —CCH₃(CH₂CH₂CH₃)₂ and

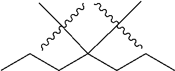 [SMILES: *C(*)(CCC)CCC]

represents —C(CH₂CH₂CH₃)₂—, while

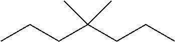

in the non-group form represents (CH₃)₂C(CH₂CH₂CH₃)₂.

In the present invention, with respect to a linking bond or group extending out from a cyclic structure, when the connecting end points to the inside of the ring instead of marking any specific ring atom, it means that the linking bond or group can be connected to any appropriate ring atom. Moreover, when the ring is part of a fused ring system, the linking bond or group can be connected to any ring atom of the fused ring system. For example,

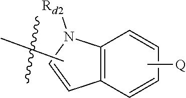

represents the group consisting of the structures with any rational chemical connections (including but not limited to

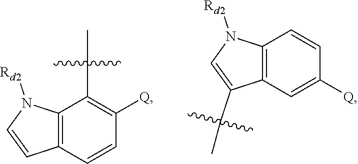

etc.).

In the present invention, the “atomic spacing” between two groups (e.g., GroupA, GroupB) refers to the minimum number of the atoms necessary for connecting the groups in the structure; e.g., the atomic spacing in GroupA-S—CH₂-GroupB is 2, and the atomic spacing in

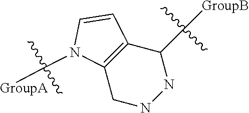

is 4.

In the present invention, the range of the number of carbon atoms of a group can be represented in the form of a subscript at the subscript position of “C”, and unless otherwise specified, the number of carbon atoms includes no contribution from substituents. For example, C₁₋₁₂ means “having 1 to 12 carbon atoms”. For example, a C₁₋₁₀ alkylene group represents any alkylene group having the number of carbon atoms in the range indicated by the subscript, i.e., any one selected from the group consisting of C₁, C₂, C₃, C₄, C₅, C₆, C₇, C₈, C₉, and C₁₀ alkylene groups, including but not limited to a linear C₁₋₁₀ alkylene group (e.g., —(CH₂)₆—) and a branched C₁₋₁₀ alkylene group (e.g., —(CH₂)₃—CH(CH₃)—(CH₂)₃—). For example, a “substituted C₁₋₁₂ alkyl group” is selected from the group consisting of C₁, C₂, C₃, C₄, C₅, C₆, C₇, C₈, C₉, C₁₀, C₁₁ and C₁₂ alkyl groups, each having at least one hydrogen atom replaced by a substituent, without particular restriction on the number of carbon atoms or heteroatoms in the substituent.

In the present invention, when a structure involved has isomers, unless otherwise specified, it can be any of the isomers. For example, with respect to a structure having both cis- and trans-isomers, it can be either a cis structure or a trans structure; with respect to a structure having E/Z isomers, it can be either an (E)-structure or a (Z)-structure; and, when optical activity is involved, a structure can be either levo or dextro.

In the present invention, if a structural representation is inconsistent with the name of the structure, then the structural representation should be prioritized.

In the present invention, the “molecular weight” represents the mass of a molecular compound; the “average molecular weight” represents the mass of a component of a compound represented by a general formula, with respect to macroscopic substances; and, unless otherwise specified, the “average molecular weight” generally refers to the “number average molecular weight” (Mₙ). As for the number average molecular weight, it can be used as not only the molecular weight of polydisperse blocks or substances but also the molecular weight of monodisperse blocks or substances. Unless otherwise specified, the unit of measurement for “molecular weight” and “average molecular weight” is dalton (Da). The molecular weight of polyethylene glycol chain can also be measured by the “degree of polymerization” which is, specifically, the number of repeat units (oxyethylene units, EO units). Accordingly, the average value and the number average value of the number of repeat units are preferably represented by “average degree of polymerization” and “number average degree of polymerization”, respectively.

In the present invention, the term “any appropriate” in “any appropriate linking group”, “any appropriate reactive group”, etc., indicates that the structures follow the basic rule of chemical structures and can facilitate the successful implementation of the preparation methods in the present invention. Chemical structures described in this manner can be regarded as having a clear and defined scope.

In the present invention, the “micro-modification” refers to a chemical modification process that can be realized by simple chemical reactions. Said simple chemical reactions mainly include protection, deprotection, salt complexation and decomplexation, ionization, protonation, deprotonation, changing leaving groups, etc.

In the present invention, the “micro-modified form” corresponds to the “micro-modification”, referring to a structural form that can be transformed into the target reactive group after simple chemical reactions such as protection, deprotection, salt complexation and decomplexation, ionization, protonation, deprotonation, changing leaving groups, etc. Said changing leaving groups includes but is not limited to the transformation of an ester form to an acyl chloride form.

In the present invention, the terms “stable” and “degradable” regarding a group are a pair of opposing concepts.

In the present invention, the term “degradable” means that a chemical bond in the present invention can be cleaved to obtain at least two independent residues. If a linking group with its structure altered after chemical changes still remains a complete linking group, then it is still within the scope of being “stable”. The conditions for degradation are not particularly limited, which can be physiological conditions in vivo, simulated physiological environments in vitro, or other conditions, preferably physiological conditions in vivo or simulated physiological environments in vitro. Said physiological conditions are not particularly limited, which can be intracellular or in the extracellular matrix, in normal physiological tissues or in pathologic tissues (e.g., tumor, inflamed tissue, etc.), and in body parts including but not limited to serum, heart, liver, spleen, lung, kidney, bone, muscle, fat, brain, lymph node, small intestine, gonad, etc. Said simulated physiological environment in vitro is not particularly limited, including but not limited to physiological saline, buffer, culture medium, etc. The degradation is not particularly limited with respect to the rate, which can be, for example, rapid degradation via enzymolysis or slow hydrolysis under physiological conditions, etc. Said physiological conditions in vivo include physiological conditions during treatment, such as ultraviolet radiation, thermal therapy, etc. The conditions for degradation include but are not limited to light, heat, low temperature, enzyme, oxidation-reduction, acidity, basicity, physiological condition, simulated physiological environment in vitro, etc., preferably light, heat, enzyme, oxidation-reduction, acidity, basicity, etc. The degradation can occur under stimulation by any above-mentioned condition. Said light condition includes but is not limited to visible light, ultraviolet light, infrared light, near-infrared light, mid-infrared light, etc. Said heat condition refers to a temperature higher than the normal physiological temperature, which is generally above 37° C. and below 45° C., preferably below 42° C. Said low temperature condition refers to a temperature below the physiological temperature of human body, preferably below 25° C., and more preferably <10° C., with specific examples such as refrigeration temperature, freezer temperature, temperature for liquid nitrogen treatment, 2˜10° C., 4˜8° C., 4° C., 0° C., −20±5° C., etc. Said enzyme is not particularly limited, and all enzymes that can be physiologically generated are included, e.g., peptidases, proteases, lyases, etc. Said oxidation-reduction is not particularly limited, e.g., oxidation-reduction transformation between a mercapto group and a disulfide bond, and hydrogenation-reduction transformation. Said acidic and basic conditions mainly refer to the pH conditions of internal body parts such as normal tissues, pathologic tissues, organs or tissues in treatment, etc.; for example, the stomach has an acidic condition, and tumor sites tend to be acidic as well. The degradation described herein can occur through metabolism in vivo (e.g., physiological effects, enzymes, oxidation-reduction, etc.), or under microenvironment stimulations (e.g., acidity and basicity) in specific in vivo sites, or under clinical therapeutic stimulations (e.g., light, heat, low temperature), etc. It should be noted that some conditions in organic chemistry that are extreme for living organisms, for example, strong acidity, strong basicity, high temperature (e.g., above 100° C.), etc., under which conditions a bond can be cleaved, are not considered to be within the scope of degradation conditions. For example, although an ether bond can be cleaved under strongly acidic conditions such as hydrobromic acid, it is always classified as a stable linking group in the present invention.

In the present invention, the term “stable” indicates that a linking group can keep existing as a complete linking group (i.e., a linking group which is stably and covalently connected to adjacent groups) and therefore it is defined as “stable”, allowing chemical changes without compromising the wholeness of the linking group. Said chemical changes are not particularly limited, including but not limited to isomerization, oxidation, reduction, ionization, protonation, deprotonation, substitution, etc. The conditions for stable existence are not particularly limited, including but not limited to light, heat, low temperature, enzyme, oxidation-reduction, neutrality, acidity, basicity, physiological condition, simulated physiological environment in vitro, etc., preferably light, heat, enzyme, oxidation-reduction, acidity, basicity, etc. The stable existence described herein indicates that, without particular stimulation (e.g., pH conditions in particular areas, and light, heat, low temperature in treatment, etc.), the stable connections can be maintained in the metabolic cycle in vivo and the molecular weight is not reduced by bond cleavage (as long as the structural integrity is maintained).

In the present invention, “stable” is not an absolute concept with respect to a specific linking group. For example, an amide bond is much more stable than an ester bond under acidic or basic conditions, and the “stable” linking group of the present invention includes the amide bond. However, the peptide bond, for example, is a kind of amide bond formed via the dehydration condensation of an α-carboxyl group of an amino acid molecule with an α-amino group of another amino acid molecule, which can be cleaved under specific enzymatic conditions and, therefore, is also classified as a “degradable” linking group. Similarly, carbamate group, thiocarbamate group, etc., are linking groups which can be stable or degradable. More commonly, carbamate group, thiocarbamate group, etc., tend to degrade slowly, while non-peptide amide bonds can remain stable in the metabolic cycle in vivo. As another example, a common ester bond can degrade under acidic or basic conditions, while an ester bond contained in a special structure can also degrade under UV exposure. As another example, even though some chemical bonds can degrade under specific enzymatic conditions, they can still be regarded as stable if their circulation in clinical use (e.g., site-directed administration) does not or basically not go through the specific enzymatic conditions.

In the present invention, in order to define more clearly the degradable properties of compound structures, a judgment criterion is provided for reference, which is, that the chemical bond examined within a limited time interval is kept to a specific percentage (e.g., 90%) as the boundary. Taking 90% as an example, the pharmacokinetic curve of the product of polyethylene glycol modification is generally used as a reference, and it is based on the percentage of the dose that meets the clinical evaluation criteria. For example, with respect to the intravenously administered PEGylated drug, when the blood concentration of the drug (in terms of active pharmaceutical ingredients, including PEGylated drugs and degraded non-PEGylated ingredients) is lower than 15% of the initial concentration (or other ratios that are more consistent with the clinical evaluation of the drug), the rest 85% is used as the base value for further evaluation; wherein, if the ratio of a linking group having unaltered chemical bonds exceeds 90%, then the linking group is considered a stable group in the present invention, otherwise it is considered a degradable group if the ratio is lower than 90%. The hydrolysis stabilization, enzymatic degradation, etc., reported in published literature are also included in the present invention. Taking hydrolysis stabilization as an example, it covers the hydrolysis rate of hydrolysis stabilization reported in published literature, preferably referring to a hydrolysis rate lower than 1-2% (usually 2%) per day under physiological conditions, in mass or molar mass. The hydrolysis rate of typical chemical bonds can be found in most standard chemical handbooks.

In the present invention, with respect to a PEGylated drug circulating in the blood, the ether bonds among the repeat units of polyethylene glycol components (—CH₂CH₂—O—CH₂CH₂—) are usually considered stable, which can provide a reference for distinguishing between “stable” and “degradable”; however, it does not serve as a critical criterion for judging. When a linking group in its environment is more stable than the ether bonds of PEG moieties, it is considered stable.

In the present invention, all compounds of the general formula (1) should be regarded as including salts thereof. The term “salt” is selected from the group consisting of acid addition salts formed by the compounds and inorganic and/or organic acids, base addition salts formed by the compounds and inorganic and/or organic bases, and combinations of any two or more thereof. When a compound of the general formula (1) contains both a basic moiety (such as, but not limited to, a pyridine or imidazole) and an acidic moiety (such as, but not limited to, a carboxylic acid), a zwitterion (“inner salt”) can be formed and included in the term “salt”. The “salt” can be pharmaceutically acceptable (i.e., non-toxic, physiologically acceptable) or otherwise. Salts of compounds of the general formula (1) can be formed by reacting the compounds with an amount (e.g., an equivalent) of acid or base in a medium such as one in which the salt precipitates or in an aqueous medium followed by lyophilization. Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecyl sulfates, ethanesulfonates, fumarates, glucoheptanoates, glycerophosphates, hemi sulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates, sulfonates, tartrates, thiocyanates, toluenesulfonates, undecanoates, etc.

Exemplary base addition salts include ammonium salts, alkali metal salts (e.g., sodium, lithium, and potassium salts), alkaline earth metal salts (e.g., calcium and magnesium salts), salts with organic bases (e.g., organic amines), and salts with amino acids (e.g., arginine, lysine). Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, tetradecyl, and stearyl chlorides, bromides, and iodides), aralkyl halides (e.g., benzyl and phenethyl bromides), etc. Said acid addition salts and base addition salts are both preferably pharmaceutically acceptable salts, and should be regarded as equivalent to the free forms of corresponding compounds of the general formula (1) for the purpose of this disclosure.

The heteroatoms in the present invention are not particularly limited, including but not limited to O, S, N, P, Si, F, Cl, Br, I, B, etc.

In the present invention, unless otherwise specified, the term “group” refers to a radical formed by a compound losing one or more atoms, and the radical contains at least 1 atom. Relative to a compound, a group formed by the compound losing part of its atoms or groups is also termed a residue. A group in the present invention does not include the hydrogen atom. A group is not particularly limited with respect to the valence, and examples include a monovalent group, a divalent group, a trivalent group, a tetravalent group, . . . , a hectovalent group, etc. Wherein, groups having a valence equal to or greater than two are collectively defined as “linking groups”. A linking group can also contain only one atom, such as the ether group (—O—) and the thioether group (—S—). Particularly, when a certain group is defined as including a linking bond, it means that the group can be non-existent and only act as a linker.

In the present invention, with respect to the valence of a group, “multivalent” means the valence is at least 3.

In the present invention, unless otherwise specified, a “linking bond” only acts as a linker, containing no atoms.

In the present invention, the term “group” can be replaced by “bond” for divalent groups, without changing its meaning. For example, a divalent ether group can be termed an ether bond, a divalent ester group can be termed an ester bond, and a divalent carbamate group can be termed a carbamate bond.

In the present invention, the term “substitution” means at least one hydrogen atom is replaced by a substituent, or a group is replaced by another group. In the present invention, the group used for substitution is termed a “substituent”. The number of atoms contained in a substituent can be 1 or greater than 1. When a substituent contains only 1 atom, it can also be termed a “substituent atom”.

In the present invention, when a group is substituted by another group, it can be that one or multiple atoms in a group are replaced by another group, wherein the other group is the substituent, or that a whole group is replaced by another group. The specific situation depends on the conventional understanding of those skilled in the art. For example, given that “a phenyl group is substituted by a chlorine atom”, combined with different additional conditions, it can be understood that the phenyl group is replaced by a chlorine atom, or that the phenyl group contains a chloride substituent. For example, given that “a hydroxyl group is substituted by -OTs”, it is generally understood that the hydroxyl group is replaced by the -OTs group in its entirety.

In the present invention, a “substituent” is a non-hydrogen atom or a group containing non-hydrogen atoms, and the non-hydrogen atom includes but is not limited to halogen atoms such as F, Cl, Br and I. A group containing non-hydrogen atoms includes but is not limited to, e.g., oxo group (═O), hydroxyl group (—OH), alkoxy group (—OR₁, wherein Rₜ is a hydrocarbon group), carboxyl group (—COOH), amine group (—NRR, wherein two R are each independently H or a hydrocarbon group), cyanide group (—CN), azido group (—N₃), C₁₋₂₀ alkyl group, and cycloalkyl group.

In the present invention, when a compound or group is “substituted”, it means that the compound or the group (including but not limited to, e.g., alkyl group, alkylene group, aliphatic hydrocarbon group, residue of aliphatic hydrocarbon derivative, residue of amino acid, or residue of monosaccharide) contains one or more substituents.

In the present invention, a compound, a group, or an atom can be simultaneously substituted and hybridized, e.g., —CH₂—CH₂—CH₂— being replaced by —CH₂—S—CH(CH₃)—.

In the present invention, the “carbon-chain linking group” refers to a linking group having carbon atoms as all of its main-chain atoms while the side chain moieties are allowed to have hydrogen atoms at main-chain carbon atoms thereof replaced by heteroatoms or heteroatom-containing groups. When a “main-chain atom” is a heteroatom, it is also termed a “main-chain heteroatom”, e.g., —S—CH₂—, —O—CH₂—, and

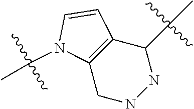 [SMILES: *C1NNCc2c1ccn2*]

are considered to have main-chain heteroatoms. A “carbon-chain linking group” can be classified into a hydrocarbon group or a hydrocarbon group having a heteroatom-containing side group; the hydrocarbon group having a heteroatom-containing side group includes but is not limited to an oxo (═O) group, a thio (═S) group, an imino group (connected to the main-chain carbon atom through a carbon-nitrogen double bond), an oxyalkyl group having an ether bond, a thioalkyl group having a thioether bond, an azaalkyl group having a tertiary amino group, etc. The main chain of a “carbon-chain linking group” comprises only saturated or unsaturated carbon atoms, including but not limited to the —C—C—C—C—, —C═C—C—C, and —C—C—C—C— forms; the side group of a carbon chain is allowed to contain heteroatoms, and the side group can be a single atom (e.g., —I, ═O), a chain (e.g., —CH₂OCH₃), or one that forms a ring with the main-chain atoms (e.g.,

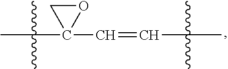 [SMILES: *C=CC1(*)CO1]

wherein the main chain is —C—C═C— and the side group is —CH₂O—).

In the present invention, unless otherwise specified, the terms “amino group” and “amine group” have the same meanings, including monovalent, divalent, trivalent, and quadrivalent neutral or cationic groups, substituted or unsubstituted. For example, the —NH₂ in CH₃—NH₂ can be termed an “amino group” or a “primary amine group”. As another example, the —NH— in CH₃—NH—CH₃ can be termed a “secondary amine group” or a “secondary amino group”, wherein the —NH—CH₃ can also be interpreted as a methyl-substituted amine group.

In the present invention, the term “amine group” includes but is not limited to primary amine groups, secondary amine groups, tertiary amine groups, and quaternary ammonium ions. Exemplary amine groups include —NRₜRₜ and —N⁺RₜRₜR₁, wherein each Rₜ is independently a hydrogen atom or any hydrocarbon group including but not limited to an alkyl group, an alkylene group, a cycloalkyl group, an alkenyl group, an alkynyl group, a phenyl group, etc.

In the present invention, the secondary amine bond and the hydrazine bond are “—NH—” and “—NH—NH—”, respectively, both capped by hydrocarbon groups or hydrocarbylene groups at both ends, e.g., —CH₂—NH—CH₂— and —CH₂—NH—NH—CH₂—. As a counterexample, —C(═O)—NH— is termed an amide bond, instead of being considered to contain a secondary amine bond.

In the present invention, the “acyl group” includes carbonyl groups and non-carbonyl acyl groups, including but not limited to, e.g., a carbonyl group, a sulfonyl group, a sulfinyl group, a phosphoryl group, a phosphiryl group, a phosphinyl group, a nitroxyl group, a nitrosyl group, a thiocarbonyl group, an imidoyl group, a thiophosphoryl group, a dithiophosphoryl group, a trithiophosphoryl group, a thiophosphiryl group, a dithiophosphiryl group, a thiophosphinyl group, a thiophosphono group, a dithiophosphono group, a thiophosphino group, etc. and preferably a carbonyl group, a thiocarbonyl group, a sulfonyl group or a sulfinyl group. Unless otherwise specified, an acyl group particularly refers to a carbonyl group.

In the present invention, the “electron-changing group” refers to a group that can change the electron density of an unsaturated structure (especially an aromatic ring structure), relative to the hydrogen atom. Taking the aromatic ring as an example, considering altogether the induction effect, conjugation effect and hyperconjugation effect of the electron-changing group on the aromatic ring, the “electron donating group” is one that increases the electron cloud density of the aromatic ring, and the “electron withdrawing group” is one that decreases the electron cloud density of the aromatic ring. Exemplary electron donating groups include lower alkoxy, lower alkylamino, di-lower alkylamino, lower alkyl, aryloxy, aralkoxy, aminoaryl, hydroxyl, amino, thiol, alkylthio groups, etc., wherein the term “lower” should be interpreted as containing 1-6 carbon atoms. Exemplary electron withdrawing groups include halo (bromo, chloro, fluoro, iodo, etc.), nitro, trihalomethyl, cyano, carboxyl, formyl, keto, azo, amide carbonyl, amide sulfonyl, formamido, sulfoxy, sulfonamide, ureido groups, etc. Moreover, a benzene ring, a double bond, etc, can be regarded as either an electron donating group or an electron withdrawing group, which depends on the combined effect of the influence on the electron cloud density.

In the present invention, unless otherwise specified, the “capping group” and the “terminal group” have the same meaning, both can be a reactive group or a non-reactive group, a targeting group or a non-targeting group. In the present invention, specific definitions of capping groups/terminal groups of polyethylene glycol chains are not particularly limited; for example, the capping group/terminal group of

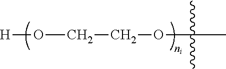

can be a hydrogen atom, a hydroxyl group, or a hydroxyethyl group; for example, the capping group/terminal group of

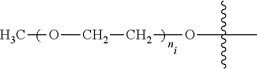

can be a methyl group, a methoxy group, or a methoxyethyl group.

In the present invention, a “group with functionality” is also termed a “functional group”, which is preferably a reactive group, a protected reactive group, or a precursor of a reactive group, etc. The term “poly” indicates that the number of functional groups is at least 3; for example, a polyol is a compound having at least 3 hydroxyl groups, a polythiol is a compound having at least 3 thiol groups, etc. What should be noted is that heterofunctional groups of other types are also allowed. For example, tris(hydroxymethyl)aminomethane is a triol containing also an amino group, and citric acid is a tricarboxylic acid containing also a hydroxyl group.

In the present invention, the “heterofunctional group” refers to a functional group which is different from a certain type of functional group; for example, with respect to a triol, the amino group contained within is a heterofunctional group relative to the hydroxyl group. The “heterofunctional group pair” is functional groups hetero to each other. The terms “heterofunctional group” and “heterofunctional group pair” are used not only for describing different functional groups existing in the same molecule, but also for describing different functional groups existing in different molecules or structures; for example, an alkynyl-containing compound reacts with an azido-containing compound, wherein the alkynyl and the azido are heterofunctional groups to each other, i.e., a “heterofunctional group pair”.

In the present invention, the “functional group source” is characterized by reactivity or potential reactivity, photosensitivity or potential photosensitivity, targeting properties or potential targeting properties. Said “potential” means the functional group source can be transformed into a reactive group after a chemical process including but not limited to functional modification (e.g., grafting, substitution, etc.), deprotection, salt complexation and decomplexation, ionization, protonation, deprotonation, changing leaving groups, etc., or can generate luminescence or targeting properties under the external stimulation of light, heat, enzymes, specific binding molecules, micro-environment in vivo, etc. Said luminescence is not particularly limited, including but not limited to visible light, fluorescence, phosphorescence, etc.

In the present invention, the “modified form of reactive group” is a precursor to a reactive group, or an active form (still a reactive group) after at least one process among oxidation, reduction, hydration, dehydration, electronic rearrangement, structural rearrangement, salt complexation and decomplexation, ionization, protonation, deprotonation, diazotization, substitution, deprotection, and change of leaving groups, or an inactive form after the reactive group is protected.

In the present invention, with respect to the preparation methods, unless otherwise specified, a reactive group also includes protected forms thereof, and the protected forms can be deprotected in any appropriate step during the practical preparation process to obtain the corresponding active forms.

In the present invention, the “hydrocarbon” refers to a hydrocarbon compound composed of carbon atoms and hydrogen atoms.

In the present invention, a hydrocarbon can be classified into an aliphatic hydrocarbon or an arene, depending on whether an aromatic ring is contained. The “aromatic hydrocarbon” is equivalent to “arene”. A hydrocarbon containing no benzene ring or other aromatic ring is defined as an aliphatic hydrocarbon. A hydrocarbon containing at least one benzene ring or other aromatic ring is defined as an arene. An arene can contain an aliphatic hydrocarbon structure, e.g., toluene, diphenylmethane, 2,3-dihydroindene, etc. When an aliphatic hydrocarbon is particularly described as “substituted”, the substituent can be an aromatic hydrocarbon group. When an aliphatic hydrocarbon contains a side group, the side group can contain an aromatic ring. For example,

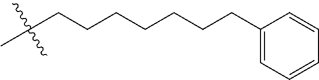 [SMILES: *CCCCCCCc1ccccc1]

can be an aromatic hydrocarbon group, or an aliphatic hydrocarbon group substituted by a benzene ring (specifically the 1-heptyl substituted by a benzene ring herein). For example,

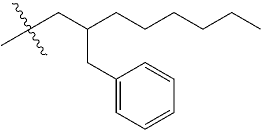 [SMILES: *CC(CCCCCC)Cc1ccccc1]

can be an aromatic hydrocarbon group, or an aliphatic hydrocarbon group substituted by a benzene ring (specifically the 2-methyl-1-octyl substituted by a benzene ring herein).

In the present invention, according to the degree of saturation, a hydrocarbon can be classified into a saturated hydrocarbon or an unsaturated hydrocarbon. Wherein, the unsaturated hydrocarbon includes hydrocarbons containing a carbon-carbon double bond, a carbon-carbon triple bond, or an aromatic ring. The unsaturated hydrocarbon is not particularly limited with respect to the degree of unsaturation, e.g., including but not limited to alkenes (containing at least one double bond), alkynes (containing at least one triple bond), conjugated dienes (containing at least one pair of conjugated double bonds), polyenes (containing two or more double bonds), etc.

In the present invention, the “hydrocarbon group” refers to a residue formed after a hydrocarbon loses at least one hydrogen atom. Depending on the number of hydrogen atoms lost, a hydrocarbon group can be classified into a monovalent hydrocarbon group (with one hydrogen atom lost), a divalent hydrocarbon group (with two hydrogen atoms lost), a trivalent hydrocarbon group (with three hydrogen atoms lost), etc.; by analogy, when n hydrogen atoms are lost, the valence state of the formed hydrocarbon group is n. Unless otherwise specified, the hydrocarbon group in the present invention particularly refers to the monovalent hydrocarbon group. Unless otherwise specified, a hydrocarbon group is substituted or unsubstituted.

In the present invention, the “heterohydrocarbon” refers to a compound formed after any carbon atom, or the carbon atom together with its adjacent hydrogen atoms, in a hydrocarbon is replaced by a heteroatom. For example, a dimethyl ether (CH₃OCH₃) is formed after a propane (CH₃CH₂CH₃) having one of its carbon atom together with the two hydrogen atoms connected are replaced by an oxygen atom. The “heterohydrocarbon group” refers to a residue formed after a heterohydrocarbon loses at least one hydrogen atom.

In the present invention, the “hydrocarbylene group” refers to a divalent hydrocarbon group.

In the present invention, the “aliphatic hydrocarbon group” refers to a residue formed after an aliphatic hydrocarbon loses at least one hydrogen atom. Unless otherwise specified, the aliphatic hydrocarbon group in the present invention particularly refers to the monovalent aliphatic hydrocarbon group. The aliphatic hydrocarbon group includes saturated aliphatic hydrocarbon groups and unsaturated aliphatic hydrocarbon groups. An aliphatic hydrocarbon group can be an alkyl hydrocarbon group, an alkenyl hydrocarbon group, or an alkynyl hydrocarbon group.

In the present invention, a hydrocarbon is not particularly limited with respect to the structure, which can be in the form of a linear structure, a branched structure, a ring-containing structure, a dendritic structure, a comb structure, or a hyperbranched structure, etc. Unless otherwise specified, the linear structure, branched structure, and ring-containing structure correspond to linear hydrocarbon, branched hydrocarbon, and cyclic hydrocarbon, respectively.

Wherein, hydrocarbons containing no ring structure are collectively termed open-chain hydrocarbons.

In the present invention, the “dendrimer-like structure” refers to an incomplete dendritic structure missing individual repeat units, and the number of missed repeat units should be smaller than 50% of the number for the last level of saturation.

The source of the hydrocarbon group in the present invention is not particularly limited, e.g., from aliphatic or aromatic hydrocarbons, saturated or unsaturated hydrocarbons, straight chain hydrocarbons, branched chain hydrocarbons or cyclic hydrocarbons, hydrocarbons or heterohydrocarbons, etc.; with respect to saturation, e.g., from alkanes, alkenes, alkynes, conjugated dienes, polyenes, etc.; with respect to cyclic hydrocarbons, e.g., from alicyclic hydrocarbons or aromatic hydrocarbons, monocyclic hydrocarbons or polycyclic hydrocarbons; with respect to heterocyclic hydrocarbons, e.g., from alicyclic hydrocarbons or aromatic heterocyclic hydrocarbons.

In the present invention, the “alkane” refers to a saturated aliphatic hydrocarbon, and the “alkyl group” refers to a hydrocarbon group formed after an alkane loses any hydrogen atom.

In the present invention, the “alkene” refers to an aliphatic hydrocarbon containing one or more carbon-carbon double bonds. As is understood by those skilled in the art, unless otherwise specified, an alkenyl group can have a cis configuration or a trans configuration, an E configuration or a Z configuration. The “alkenyl group” refers to a hydrocarbon group formed after an alkene loses any hydrogen atom. For example, the “C₂₋₈ alkenyl group” refers to a hydrocarbon group formed after an alkene loses any hydrogen atom, wherein the alkene is substituted or unsubstituted, linear, branched or cyclic, and contains 2-8 carbon atoms and at least one carbon-carbon double bond.

In the present invention, the “alkene” is an aliphatic hydrocarbon containing one or more carbon-carbon triple bonds. The “alkynyl group” refers to a hydrocarbon group formed after an alkyne loses any hydrogen atom.

In the present invention, the “alkylene”, i.e., the divalent alkyl group, includes open-chain alkylene groups and divalent cycloalkyl groups. An open-chain alkylene group refers to a divalent alkyl group without a ring structure, and a divalent cycloalkyl group refers to a divalent alkyl group having a ring structure.

In the present invention, ring atoms are the atoms that jointly constitute the ring skeleton.

In the present invention, the “unsaturated hydrocarbon group” refers to a hydrocarbon group formed after an unsaturated hydrocarbon loses a hydrogen atom. The hydrocarbon group formed after an unsaturated hydrocarbon loses a hydrogen atom can be classified into an alkenyl group, an alkynyl group, etc. Unless otherwise specified, an alkenyl or alkynyl group can be formed after an unsaturated hydrocarbon loses a hydrogen atom at an unsaturated or saturated carbon site. Particularly, an unsaturated hydrocarbon loses a hydrogen atom at an unsaturated carbon site leads to the formation of an alkenyl or alkynyl group (e.g., propenyl, propynyl), while losing a hydrogen atom at a saturated carbon site leads to the formation of an alkenyl or alkynyl hydrocarbon group (e.g., allyl, propargyl). The carbon site at which a hydrogen atom is lost can also be indicated by numbering, e.g.,

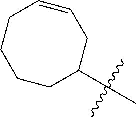 [SMILES: *C1C/C=C\CCCC1]

is 4-cyclooctenyl,

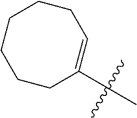 [SMILES: */C1=C/CCCCCC1]

is 1-cyclooctenyl.

In the present invention, the source of an amino acid is not particularly limited unless otherwise specified, which can be a natural source, a non-natural source, or a mix of both. The structural type of an amino acid in the present invention is not particularly limited unless otherwise specified, which can be L-type, D-type, or a mix of both.

In the present invention, the definitions and illustrations of the skeletons of amino acids, the skeletons of amino acid derivatives, and the skeletons of cyclic monosaccharides in documents including CN104877127A, WO/2016/206540A, CN201610252378.X (CN106967213A), CN201710125672.9, CN201710126727.8 and all cited documents, are hereby incorporated by reference. Unless otherwise specified, the skeletons are also residues. Wherein, the residue of amino acid specifically refers to the residue formed by losing a carboxylic hydroxyl group (including every carboxylic hydroxyl group at the C-terminus as well as the carboxylic hydroxyl group in the pendant group of aspartic acid or glutamic acid), a hydrogen atom in a hydroxyl group, a hydrogen atom in a phenolic hydroxyl group (e.g., tyrosine), a hydrogen atom in a thiol group (e.g., cysteine), a hydrogen atom at a nitrogen atom (including every hydrogen atom at the N-terminus as well as the hydrogen atom in an amino group of a pendant group such as the hydrogen atom in the F-amino group of lysine or ornithine and the hydrogen atom in the amino group of the pendant ring of histidine or tryptophan), an amino group in an amide (e.g., asparagine, glutamine), an amino group in a pendant group of a guanidine, or a hydrogen atom in an amino group. Besides having the essential characteristics of an amino acid, a residue of amino acid derivative also has atomic or group moieties with the essential characteristics of a non-amino acid.

In the present invention, the term “bio-related substance” includes but is not limited to the substances described, listed, or referred to in documents including CN104877127A, WO/2016/206540A, CN201610252378.X (CN106967213A), CN201710125672.9, CN201710126727.8 and all cited documents. In general, bio-related substances include but are not limited to the following substances: drugs, proteins, polypeptides, oligopeptides, protein mimics, fragments and analogs, enzymes, antigens, antibodies and their fragments, receptors, small molecule drugs, nucleosides, nucleotides, oligonucleotides, antisense oligonucleotides, polynucleotides, nucleic acids, aptamers, polysaccharides, proteoglycans, glycoproteins, steroids, steroid compounds, lipid compounds, hormones, vitamins, phospholipids, glycolipids, dyes, fluorescent substances, targeting factors, targeting molecules, cytokines, neurotransmitters, extracellular matrix substances, plant or animal extracts, viruses, vaccines, cells, vesicles, liposomes, micelles, etc. Said bio-related substances refer to not only themselves but also their precursors, activated states, derivatives, isomers, mutants, analogs, mimics, polymorphs, pharmaceutically acceptable salts, fusion proteins, chemically modified substances, genetically recombined substances, and also the corresponding agonists, activating agents, activators, inhibitors, antagonists, modulators, receptors, ligands or ligand groups, antibodies and fragments thereof, acting enzymes (e.g., kinases, hydrolases, lyases, oxoreductases, isomerases, transferases, deaminases, deiminases, invertases, synthetases, etc.), enzyme substrates (e.g., coagulation cascade protease substrates, etc.), etc. Said derivatives include but are not limited to the derivatives of glycosides, nucleosides, amino acids, and polypeptides. Chemically modified products with newly formed reactive groups, i.e., modified products obtained by changing the types of reactive groups via modification or by introducing extra structures such as functional groups, reactive groups, amino acids or derivatives thereof, polypeptides, etc., are all within the scope of chemically modified substances of bio-related substances. Before or after bio-related substances combine with functionalized compounds, it is allowed that there are target molecules to be combined with, appendages, or delivery vectors to form modified bio-related substances or complex bio-related substances. Wherein, the pharmaceutically acceptable salts can be inorganic salts such as hydrochlorides, sulfates, and phosphates, or organic salts such as oxalates, malates, citrates, etc. Wherein, the “drug” in the present invention includes any agents, compounds, compositions, and mixtures with physiological or pharmacological effects in vivo or in vitro, and the effects are usually beneficial. Said drug is not particularly limited with respect to the type, including but not limited to pharmaceuticals, vaccines, antibodies, vitamins, food, food additives, nutritional supplements, nutraceuticals, and other agents providing beneficial effects. Said “drug” is not particularly limited with respect to the range of physiological or pharmacological effects in vivo, which can be effective for the whole body or only at local sites. Said “drug” is not particularly limited with respect to the activity, mainly an active substance capable of interacting with other substances or an inert substance which is non-interactive; however, the inert substance can be transformed into an active form by in vivo effects or certain stimulations. Wherein, “small molecule drugs” are bio-related substances with molecular weight not exceeding 1000 Da, or small molecule mimics or active fragments of any bio-related substance.

In the present invention, the “residue of amino acid” includes the amino acid with a hydrogen atom removed from its amino group and/or a hydroxyl group removed from its carboxyl group and/or a hydrogen atom removed from its thiol group and/or its amino group being protected and/or its carboxyl group being protected and/or its thiol group being protected. Imprecisely, a residue of amino acid can also be termed an amino acid.

In the present invention, unless otherwise specified, the term “monosaccharide group” refers to the residue of monosaccharide, i.e., the skeleton of monosaccharide, including open-chain monosaccharide groups and cyclic monosaccharide groups (e.g., furanose ring and pyranose ring).

Monosaccharide groups in the present invention can be selected from the residues of compounds including but not limited to monosaccharides, sugar alcohols, deoxy sugars, amino sugars, amino sugar derivatives (e.g., amide derivatives), sugar acids, and glycosides, having open-chain or cyclic structures. Examples include the amino residue formed by removing an amino hydrogen atom of an amino sugar, the acyl group formed by removing a carboxylic hydroxyl group of a sugar acid, etc. Said monosaccharides include but are not limited to aldoses (polyhydroxy aldehydes) and ketoses (polyhydroxy ketones). Examples also include alkyl ether derivatives, specifically, e.g., methyl ether derivatives, and more specifically, e.g., quebrachitol. Monosaccharide groups in the present invention are not particularly limited with respect to the number of carbon atoms, including but not limited to tetroses, pentoses, hexoses, and heptoses, preferably pentoses and hexoses. Wherein, exemplary tetroses, pentoses, hexoses, heptoses, sugar alcohols, deoxy sugars, amino sugars, amide derivatives of amino sugars, sugar acids, glycosides, etc., include but are not limited to the structures disclosed in CN106967213A.

In the present invention, the “arginine-glycine-aspartic acid peptide mimic”, i.e., RGD peptide mimic, includes but is not limited to the structures disclosed in CN1606566A.

In the present invention, the term “targeted delivery” or the verb form “target” refers to the process of facilitating the delivery of an agent to a specific organ, tissue, cell, and/or intracellular compartment (referred to as a target site), to make the target site receive more cargo than any other organ, tissue, cell or intracellular compartment (referred to as the non-target site). A targeted delivery can be detected by methods known in the art, e.g., by comparing the concentration of the agent delivered in the target cell population to the concentration of the agent delivered in the non-target cell population after systemic administration. In some embodiments, the targeted delivery results in at least a 2-fold higher concentration at the targeted location compared to the non-targeted location.

In the present invention, the “targeting group” refers to a group capable of interacting with a complementary binding moiety at a desired location and/or under desired conditions. For example, complementary binding moieties can be ligands and anti-ligands (e.g., streptavidin and biotin, protein A or G and the Fe region of immunoglobulin), ligands and receptors (e.g., small molecule ligands and their receptors, or sugar-lectin interactions), phage display-derived peptides, complementary nucleic acids (e.g., DNA hybrids, RNA hybrids, DNA/RNA hybrids, etc.), and aptamers. Other exemplary complementary binding moieties include, but are not limited to, moieties exhibiting complementary charges, hydrophobicity, hydrogen bonding, covalent bonding, van der Waals forces, reactive chemistry, electrostatic interactions, magnetic interactions, etc.

In a specific embodiment of the present invention, included is the use of liposomes or LNPs for therapeutic purposes, especially the use of liposomes or LNPs with specific targeting effects. The specific binding can be realized by methods known in the art, including but not limited to, size selection of liposomes or LNPs and targeted modification on the surface of liposomes or LNPs, after which the liposomes or LNPs bind to cells or target receptors in a biological tissue.

The targeted modification in the present invention includes but is not limited to binding/attaching targeting ligands to the surface of liposomes or LNPs through various existing technical means.

In the present invention, the “targeting ligand” or “targeting agent” specific for a particular receptor (receptor agent or ligand) refers to any compound that is a specific binding partner of a specific binding pair where the other binding partner is a receptor. The receptor can be associated with the cell membrane or surface or exist in a soluble form, and can be present intracellularly and/or extracellularly in a subject (preferably a mammalian subject, such as a human or an animal). Examples of receptors include, but are not limited to, membrane receptors, soluble receptors, cloned or recombinant receptors, clan CD cysteine proteases and other proteases and enzymes, hormone receptors, drug receptors, transmitter receptors, autocrine receptors, cytokine receptors, antibodies, antibody fragments, engineered antibodies, antibody mimics, molecular recognition units, adhesion molecules, lectins, integrins, and selectins. Typically, the binding affinity between a receptor and its ligand can be at least 10⁻⁵ M, preferably 10⁻⁷ M and higher, for example, from about 10⁻⁸ M to about 10⁻¹² M. Specifically, the receptor agent or ligand includes, but is not limited to, peptides or polypeptides (including derivatives such as aza-peptide derivatives, derivatives partially or exclusively containing D-amino acids, glycopeptides, etc.), proteins (including glycoproteins or phosphoproteins), carbohydrates, glycolipids, phospholipids, oligonucleotides, polynucleotides, aptamers, Spiegelmers, vitamins (e.g., vitamin B9 (folic acid), vitamin B12), antigens and fragments thereof, haptens, receptor agonists, partial agonists, mixed agonists, antagonists, drugs, chemokines, hormones (e.g., LH, FSH, TRH, TSH, ACTH, CRH, PRH, MRH, MSH, glucagon, prolactin, transferrin: lactoferrin, angiotensin; histamine; insulin; lectin) transmitter, endocrines, growth factors (e.g., PDGF, VEGF, EGF, TGFα, TBFβ, GM-CSF, G-CSF, M-CSF, FGF, IGF, bombesin, thrombopoietin, erythropoietin, oncostatin, and endothelial 1), cytokines including interleukins (e.g. interleukins 1 to 15), lymphokines and cell signaling molecules, e.g., tumor necrosis factors (e.g. tumor necrosis factor α and β) and interferons (e.g. interferon α, β and γ), cofactors, coenzymes, cofactors, regulatory factors, or any other naturally occurring or synthetic organic molecules that can specifically bind receptors, including fragments, analogs, and derivatives thereof that retain the same binding properties.

In the present invention, targeting ligands can be used to direct (or target) liposomes or LNPs to specific types of cells such as cancer cells, cells of specific tissues, or cells of specific organs, e.g., liver cells (see, e.g., U.S. Pat. No. 6,316,024 and US6, 214, 388; Allen et al., Biochim. Biophys. Acta, 1995, 1237, 99-108; Blume et al., Biochim. Biophys. Acta, 1993, 1149, 180-184). This can be accomplished by utilizing receptors that are overexpressed in specific cell types, wherein the receptors include, for example, the folate receptor (overexpressed in various tumor tissues including breast, ovarian, cervical, colorectal, renal, and nasopharyngeal tumors), epidermal growth factor receptor (EGFR, overexpressed in anaplastic thyroid carcinoma and breast and lung tumors), metastin receptor (overexpressed in papillary thyroid carcinoma), ErbB family receptor tyrosine kinase (overexpressed in important breast cancer subtypes), human epidermal growth factor receptor-2 (Her2/neu) (overexpressed in breast cancer), tyrosine kinase-18-receptor (c-Kit) (overexpressed in sarcomatoid renal carcinoma), HGF receptor c-Met (overexpressed in esophageal adenocarcinoma), CXCR4 and CCR7 (overexpressed in breast cancer), endothelin-A receptor (overexpressed in prostate cancer), peroxisome proliferator-activated receptor 6 (PPAR-6) (overexpressed in most colorectal cancer tumors), PDGFR A (overexpressed in ovarian cancer), BAG-1 (overexpressed in various lung cancers), soluble TGFβ receptor type II (overexpressed in pancreatic cancer), asialoglycoprotein receptor (overexpressed in hepatocytes), αvβ3 integrin receptor (overexpressed in growing tumor vasculature), etc.

The choice of receptor agent or ligand for use in the present invention will be determined on the basis of the characteristics of the disease, condition, or infection to be detected and/or treated. Preferred receptor agents or ligands include vitamins (e.g., folic acid or fragments thereof), pteroic acid derivatives, peptides (including derivatives such as aza-peptide derivatives), proteins, and carbohydrates.

In the present invention, any reagent that selectively binds to a specific receptor cell or tissue to be treated or tested can be attached to liposomes or LNPs and serve as a targeting ligand or receptor ligand. Preferably, the targeting ligand is attached to the surface of liposomes or LNPs through a long-chain linker (e.g., a polymer, preferably a polyethylene glycol component). For example, folate-type conjugates have been used to provide methods for the targeted delivery of therapeutic compounds for the treatment and/or diagnosis of disease, allowing a reduction in the required dose of the therapeutic compound (see, e.g., WO02/094185, U.S. Pat. No. 6,335,434, WO99/66063, U.S. Pat. No. 5,416,016). As another example, the use of galactose- and galactosamine-type conjugates to transport exogenous compounds across cell membranes can provide a method for the targeted delivery in the treatment of liver diseases (e.g., HBV and HCV infections) or hepatocellular carcinoma, while allowing a reduction in the required dose of the therapeutic compound needed in the treatment (see, e.g., U.S. Pat. No. 6,030,954).

In the present invention, the “antigen display system” refers to a naturally occurring or synthetic system that can present at least one antigen (or a fragment thereof) and enable at least one antigen (or a fragment thereof) to be recognized or bound by immune effector molecules (e.g., T-cell antigen receptor on the surface of T cells), or present at least one antigen (or a fragment thereof) in the form of an antigen-MHC complex that can be recognized by specific effector cells of the immune system and thereby induces an effective cellular immune response toward the presented antigens.

Compounds, compositions, formulations, and methods of the present invention can be used to deliver bioactive substances to one or more of the following physiological sites of a patient: liver or liver cells (such as hepatocytes), kidney or kidney cells, tumor or tumor cells, CNS or CNS cells (central nervous system, such as brain and/or spinal cord), PNS or PNS cells (peripheral nervous system), lung or lung cells, blood vessels or blood vessel cells, skin or skin cells (such as dermal cells and/or follicular cells), eye or eye cells (such as macula, fovea, cornea, retina), ear or ear cells (such as inner ear, middle ear and/or outer ear cells).

In the present invention, the targeted sites are not particularly limited, including but not limited to brain, lung, kidney, stomach, liver, pancreas, breast, prostate, thyroid, uterus, ovary, nasopharynx, esophagus, rectum, colon, small intestine, gallbladder, bladder, bone, sweat gland, skin, blood vessel, lymph, joint, soft tissue, etc. The targeted tissue is not particularly limited with respect to the characteristics, including but not limited to tumor tissue, inflammatory tissue, diseased tissue, etc.

In some specific embodiments of the present invention, the reaction process also involves “protection” and “deprotection” of relevant groups. To avoid the influence of a certain reactive group on the reaction, the reactive group is usually protected. In some specific embodiments of the present invention, when 2 or more reactive groups are present, the target reactive group should be selectively used for the reaction, and therefore the other reactive groups should be protected. The protecting groups not only remain stable during the target reaction process but can also, as needed, be removed by conventional technical means in the art.

In the present invention, the “protection” of a reactive group refers to a strategy for reversibly transforming the reactive group in need of protection to an inert group (i.e., non-reactive group) using particular reagents. Within a protected group, the moiety that distinguishes the protected form from the unprotected form is regarded as the “protecting group”. For example, -OTBS is a protected form of hydroxyl group (—OH), wherein the -TBS group is the hydroxyl protecting group.

In the present invention, the terms “deprotection” and “removal of protection” have the same meaning, both referring to the process of transforming a protected group to its unprotected form.

In the present invention, the term “hydroxyl protecting group” includes all the groups that can be used as common hydroxyl protecting groups. A hydroxyl protecting group is preferably an alkanoyl (e.g., acetyl, butyryl), aromatic alkanoyl (e.g., benzoyl), benzyl, triphenylmethyl, trimethylsilyl, t-butyldimethylsilyl, allyl, acetal, or ketal group. The removal of acetyl groups is generally carried out under basic conditions, most commonly by the ammonolysis with NH₃/MeOH or by the methanolysis catalyzed by methanol anions. Benzyl groups can be easily removed via palladium-catalyzed hydrogenolysis in a neutral solution at room temperature, or via reduction reaction with metallic sodium in ethanol or liquid ammonia. Triphenylmethyl groups are generally removed via catalytic hydrogenolysis. Trimethylsilyl groups are generally removed using reagents containing fluoride ions (e.g., tetrabutylammonium fluoride/anhydrous THF, etc.). The t-butyldimethylsilyl ether is relatively stable, which can withstand ester hydrolysis conditions with alcoholic potassium hydroxide as well as mild reduction conditions (e.g., Zn/CH₃OH, etc.), but can be removed by fluoride ions (e.g., Bu₄N⁺F⁻) in a tetrahydrofuran solution or by aqueous acetic acid at room temperature. The protection of diols preferably forms dioxolanes, dioxanes, cyclic carbonates, or cyclic borates.

In the present invention, the term “thiol protecting group” includes all the groups that can be used as common thiol protecting groups. Similar to hydroxyl groups, thiol groups can be protected in the form of thioethers or thioesters. Thiol protecting groups are preferably tert-butyl, benzyl, substituted benzyl, benzhydryl, substituted benzhydryl, trityl, acetyl, benzoyl, tert-butoxycarbonyl, benzyloxycarbonyl, thioacetal, or thioketal groups. The deprotection of thioethers can be realized by acid-catalyzed reduction using Na/NH₃, or by reactions with heavy metal ions (e.g., Ag⁺, Hg⁺) followed by treating with hydrogen sulfide. Some groups such as hemi-thioacetals containing S-diphenylmethyl, S-triphenylmethyl thioether, S-2-tetrahydropyranyl, or S-isobutoxymethyl groups can be oxidized to disulfides by (SCN)₂, iodine, or thionyl chloride, and further reduced to thiols. The formation of thioester and the corresponding deprotection methods are the same as those of carboxylates.

In the present invention, the term “carboxyl protecting group” refers to a protecting group that can be transformed into a carboxyl group via hydrolysis or via the deprotection of the carboxyl protecting group. A carboxyl protecting group is preferably an alkyl (e.g., methyl, ethyl, and butyl) or aralkyl (e.g., benzyl) group, and more preferably a butyl (tBu), methyl (Me), or ethyl (Et) group. In the present invention, the “protected carboxyl group” is a group formed by protecting a carboxyl group with an appropriate carboxyl protecting group, preferably a methoxycarbonyl group, an ethoxycarbonyl group, a t-butyloxycarbonyl group, or a benzyloxycarbonyl group. Said carboxyl protecting groups can be removed through hydrolysis catalyzed by acids or alkalis, or through pyrolysis reactions occasionally. For example, t-butyl groups can be removed under mild acidic conditions, and benzyl groups can be removed by hydrogenolysis. The reagent used for the removal of a carboxyl protecting group is selected from the group consisting of TFA, H₂O, LiGH, NaOH, KOH, MeOH, EtOH, and combinations thereof, preferably the combination of TFA and H₂O, the combination of LiGH and MeOH, or the combination of LiGH and EtOH. The deprotection of a protected carboxyl group can produce the corresponding free acid, which could be carried out in the presence of an alkali that forms pharmaceutically acceptable salts with the free acid generated during the deprotection.

In the present invention, the term “amino protecting group” is equivalent to “amine protecting group”, including all the groups that can be used as common amino/amine protecting groups, such as aryl C₁₋₆ alkyl groups, C₁₋₆ alkoxy C₁₋₆ alkyl groups, C₁₋₆ alkoxycarbonyl groups, aryloxycarbonyl groups, C₁₋₆ alkylsulfonyl groups, arylsulfonyl groups, methylsilyl groups, etc. An amino protecting group is preferably Boc (t-butoxycarbonyl), Moz (p-methoxybenzyloxycarbonyl), or Fmoc (9-fluorenylmethoxycarbonyl). The reagent used for the removal of an amino protecting group includes but is not limited to TFA, H₂O, LiGH, MeOH, EtOH, and combinations thereof, preferably the combination of TFA and H₂O, the combination of LiGH and MeOH, or the combination of LiGH and EtOH. The reagent used for the removal of Boc can be TFA or HCl/EA, preferably TFA. The reagent used for the removal of Fmoc can be the solution of 20% piperidine in N,N-dimethylformamide (DMF).

In the present invention, the term “alkynyl protecting group” includes all the groups that can be used as common alkynyl protecting groups, preferably a trimethylsilyl (TMS), triethylsilyl, tert-butyldimethylsilyl (TBS), or biphenyldimethylsilyl group. The TMS-protected alkynyl groups can be easily deprotected under basic conditions, for example, K₂CO₃/MeOH or KOH/MeOH. The TBS-protected alkynyl groups can be deprotected in the solution of tetra-n-butylammonium fluoride in tetrahydrofuran (TBAF/THF).

In the present invention, hydroxyl groups that can be protected by hydroxyl protecting groups are not particularly limited, e.g., alcoholic hydroxyl groups and phenolic hydroxyl groups. Amino/amine groups that can be protected by amino protecting groups are not particularly limited, e.g., amino groups of primary amines, secondary amines, hydrazines, and amides. Amine groups in the present invention are not particularly limited, including but not limited to primary amine groups, secondary amine groups, tertiary amine groups, and quaternary ammonium ions.

In the present invention, the deprotection of protected hydroxyl groups is related to the type of hydroxyl protecting group. Said type of hydroxyl protecting group is not particularly limited; for example, benzyl, silyl ether, acetal, or tert-butyl groups can be used to protect terminal hydroxyl groups, and the corresponding deprotection methods include but are not limited to the following:

A: Removal of benzyl groups

The removal of benzyl groups can be achieved via hydrogenation using hydrogenation reducing agents and hydrogen donors. The water content in this reaction system should be less than 1% so that the reaction can proceed smoothly.

The catalyst for hydrogenation reduction is not particularly limited, preferably palladium or nickel. The catalyst support is not particularly limited, preferably aluminum oxide or carbon, and more preferably carbon. The amount of palladium used is 1 to 100 wt % relative to compounds containing protected hydroxyl groups, preferably 1 to 20 wt % relative to compounds containing protected hydroxyl groups.

The reaction solvent is not particularly limited, as long as it can dissolve both the starting materials and the products, but is preferably methanol, ethanol, ethyl acetate, tetrahydrofuran, or acetic acid, more preferably methanol. The hydrogen donor is not particularly limited, but is preferably gaseous hydrogen, cyclohexene, 2-propanol, or ammonium formate, etc. The reaction temperature is preferably in the range of 25 to 40° C. The reaction time is not particularly limited, which is negatively correlated with the amount of catalyst used and preferably in the range of 1 to 5 hours.

B: Deprotection of acetals and ketals

The compounds used for protecting hydroxyl groups in the form of acetals or ketals are preferably ethyl vinyl ether, tetrahydropyran, acetone, 2,2-dimethoxypropane, or benzaldehyde, etc. The deprotection of such acetals or ketals can be realized under an acidic condition wherein the pH of the solution is preferably 0 to 4. The acid used is not particularly limited, but is preferably acetic acid, phosphoric acid, sulfuric acid, hydrochloric acid, or nitric acid, and more preferably hydrochloric acid. The reaction solvent is not particularly limited, as long as it can dissolve the reactants and the products, but is preferably water. The reaction temperature is preferably 0 to 30° C.

C: Deprotection of silyl ethers

The protected hydroxyl groups in the form of silyl ethers include trimethylsilyl ether, triethylsilyl ether, tert-butyldimethylsilyl ether, tert-butyldiphenylsilyl ether, etc. The deprotection of such silyl ethers uses compounds containing fluoride ions which are preferably tetrabutylammonium fluoride, tetraethylammonium fluoride, hydrofluoric acid, or potassium fluoride, and more preferably tetrabutylammonium fluoride or potassium fluoride. The molar equivalent of the fluorine-containing compound used is 5 to 20 times that of the protected hydroxyl group, preferably 8 to 15 times. If the molar equivalent of the fluorine-containing compound used is less than 5 times that of the protected hydroxyl group, the deprotonation will be incomplete. If the molar equivalent of the deprotection reagent used exceeds 20 times that of the protected hydroxyl group, the excess reagents or compounds will bring difficulty to purification and will possibly enter the subsequent steps to cause side reactions. The reaction solvent is not particularly limited, as long as it can dissolve the reactants and the products, but is preferably an aprotic solvent and more preferably tetrahydrofuran or dichloromethane. The reaction temperature is preferably 0 to 30° C.; if the temperature is lower than 0° C., the reaction rate will be relatively slow and the protecting groups will not be completely removed.

D: Removal of tert-butyl groups

The removal of tert-butyl groups is carried out under an acidic condition wherein the pH of the solution is preferably 0 to 4. The acid used is not particularly limited, but is preferably acetic acid, phosphoric acid, sulfuric acid, hydrochloric acid, or nitric acid and more preferably hydrochloric acid. The reaction solvent is not particularly limited, as long as it can dissolve the reactants and the products, but is preferably water. The reaction temperature is preferably 0 to 30° C.

In the present invention, the term “activation of carboxyl group” refers to the activation of carboxyl groups with carboxyl activating agents. The activated carboxyl groups can promote condensation reactions, for example, by inhibiting the generation of racemic impurities, by accelerating the reactions through catalysis, etc. The “carboxyl activating group” is a residue of a carboxyl activating agent. Said carboxyl activating agent is selected from the group consisting of N-hydroxysuccinimide (NHS), 1-ethyl-3-(3-dimethylaminopropyl)carbodiimide hydrochloride (EDCI), N-hydroxy-5-norbornene-2,3-dicarboximide (HONb), N,N′-dicyclohexylcarbodiimide (DCC), and combinations thereof, preferably the combination of NHS/EDCI, NHS/DCC, or HONb/DCC, and most preferably the combination of NHS/EDCI.

In the present invention, the condensing agents used in reactions are not particularly limited, preferably selected from the group consisting of N,N′-dicyclohexylcarbodiimide (DCC), 1-ethyl-3-(3-dimethylaminopropyl)carbodiimide hydrochloride (EDC-HCl), o-(7-azabenzotriazol-1-yl)-N,N,N′,N′-tetramethyluronium hexafluorophosphate (HATU), and o-(benzotriazol-1-yl)-N,N,N′,N′-tetramethyluronium hexafluorophosphate (HBTU), most preferably DDC. Generally, the molar equivalent of the condensing agent used is 1 to 20 times that of carboxylic acids, preferably 5 to 10 times. Appropriate catalysts (e.g., 4-dimethylaminopyridine (DMAP)) can be added in this reaction.

In the present invention, the oxidizing agent used in the reaction is not particularly limited, as long as it is a compound or a combination of various compounds that can increase the valence of the substrate, preferably selected from the group consisting of phenyliodine bis(trifluoroacetate), 1,4-benzoquinone, benzyltrimethylammonium tribromide, pyridinium dichromate, potassium dichromate, ozone, oxygen, hypofluorous acid, sodium hypochlorite, cobalt(m) acetate, cobalt acetate, manganese acetate, palladium acetate, copper acetate, monoperoxyphthalic acid, iodine, N-iodosuccinimide, iodoxybenzene, 2-iodoxybenzoic acid, dimethyldioxirane, dimethyl sulfoxide-oxalyl chloride, dimethyl sulfoxide-acetic anhydride, DDQ, dichlorotris(triphenylphosphine)ruthenium, manganese dioxide, diacetoxyiodobenzene, periodate, sodium periodate, sodium periodate-osmium tetroxide, potassium permanganate, sodium perborate, peroxybenzoic acid, dibenzoyl peroxide, nickel peroxide, hydrogen peroxide, cumene hydroperoxide, tert-butanol peroxide, peracetic acid, m-chloroperoxybenzoic acid, N-chlorosuccinimide, pyridinium chlorochromate, palladium chloride-copper chloride, hydrogen peroxide-urea complex, triphenylmethyltetrafluoroborate, tributyltin oxide, cobalt trifluoride, vanadium oxytrifluoride, chromium trioxide, manganese triacetate, TEMPO, diammonium cerium nitrate, bromine, pyridine N-oxide, silver oxide, O-ethylperoxycarbonic acid, manganese acetylacetonate, vanadyl acetylacetonate, aluminum isopropoxide, potassium peroxymonosulfate, dichloroiodobenzene, etc., and combinations thereof, and more preferably selected from the group consisting of oxygen, sodium hypochlorite, hydrogen peroxide, dichloroiodobenzene, potassium peroxymonosulfate, etc., and combinations thereof. The molar equivalent of the oxidizing agent used is 1 to 50 times that of the hydroxyl groups in intermediate compounds, preferably 1 to 20 times, and more preferably 5 to 10 times.

In the present invention, the reducing agent used in the reaction is not particularly limited, as long as the Schiff base formed via the reaction between an amine and an aldehyde or ketone can be reduced to an amino group; the reducing agent is preferably selected from the group consisting of sodium borohydride, sodium cyanoborohydride, lithium aluminum hydride, borane, diborane, diisobutylaluminum hydride, diisopinocampheylborane, lithium borohydride, zinc borohydride, borane-pyridine, borane-dimethyl sulfide, borane-tetrahydrofuran, etc., and combinations thereof, and more preferably sodium cyanoborohydride. The molar equivalent of the reducing agent used is 1 to 50 times that of the amino groups to be modified, preferably 1 to 20 times, and more preferably 5 to 10 times.

In the present invention, the reaction temperature is 0 to 200° C., preferably 0 to 100° C. The reaction time is preferably 10 min to 48 h, and more preferably 30 min to 24 h. The obtained product can be purified by a purification method such as extraction, recrystallization, adsorption treatment, precipitation, reverse precipitation, membrane dialysis, supercritical extraction, etc.

In the present invention, reactions may be solvent-free or with aprotic solvents; the aprotic solvent includes toluene, benzene, xylene, acetonitrile, ethyl acetate, diethyl ether, tert-butyl methyl ether, tetrahydrofuran, chloroform, dichloromethane, dimethylsulfoxide, dimethylformamide, and dimethylacetamide, and is preferably tetrahydrofuran, dichloromethane, dimethylsulfoxide, or dimethylformamide.

In the present invention, the base used in the reaction can be an organic base (e.g., triethylamine, pyridine, 4-dimethylaminopyridine, imidazole, or N,N-diisopropylethylamine), preferably triethylamine or pyridine. The molar equivalent of the base used is 1 to 50 times that of carboxylic acids, preferably 1 to 10 times, and more preferably 1 to 3 times.

In the present invention, the repeat unit of polyethylene glycol is the oxyethylene unit, i.e., —CH₂CH₂O— or —OCH₂CH₂—, also termed the EO unit. The number of repeat units is also termed the number of EO units. The average number of repeat units is also termed the average number of EO units, which is preferably the number-averaged mean value.

In the present invention, for polydisperse cases, the term “identical”, “same” or “equal” (including other forms of equivalent expressions) with respect to the molecular weight/degree of polymerization of a single compound molecule and the number average molecular weight/number average degree of polymerization of a compound component in macroscopic matter, unless otherwise specified, does not indicate a strict equality but a proximity or approximate equality of values, wherein the proximity or approximate equality preferably corresponds to a deviation within 10% and the preset value is generally used as the base value.

In the present invention, for monodisperse cases, the same or equal numbers of oxyethylene units with respect to a single compound molecule or a general formula are strictly equal in value. For example, provided that the number of EO units of a certain PEG component is 11, the value 12 is not within the preset scope. However, in order to obtain a compound component having the preset number of EO units, particular preparation methods might be used, and therefore the macroscopic matter obtained could also contain components having other numbers of EO units besides the component having the target number of EO units due to the limitations of preparation or purification methods; in this case, if the deviation between the average number of EO units and the preset number of EO units is within ±5% (base value≥10) or within ±0.5 (base value<10), it is considered to have obtained a monodisperse macroscopic matter containing the target component. Moreover, if the content of components having the number or average number of EO units within the allowed deviation reaches specific percentages (preferably ≥90%, more preferably >95%, more preferably >96%, more preferably >98%, and more preferably 99-100%), it is also considered to have obtained the monodisperse macroscopic matter containing the target component; even if the aforementioned content in percentage is not met, the product as well as the component in the form of main product, co-product or by-product, which are insufficient in content, are all within the scope of the present invention as long as they are prepared using the preparation methods of the present invention or any similar methods adopting basically the same ideas, with or without isolation or purification.

In the present invention, when Da, kDa, the number of repeat units, or the number of EO units is used to describe the molecular weight of a compound of general formula for polydisperse components, the value of molecular weight is allowed to be within a certain range around the given value (with endpoints included, and preferably within 10%) with respect to a single compound molecule. When the number of oxyethylene units is used to describe the preset molecular weight of a compound of general formula for monodisperse components, the number is not a variable value in a range but a discrete point while the average number of EO units of the prepared product could be variable within a certain range (within ±10% or ±1, preferably within ±5% or +0.5) due to non-uniform molecular weights. For example, provided that the molecular weight of an mPEG (methoxy polyethylene glycol) is 5 kDa, it means that the molecular weight of a single molecule of general formula is in the range of 4500-5500 Da and the average molecular weight of the corresponding components in the product is 5 kDa; i.e., the product with the average molecular weight in the range of 4500-5500 Da is regarded as the target product, and only the components with molecular weight in the range contribute to the content of the target component. As another example, provided that an mPEG is designed to have 22 oxyethylene units, all the compound molecules of general formula should have the number of EO units being strictly 22 while the prepared product could be a mixture of compounds having different numbers of EO units being 20, 21, 22, 23, or 24; meanwhile, if the average number of EO units is within the range of 22±2.2 (preferably in the range of 22±1.1), then the target component is considered obtained and all the components with molecular weight within the range can be regarded as the target component for calculation of purity.

In the present invention, unless otherwise specified, the term “mPEG” refers to a polyethylene glycol chain capped with a methoxy group, having the structure of

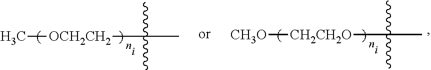

wherein nᵢ is the degree of polymerization of the polyethylene glycol chain and is an integer selected from 1-250.

In the present invention, a product with PDI<1.005 is regarded as monodisperse (PDI=1).

In the present invention, the number of repeat units in a “single-chain component” is at least 2.

In the present invention, the “lipid” includes but is not limited to esters of fatty acids and is generally characterized by poorer solubility in water and being soluble in many nonpolar organics. Although lipids usually have poorer solubility in water, some types of lipids (e.g., lipids modified with polar groups, such as DMG-PEG2000) have limited water solubility and can be dissolved in water under certain conditions. The known types of lipids include biomolecules such as fatty acids, waxes, sterols, fat-soluble vitamins (e.g., vitamins A, D, E, and K), monoglycerides, diglycerides, triglycerides, and phospholipids.

In the present invention, lipids include simple esters, compound esters, and derived lipids. Said simple esters are esters formed by fatty acids and alcohols, which can also be classified into three subcategories: fats, oils, and waxes. Said compound esters are “lipoid compounds” which are also termed “lipoids”, including phospholipids, sphingolipids, glycolipids, steroids, sterols, and lipoproteins. Said derived lipids include simple lipid derivatives and compound lipid derivatives, having the general properties of lipids.

In the present invention, lipids can be synthetic or derived (separated or modified) from natural sources or compounds.

In the present invention, the term “liposome” refers to a closed vesicle formed by the self-assembly of lipids, having one or more lipid bilayer structures.

In the present invention, the term “lipid nanoparticle” or “LNP” refers to a nano-sized (e.g., 1 to 1000 nm) particle that contains one or more types of lipid molecules. In the present invention, the LNP can further contain at least one type of non-lipid payload molecule (e.g., one or more types of nucleic acid molecules). In some embodiments, the LNP contains a non-lipid payload molecule either partially or completely encapsulated inside a lipid shell. Particularly, in some embodiments, the payload is a negatively charged molecule (e.g., mRNA encoding a viral protein), and the lipid components of the LNP include at least one type of cationic lipid and at least one type of PEGylated lipid. It can be expected that the cationic lipids can interact with the negatively charged payload molecules and facilitate the incorporation and/or encapsulation of the payload into the LNP during LNP formation. As provided herein, other lipids that can be part of an LNP include but are not limited to neutral lipids and steroid lipids. In some embodiments, the LNP of the present invention includes one or more kinds of PEGylated lipids of the formula (1) described herein.

In the present invention, the term “cation” refers to a structure that is positively charged either permanently or non-permanently in response to certain conditions (e.g., pH). Therefore, cations include not only permanent cations but also those cationisable. Permanent cations refer to the corresponding compounds, groups, or atoms that are positively charged under conditions of any pH value or hydrogen ion activity of its environment. As a typical example, the presence of a quaternary nitrogen atom results in a positive charge. When a compound carries multiple positive charges, it can also be termed a polycation. A cationisable substance refers to a compound, group, or atom that is positively charged at a lower pH and uncharged at a higher pH of its environment. Moreover, in non-aqueous environments where the pH cannot be determined, a cationisable compound, group, or atom is positively charged at a high concentration of hydrogen ions and uncharged at a low concentration or activity of hydrogen ions. It depends on the individual properties of the cationisable or polycationisable compound, in particular the pKa of the respective cationisable group or atom, at which pH or hydrogen ion concentration the compound is charged or uncharged. In diluted aqueous environments, the fraction of cationisable compounds, groups, or atoms that are positively charged may be estimated using the so-called Henderson-Hasselbalch equation which is well-known to a person skilled in the art. In some embodiments, a cationisable compound or its moiety is positively charged at the physiological pH (e.g., approximately 7.0-7.4). In some preferred embodiments, a cationisable compound or its moiety is neutral at the physiological pH (e.g., approximately 7.0-7.4), but becomes positively charged at a lower pH (e.g., approximately 5.5-6.5). In some embodiments, the preferred range of pKa of the cationisable compound or its moiety is from approximately 5 to approximately 7.

In the present invention, the term “cationic lipid” refers to a lipid that is positively charged at any pH or hydrogen ion activity of its environment, or a cationisable lipid which can be positively charged in response to the pH or hydrogen ion activity of its environment (e.g., the expected environment for its use). Therefore, the term “cationic” includes the scopes of both “permanently cationic” and “cationisable”. In some embodiments, the positive charge of a cationic lipid originates from the existence of a quaternary nitrogen atom. In some embodiments, cationic lipids include zwitterionic lipids that can be positively charged in the expected environment for their use (e.g., at the endosomal pH). In some embodiments, if a liposome or LNP contains cationisable lipids, it is preferred that about 1% to 100% cationisable lipids are cationized at a pH of about 1 to 9, preferably 4 to 9, 5 to 8, or 6 to 8, and more preferably at the endosomal pH (e.g., about 5.5 to 6.5). Cationic lipids include but are not limited to N,N-dioleyl-N,N-dimethylammonium chloride (DODAC), N,N-distearyl-N,N-dimethylammonium bromide (DDAB), N-[1-(2,3-dioleoyloxy)propyl]-N,N,N-trimethylammonium chloride (DOTAP), N-[1-(2,3-dioleyloxy)propyl]-N,N,N-trimethylammonium chloride (DOTMA) N,N-dimethyl-2,3-dioleyloxy-1-(dimethylamino)propane (DODMA), 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), the cationic lipids disclosed in CN113402405A, and mixtures thereof.

In the present invention, the term “PEGylated lipid” refers to a molecule containing both lipid and polyethylene glycol moieties. PEGylated lipids not only include those of the general formula (1) of the present invention, but also include but are not limited to polyethylene glycol-1,2-dimyristoyl-sn-glycerol (PEG-DMG), polyethylene glycol-1,2-distearoyl-sn-glycero-3-phosphoethanolamine (PEG-DSPE), PEG-cholesterol, polyethylene glycol-diacylglycamide (PEG-DAG), polyethylene glycol-dialkyloxypropyl (PEG-DAA), and specifically, PEG500-dipalmitoylphosphatidylcholine, PEG2000-dipalmitoylphosphatidylcholine, PEG500-distearylphosphatidylethanolamine, PEG2000-distearylphosphatidylethanolamine, PEG500-1,2-oleoylphosphatidylethanolamine, PEG2000-1,2-oleoylphosphatidylethanolamine, PEG2000-2,3-distearoylglycerol (PEG-DMG), etc.

In the present invention, the term “neutral lipid” refers to any of the many types of lipid substances existing in the form of uncharged species or neutral zwitterionic species at a chosen pH, preferably phospholipid. Such lipids include but are not limited to 1,2-dilinoleoyl-sn-glycero-3-phosphocholine (DLPC), 1,2-dimyristoyl-sn-glycero-phosphocholine (MPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dioleoylphosphatidylserine (DOPS), dipalmitoylphosphatidylglycerol (DPPG), palmitoyloleoylphosphatidylethanolamine (POPE), distearoylphosphatidylethanolamine (DSPE), dipalmitoylphosphatidylethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), and compositions thereof. Neutral lipids can be synthetic or naturally sourced.

In the present invention, a “steroid lipid” is selected from the group consisting of cholesterol, coprostanol, sitosterol, ergosterol, campesterol, stigmasterol, brassicasterol tomatidine, ursolic acid, α-tocopherol, and compositions thereof.

In the present invention, a liposome/lipid nanoparticle can be termed a “PEGylated liposome/lipid nanoparticle” when containing PEGylated lipids, a “cationic liposome/lipid nanoparticle” when containing cationic lipids, and either a “PEGylated liposome/lipid nanoparticle” or a “cationic liposome/lipid nanoparticle” when containing both PEGylated lipids and cationic lipids.

In the present invention, the term “N/P ratio” refers to the molar ratio of the nitrogen atoms in cationic lipids to the phosphate groups in nucleic acids.

In the present invention, the term “nucleic acid” refers to DNA, RNA, or a modified form thereof, including purine or pyrimidine bases in DNA (adenine “A”, cytosine “C”, guanine “G”, thymine “T”), and purine or pyrimidine bases in RNA (adenine “A”, cytosine “C”, guanine “G”, uracil “U”).

In the present invention, the term “RNA” refers to a ribonucleic acid that may be naturally or non-naturally existing. For example, an RNA may include modified and/or non-naturally existing components such as one or more nucleobases, nucleosides, nucleotides, or linkers. An RNA may include a cap structure, a chain terminating nucleoside, a stem loop, a polyadenylation sequence and/or a polyadenylation signal. An RNA can have a nucleotide sequence encoding a polypeptide of interest. For example, an RNA can be a messenger RNA (mRNA). Translation of an mRNA encoding a particular polypeptide, for example, the in vivo translation of an mRNA inside a mammalian cell, can produce the encoded polypeptide. The RNA can be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, single guide RNA (sgRNA), cas9 mRNA, and mixtures thereof.

In the present invention, an antisense oligonucleotide or small interfering RNA (siRNA) can inhibit the expression of the target gene and the target protein in vitro or in vivo.

In the present invention, FLuc mRNA can express the luciferase protein which emits bioluminescence in the presence of a fluorescein substrate, and therefore FLuc is commonly used in the culture of mammalian cells to measure gene expression and cell activity.

In the present invention, the term “inhibiting the expression of a target gene” refers to the ability of nucleic acids to silence, reduce, or inhibit the expression of a target gene. To examine the extent of gene silencing, a test sample (e.g., a sample of cells in culture expressing the target gene) is contacted with nucleic acids that inhibit the expression of the target gene. The expression of the target gene in the test sample or test animal is compared to that in a control sample (e.g., a sample of cells in culture expressing the target gene) which is not contacted with or administered the nucleic acids. The expression of the target gene in the control sample can be assigned a value of 100%. In particular embodiments, inhibition of the expression of the target gene is achieved when the level of target gene expression in the test sample relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%.

In the present invention, methods for determining the level of target gene expression include but are not limited to dot blots, northern blots, in situ hybridization, ELISA, immunoprecipitation, enzyme function, and phenotypic assays.

In the present invention, the term “transfection” refers to the introduction of a species (e.g., RNA) into a cell. Transfection may occur, for example, in vitro, ex vivo, or in vivo.

In the present invention, the term “antigen” typically refers to a substance that can be recognized by the immune system, preferably recognized by the adaptive immune system, and can trigger an antigen-specific immune response, for example, forming antibodies and/or antigen-specific T cells as a part of the adaptive immune response. Typically, the antigen may be or may contain a peptide or protein which can be presented to T cells by MHC. In the sense of the present invention, the antigen may be a translation product of the provided nucleic acid molecule (preferably mRNA as defined herein). In this context, fragments, variants, and derivatives of peptides and proteins containing at least one epitope are also regarded as antigens.

In the present invention, the term “delivery” refers to providing an entity to the target, for example, delivering drugs and/or therapeutic agents and/or prophylactic agents to subjects that are tissues and/or cells of human and/or other animals.

In the present invention, the term “pharmaceutically acceptable carrier” refers to a diluent, adjuvant, excipient, or vehicle administered together with the therapeutic agent, which is, within the scope of sound medical judgement, suitable for contact with tissues of human and/or other animals without causing any excessive toxicity, irritation, allergic reaction, or other problems or complications corresponding to rational benefit/risk ratios. Pharmaceutically acceptable carriers that can be used in the pharmaceutical compositions in the present invention include but are not limited to sterile liquids, such as water and oil, including oils from petroleum, animals, vegetables, or synthetics, e.g., peanut oil, soybean oil, mineral oil, sesame oil, etc. When the pharmaceutical composition is administered intravenously, an exemplary carrier is water. Physiological saline, glucose, and aqueous glycerol solution can also be used as liquid carriers, especially for injection. Suitable pharmaceutical excipients include starch, glucose, lactose, sucrose, gelatin, maltose, chalk, silica gel, sodium stearate, glyceryl monostearate, talc, sodium chloride, defatted milk powder, glycerol, propylene glycol, water, ethanol, etc. Said compositions may also contain a small amount of humectant, emulsifier, or pH buffer as needed. Oral preparations may contain standard carriers, such as pharmaceutical grade mannitol, lactose, starch, magnesium stearate, sodium saccharin, cellulose, magnesium carbonate, etc. Specifically, excipients include but are not limited to anti-adherents, antioxidants, binders, coatings, compression aids, disintegrants, dyes (pigments), emollients, emulsifiers, fillers (diluents), film formers or coatings, flavors, fragrances, glidants (flow enhancers), lubricants, preservatives, printing inks, sorbents, suspending or dispersing agents, sweeteners, and water for hydration. More specifically, excipients include but are not limited to butylated hydroxytoluene (BHT), calcium carbonate, calcium phosphate, calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, phenyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (α-tocopherol), vitamin C, and xylitol.

In the present invention, pharmaceutical compositions can act systematically or locally. For this purpose, they can be administered via appropriate routes such as injection (e.g., intravenous, intraarterial, subcutaneous, intraperitoneal, and intramuscular injections, including instillation) or transdermal delivery, or via oral, buccal, transnasal, transmucosal, or topical routes, or in the form of ophthalmic preparation, or by inhalation. Regarding these routes of administration, the pharmaceutical compositions of the present invention can be administered in suitable dosage forms. Said dosage forms include but are not limited to tablets, capsules, lozenges, hard sugar agents, powders, sprays, creams, ointments, suppositories, gels, pastes, lotions, ointments, aqueous suspensions, injectable solutions, elixirs, and syrups.

In the present invention, vaccines are preventive or therapeutic materials that provide at least one antigen or antigenic function. An antigen or antigenic function can stimulate the body's adaptive immune system to provide an adaptive immune response.

In the present invention, treatment refers to patient management and care in order to resist diseases, obstacles, or symptoms, which is intended to delay the development of diseases, obstacles, or symptoms, reduce or alleviate symptoms and complications, and/or cure or eliminate diseases, obstacles, or symptoms. The patients to be treated are preferably mammals, especially humans.

### 1.2. Non-Linear PEGylated Lipids

An embodiment of the present invention is as follows:

A PEGylated lipid having the structure represented by the general formula (1):

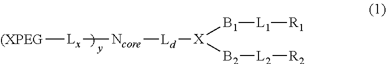

wherein, X is —CRₐ< or

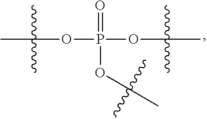 [SMILES: *OP(=O)(O*)O*]

and Rₐ is H or a C₁₋₁₂ alkyl group;
B₁ and B₂ are each independently a linking bond or a C₁₋₂₀ alkylene group;
L₁ and L₂ are each independently a linking bond or a divalent linking group;
R₁ and R₂ are each independently a C₁₋₅₀ aliphatic hydrocarbon group or a C₁₋₅₀ residue of aliphatic hydrocarbon derivative, containing 0-10 heteroatoms; the heteroatom is B, O, N, Si, P or S;
L_{d} is a linking bond or a divalent linking group;
N_{core} is a multivalent group having a valence of y+1, and contains a trivalent nitrogen-atom branching core connected to L_{d};
y is 2, 3, 4, 5, 6, 7, 8 or 9, or y>10;
y instances of Lₓ are each independently a linking bond or a divalent linking group;
XPEG is a polyethylene glycol component; y instances of XPEG each independently contains one, two, three or four RPEG; RPEG is a single-chain polyethylene glycol component containing at least 4 EO units, and the EO unit is —CH₂CH₂O— or —OCH₂CH₂—; RPEG in the same XPEG have the same terminal group T; T is a hydrogen atom, an alkyl group or R₀₁-L₀₁-, wherein L₀₁ is a linking bond or a divalent linking group, and R₀₁ is a functional group that can interact with bio-related substances;
the alkyl group, alkylene group, aliphatic hydrocarbon group, and residue of aliphatic hydrocarbon derivative are each independently substituted or unsubstituted;
the PEGylated lipid is monodisperse or polydisperse;
or a salt, tautomer, stereoisomer or solvate thereof.

In the present invention, a compound of the general formula (1) can exist in an unsolvated or solvated form including the hydrated form. In general, for the purpose of the present disclosure, the solvated forms having pharmaceutically acceptable solvents (e.g., water, ethanol, etc.) are equivalent to the unsolvated forms.

In the present invention, a compound of the general formula (1) and its salts and solvates can exist in their tautomeric forms including those from keto-enol tautomerism, amide-imidic acid tautomerism, lactam-lactim tautomerism, enamine-imine tautomerism, proton transfer tautomerism and valence tautomerism.

In the present invention, a compound of the general formula (1) should be considered as including its salts, tautomers, stereoisomers and solvates.

In one specific embodiment of the present invention, the structure of PEGylated lipid is represented by the general formula (2) or (3):

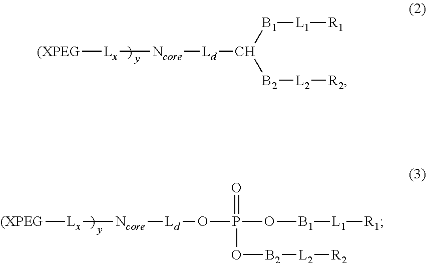

wherein, y is 2 or 3;
when y is 2, N_{core} is selected from the group consisting of

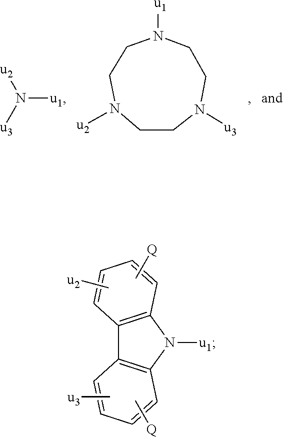

when y is 3, N_{core} is

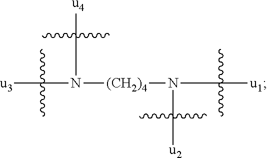

wherein, u₁, u₂, u₃, and u₄ are each independently a linking bond connected to L_{d} or Lₓ, and any two of u₁, u₂, u₃, and u₄ are not simultaneously connected to the same L_{d} or Lₓ;
wherein, Q is an electron-changing group, the number of which is 0, 1 or greater than 1; when the number of Q is greater than 1, any two Q have the same or different structures; the number of Q is preferably 0;
wherein, T is preferably a methyl group;
wherein, the definitions of XPEG Lₓ, L_{d}, B₁, B₂, L₁, L₂, R₁, and R₂ are the same as those of the general formula (1).

In the present invention, unless otherwise specified, all divalent linking groups are not particularly limited with respect to the stability. Any divalent linking group itself or one that consists of the divalent linking group and its adjacent heteroatom-containing groups are each independently a stable linking group or a degradable linking group.

In the present invention, unless otherwise specified, all divalent linking groups are not particularly limited with respect to the structure which can be a linear structure, a branched structure or a ring-containing structure. The number of non-hydrogen atoms in a divalent linking group is not particularly limited, each independently selected from integers of 0-20, preferably selected from integers of 1-10; wherein, the non-hydrogen atom is C, O, S, N, P, Si or B; when the number of non-hydrogen atoms is greater than 1, there are 1 type, 2 types, or more than 2 types of non-hydrogen atoms, and the non-hydrogen atoms are a combination of carbon and carbon atoms, a combination of carbon and hetero atoms, or a combination of hetero and hetero atoms.

### 1.2.1. L₁, L₂

In one specific embodiment of the present invention, L₁ and L₂ correspond to any of the following cases:
Case (1): one of L₁ and L₂ is a linking bond, and the other is a divalent linking group;
Case (2): both L₁ and L₂ are linking bonds;
Case (3): both L₁ and L₂ are divalent linking groups, and L₁ and L₂ have the same or different structures;
in any of Case (1), Case (2) and Case (3), the divalent linking group is selected from the group consisting of —CH₂—, —O—, —S—, —C(═O)—, —NR_{c}—, and combinations thereof, wherein, R_{c} is, at each occurrence, independently a hydrogen atom or a C₁₋₅ alkyl group;
preferably, L₁ and L₂ are each independently selected from the group consisting of a linking bond, —O—, —NHC(═O)—, —C(═O)NH—, —OC(═O)—, —C(═O)O—, —OC(═O)O—, —C(═O)—, —C(═O)O—(CH₂)ₓ, —OC(═O)—, —C(═O)O—(CH₂)ₓ— C(═O)O—, —OC(═O)—(CH₂)ₓ—OC(═O)—, —OC(═O)—(CH₂)ₓ—C(═O)O—, —C(═O)NH—(CH₂)ₓ—C(═O)O—, —C(═O)NH—(CH₂)ₓ—OC(═O)—, —NHC(═O)—(CH₂)ₓ—C(═O)O—, —NHC(═O)—(CH₂)ₓ—OC(═O)—, —C(═O)NH—(CH₂)ₓ—C(═O)NH—, —C(═O)NH—(CH₂)ₓ—NHC(═O)—, —NHC(═O)—(CH₂)ₓ—C(═O)NH—, and —NHC(═O)—(CH₂)ₓ—NHC(═O)—; wherein x is an integer in the range of 2-8.

### 1.2.2. B₁, B₂

In one specific embodiment of the present invention, B₁ and B₂ correspond to any of the following cases:
Case (1): one of B₁ and B₂ is a linking bond, and the other is a C₁₋₂₀ alkylene group;
Case (2): both B₁ and B₂ are linking bonds;
Case (3): both B₁ and B₂ are C₁₋₂₀ alkylene groups, and B₁ and B₂ have the same or different structures;
the C₁₋₂₀ alkylene group has 0-4 hydrogen atoms replaced by 0-4 R_{q}; R_{q} is, at each occurrence, independently selected from the group consisting of —(CH₂)_{tq}C[(CH₂)_{tq}H]₃, —(CH₂)_{tq}O(CH₂)_{tq}H, —(CH₂)_{tq}S(CH₂)_{tq}H, and —(CH₂)_{tq}N[(CH₂)_{tq}H]₂, wherein tq is, at each occurrence, independently an integer in the range of 0-4; preferably, R_{q} is, at each occurrence, independently —OH or —CH₃;
preferably, B₁ and B₂ are each independently selected from the group consisting of a linking bond, a methylene group, an ethylene group, a propylene group, a butylene group, a pentylene group, a hexylene group, a heptylene group, and an octylene group.

### 1.2.3. R₁, R₂

In one specific embodiment of the present invention, R₁ and R₂ are each independently selected from the group consisting of R_{L}, R_{B}, and Rᵣ; R₁ and R₂ each independently contains 0-10 Rₘ substituents; Rₘ is, at each occurrence, independently a linear, branched or ring-containing C₁₋₈ hydrocarbon group, preferably a methyl group;
R₁ and R₂ each independently contains 0-4 carbon-carbon double bonds and/or 0-4 carbon-carbon triple bonds;
R_{L} is selected from the group consisting of the following structures and cis-/trans-isomers thereof:

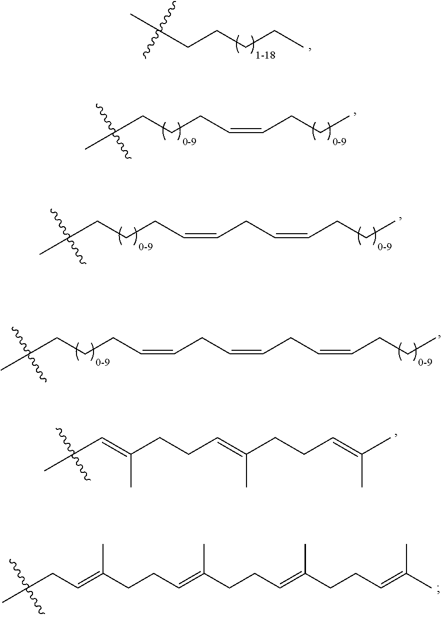

R_{L} is preferably selected from the group consisting of the following structures:

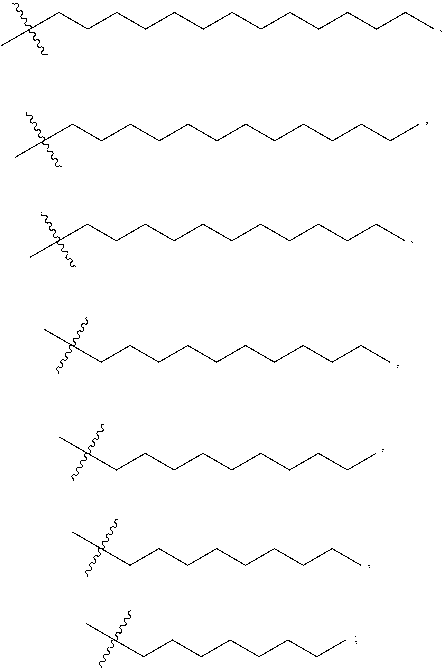

wherein, the structure of R_{B} is

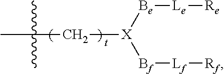

wherein X is CH or N; the t of R_{B} is an integer in the range of 0-5; Bₑ and B_{f} are each independently a linking bond or a C₁₋₁₀ alkylene group; Lₑ and L_{f} are each independently a linking bond, —O—, —OC(═O)—, —C(═O)O—, —NHC(═O)—, or —C(═O)NH—; Rₑ and R_{f} are each independently a C₁₋₁₂ alkyl group;
R_{B} is preferably selected from the group consisting of the following structures:

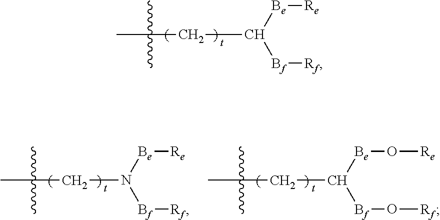

R_{B} is more preferably selected from the group consisting of the following structures:

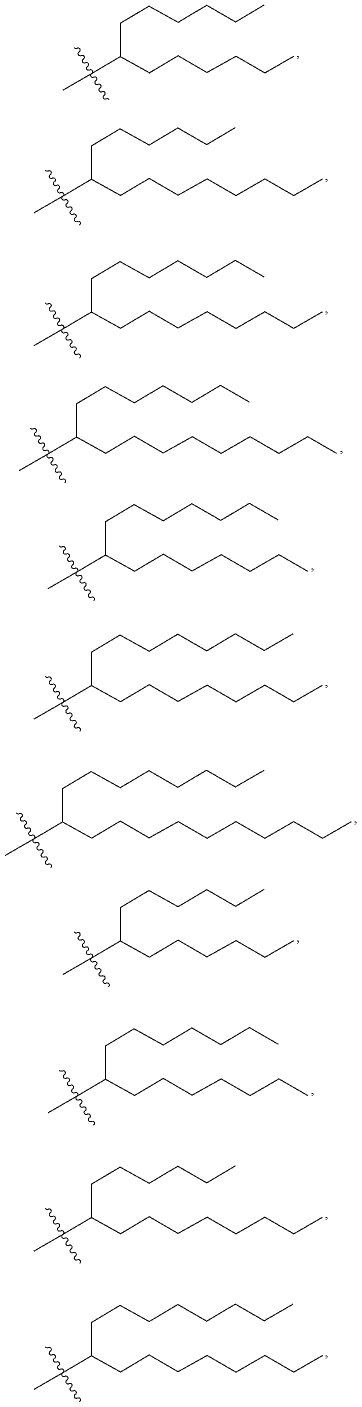

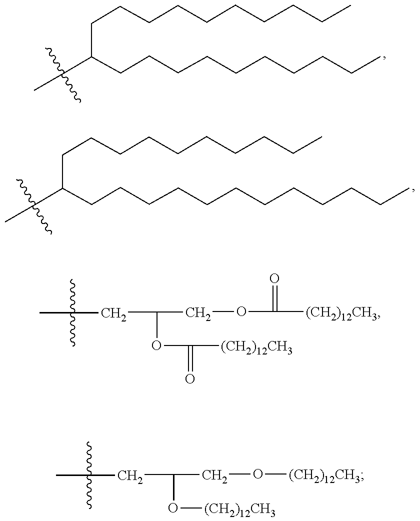

wherein, Rᵣ is a ring-containing C₄₋₃₀ alkyl group or C₄₋₃₀ heteroalkyl group, preferably

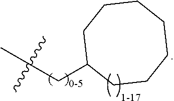

### 1.2.4. XPEG, RPEG

In one specific embodiment of the present invention, y instances of XPEG are independent of each other, having the same structure or a combination of two or more different structures; each XPEG independently has a linear or non-linear structure; the non-linear structure corresponds to any of the following cases:
Case (1): constructed from any multivalent residue of the following small molecules: a multifunctional small molecule, a multifunctional small molecule containing a heterofunctional group, and an amino acid containing at least one primary amino group;
Case (2): constructed from any of the following non-linear combinations: a branched structure, a comb structure, a dendritic structure, a dendrimer-like structure, a cyclic structure, a hyperbranched structure, and combinations of any two or more thereof;
wherein, the multifunctional small molecule is preferably a polyol, a polythiol, a polycarboxylic acid, a primary polyamine, or a secondary polyamine;
wherein, the multifunctional small molecule containing a heterofunctional group is preferably a polyol containing a heterofunctional group, a polythiol containing a heterofunctional group, a polycarboxylic acid containing a heterofunctional group, a primary polyamine containing a heterofunctional group, or a secondary polyamine containing a heterofunctional group;
wherein, the amino acid containing at least one primary amino group is preferably glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, ornithine, or citrulline;
each non-linear structure is preferably a branched structure, and x is equal to 2; it is further preferred that the non-linear structure is constructed from any trivalent residue of the following structures: a triol, a tri-thiol, a primary triamine, a secondary triamine, a tricarboxylic acid, a trisulfonate, a triisocyanate, a trifunctional small molecule containing a heterofunctional group, and an amino acid containing at least one primary amino group;
preferably, the structures of XPEG are all linear structures, or all non-linear structures; it is further preferred that every XPEG have the same structure.

In one specific embodiment of the present invention, each of y instances of XPEG independently has a linear or non-linear structure; the linear structure contains one RPEG; the non-linear structure contains two, three, or four RPEG and also contains a trivalent, tetravalent or pentavalent branching core, wherein the branching core is preferably selected from the group consisting of the following structures:

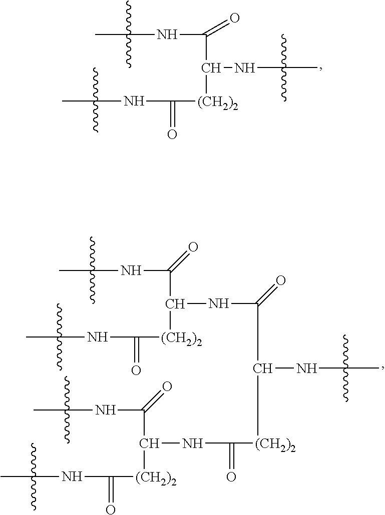

wherein the right end is connected to Lₓ.

In one specific embodiment of the present invention, the structure of RPEG is

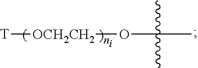

wherein, nᵢ is the degree of polymerization of the polyethylene glycol chain, being an integer in the range of 4˜250, wherein the polyethylene glycol chain is polydisperse or monodisperse; the i in nᵢ is an integer selected from 1 to m, and m is equal to the total number of RPEG in the PEGylated lipid.

In one specific embodiment of the present invention, the aforementioned nᵢ of quantity m are each independently an integer in the range of 4-100, preferably an integer in the range of 10-60, more preferably an integer in the range of 10-45, and most preferably 10, 11, 12, 20, 21, 22, 23, 24, or 25.

In one specific embodiment of the present invention, the number average molecular weight of RPEG is selected from 0.5 kDa-20 kDa, preferably 0.5 kDa-5 kDa, and more preferably 0.5 kDa, 1 kDa, 2 kDa, or 5 kDa.

### 1.2.5. T, R₀₁, L₀₁

In one specific embodiment of the present invention, T is a hydrogen atom.

In one specific embodiment of the present invention, T is a methyl group.

In one specific embodiment of the present invention, T is R₀₁-L₀₁-; wherein, L₀₁ is a linking bond, or selected from the group consisting of —CH₂—, —O—, —S—, —C(═O)—, —NH—, and combinations thereof;
L₀₁ is preferably selected from the group consisting of a linking bond, —(CH₂)—, —NH(CH₂)—, —NH(CH₂)ₜC(═O)NH(CH₂)ₜ—, —O(CH₂)ₜ—, —NH(CH₂)C(═O)O(CH₂)ₜ—, —OC(═O)(CH₂)—, —OC(═O)O(CH₂)ₜ—, —OC(═O)(CH₂)C(═O)—, and —(CH₂)ₜC(═O)NH(CH₂)ₜ; the left end of L₀₁ is connected to R₀₁; the t of L₀₁ is an integer in the range of 1 to 4, preferably 1 or 2;
L₀₁ is more preferably —NHCH₂CH₂— or —OCH₂CH₂—.

In one specific embodiment of the present invention, L₀₁ is a linking bond, or selected from the group consisting of a hydrocarbylene group, a heteroatom-containing divalent hydrocarbon group, —O—, —S—, —S—S—, —C(═O)—, —C(═O)O—, —OC(═O)—, —OC(═O)O—, —NH—, —C(═O)NH—, —NHC(═O)—, —OC(═O)NH—, —NHC(═O)O—, and combinations thereof.

In one specific embodiment of the present invention, R₀₁ a functional group that can interact with bio-related substances, wherein the interaction is selected from the group consisting of covalent bond formation, hydrogen bond formation, fluorescence effect, and targeting effect; R₀₁ is selected from the group consisting of reactive groups, modified forms of reactive groups, therapeutically targeting functional groups, and fluorescent functional groups; wherein, the modified forms are selected from the group consisting of the precursors of reactive groups, the active forms to which reactive groups are precursors, the active forms in which reactive groups are substituted, and the inactive forms in which reactive groups are protected; wherein, the precursors of reactive groups refer to the structures which can be transformed into the reactive groups through at least one process among oxidation, reduction, hydration, dehydration, electronic rearrangement, structural rearrangement, salt complexation and decomplexation, ionization, protonation, and deprotonation.

In one specific embodiment of the present invention, the aforementioned R₀₁ is selected from the group consisting of the functional groups in the following classes A˜I and modified forms thereof:
Class A: active ester group, analogous structure of active ester group; wherein, the active ester group is selected from the group consisting of a succinimidyl active ester group, a p-nitrophenyl active ester group, an o-nitrophenyl active ester group, a 1,3,5-trifluorophenyl active ester group, a 1,3,5-trichlorophenyl active ester group, a 1,3,5-tribromophenyl active ester group, a 1,3,5-triiodophenyl active ester group, a pentafluorophenyl active ester group, an imidazole active ester group, a benzotriazole active ester group, a thiazolidine-2-thione active ester group, a pyrrolidine-2-thione active ester group, a 2-mercaptobenzothiazole active ester group, and a 1-oxo-3-thioxoisoindoline active ester group; and wherein, the analogous structure of active ester group is an active carboxylate group or an active acyl group;
Class B: carboxyl group, protected carboxyl group, sulfonic acid group, sulfonate group, sulfinic acid group, sulfinate group, sulfenic acid group, ester group, thioester group, dithioester group, carbonate group, thiocarbonate group, dithiocarbonate group, trithiocarbonate group, xanthate group, tetrathiodiester group, sulfone group, sulfoxide group, methacryloyl group, hydroxamic acid group, thiohydroxamic acid group, sulfonyl halide group, thiocarboxy group;
Class C: aldehyde group, hydrated aldehyde group, thioaldehyde group, acyl halide group, ketone group, hydrated ketone group, thione group, hydrated thione group, glyoxal group, acetal group, monothioacetal group, dithioacetal group, ketal group, monothioketal group, dithioketal group, hemiacetal group, thiohemiacetal group, hemiketal group, orthoacid group, protected orthoacid group, orthoester group, cyanate group, thiocyanate group, isocyanate group, isothiocyanate group, oxazoline group, isoxazoline group;
Class D: primary amino group, secondary amino group, protected amino group, hydroxylamine group, thiol group, disulfide group, halogen atom, haloacetamide group, ammonium salt, hydrazino group, tetramethylpiperidinyloxy group, dioxapiperidinyloxy group, O-carbonyl hydroxylamine group, amide group, imide group, hydrazide group, sulfonyl hydrazide group, hydrazone group, imine group, enamino group, alkynylamine group, carbamate group, thiocarbamate group, dithiocarbamate group;
Class E: urea group, thiourea group, guanidino group and its protonated form, amidine group and its protonated form, anhydride group, squaric acid group, squarate group, semi-squaric acid group, semi-squarate group, imidazole-1-carboxamide group, imidate group, nitrone group, aldoxime group, ketoxime group;
Class F: maleimide group, furan-protected maleimide group, acrylate group, N-acrylamide group, N-methacrylamide group, methacrylate group, maleamic acid group, 1,2,4-triazoline-3,5-dione group, linear azo compound group, cyclic azo compound group;
Class G: alkenyl group, alkenylhydrocarbon group, cycloalkenyl group, alkynyl group, alkynylhydrocarbon group, protected alkynyl group, cycloalkynl group, linear conjugated diene group, cyclic conjugated diene group, heteroatom-containing cyclic conjugated diene group, epoxy group, 1,2,4,5-tetrazine group, azido group, nitrile oxide group, cyano group, isocyano group, diazo group, diazonium ion, azo oxide group, nitrilimine group, aldimine N-oxide group, tetrazolyl group, 4-acetyl-2-methoxy-5-nitrophenoxy group and its diazotized form, imidazole group, indolyl group; wherein, the cycloalkenyl group is selected from the group consisting of a cyclooctenyl group, a norbornenyl group, a norbornadienyl group, an oxa norbornenyl group, and an oxa norbornadienyl group;
Class H: hydroxyl group, protected hydroxyl group, protected dihydroxyl group, siloxy group, trihydroxysilyl group, protected trihydroxysilyl group; wherein, the hydroxyl group is selected from the group consisting of an alcoholic hydroxyl group, a phenolic hydroxyl group, an enolic hydroxyl group, and a hemiacetal hydroxyl group;
Class I: monosaccharide group, selected from the group consisting of the residues of allose, altrose, arabinose, cladinose, erythrose, erythrulose, fructose, fucitol, fucosamine, fucose, fuculose, galactosamine, galactosaminitol, N-acetyl-galactosamine, galactose, glucosamine, N-acetyl-glucosamine, glucosaminitol, glucose, glucose-6-phosphate, gulose glyceraldehyde, L-glycero-D-manno-heptose, glycerol, glyceraldehyde, dihydroxyacetone, gulose, idose, lyxose, mannosamine, mannose, mannose-6-phosphate, mannoheptulose, allulose, quinolose, quinosamine, rhamnitol, rhamnosamine, rhamnose, ribose, ribulose, deoxyribose, sedoheptulose, sorbose, tagatose, talose, tartaric acid, threose, xylose, xylulose, and functional derivatives thereof, the monosaccharide group is in D-configuration or L-configuration, cyclic or linear, substituted or unsubstituted;
wherein, the protected hydroxyl group is preferably selected from the group consisting of an ether group, a silicon ether group, an ester group, a carbonate group, and a sulfonate group; the protected amino group is preferably selected from the group consisting of a carbamate group, an amide group, an imide group, a N-alkylamine group, a N-arylamine group, an imine group, an enamine group, an imidazole group, a pyrrole group, and an indole group; the protected thiol group is preferably selected from the group consisting of a thioether group, a disulfide group, a silicon sulfide group, and a thioester group; the protected carboxyl group is preferably the carboxyl group protected by a group selected from the group consisting of a methyl group, an ethyl group, a tert-butyl group, and a benzyl group; the protected alkynyl group is preferably the alkynyl group protected by a silyl group; the protected dihydroxyl group preferably has the structure where the protecting group and two oxygen atoms form a five-membered or six-membered cyclic acetal structure; the dihydroxyl protecting group is preferably a methylene or substituted methylene group, and more preferably selected from the group consisting of a methylene group, a 1-methylmethylene group, a 1,1-dimethylmethylene group, a 1,1-cyclopentylene group, a 1,1-cyclohexylene group, a 1-phenylmethylene group, and a 3,4-dimethylphenylmethylene group.

In one specific embodiment of the present invention, the aforementioned R₀₁ is selected from the group consisting of the functional groups of the following classes A˜I and modified forms thereof:

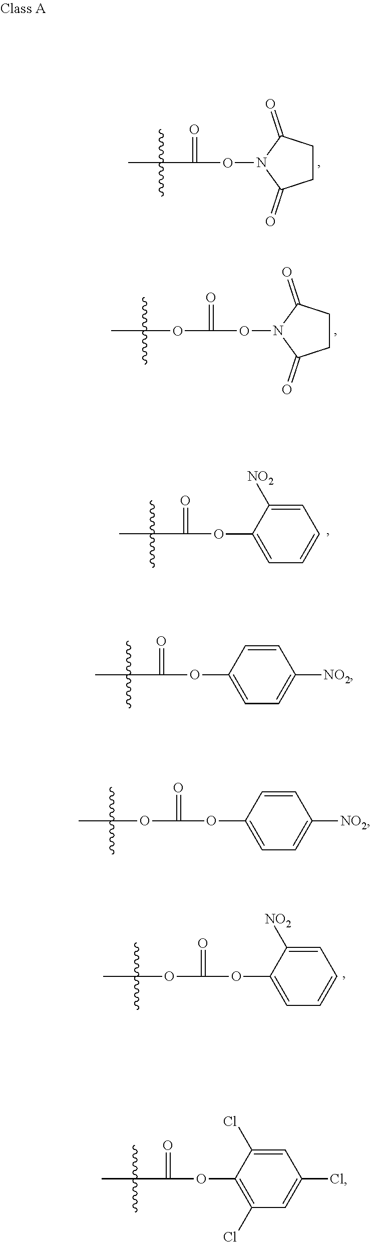

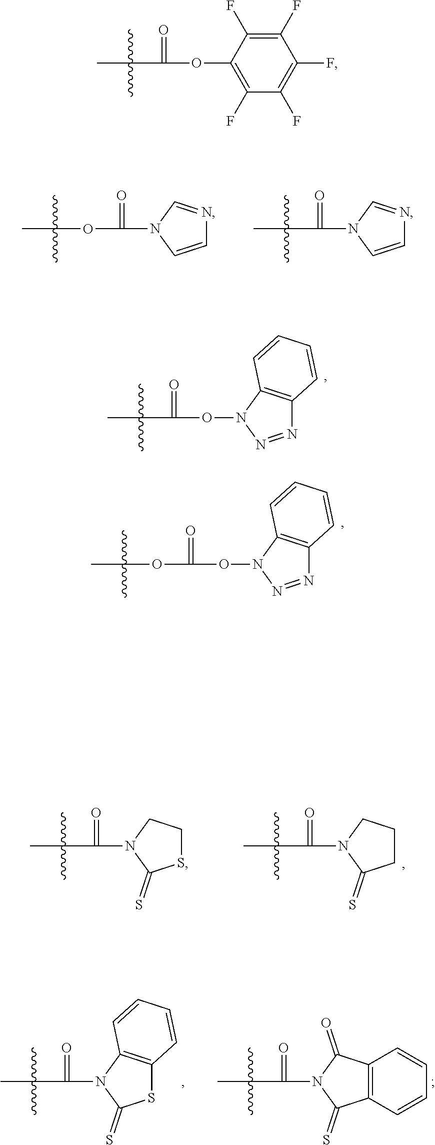

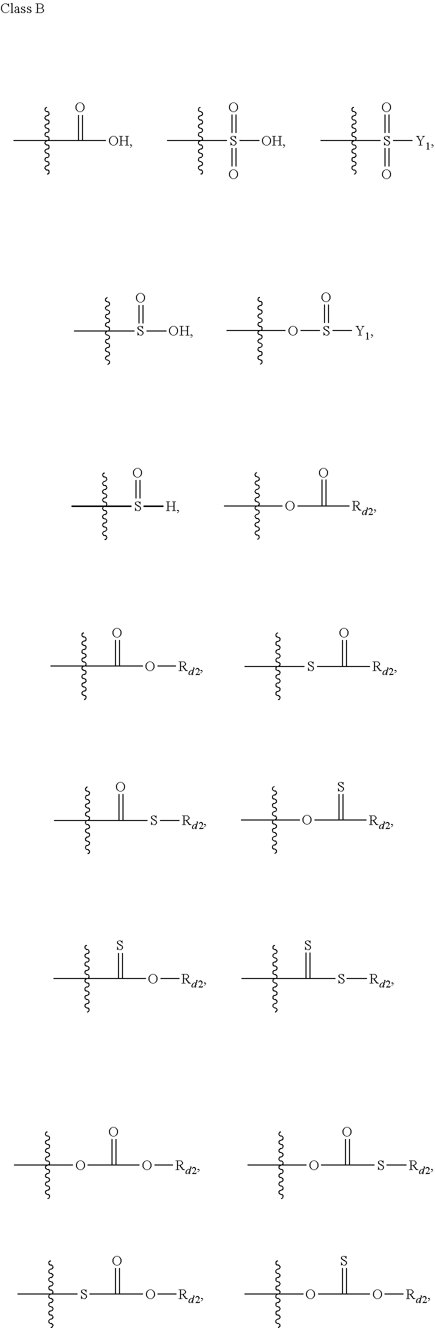

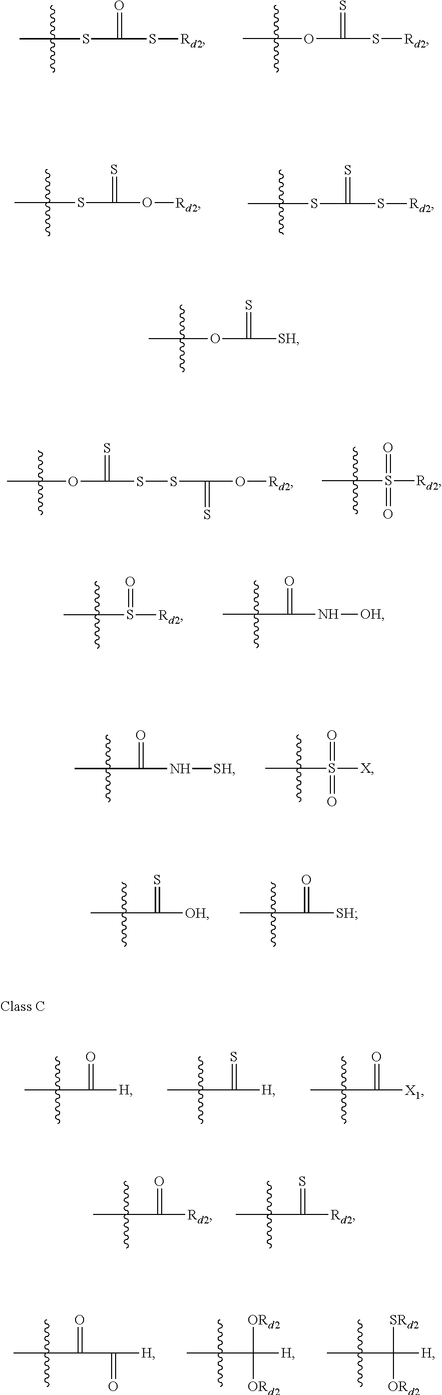

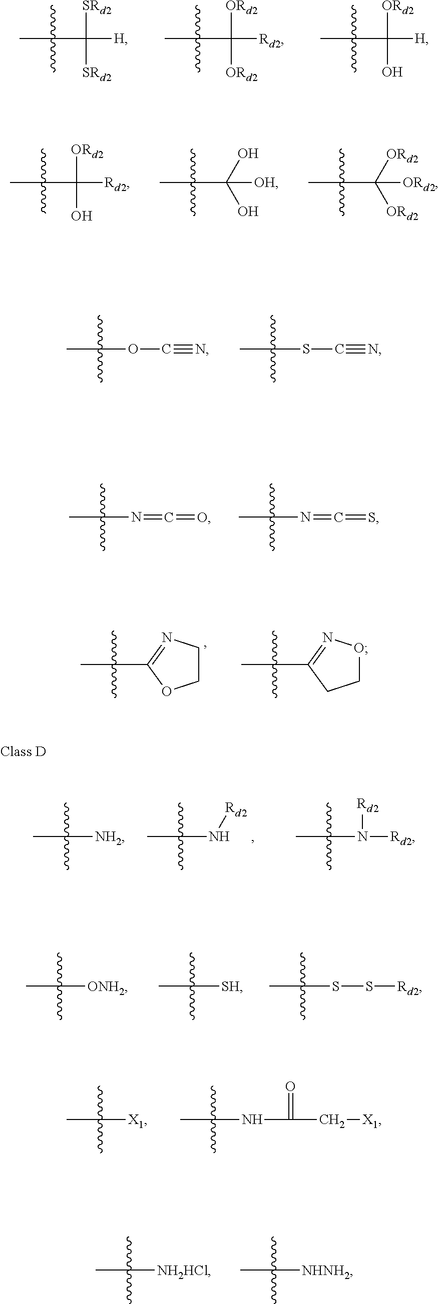

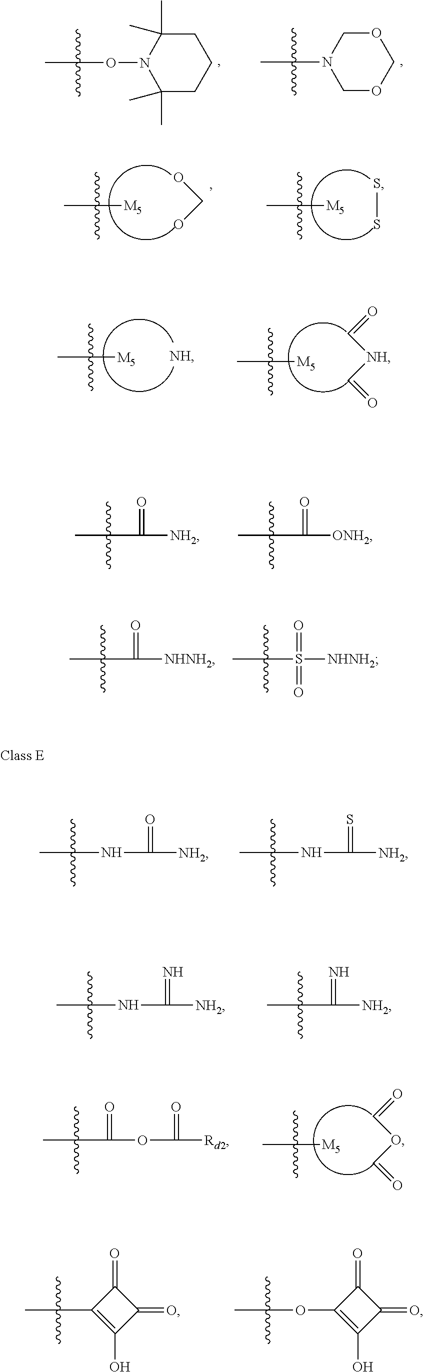

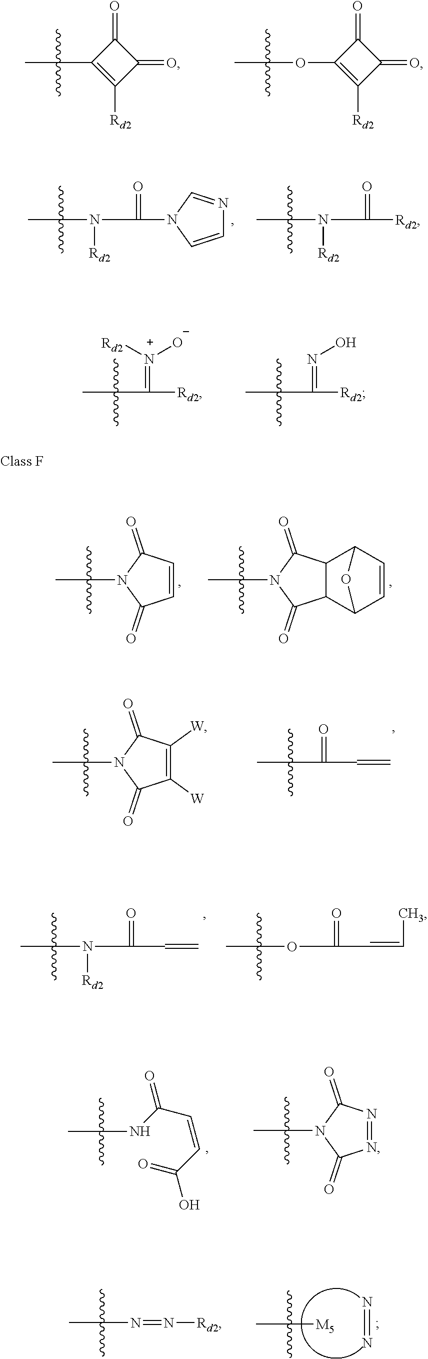

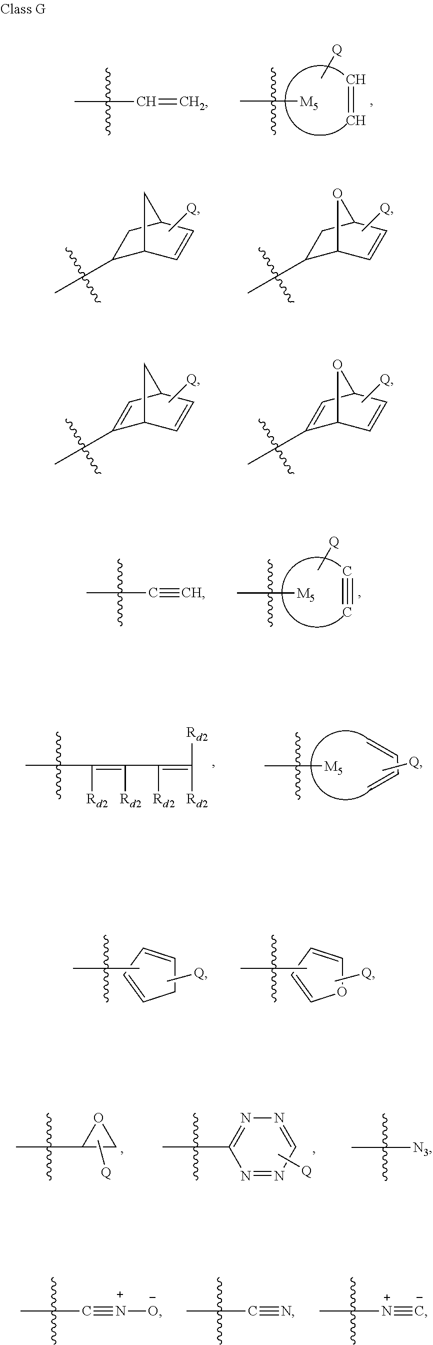

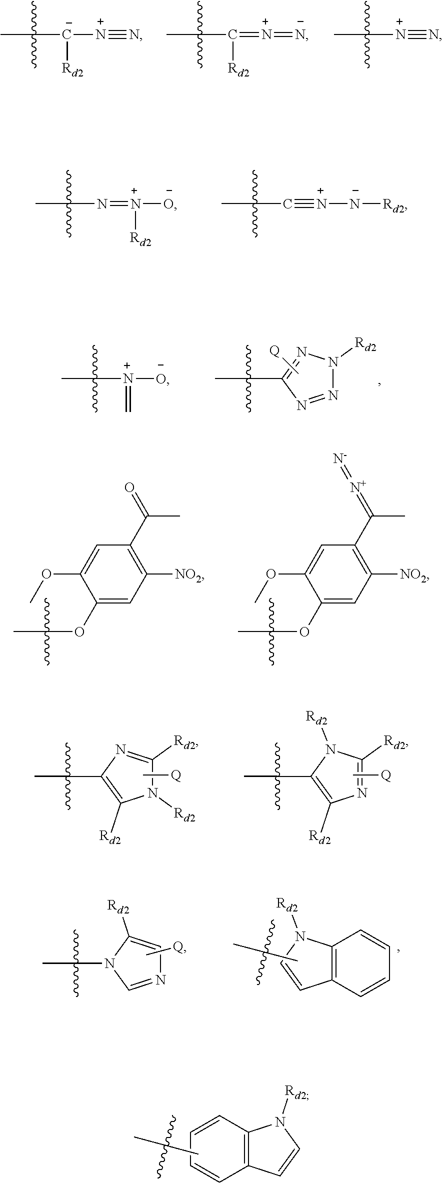

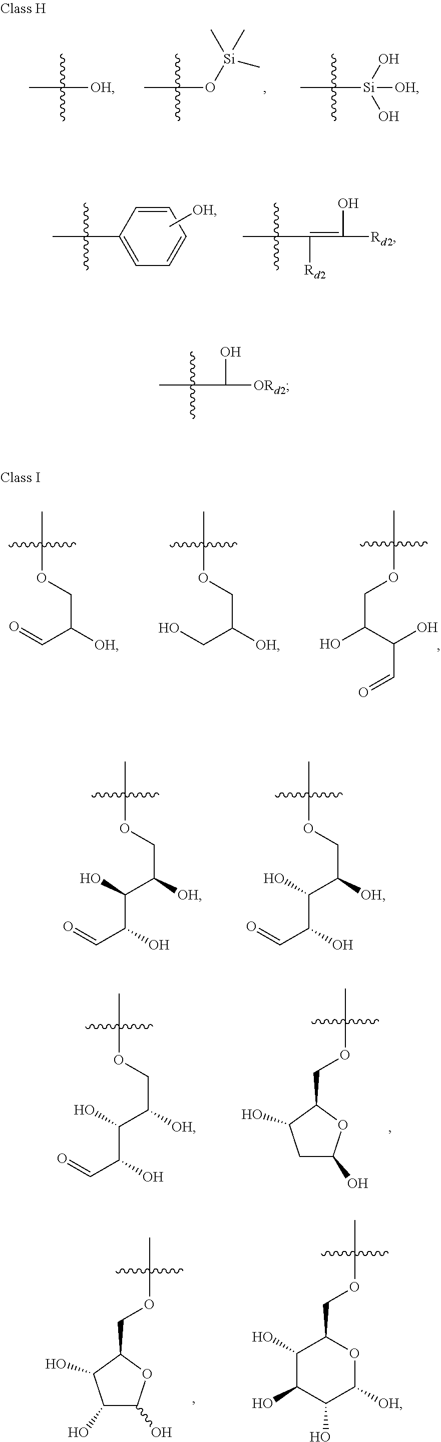

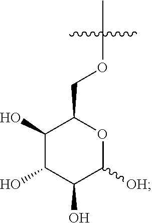

wherein, X is a halogen atom selected from the group consisting of fluorine, chlorine, bromine, and iodine atoms;
wherein, Y₁ is selected from the group consisting of C₁₋₅ alkyl, vinyl, phenyl, benzyl, p-methylphenyl, 4-(trifluoromethoxy)phenyl, trifluoromethyl, and 2,2,2-trifluoroethyl groups;
wherein, R_{d2} is an organic group and is preferably, at each occurrence, independently selected from the group consisting of C₁₋₅ alkyl, C₂₋₅ alkenyl, C₂₋₅ alkynyl, and phenyl groups, the alkyl, alkenyl, alkynyl and phenyl groups are each independently substituted or unsubstituted;
wherein, W is a leaving group selected from the group consisting of —F, —Cl, —Br, —I, and —SPh;
wherein, M₅ is a ring atom selected from the group consisting of a carbon atom, a nitrogen atom, a phosphorus atom, and a silicon atom; the cyclic structure containing M₅ is a 3-30 membered ring, preferably a 3-20 membered ring, more preferably a 3-16 membered ring, and more preferably a 5˜16 membered ring; the cyclic structure is selected from the group consisting of the following structures, substituted forms thereof, and heterosubstituted forms thereof: cyclohexane, furanose ring, pyranose ring, benzene, tetrahydrofuran, pyrrolidine, thiazolidine, cyclohexene, tetrahydropyran, piperidine, 1,4-dioxane, pyridine, pyridazine, pyrimidine, pyrazine, 1,3,5-triazine, 1,4,7-triazacyclononane, cyclotripeptide, indene, indane, indole, isoindole, purine, naphthalene, dihydroanthracene, xanthene, thioxanthene, dihydrophenanthrene, 10,11-dihydro-5H-dibenzo[a,d]cycloheptane, dibenzocycloheptene, 5-dibenzosuberenone, quinoline, isoquinoline, fluorene, carbazole, iminodibenzyl, acenaphthene, dibenzocyclooctyne, and aza-dibenzocyclooctyne;
wherein,

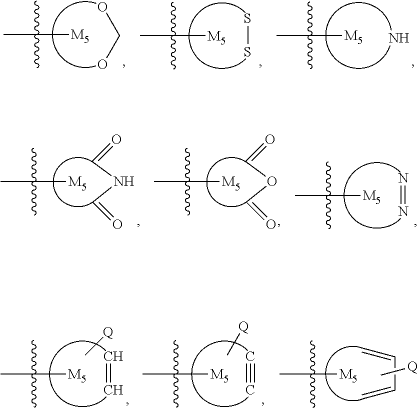

are cyclic structures of which the ring skeletons contain an acetal group, a disulfide bond, an amine group, an imide group, an anhydride group, an azo group, a carbon-carbon double bond, a carbon-carbon triple bond, and a conjugated diene, respectively; the cyclic structure is selected from the group consisting of a carbocycle, a heterocycle, a benzoheterocycle, a substituted carbocycle, a substituted heterocycle, and a substituted benzoheterocycle;
wherein, Q is an atom or substituent that promotes the inductive or conjugate effect of electrons of unsaturated bonds; there can be one or more Q when it is connected to a ring; when multiple Q are present, the structures of Q are identical or a combination of two or more different structures; when Q is a substituent, the structure of which is linear, branched with a pendant group, or ring-containing;
the modified form is selected from the group consisting of the precursors of reactive groups, the active forms to which reactive groups are precursors, the active forms in which reactive groups are substituted, and the inactive forms in which reactive groups are protected; wherein, the precursors of reactive groups refer to the structures which can be transformed into the reactive groups through at least one process among oxidation, reduction, hydration, dehydration, electronic rearrangement, structural rearrangement, salt complexation and decomplexation, ionization, protonation, and deprotonation.

In one specific embodiment of the present invention, R₀₁ is a reactive group selected from the group consisting of a hydroxyl group, a thiol group, an active ester group, an active carbonate group, a sulfonate group, an amino group, a maleimide group, a succinimide group, a carboxyl group, an acyl chloride group, an aldehyde group, an azido group, a cyano group, an alkenyl group, an alkynyl group, an epoxyalkyl group, a rhodamine group, a folate residue, a biotin residue, a monosaccharide group, and a polysaccharide group, or a modified form thereof; the modified form is selected from the group consisting of the precursors of reactive groups, the active forms to which reactive groups are precursors, the active forms in which reactive groups are substituted, and the inactive forms in which reactive groups are protected; wherein, the precursors of reactive groups refer to the structures which can be transformed into the reactive groups through at least one process among oxidation, reduction, hydration, dehydration, electronic rearrangement, structural rearrangement, salt complexation and decomplexation, ionization, protonation, and deprotonation; R₀₁ is preferably selected from the group consisting of the following structures:

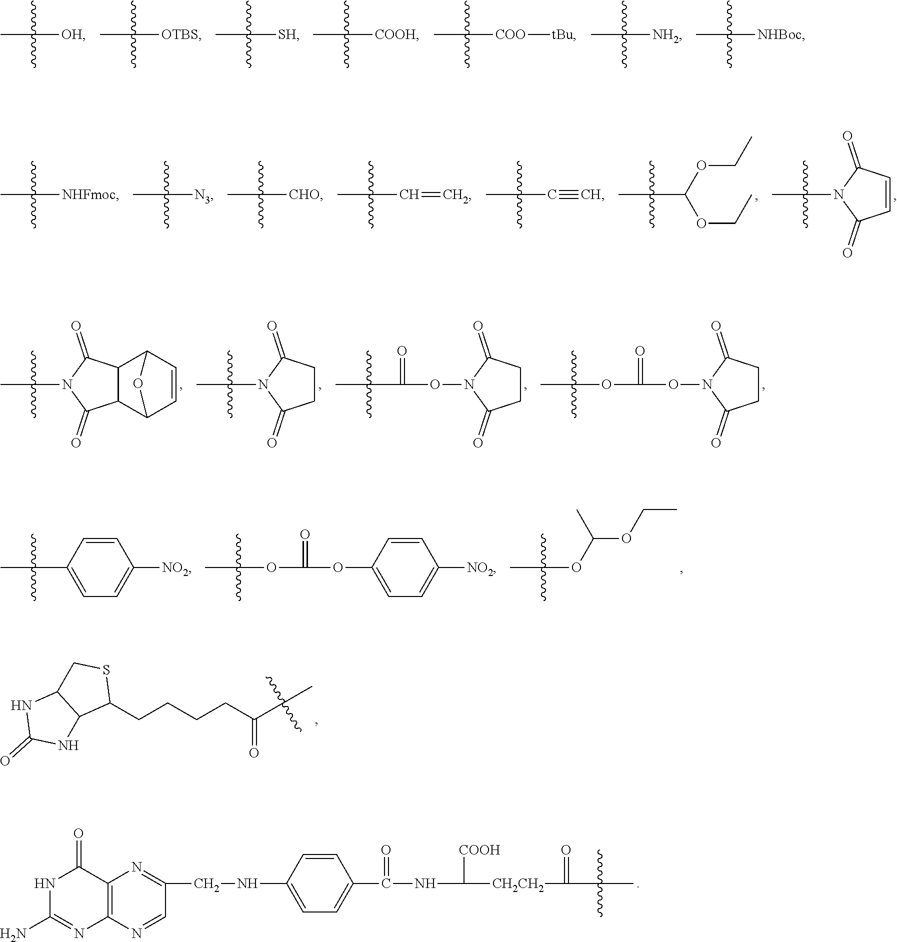

### 1.2.6. Lₓ, L_{d}

In one specific embodiment of the present invention, y instances of Lₓ are each independently selected from the group consisting of a linking bond, a hydrocarbylene group, a heteroatom-containing divalent hydrocarbon group, and combinations thereof, the heteroatom is B, O, N, Si, P, or S; any Lₓ together with the divalent linking group —O— in XPEG form the divalent linking group —O-Lₓ;
preferably, each of y instances of Lₓ independently corresponds to any of the following cases:
Case (1): Lₓ is a linking bond;
Case (2): Lₓ is a carbon-chain linking group, and is selected from the group consisting a hydrocarbylene group, a carbonyl group, a carbon-chain linking group with a heteroatom-containing side group, and combinations thereof,
Case (3): Lₓ is a linking group containing a heteroatom in the main chain; it is preferred that Lₓ or —O-Lₓ is selected from the group consisting of an ether group, a thioether group, a disulfide bond, a diselenide bond, an ester group, a monothioester group, a dithioester group, a carbonate group, a monothiocarbonate group, a dithiocarbonate group, a trithiocarbonate group, a secondary amino group, an amide group, a carbamate group, a monothiocarbamate group, a dithiocarbamate group, an imine group, a triazole linking group, a 4,5-dihydroisoxazole linking group, a 2,5-dioxopyrrolidine linking group, and combinations of any one, two or more thereof and hydrocarbylene groups; it is more preferred that Lₓ or —O-Lₓ is selected from the group consisting of an ether group, an ester group, a disulfide bond, an amide group, and a carbamate group;
Case (4): Lₓ or —O-Lₓ contains a linking group formed by a coupling reaction. Lₓ or —O-Lₓ is a linking group formed by an alkylation reaction, an amidation reaction, an esterification reaction, a thioesterification reaction, a click reaction, a cycloaddition reaction, a Diels-Alder addition reaction or a 1,3-dipolar cycloaddition reaction, or a combination of any one or more of the linking groups and hydrocarbylene groups;
more preferably, each of y instances of Lₓ or —O-Lₓ is independently selected from the group consisting of a linking bond, a hydrocarbylene group, —O—, —S—, —S—S—, —C(═O)—, —C(═O)O—, —OC(═O)—, —OC(═O)O—, —NH—, —C(═O)NH—, —NHC(═O)—, —OC(═O)NH—, —NHC(═O)O—, and combinations thereof,
the hydrocarbylene group is preferably —(CH₂)₅—, —(CH₂)ₜ₁—CH(R_{Ld})—(CH₂)₂—, or —(CH₂)ₜ₁—C(R_{Ld})₂—(CH₂)ₜ₂—; wherein, t₁ and t₂ are each independently an integer in the range of 0-4, the sum of t₁ and t₂ is an integer in the range of 0-4, and R_{Ld} is a C₁₋₁₅ hydrocarbon group or —OX₂, wherein X₂ is selected from the group consisting of a hydrogen atom, a methyl group, an ethyl group, a n-propyl group, an isopropyl group, a tert-butyl group, a pentyl group, a hexyl group, an allyl group, a trityl group, a phenyl group, a benzyl group, a nitrobenzyl group, a p-methoxybenzyl group, and a trifluoromethylbenzyl group.

In one specific embodiment of the present invention, y instances of Lₓ are each independently selected from the group consisting of a linking bond, a hydrocarbylene group, a heteroatom-containing divalent linking group, and combinations thereof;
the heteroatom-containing divalent linking group is preferably —O—, —S—, —S—S—, —C(═O)—, —C(═O)O—, —OC(═O)—, —OC(═O)O—, —NH—, —C(═O)NH—, —NHC(═O)—, —OC(═O)NH—, or —NHC(═O)O—;
the hydrocarbylene group is preferably a C₁₋₅ alkylene group;
preferably, each Lₓ is independently selected from the group consisting of —(CH₂)₂—, —C(═O)—, —CH₂C(═O)—, —C(═O)CH₂—, —C(═O)(CH₂)₂—, —CH₂CH₂C(═O)OCH₂CH₂—, —C(═O)NH—, —C(═O)NH(CH₂)₃—, and —C(═O)(CH₂)₂OC(═O)—, wherein the right end of Lₓ is connected to N_{core}.

In one specific embodiment of the present invention, L_{d} is a linking bond, a hydrocarbylene group, or any of the following classes:
(i) Covalent linking groups formed by coupling reactions involving any reactive group selected from the group consisting of amino, hydroxyl, carboxyl, mercapto, disulfide, diselenide, aldehyde, ketocarbonyl, guanidino, amido, azido, alkynyl, alkenyl, imidazolyl, and indolyl groups;
(ii) Amide group, secondary amino group, divalent tertiary amino group, carbamate group, thiocarbamate group, imine group, enamino group, aminoguanidino group, carbamimidamido group, imidate group, thioimidate group, ester group, carbonyl group, benzyloxycarbonyl group, thioester group, anhydride linking group, hydrazide group, acylhydrazone group, imide linking group, ether group, thioether group, disulfide bond, diselenide bond, guanidine-containing linking group, imidazole-containing linking group, triazole-containing linking group, 4,5-dihydroisoxazole linking group, vinyl ether bond, carbonate group, thiocarbonate group, dithiocarbonate group, trithiocarbonate group, dithiocarbamate group, acetal group, cyclic acetal group, thioacetal group, dithioacetal group, hemiacetal group, thiohemiacetal group, ketal group, thioketal group, oxime bond, thiooxime ether bond, semicarbazone bond, thiosemicarbazone bond, thiocarbohydrazide group, azocarbohydrazide group, azothiocarbohydrazide group, hydrazino formate group, hydrazino thioformate group, carbohydrazide group, thiocarbohydrazide group, allophanate group, thioallophanate group, guanidino group, amidino group, sulfonic acid group, sulfonate group, sulfinate group, orthoester group, phosphate group, phosphirate group, phosphinate group, phosphonate group, phosphosilicate group, silicate group, hydrazino group, urea bond, thiourea bond, isourea bond, isothiourea bond, thioamide group, sulphonamide group, phosphamide group, phosphoramidite group, pyrophosphamide group, cyclophosphamide group, ifosfamide group, thiophosphamide group, azaacetal group, azacycloacetal group, azathiaacetal group, azahemiacetal group, azaketal group, azacycloketal group, azathiaketal group, and combinations of any one or more thereof and hydrocarbylene groups;
the hydrocarbylene group is linear or contains a side chain, and is preferably a C₁₋₁₀ hydrocarbylene group; the side chain is a C₁₋₅ hydrocarbon group or —OX₂, wherein X₂ is selected from the group consisting of a hydrogen atom, a methyl group, an ethyl group, a n-propyl group, an isopropyl group, a tert-butyl group, a pentyl group, a hexyl group, an allyl group, a trityl group, a phenyl group, a benzyl group, a nitrobenzyl group, a p-methoxybenzyl group, and a trifluoromethylbenzyl group;
preferably, L_{d} is selected from the group consisting of a linking bond, a hydrocarbylene group, an ether group, a thioether group, an ester group, a carbonate group, a secondary amino group, an amide group, a carbamate group, a disulfide bond, an imine group, a combination of any one thereof and hydrocarbylene groups, and a combination of any two or more thereof and hydrocarbylene groups; more preferably, L_{d} is selected from the group consisting of a linking bond, an ether group, an ester group, a secondary amine group, an amide group, a combination of ether and hydrocarbylene groups, a combination of ester and hydrocarbylene groups, a combination of secondary amine and hydrocarbylene groups, a combination of amide and hydrocarbylene groups, a combination of ester, amide and hydrocarbylene groups, and a combination of ether, secondary amine and hydrocarbylene groups, and furtherly, preferably selected from the group consisting of a linking bond, —C(═O)O—, —OC(═O)—, —C(═O)NH—, —NHC(═O)—, —C(═O)O—Z—, —OC(═O)O—Z—, —C(═O)NH—Z—, —NHC(═O)—Z—, —C(═O)NH—Z—C(═O)O—Z—, and —O—Z—NH—Z—, wherein Z is, at each occurrence, independently a C₁₋₁₀ hydrocarbylene group and preferably a linear C₁₋₁₀ alkylene group.

In one specific embodiment of the present invention, L_{d} is selected from the group consisting of a linking bond, —CH₂—, —(CH₂)₂—, —CH₂C(═O)O—, —CH₂C(═O)OCH₂—, —(CH₂)₂C(═O)O—, —(CH₂)₂C(═O)OCH₂—, —(CH₂)₃OC(═O)—, —CH₂C(═O)NH—, —CH₂C(═O)NHCH₂—, —(CH₂)₂C(═O)NH—, —(CH₂)₂C(═O)NHCH₂—, —(CH₂)₃NHC(═O)—, —CH₂C(═O)NHCH₂C(═O)OCH₂—, and —(CH₂)₃O(CH₂)₃NHCH₂—.

### 1.2.7. N_{core}

In one specific embodiment of the present invention, N_{core} is an atomic, branched or cyclic multivalent core structure, and the multivalent atomic core is a trivalent nitrogen atomic core; when N_{core} is the multivalent branched core structure or the multivalent cyclic core structure, it contains at least one trivalent nitrogen atomic core and preferably corresponds any of the following cases:
Case (1): constructed from any multivalent residue of the following small molecules: a multifunctional small molecule, a hetero-multifunctional small molecule, and an amino acid;
Case (2): constructed from any of the following non-linear structures: a branched structure, a comb structure, a dendritic structure, a dendrimer-like structure, a cyclic structure, a hyperbranched structure, and combinations thereof,
wherein, the multifunctional small molecule is preferably a polyol, a polythiol, a polycarboxylic acid, a primary polyamine, or a secondary polyamine;
wherein, the hetero-multifunctional small molecule is preferably a polyol containing a heterofunctional group, a polythiol containing a heterofunctional group, a polycarboxylic acid containing a heterofunctional group, a primary polyamine containing a heterofunctional group, or a secondary polyamine containing a heterofunctional group;
wherein, the amino acid is preferably selected from the group consisting of glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, ornithine and citrulline, more preferably aspartic acid, glutamic acid, lysine, ornithine or glycine;
N_{core} is more preferably a residue of amino acid, a residue of dimeric amino acid, or a residue of poly-amino acid; the poly-amino acid contains at least 3 amino acid units; the amino acid units in the dimeric amino acid and those in the poly-amino acid are independent of each other, and any two amino acid units have identical or different structures.

In one specific embodiment of the present invention, y is 2, and N_{core} contains any type of trivalent core structure selected from the group consisting of atomic, branched and cyclic ones; wherein, a cyclic trivalent core is selected from the group consisting of aliphatic-ring, aromatic-ring, sugar-ring, and condensed-ring trivalent cores;
N_{core} is preferably any structure from any of the following classes:
(i) cyclic trivalent core structure, wherein the cyclic structure is a 3˜50 membered ring, preferably a 3˜32 membered ring, more preferably a 3˜18 membered ring, and more preferably a 5˜18 membered ring;
(ii) cyclic trivalent core structure containing nitrogen atoms, wherein the cyclic structure is selected from the group consisting of pyrrolidine, piperidine, pyrazine, 1,4,7-triazacyclononane, cyclic tripeptide, indole, isoindole, purine, carbazole, iminodibenzyl, azadibenzocyclooctyne, substituted forms thereof, and heterosubstituted forms thereof,
(iii) constructed from any trivalent residue of the following structures: a triol, a tri-thiol, a primary triamine, a secondary triamine, a tricarboxylic acid, a trisulfonate, a triisocyanate, a hetero-trifunctional small molecule, and an amino acid; the hetero-trifunctional small molecule is a heterofunctional small molecule containing three functional groups, which contains two different types of functional groups, wherein the number of one type is 1 and the number of the other type is 2.

In one specific embodiment of the present invention, y is 3, and N_{core} contains an atomic, branched, or cyclic tetravalent core structure or contains a combination of two trivalent core structures; N_{core} is preferably selected from the group consisting of the following structures:

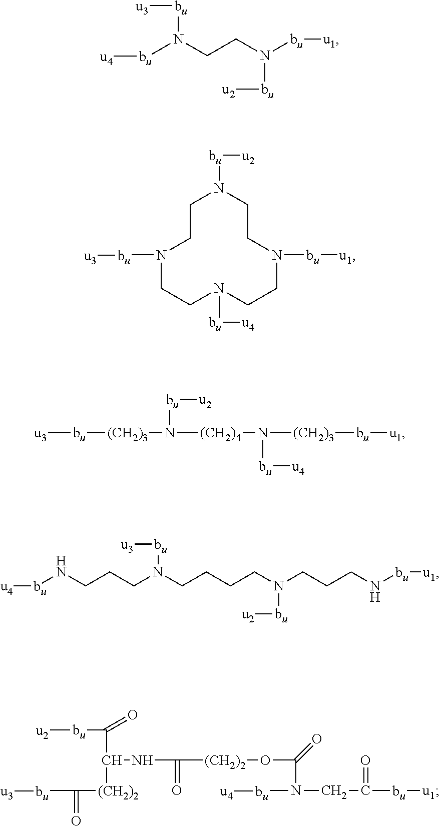

wherein, u₁, u₂, u₃ and u₄ are all linking bonds; u₁, u₂, u₃ and u₄ are each independently connected to L_{d} or any Lₓ, and any two of u₁, u₂, u₃ and u₄ are not simultaneously connected to either L_{d} or any Lₓ; every bᵤ is each independently a linking bond or a C₁₋₆ divalent linking group, preferably —(CH₂)₀₋₆—.

In one specific embodiment of the present invention, y≥4, the valence of N_{core}>5, and N_{core} is selected from the group consisting of a comb structure, a dendritic structure, a branched structure, a hyperbranched structure and a cyclic structure;
the dendritic structure is selected from the group consisting of the following structures:

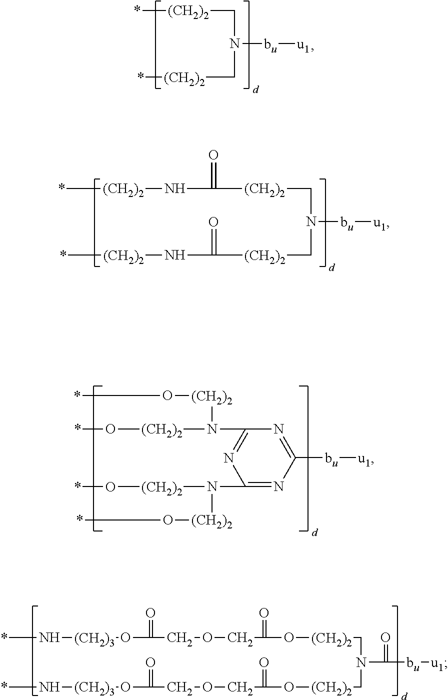

wherein, u₁ is a linking bond connected to L_{d}, and the asterisk marker * in the structures represents a linking bond connected to a polyethylene glycol component; d represents the number of generations in the dendritic combination, and d is selected from the group consisting of 2, 3, 4, 5 and 6; bᵤ is —(CH₂)₀₋₆—;
the branched structure, hyperbranched structure or comb structure is selected from the group consisting of a trivalent core structure, a tetravalent core structure, a pentavalent core structure, and combinations thereof, when the combination is a combination of trivalent core structures, the number of the trivalent core structures is two or greater; the branched structure, hyperbranched structure or comb structure is preferably a residue of polymer formed by any one or more types of amino acids or derivatives thereof, or an oxidized form of the residue;
the cyclic structure is any of the following structures: the residue of a cyclopeptide composed of basic amino acids or derivative thereof, and the skeleton of 1,4,7-tri-tert-butoxycarbonyl-1,4,7,10-tetraazacyclododecane.

In one specific embodiment of the present invention, N_{core} is selected from the group consisting of

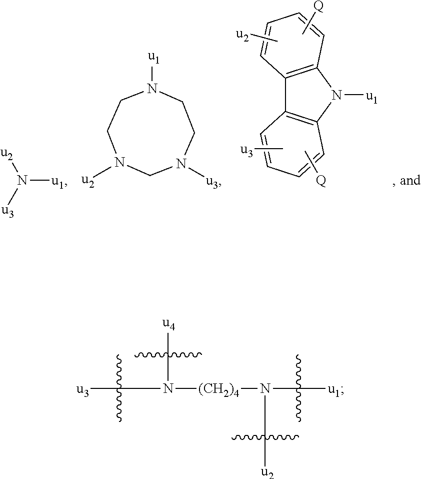

wherein, u₁, u₂, u₃ and u₄ are each independently a linking bond connected to L_{d} or Lₓ, and any two of u₁, u₂, u₃ and u₄ are not simultaneously connected to the same L_{d} or Lₓ;
wherein, Q is an electron-changing group, the number of which is 0, 1 or greater than 1; when the number of Q is greater than 1, any two Q have the same or different structures; the number of Q is preferably 0.

### 1.2.8. Examples of Specific Structures

In one specific embodiment of the present invention, the structure of PEGylated lipid is selected from the group consisting of the following structures:

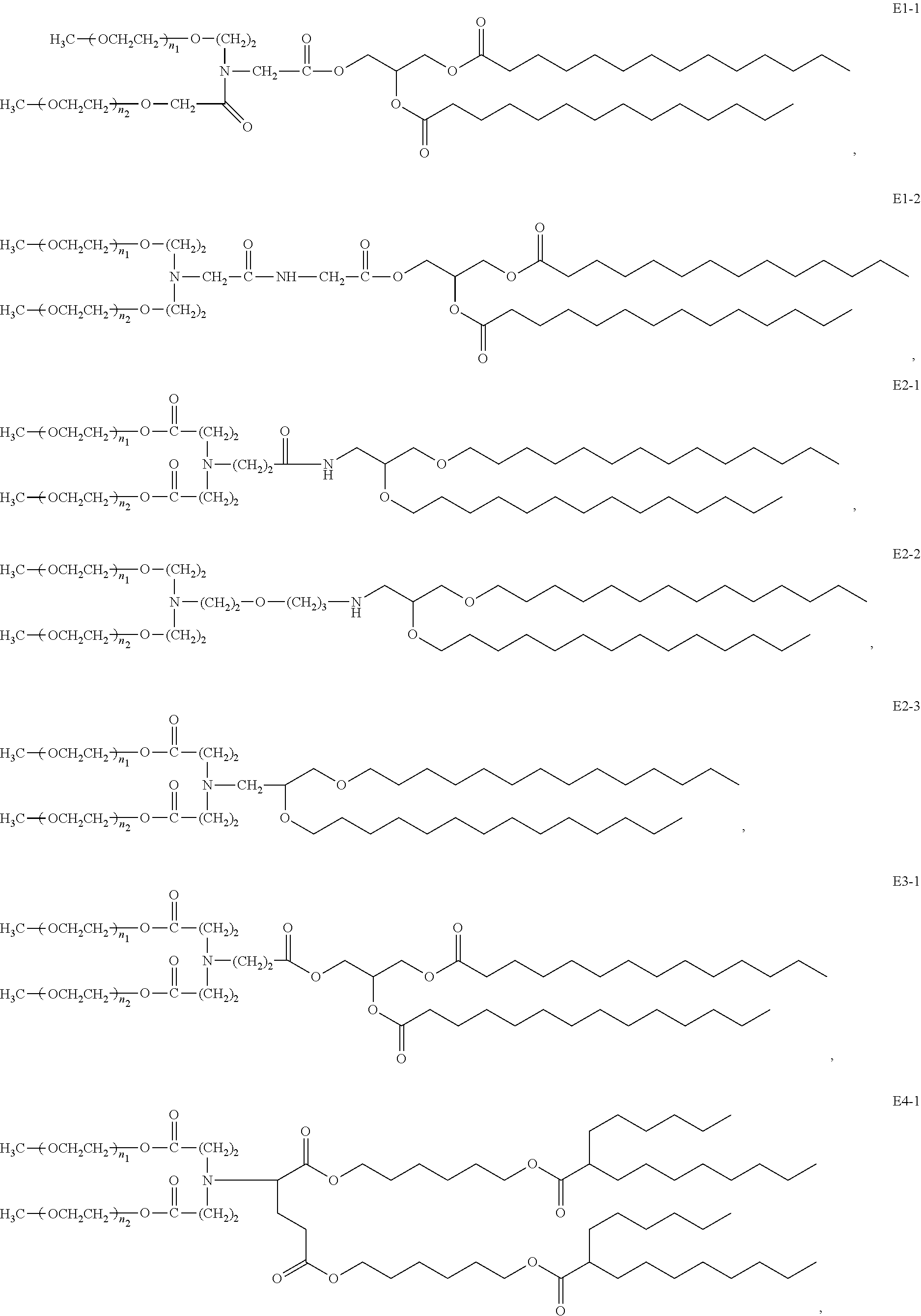

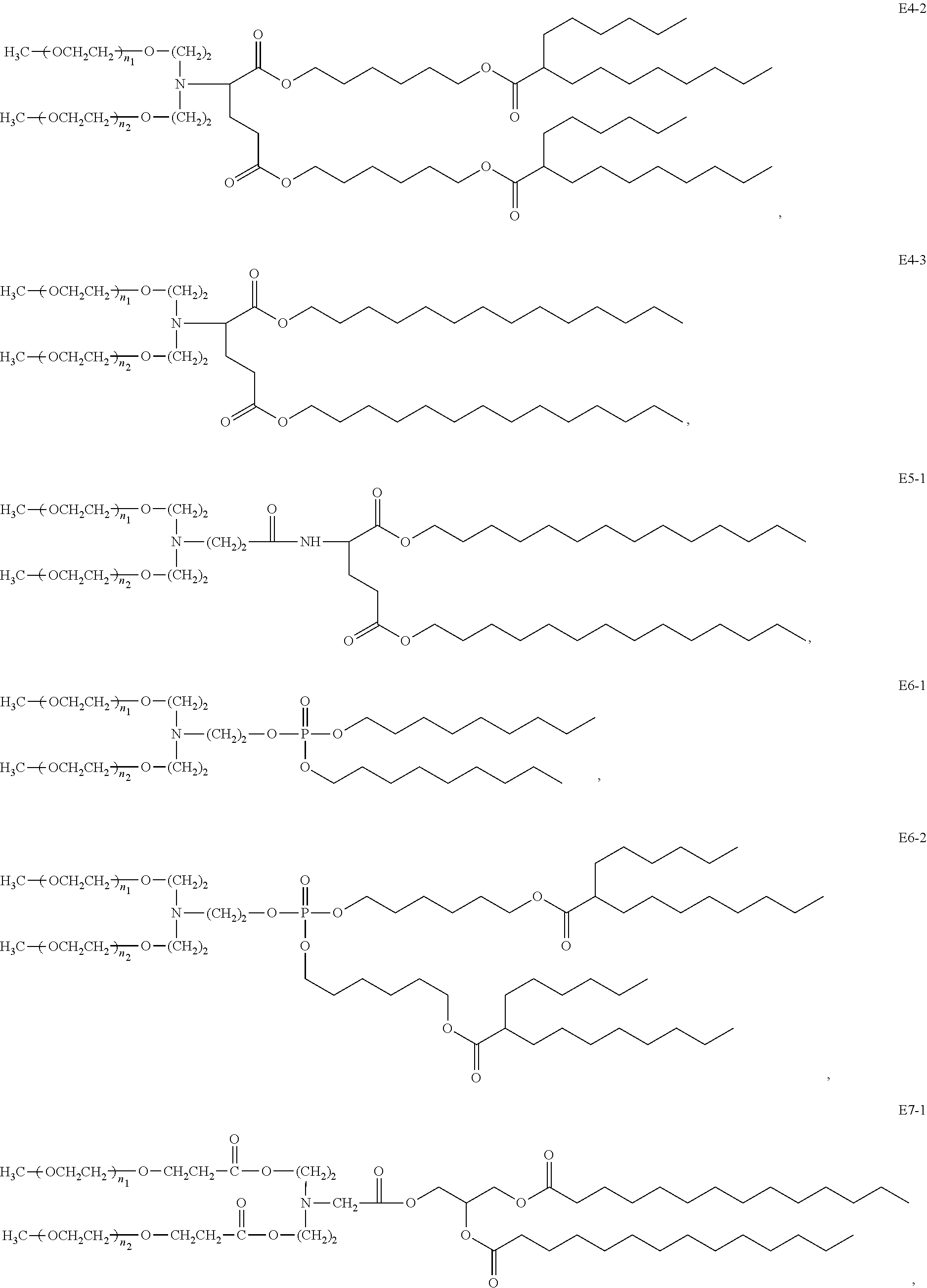

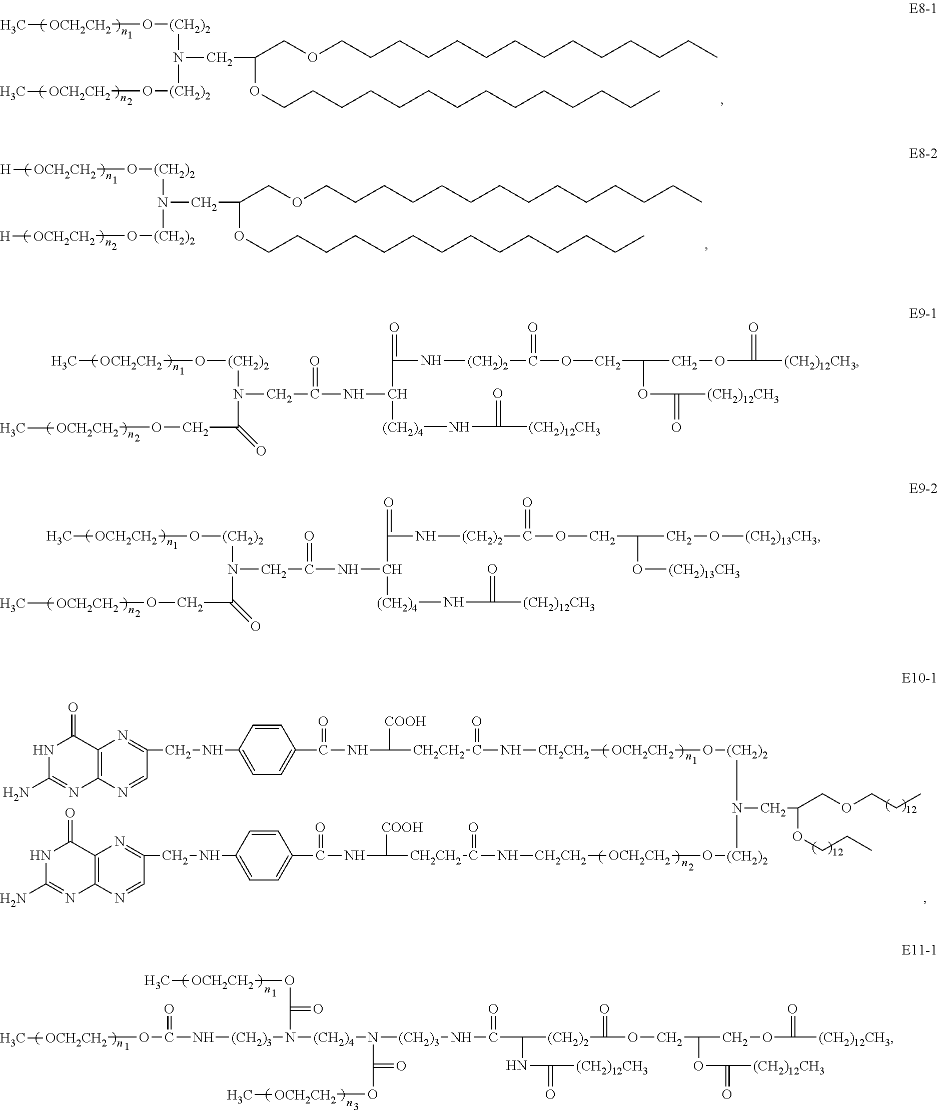

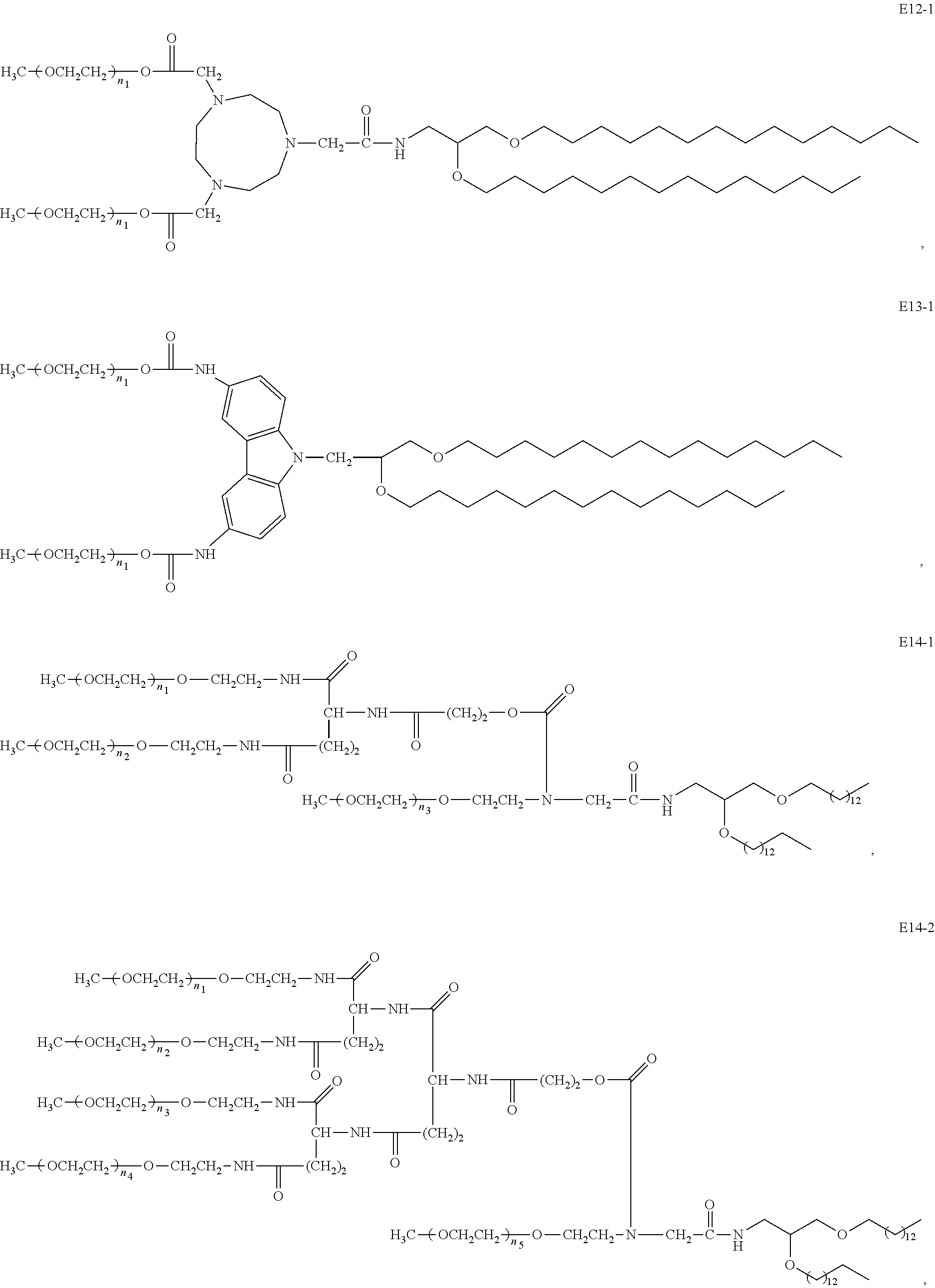

### 1.3. Preparation Methods of PEGylated Lipids

In one specific embodiment of the present invention,
provided herein is a preparation method of any aforementioned PEGylated lipid, wherein the PEGylated lipid is prepared according to the formula (4) or (5):

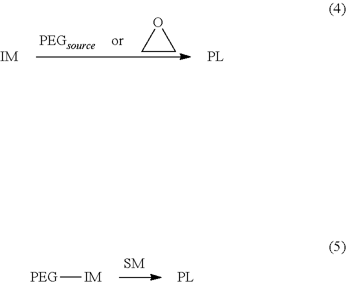

wherein, IM is a low-molecular-weight lipid intermediate, PEG_{source} is an active polyethylene glycol derivative, PEG-IM is a bifunctional, non-linear polyethylene glycol derivative intermediate, SM is a small molecule compound,

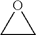

is ethylene oxide, PL is the PEGylated lipid.

In one specific embodiment of the present invention, the structure of the aforementioned IM is selected from the group consisting of the following structures and protected forms thereof, wherein the protected form contains a protected amino group or hydroxyl group:

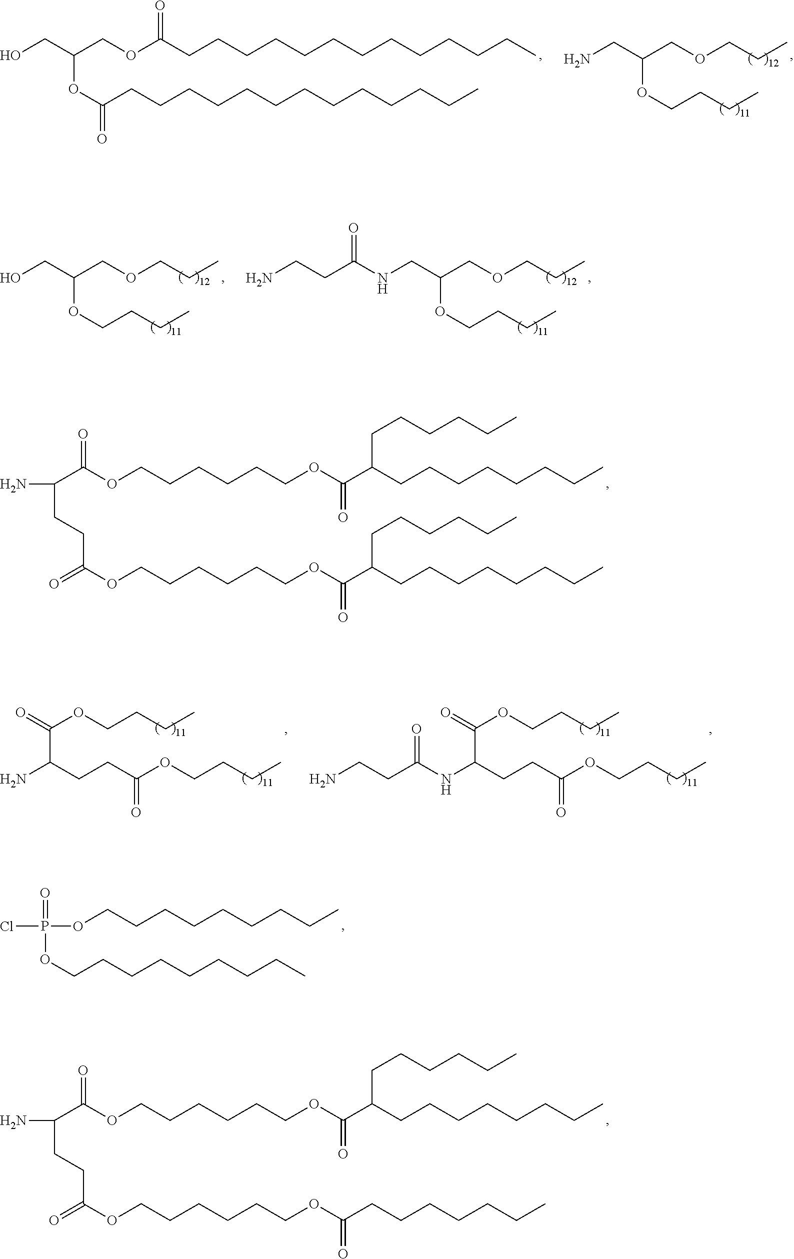

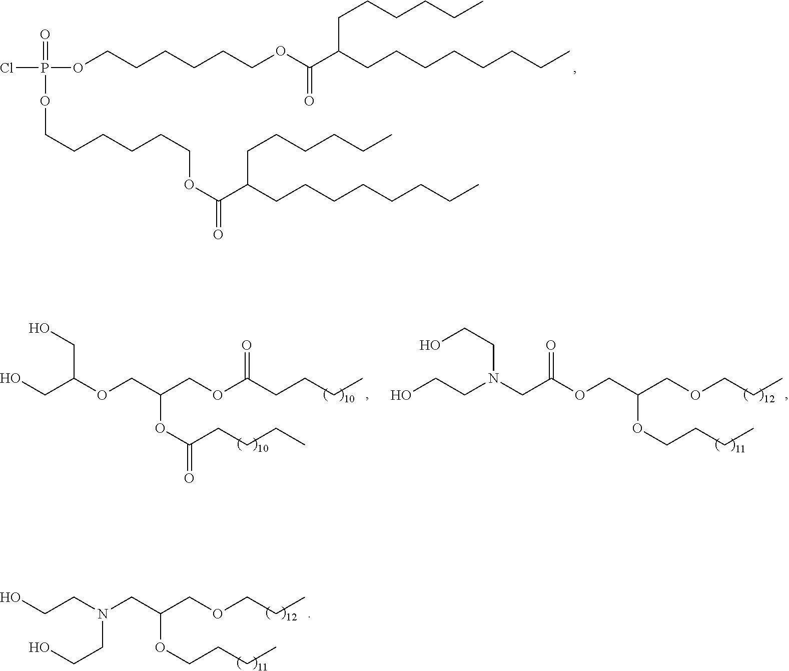

In one specific embodiment of the present invention, the aforementioned IM reacts with PEG_{source} to obtain PL; wherein, PEG_{source} is selected from the group consisting of the following structures and protected forms thereof:

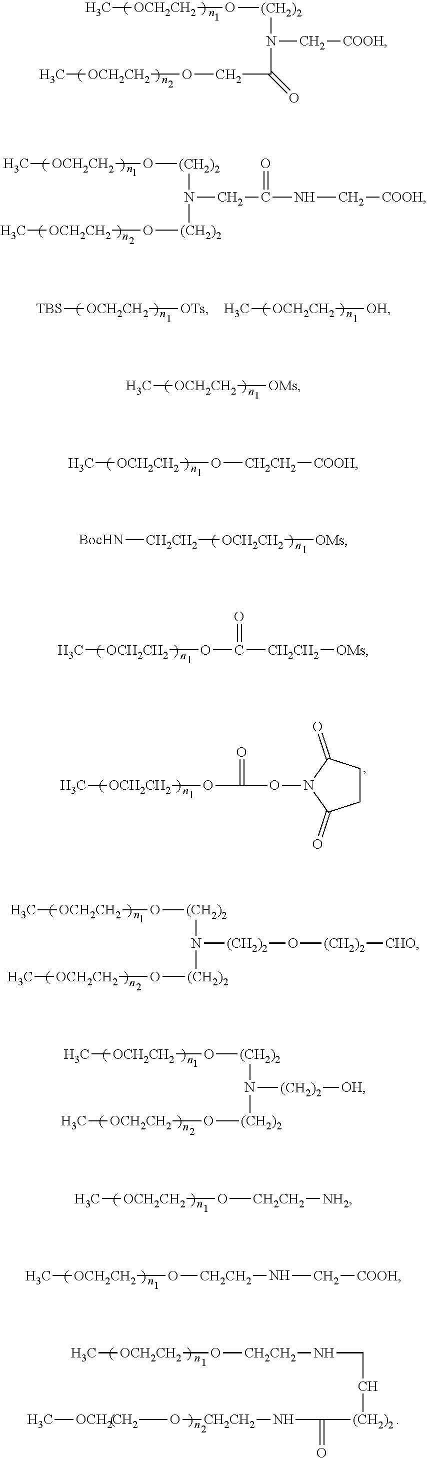

In one specific embodiment of the present invention, the aforementioned IM acts as an initiator and initiates the polymerization of ethylene oxide after deprotonation to obtain PL; IM is selected from the group consisting of the following structures:

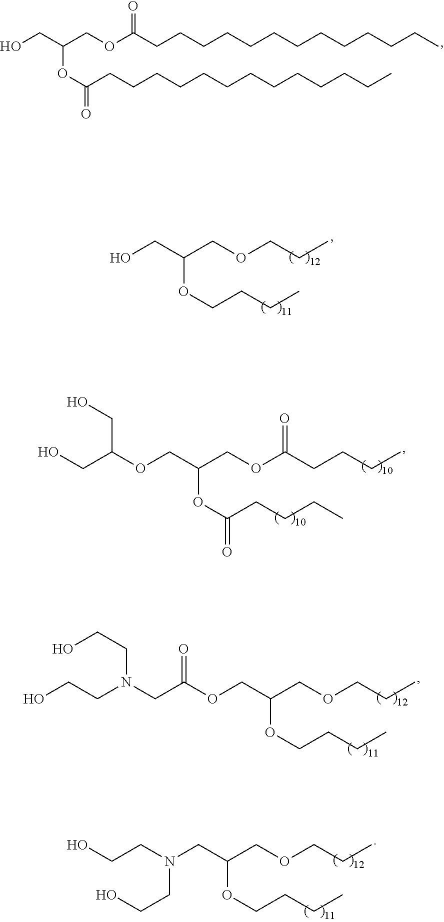

In one specific embodiment of the present invention, the aforementioned PEG-IM is selected from the group consisting of the following structures and protected forms thereof:

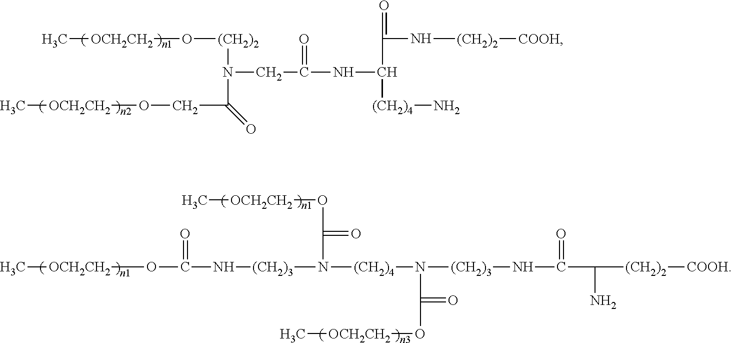

In one specific embodiment of the present invention, the aforementioned PEG-IM and SM undergo coupling reactions to obtain PL; wherein, SM is selected from the group consisting of the following structures:

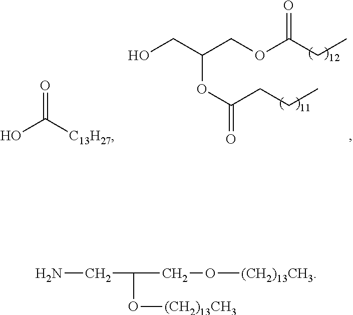

In one specific embodiment of the present invention, a PEGylated lipid can be obtained from a preparation process containing coupling reactions and/or polymerization reactions; the coupling reaction is not particularly limited with respect to the range of options, as long as two identical or different reactive groups can form a covalent linking group through the reaction; a preparation process can contain a single-step or stepwise coupling reaction, and each step of the coupling reaction is preferably, independently selected from the group consisting of alkylation reaction, condensation reaction, amidation reaction, esterification reaction, thioesterification reaction, ring-opening reaction, ring-closing condensation reaction, addition reaction, cycloaddition reaction, addition reaction of α,β-unsaturated bonds, addition reaction of alkynes, Schiff base reaction-reduction reaction, click reaction, azide-alkyne addition reaction, 1,3-dipolar cycloaddition reaction, Diels-Alder addition reaction, thiol-yne reaction, thiol-ene reaction, thiol-vinyl reaction, and condensation reaction. The reaction conditions for the coupling reactions are relevant to the type of the covalent linking groups formed in the reactions, which may include the prior art. The valence state of a covalent linking group obtained from the coupling reactions could be divalent or trivalent, and preferably divalent. Groups obtained from the coupling reactions can be stable groups or degradable groups;
the polymerization reaction includes at least the following two steps: deprotonation of the small molecule initiator, and polymerization of ethylene oxide; the small molecule initiator can be a readily available starting material, or an intermediate in the preparation process;
in the preparation process, when coupling and polymerization reactions both exist, the order in which the coupling and polymerization reactions take place is not limited.

In the present invention, the starting materials used in every preparation methods can be obtained by purchase or synthesis.

In the present invention, with respect to the preparation method of monodisperse PEGylated lipids, the monodisperse starting materials containing polyethylene glycol components can be replaced by polydisperse starting materials containing the same components, therefore obtaining the corresponding polydisperse products; similarly, with respect to the preparation method of polydisperse PEGylated lipids, the polydisperse starting materials containing polyethylene glycol components can be replaced by monodisperse starting materials containing the same components, therefore obtaining the corresponding monodisperse products.

In one specific embodiment of the present invention, polyethylene glycol starting materials including but not limited to linear/non-linear polyethylene glycols and derivatives thereof can be prepared by referring to the methods in CN108530637B, CN110591079A, CN108659227A, CN108530617B, CN1243779C, or CN101029131A.

The intermediates and end-products prepared in the present invention can be purified by a purification method including but not limited to extraction, recrystallization, adsorption treatment, precipitation, reverse precipitation, membrane dialysis, supercritical extraction, etc. Characterization methods including but not limited to NMR, electrophoresis, UV-visible spectrophotometer, FTIR, AFM, GPC, HPLC, MALDI-TOF, circular dichroism spectroscopy, etc., can be used in the characterization and determination of the structure and molecular weight of end-products.

In the present invention, the following reaction schemes described in 1.3.1 to 1.3.5 do not represent the actual complete preparation routes. The actual preparation process can also include any necessary procedures which are familiar to those skilled in the art, such as micro-modification, protection/deprotection, intermediate preparation, workup, purification, etc.

### 1.3.1. Preparation of Low-Molecular-Weight Lipid Intermediates

In one specific embodiment of the present invention, PEGylated lipids are prepared using low-molecular-weight lipid intermediates; the structure of the low-molecular-weight lipid intermediate (IM) is selected from the group consisting of the following:

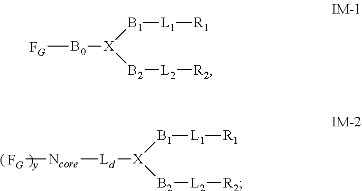

wherein, B₀ is selected from the group consisting of a linking bond, a hydrocarbylene group, a heteroatom-containing divalent hydrocarbon group, —O—, —S—, —S—S—, —C(═O)—, —C(═O)O—, —OC(═O)—, —OC(═O)O—, —NH—, —C(═O)NH—, —NHC(═O)—, —OC(═O)NH—, —NHC(═O)O—, and combinations thereof, preferably a linking bond or hydrocarbylene group; wherein, F_{G} is a reactive group or protected form thereof, which can participate in a covalent reaction to produce a divalent linking group, either directly, or after deprotection, or after activation; when the number of F_{G} in a single molecule is greater than 1, any two F_{G} have identical or different structures; the structure of F_{G} in IM-1 is identical or different from that of any F_{G} in IM-2; F_{G} is preferably selected from the group consisting of a hydrogen atom, an amino group, a carboxyl group, an azido group, a hydroxyl group, a halide group, an aldehyde group, an alkenyl group, an alkynyl group, a succinimidyl group, a maleimide group, and a thiol group, or a combination of alkylene groups and any aforementioned reactive group or protected form thereof (e.g., —CH₂CH₂NH₂ is a combination of an ethylene group and an amino group, also belonging to F_{G});
wherein, IM-1 contains 1 F_{G}, and is preferably selected from the group consisting of the following structures:

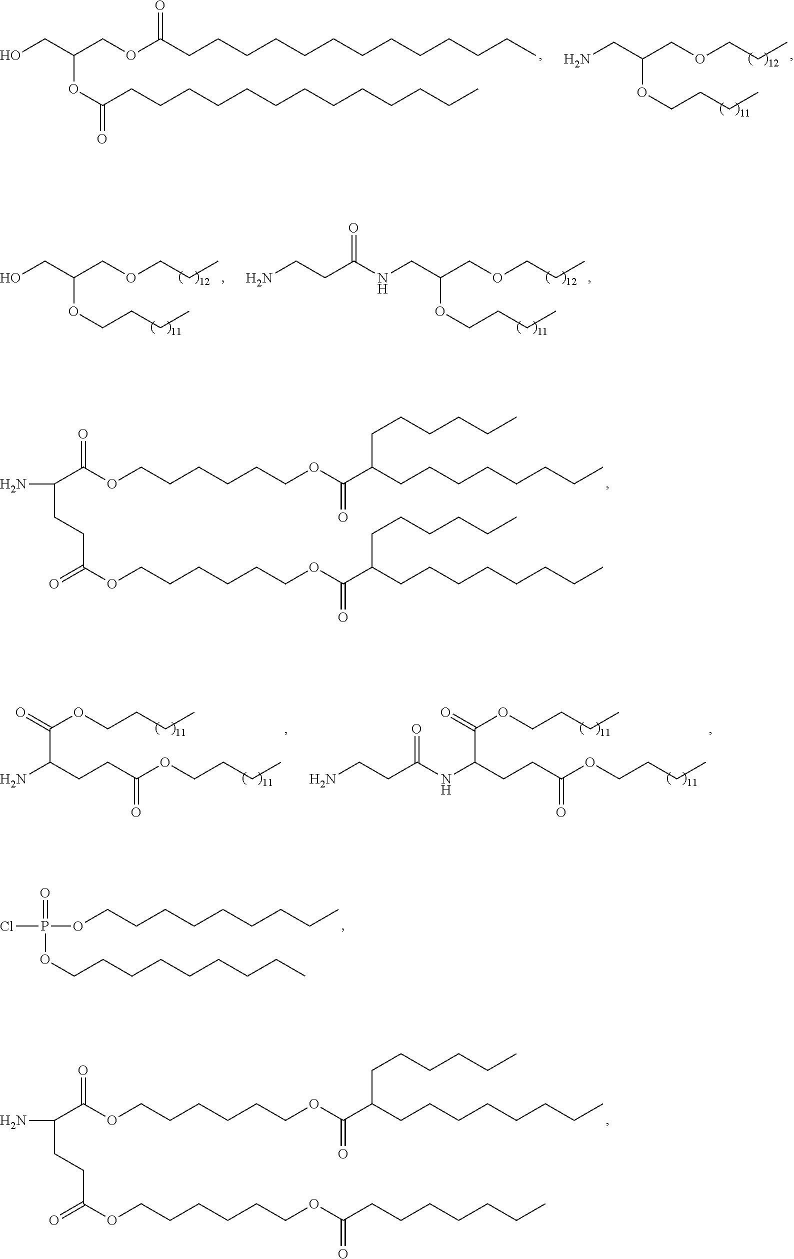

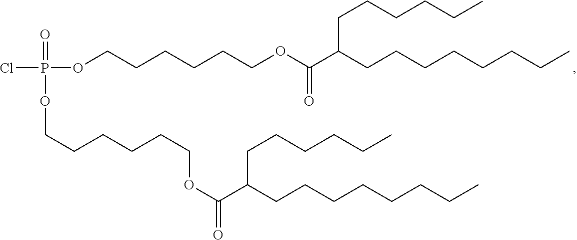 [SMILES: CCCCCCCCC(CCCCCC)C(=O)OCCCCCCOP(=O)(Cl)OCCCCCCOC(=O)C(CCCCCC)CCCCCCCC]

and the corresponding forms in which the terminal reactive groups are protected;
wherein, IM-2 contains y F_{G}, and is preferably selected from the group consisting of the following structures:

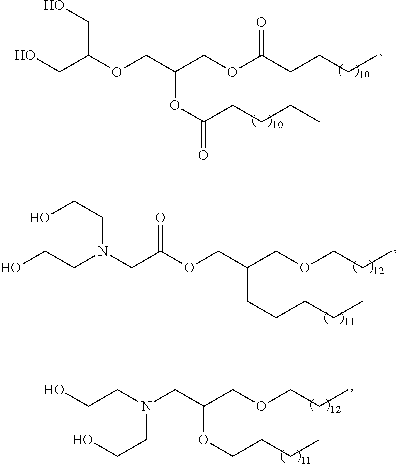

the corresponding forms in which the terminal reactive groups are protected.

### 1.3.1.1. Preparation of Low-Molecular-Weight Lipid Intermediate IM-1

In a more specific embodiment of the present invention, the low-molecular-weight lipid intermediate IM-1 can be obtained through any of the following methods:

### Method (1)

Step 1: the reactive group F_{G1} of the small molecule A-5 reacts with the reactive group F_{N3} of the small molecule A-6 to form the divalent group L₁, therefore obtaining the intermediate IM-1′;
Step 2: the reactive group F_{G2} of the intermediate IM-1′ reacts with the reactive group F_{N4} of the small molecule A-7 to form the divalent linking group L₂, therefore obtaining the low-molecular-weight lipid intermediate IM-1;
wherein, L₁ and L₂ have the same or different structures, R₁ and R₂ have the same or different structures, B₁ and B₂ have the same or different structures, F_{G1} and F_{G2} have the same or different structures, and F_{N3} and F_{N4} have the same or different structures;
when L₁=L₂, R₁=R₂, F_{G1}=F_{G2}, and F_{N3}=F_{N4}, the step 1 and step 2 are both carried out or not carried out; when the step 1 and step 2 are both not carried out, the optional step is carried out: the reactive groups F_{G1} and F_{G2} of the small molecule A-5 both react with the reactive group F_{N3} of the small molecule A-6, or both react with the reactive group F_{N4} of the small molecule A-7, to form the divalent groups L₁ and L₂, therefore obtaining the low-molecular-weight lipid intermediate IM-1;
the steps are as follows:

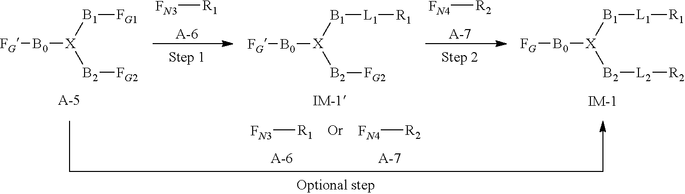

wherein, the small molecule A-5 also contains the terminal group F_{G}′, and the structure of F_{G}′ is the same as or different from that of F_{G}; when the structures of F_{G}′ and F_{G} are different, F_{G}′ can be transformed into F_{G} through micro-modifications, and the micro-modification is selected from the group consisting of the following chemical reactions and combinations thereof: deprotection, salt complexation and decomplexation, ionization, protonation, deprotonation, and changing leaving groups; preferably, F_{G}′ is a protected form of F_{G};

For example, in Example 1.1, S1-1 corresponds to A-5 in the above-described routes, S1-2 corresponds to A-6 or A-7 in the above-described routes, and S1-3 (corresponding to IM-1) is obtained through the optional step:

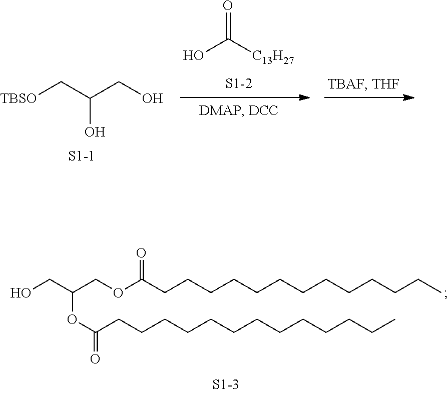

### Method (2)

Step 1: the reactive group F_{G1} of the small molecule A-5′ reacts with the reactive group F_{N3} of the small molecule A-6 to form the divalent group L₁, therefore obtaining the intermediate INT-1;
Step 2: the reactive group F_{G2} of the intermediate INT-1 reacts with the reactive group F_{N4} of the small molecule A-7 to form the divalent linking group L₂, therefore obtaining the intermediate INT-2;
wherein, L₁ and L₂ have the same or different structures, R₁ and R₂ have the same or different structures, B₁ and B₂ have the same or different structures, F_{G1} and F_{G2} have the same or different structures, and F_{N3} and F_{N4} have the same or different structures;
when L₁=L₂, R₁=R₂, F_{G1}=F_{G2}, and F_{N3}=F_{N4}, the step 1 and step 2 are both carried out or not carried out; when the step 1 and step 2 are both not carried out, the optional step is carried out: the reactive groups F_{G}i and F_{G2} of the small molecule A-5′ both react with the reactive group F_{N3} of the small molecule A-6, or both react with the reactive group F_{N4} of the small molecule A-7, to form the divalent groups L₁ and L₂, therefore obtaining the intermediate INT-2;
Step 3: the reactive group F_{A}′ of the intermediate INT-2 reacts with the reactive group F_{B}′ of the small molecule A-8 to form a divalent linking group which, together with Z_{F} and B₀′, form the divalent linking group B₀, therefore obtaining the low-molecular-weight lipid intermediate IM-1; wherein, Z_{F} and B₀′ are both divalent linking groups, preferably hydrocarbylene groups;
the steps are as follows:

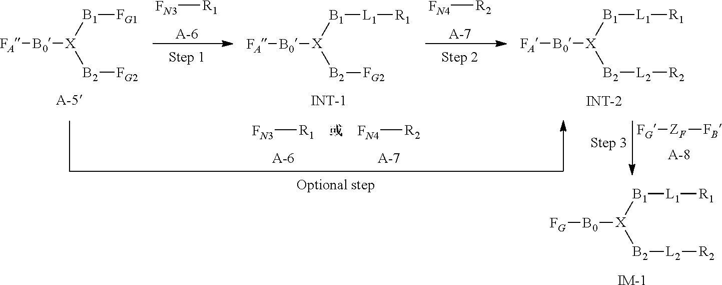

wherein, the small molecule A-5′ also contains the terminal group F_{A}″, and the structure of F_{A}″ is the same as or different from that of F_{A}′; when the structures of F_{A}″ and F_{A}′ are different, F_{A}″ can be transformed into F_{A}′ through micro-modifications, and the micro-modification is selected from the group consisting of the following chemical reactions and combinations thereof: deprotection, salt complexation and decomplexation, ionization, protonation, deprotonation, and changing leaving groups; preferably, F_{A}″ is a protected form of F_{A}′;
wherein, the small molecule A-8 also contains the terminal group F_{G}′, and the structure of F_{G}′ is the same as or different from that of F_{G}; when the structures of F_{G}′ and F_{G} are different, F_{G}′ can be transformed into F_{G} through micro-modifications, and the micro-modification is selected from the group consisting of the following chemical reactions and combinations thereof: deprotection, salt complexation and decomplexation, ionization, protonation, deprotonation, and changing leaving groups; preferably, F_{G}′ is a protected form of F_{G};

For example, in Example 2.1, S2-2 corresponds to A-5′ in the above-described routes, S2-1 corresponds to A-6 or A-7 in the above-described routes, and S2-3 (corresponding to INT-2 in the above-described routes) is obtained through the optional step; S2-4 (corresponding to A-8 in the above-described routes) and S2-3 undergo the Step 3 to obtain S2-5 (corresponding to IM-1):

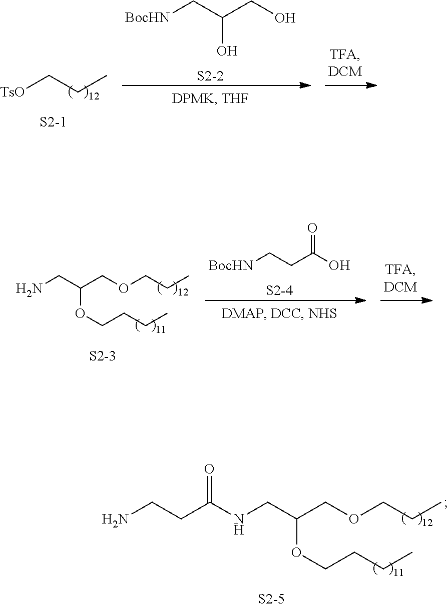

the reactive groups F_{G1} and F_{G2} in the methods (1) and (2) are preferably selected from the group consisting of a hydrogen atom, an amino group, a carboxyl group, a hydroxyl group, a halide group, an aldehyde group, an alkenyl group, an alkynyl group, a succinimidyl group, a maleimide group, a thiol group, and protected forms thereof, wherein the protected form participates in the reaction after deprotection;
the definitions of the aforementioned X, B₁, B₂, L₁, L₂, R₁, R₂, F_{G}, and B₀ are consistent with those in the formula (1) and the formula IM-1, and will not be repeated here.

### 1.3.1.2. Preparation of Low-Molecular-Weight Lipid Intermediate IM-2

In a more specific embodiment of the present invention, the low-molecular-weight lipid intermediate IM-2 is obtained through the following method:

The reactive group F_{G} of the low-molecular-weight lipid intermediate IM-1 reacts with the reactive group F_{G3} of the small molecule A-1 to form a divalent linking group which, together with the B₀ in IM-1 and the B₀ in A-1, form the divalent linking group L_{d}, therefore obtaining the low-molecular-weight lipid intermediate IM-2; wherein, the reactive group F_{G3} is preferably selected from the group consisting of a hydrogen atom, an amino group, a carboxyl group, a hydroxyl group, a halide group, an aldehyde group, an alkenyl group, an alkynyl group, a succinimidyl group, a maleimide group, a thiol group, and protected forms thereof, wherein the protected form participates in the reaction after deprotection;

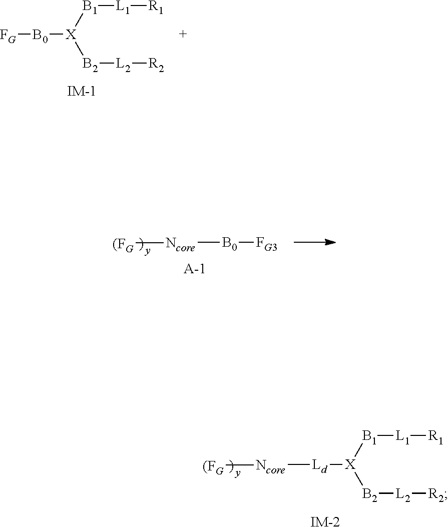

wherein, the definitions of the aforementioned X, B₁, B₂, L₁, L₂, R₁, R₂, F_{G}, and B₀ are consistent with those in the formula (1) and the formula IM-1, and will not be repeated here.

### 1.3.2. Introduction of Polyethylene Glycol Components

In a more specific embodiment of the present invention, the preparation route also contains the step of introducing XPEG components; the step is selected from any of the following methods:

Method (1): the active polyethylene glycol derivative PEG_{source} and the low-molecular-weight lipid intermediate IM-1 or IM-2 undergo a single-step or stepwise coupling reaction to introduce y instances of XPEG components to obtain a structure represented by the general formula (1), either directly or after micro-modification at the terminals; the steps are as follows:

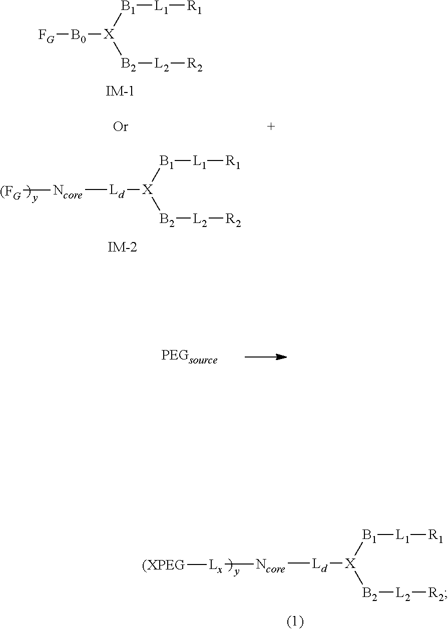

wherein, PEG_{source} contains at least one RPEG single-chain component;
furthermore, any of the following cases is preferred:
Case I: PEG_{source-1} and the low-molecular-weight lipid intermediate IM-1 undergo a single-step coupling reaction to introduce y instances of XPEG components at one time; wherein, PEG_{source-1} contains a non-linear structure composed of y instances of XPEG components, and the reactive group F_{G4} of PEG_{source-1} reacts with the F_{G} of IM-1 to form a divalent linking group which, together with B₀, form the divalent linking group L_{d}; the non-linear structure is selected from the group consisting of a branched structure, a comb structure, a dendritic structure, a dendrimer-like structure, a hyperbranched structure, and a cyclic structure; the step is as follows:

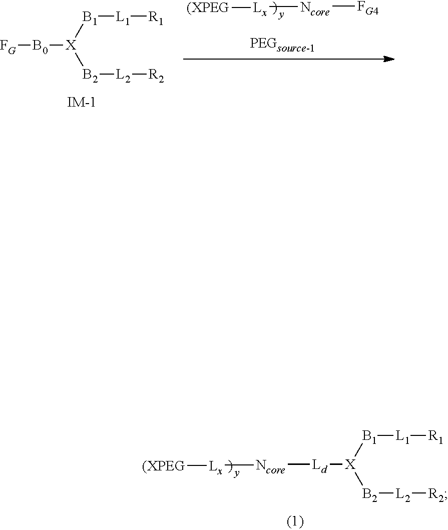

For example, in Example 1.1, S1-3 corresponds to IM-1, S1-4 corresponds to PEG_{source-1}, both of which undergo the above route to obtain E1-1 (corresponding to the general formula (1)):

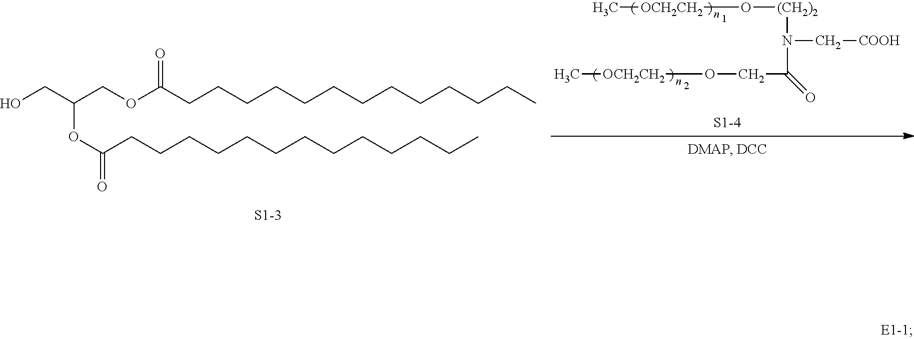

Case II: PEG_{source-2} and the low-molecular-weight lipid intermediate IM-2 undergo a single-step or stepwise coupling reaction to introduce y instances of XPEG components; wherein, PEG_{source-2} contains only one XPEG component, and the reactive group F_{G5} of PEG_{source-2} reacts with the F_{G} of IM-2 to form a divalent linking group which, together with B₀, form the divalent linking group Lₓ; the steps are as follows:

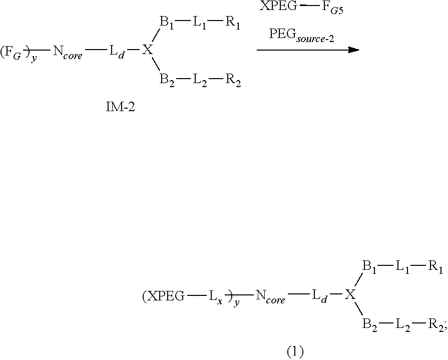

For example, in Example 2.1, S2-5 corresponds to IM-2, and S2-6 corresponds to PEG_{source-2}, which undergo the above route to obtain E2-1 (corresponding to the general formula (1)):

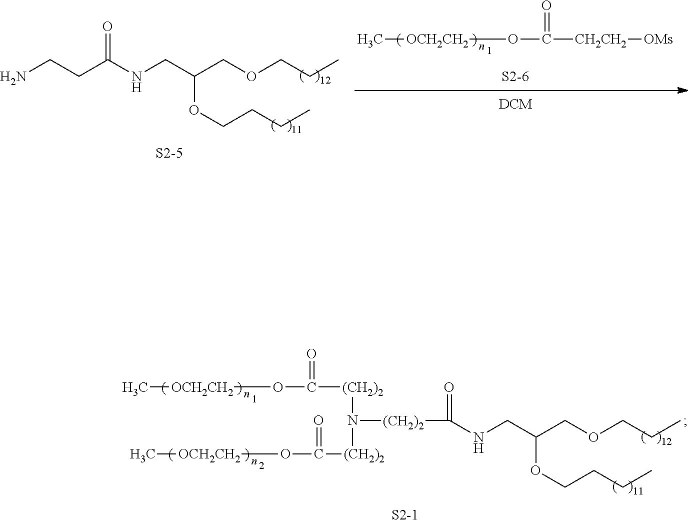

Method (2): the low-molecular-weight lipid intermediate IM-2 is used as a small-molecule initiator, and initiates the polymerization of ethylene oxide after deprotonation. The obtained polymerization product is a mixture of alcohol and oxyanion, which is subjected to a capping reaction after complete deprotonation, and a structure represented by the general formula (1) is obtained, either directly or after micro-modification at the terminals.

Specific examples include Example 8.1 and Example 8.2:

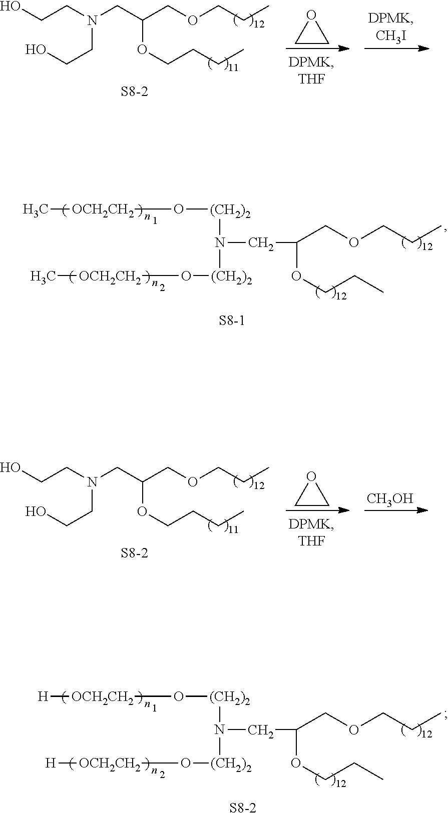

The base used for the deprotonation is not particularly limited, preferably metallic sodium, potassium, sodium hydride, potassium hydride, sodium methoxide, potassium methoxide, lithium naphthalene, n-butyllithium, tert-butyllithium, potassium tert-butoxide or diphenylmethyl potassium, more preferably metallic sodium, potassium, or diphenylmethyl potassium, most preferably diphenylmethyl potassium;
the micro-modification at the terminals in methods (1) and (2) is selected from the group consisting of the following chemical reactions and combinations thereof: deprotection, salt complexation and decomplexation, ionization, protonation, deprotonation, and changing leaving groups.

### 1.3.3. Use of Bifunctional PEG Intermediate

In one specific embodiment of the present invention, PEGylated lipids are prepared using bifunctional, non-linear polyethylene glycol derivative intermediates; the bifunctional, non-linear polyethylene glycol derivative intermediate has a structure represented by PEG-IM as follows:

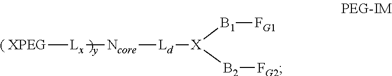

PEG-IM undergoes coupling reactions with small molecules to obtain the non-linear PEGylated lipid represented by the general formula (1); the steps are as follows:

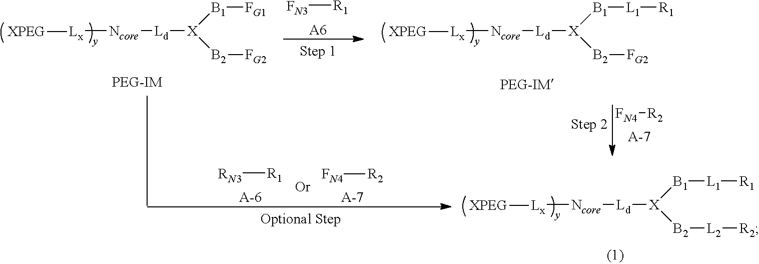

Step 1: the reactive group F_{G1} of PEG-IM reacts with the reactive group F_{N3} of the small molecule A-6 to form the divalent linking group L₁, therefore obtaining the intermediate PEG-IM-1′;

Step 2: the reactive group F_{G2} of the intermediate PEG-IM-1′ reacts with the reactive group F_{N4} of the small molecule A-7 to form the divalent linking group L₂, therefore obtaining the non-linear PEGylated lipid represented by the general formula (1);
wherein, L₁ and L₂ have the same or different structures, R₁ and R₂ have the same or different structures, B₁ and B₂ have the same or different structures, F_{G1} and F_{G2} have the same or different structures, and F_{N3} and F_{N4} have the same or different structures;
when L₁=L₂, R₁=R₂, F_{G1}=F_{G2}, and F_{N3}=F_{N4}, the step 1 and step 2 are both carried out or not carried out; when the step 1 and step 2 are both not carried out, the optional step is carried out: the reactive groups F_{G1} and F_{G2} of PEG-IM both react with the reactive group F_{N3} of the small molecule A-6, or both react with the reactive group F_{N4} of the small molecule A-7, to form the divalent groups L₁ and L₂, therefore obtaining the non-linear PEGylated lipid represented by the general formula (1);
wherein, the reactive groups F_{G1} and F_{G2} are preferably selected from the group consisting of a hydrogen atom, an amino group, a carboxyl group, a hydroxyl group, a halide group, an aldehyde group, an alkenyl group, an alkynyl group, a succinimidyl group, a maleimide group, a thiol group, and protected forms thereof, wherein the protected form participates in the reaction after deprotection;

For example, in Example 9.1, S9-1 corresponds to PEG-IM, S1-2 corresponds to A-6, S9-2 corresponds to PEG-IM′, and S1-3 corresponds to A-7, which undergo the Step 1 and Step 2 in the above route to obtain E9-1 (corresponding to the general formula (1)):

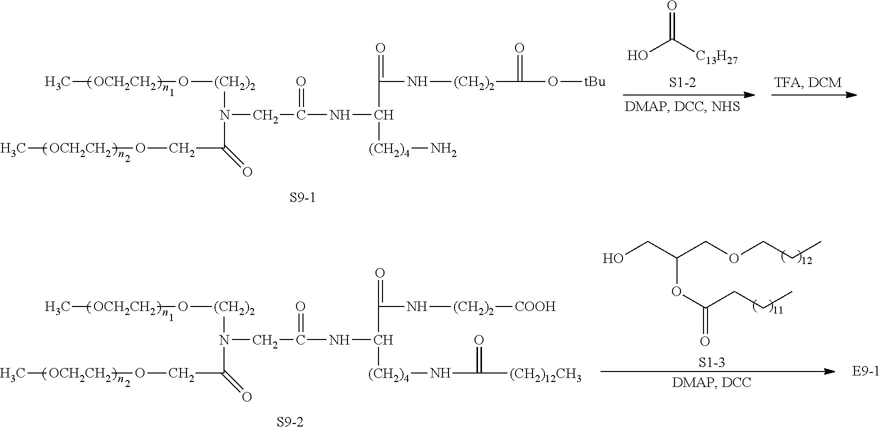

### 1.3.4. Alkylation Reactions

In the present invention, alkylation reactions are preferably those based on hydroxyl groups, thiol groups, or amine groups, corresponding to the formation of ether bonds, thioether bonds, and secondary/tertiary amine groups, respectively. Examples are as follows:

(1) Alkylation Reaction of Amine Substrates with Sulfonates or Halides

In the presence of a base, an amine intermediate can be obtained via the nucleophilic substitution with a sulfonate derivative or halide on the amine substrate. Wherein, the molar equivalent of the sulfonate or halide is 1 to 50 times that of the amine substrate, preferably 1 to 5 times. When the molar equivalent of the sulfonate or halide is less than 1 fold of that of the amine substrate, the substitution might be incomplete, and the purification could be difficult. When the molar equivalent of the sulfonate or halide exceeds 50 times that of the amine substrate, the excess sulfonate or halide tends to bring difficulty in the purification process, and might be brought into the subsequent step and therefore increase the amount of side reactions which further increases difficulty in the purification.

The resulting product is a mixture of amine intermediate and excess sulfonate or halide, and can be purified by methods such as anion exchange resin, osmosis, ultrafiltration, etc. Wherein, the anion exchange resin is not particularly limited, as long as the target product can undergo ion-exchange and adsorption in the resin, and is preferably the ion exchange resin of tertiary amines or quaternary ammonia salts with the matrix being dextran, agarose, polyacrylate, polystyrene, or poly(diphenylethylene), etc. The solvents used for osmosis or ultrafiltration are not limited, generally water or organic solvents. Said organic solvent is not particularly limited, as long as the product can be dissolved within, but is preferably dichloromethane, chloroform, etc.

The reaction solvent is not limited, preferably an aprotic solvent such as toluene, benzene, xylene, acetonitrile, ethyl acetate, tetrahydrofuran, chloroform, dichloromethane, dimethylsulfoxide, dimethylformamide, and dimethylacetamide, and more preferably dimethylformamide, dichloromethane, dimethylsulfoxide, or tetrahydrofuran.

The base used can be an organic base (e.g., triethylamine, pyridine, 4-dimethylaminopyridine, imidazole, diisopropylethylamine) or an inorganic base (e.g., sodium carbonate, sodium hydroxide, sodium bicarbonate, sodium acetate, potassium carbonate, potassium hydroxide), preferably an organic base, and more preferably triethylamine or pyridine. The molar equivalent of the base used is 1 to 50, preferably 1 to 10, more preferably 3 to 5 times that of the sulfonate or halide.

(2) Alkylation reaction of amine substrates with aldehyde derivatives

The amine substrate reacts with an aldehyde derivative to obtain an imine intermediate, which is followed by obtaining an intermediate using reducing agents. Wherein, the molar equivalent of the aldehyde derivative is 1 to 20, preferably 1 to 2, more preferably 1 to 1.5 times that of the amine substrate. When the molar equivalent of the aldehyde derivative exceeds 20 times that of the amine substrate, the excess reagent tends to cause difficulty in the purification process, and might be brought into the subsequent step and therefore increase difficulty in the purification. When the molar equivalent of the aldehyde derivative is less than 1 fold of that of the amine substrate, the reaction might be incomplete, causing further difficulty in the purification. Wherein, the resulting product can be obtained after purification by methods such as cation exchange resin, osmosis, ultrafiltration, etc. Said cation exchange resin is not particularly limited, as long as it can undergo ion-exchange with quaternary ammonium cations and then realize the isolation. The solvents used for osmosis or ultrafiltration treatment are not limited, generally water or organic solvents. Said organic solvent is not particularly limited, as long as the product can be dissolved within, but is preferably dichloromethane, chloroform, etc.

The reaction solvent is not limited, preferably an organic solvent such as methanol, ethanol, water, toluene, benzene, xylene, acetonitrile, ethyl acetate, tetrahydrofuran, chloroform, dichloromethane, dimethylsulfoxide, dimethylformamide, dimethylacetamide, etc., and more preferably water or methanol.

The reducing agent is not particularly limited, as long as the imine can be reduced to an amine, but is preferably sodium borohydride, lithium aluminum hydride, sodium cyanoborohydride, or Zn/AcOH, etc., and more preferably sodium cyanoborohydride. The amount of the reducing agent used is generally 0.5 to 50 times, preferably 1 to 10 times that of the aldehyde derivative.

### 1.3.5. End-Functionalization of RPEG

In one specific embodiment of the present invention, all the RPEG ends of the PEGylated lipid contain the same functional group R₀₁, and R₀₁ is preferably introduced by the active polyethylene glycol derivative PEG_{source} in the unprotected or protected form.

In one specific embodiment of the present invention, starting materials include the active polyethylene glycol derivative PEG_{source}. The structure of PEG_{source} can be any specific structure of monofunctional non-linear polyethylene glycol documented in CN108530637B, CN110591079A, CN108659227A, CN108530617B, and CN1243779C.

In one specific embodiment of the present invention, the end-functionalization can be further applied to a PEGylated lipid of the general formula (1), (2), or (3), and the obtained structure after the end-functionalization still belongs to the general formula (1), (2), or (3). The method for the end-functionalization is not particularly limited but is related to the type of the final functional group or its protected form, mainly including the functionalization of the terminal hydroxyl group and the transformation from a reactive group to the target functional group or its protected form.

In the present invention, specific preparation methods of the functionalization of the terminal hydroxyl group include but are not limited to those documented in the paragraphs [0960]-[1205] in CN104530417A.

In the present invention, the transformation from a reactive group to the target functional group or its protected form can be realized through any of the following methods:

Method 1: direct modification. The direct modification based on a reactive group produces the target functional group or its protected form. Examples include the transformation of a carboxyl group into an acyl halide group, a hydrazide group, an ester group, a thioester group, or a dithioester group, the transformations of a hydroxyl group, a thiol group, an alkynyl group, an amino group, a carboxyl group, etc., into the corresponding protected forms, and the modifications of a hydroxyl group, an amino group, etc., with anhydrides.

Method 2: coupling reaction between two reactive groups. A heterofunctional reagent is used as a starting material containing 1 type of reactive group as well as the target functional group, and the reactive group reacts with the terminal reactive group of RPEG to introduce the target functional group or its protected form. The reaction of two reactive groups is not particularly limited with respect to the manner or method, and the reaction conditions are relevant to the type of the divalent linking group formed in the reaction. Prior arts such as alkylation, addition of alkenes, addition of alkynes, Schiff base reaction-reduction reaction, condensation, etc., can be used herein. Wherein, the alkylation reaction is preferably based on thiol group or amino group, corresponding to the formation of thioether bond and secondary or tertiary amino group, respectively. Wherein, the condensation reaction includes but is not limited to those forming ester groups, thioester groups, amide groups, imine bonds, hydrazone bonds, carbamate groups, etc. The target functional group or its protected form can also be introduced via click reactions using starting materials including a heterofunctional reagent containing not only a reactive group selected from the group consisting of an azido group, an alkynyl group, an alkenyl group, a tri-thioester group, a thiol group, a diene group, a furyl group, a 1,2,4,5-tetrazinyl group, a cyanate group, etc., but also the target functional group or its protected form. The reaction of two reactive groups is accompanied by the formation of new bonds, and typical representatives of newly formed divalent linking groups include amide bond, urethane bond, ester group, secondary amine bond, thioether bond, triazole group, etc.

Method 3: combination of direct modification and coupling reaction. The target functional group or its protected form can be obtained via the combination.

### 1.4. Lipid Compositions, Lipid Pharmaceutical Compositions and Formulations Thereof, Liposomes, and Lipid Nanoparticles

In one embodiment of the present invention,
provided herein is a lipid composition containing any aforementioned PEGylated lipid.

In a more specific embodiment of the present invention, the lipid composition contains another one or more types of lipids selected from the group consisting of phospholipid, steroid lipid, and PEGylated lipid, corresponding to any of the following cases:
Case (1): the lipid composition also contains a phospholipid;
Case (2): the lipid composition also contains a steroid lipid;
Case (3): the lipid composition also contains a PEGylated lipid;
Case (4): the lipid composition also contains a phospholipid and a steroid lipid;
Case (5): the lipid composition also contains a phospholipid and a PEGylated lipid;
Case (6): the lipid composition also contains a steroid lipid and a PEGylated lipid;
Case (7): the lipid composition also contains a phospholipid, a steroid lipid, and a PEGylated lipid;
and preferably contains another three types of lipids which are phospholipid, steroid lipid, and PEGylated lipid, simultaneously.

In a more specific embodiment of the present invention, the phospholipid contained in the lipid composition is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dioleoylphosphatidylserine (DOPS), dipalmitoylphosphatidylglycerol (DPPG), palmitoyloleoylphosphatidylethanolamine (POPE), distearoylphosphatidylethanolamine (DSPE), dipalmitoylphosphatidylethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), and compositions thereof, the phospholipid can be synthetic or naturally sourced.

In a more specific embodiment of the present invention, the steroid lipid contained in the lipid composition is selected from the group consisting of cholesterol, coprostanol, sitosterol, ergosterol, campesterol, stigmasterol, brassicasterol tomatidine, ursolic acid, α-tocopherol, and compositions thereof.

In a more specific embodiment of the present invention, the cationic lipid contained in the lipid composition is selected from the group consisting of N,N-dioleyl-N,N-dimethylammonium chloride (DODAC), N,N-distearyl-N,N-dimethylammonium bromide (DDAB), N-[1-(2,3-dioleoyloxy)propyl]-N,N,N-trimethylammonium chloride (DOTAP), N-[1-(2,3-dioleyloxy)propyl]-N,N,N-trimethylammonium chloride (DOTMA), N,N-dimethyl-2,3-dioleyloxy-1-(dimethylamino)propane (DODMA), 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), ((4-hydroxybutyl)azanediyl)bis(hexane-6,1-diyl)bis(2-hexyldecanoate) (ALC-0315), heptadecan-9-yl 8-((2-hydroxyethyl)(6-oxo-6-(undecyloxy)hexyl)amino)octanoate (SM102),

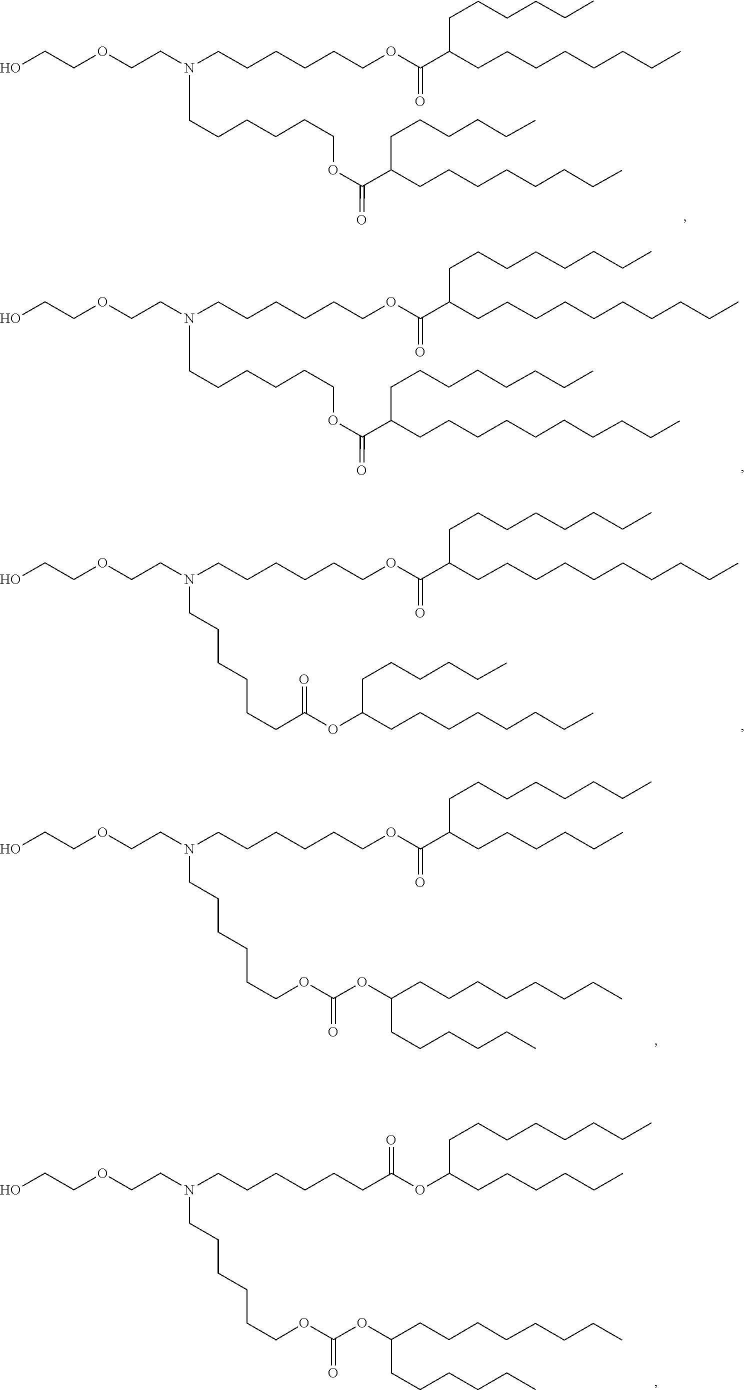 [SMILES: CCCCCCCCC(CCCCCC)C(=O)OCCCCCCN(CCCCCCOC(=O)C(CCCCCC)CCCCCCCC)CCOCCO ; CCCCCCCCCCC(CCCCCCCC)C(=O)OCCCCCCN(CCCCCCOC(=O)C(CCCCCCCC)CCCCCCCCCC)CCOCCO ; CCCCCCCCCCC(CCCCCCCC)C(=O)OCCCCCCN(CCCCCCC(=O)OC(CCCCCC)CCCCCCCC)CCOCCO ; CCCCCCCCC(CCCCCC)OC(=O)OCCCCCCN(CCCCCCOC(=O)C(CCCCCC)CCCCCCCC)CCOCCO ; CCCCCCCCC(CCCCCC)OC(=O)CCCCCCN(CCCCCCOC(=O)OC(CCCCCC)CCCCCCCC)CCOCCO]

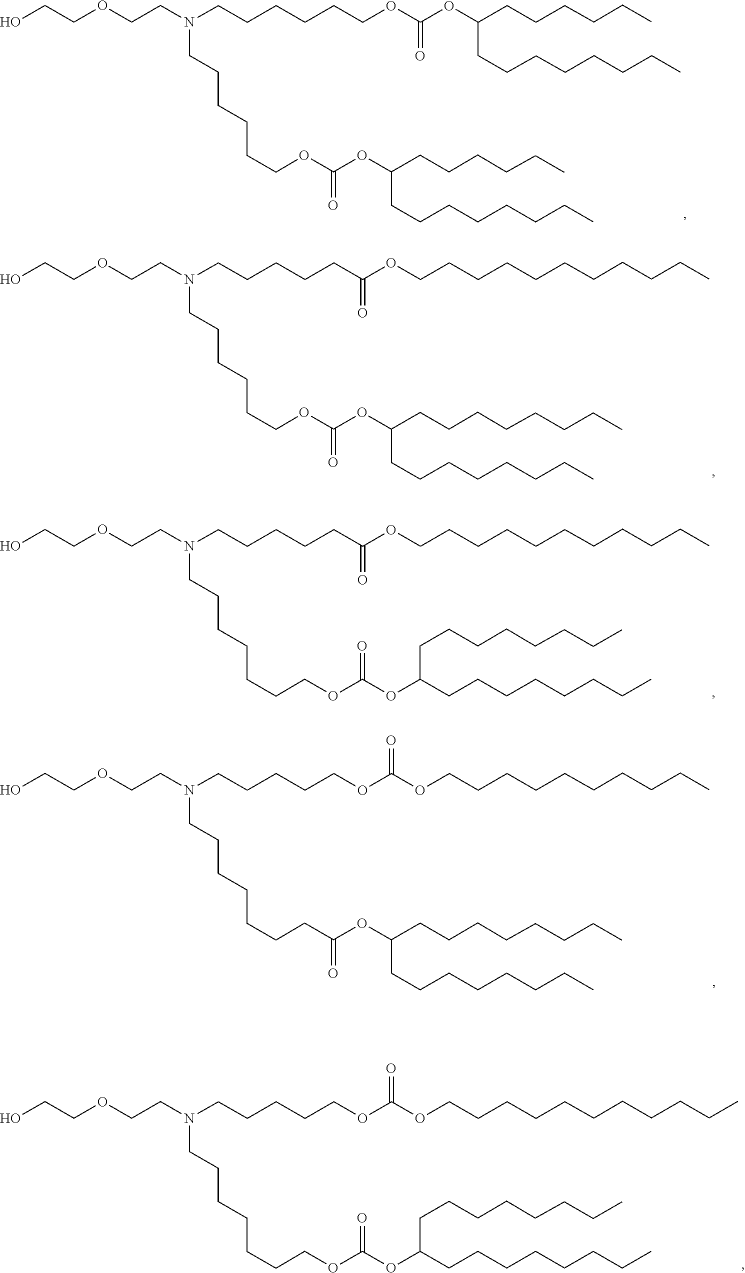

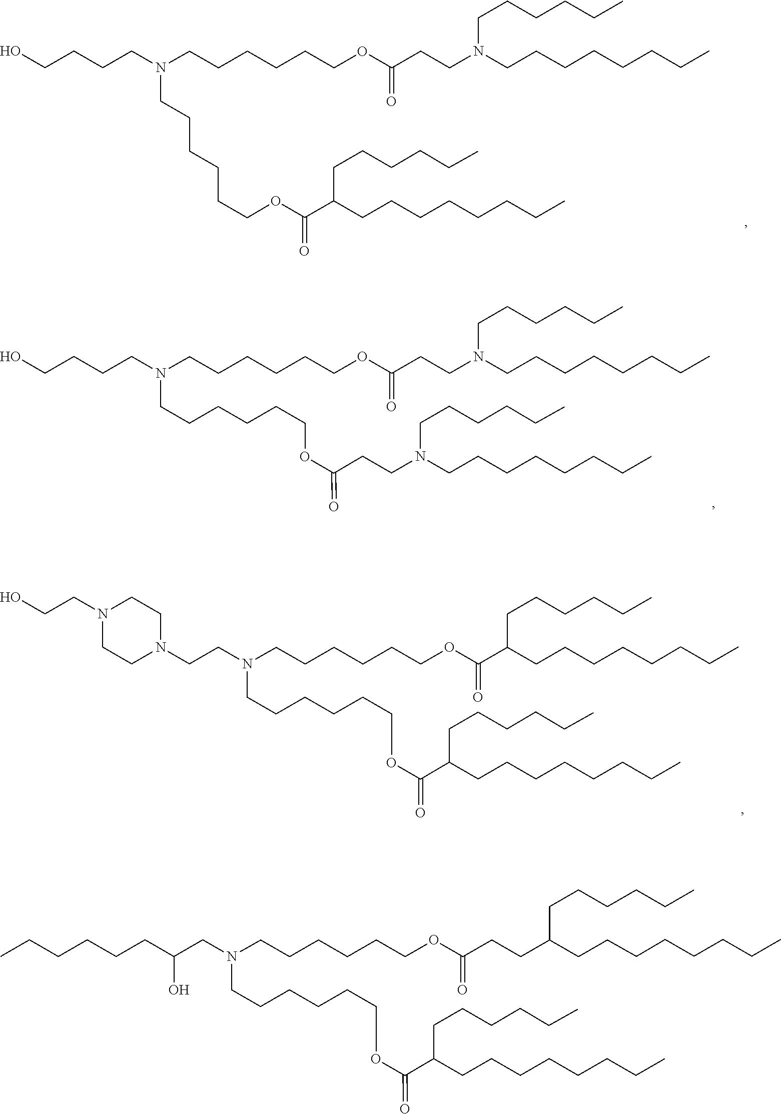

and compositions thereof.

In a more specific embodiment of the present invention, the molar percentages of PEGylated lipid, cationic lipid, phospholipid, and steroid lipid in total lipids of the lipid composition are not particularly limited; wherein,
the molar percentage of PEGylated lipid in total lipids is 0.5-5%, preferably 1-3%, and more preferably 1.5%, 1.6%, 1.7%, 1.8%, or 1.9%;
the molar percentage of cationic lipid in total lipids is 30-65%, preferably 35%, 40%, 45%, 46%, 47%, 48%, 49%, 50%, or 55%;
the molar percentage of phospholipid in total lipids is 7.5-13%, preferably 8%, 9%, 10%, 11%, or 12%;
the molar percentage of steroid lipid in total lipids is 35-50%, preferably 40%, 41%, 42%, 43%, 44%, 45%, 46%, 47%, 48%, 49%, or 50%.

Compounds, compositions, formulations, and methods of the present invention can be used to deliver bioactive substances to one or more of the following physiological sites of a patient: liver or liver cells (such as hepatocytes), kidney or kidney cells, tumor or tumor cells, CNS or CNS cells (central nervous system, such as brain and/or spinal cord), PNS or PNS cells (peripheral nervous system), lung or lung cells, blood vessels or blood vessel cells, skin or skin cells (such as dermal cells and/or follicular cells), eye or eye cells (such as macula, fovea, cornea, retina), ear or ear cells (such as inner ear, middle ear and/or outer ear cells).

In one embodiment of the present invention,
provided herein is a lipid pharmaceutical composition containing any aforementioned lipid composition and a drug selected from the group consisting of nucleic acid drug, genetic vaccine, anti-neoplastic drug, small molecule drug, peptide drug, and protein drug.

In one specific embodiment of the present invention, the drug contained in the lipid pharmaceutical composition is a nucleic acid drug selected from the group consisting of DNA, RNA, antisense nucleic acid, plasmid, interfering nucleic acid, aptamer, antagomir, and ribozyme, wherein the RNA is selected from the group consisting of mRNA, saRNA, circRNA, miRNA, and siRNA; the nucleic acid drug is preferably selected from the group consisting of DNA, mRNA, miRNA, and siRNA.

In one specific embodiment of the present invention, the drug contained in the lipid pharmaceutical composition includes but is not limited to doxorubicin, mitoxantrone, camptothecin, cisplatin, bleomycin, cyclophosphamide, streptozotocin, actinomycin D, vincristine, vinblastine, cytosine arabinoside, anthracycline, nitrogen mustard, tioteppa, chlorambucil, rachelmycin, melphalan, carmustine, romustine, busulfan, dibromannitol, mitomycin C, cis-diammineplatinum(II) dichloride, methotrexate, 6-mercaptopurine, 6-thioguanine, cytosine arabinoside, 5-fluorouracil dacarbazine, dibucaine, chlorpromazine, propranolol, demorol, labetalol, clonidine, hydralazine, imipramine, amitriptyline, doxepin, phenytoin, diphenhydramine, chlorpheniramine, promethazine, gentamicin, ciprofloxacin, cefoxitin, miconazole, terconazole, econazole, isoconazole, butoconazole, clotrimazole, itraconazole, nystatin, naftifine, amphotericin B, antiparasitic agents, hormones, hormone antagonists, immunomodulators, neurotransmitter antagonists, glaucoma drugs, vitamins, tranquilizers, imaging agents, taxol, cytochalasin B, gramicidin D, ethidium bromide, emetine, mitomycin, etoposide, teniposide, colchicine, daunorubicin, quinizarin, mithramycin, 1-dihydrotestosterone, glucocorticoid, procaine, tetracaine, lidocaine, puromycin, maytansinoid, and oxaliplatin.

In one specific embodiment of the present invention, the drug contained in the lipid pharmaceutical composition is a nucleic acid drug; the N/P ratio with respect to the cationic lipid and nucleic acid in the composition is preferably (0.1˜100):1, more preferably (0.2˜30):1, and most preferably (0.5˜20):1.

In one specific embodiment of the present invention, the lipid pharmaceutical composition is used as a medicine selected from the group consisting of drugs for treating cancer, anti-infective agents, antibiotic agents, antiviral agents, antifungal agents, and vaccines.

In one specific embodiment of the present invention, the lipid pharmaceutical composition is an LNP-pharmaceutical composition, an LPP-pharmaceutical composition, or a PNP-pharmaceutical composition, preferably an LNP-pharmaceutical composition, more preferably an LNP-nucleic acid pharmaceutical composition, and more preferably an LNP-mRNA composition.

In the present invention, a lipid pharmaceutical composition can form different structures; wherein, the “LNP-pharmaceutical composition” forms the structure of a lipid nanoparticle (LNP), the “LPP-pharmaceutical composition” forms the structure of a lipopolyplex (LPP), and the “PNP-pharmaceutical composition” forms the structure of a polypeptide nanoparticle (PNP); wherein, the “LNP-nucleic acid pharmaceutical composition” forms the structure of a lipid nanoparticle, and the drug contained is a nucleic acid drug; wherein, the “LNP-mRNA composition” is a kind of LNP-nucleic acid pharmaceutical composition, and the nucleic acid drug contained is mRNA.

In one embodiment of the present invention:
provided herein is a liposome or lipid nanoparticle containing any aforementioned lipid composition.

In one embodiment of the present invention,
provided herein is a formulation of lipid pharmaceutical composition containing any aforementioned lipid pharmaceutical composition and a working solution, and the working solution is a pharmaceutically acceptable diluent or excipient; the diluent or excipient is preferably deionized water, ultrapure water, phosphate buffer, or physiological saline, more preferably phosphate buffer or physiological saline, and most preferably physiological saline; wherein, the ratio of lipid composition to working solution is preferably (0.05˜20) g:100 mL, more preferably (0.1˜10) g:100 mL, and most preferably (0.2˜5) g:100 mL.

In one specific embodiment of the present invention, the preparation of the formulation of lipid pharmaceutical composition includes the following steps:
(1) equilibrate the lipid components in the diluent or excipient;
(2) add the drug to the mixture containing the equilibrated liposome/lipid nanoparticle and the diluent or excipient, for complexation;
wherein, the equilibration time is preferably 0.1˜12 h, more preferably 0.2˜6 h, and more preferably 0.5˜3 h; the complexation time is preferably 0.1˜12 h, more preferably 0.2˜5 h, and more preferably 0.5˜2 h.

In one specific embodiment of the present invention, the preparation of LNP-nucleic acid pharmaceutical composition includes the following steps:
(1) dissolve the lipid components with an organic solvent to obtain an organic phase solution;
(2) add the nucleic acid drug to a buffer solution to obtain an aqueous phase solution;
(3) mix the organic phase solution with the aqueous phase solution to obtain the LNP-nucleic acid pharmaceutical composition, wash the mixture using ultrafiltration to remove the organic solvent and free molecules, and finally perform a filtration using a sterile filter for further use;
wherein, the organic solvent is preferably selected from the group consisting of methanol, ethanol, propanol, tert-butanol, acetonitrile, dimethyl sulfoxide, N,N-dimethylformamide, N,N-dimethylacetamide, N-methylpyrrolidone, and mixtures thereof; the buffer solution is preferably a citrate buffer which further preferably has the concentration of 5-80 mM and the pH of 2-6, more preferably the concentration of 10-50 mM and the pH of 3-5; the volumetric ratio of organic phase solution to aqueous phase solution is preferably 1:1-10, more preferably 1:2 or 1:3.

In one specific embodiment of the present invention, the lipid pharmaceutical composition forms a lipid nanoparticle containing a drug (LNP-pharmaceutical composition), and the particle size of lipid nanoparticle is controlled by ultrasonic, extrusion, or microfluidic devices; the particle size is 1-1000 nm, preferably 20-500 nm, more preferably 60-200 nm, and most preferably 60-150 nm.

### 2. EXAMPLES

The following specific examples are for further description of the preparation of non-linear PEGylated lipids, lipid pharmaceutical compositions containing the non-linear PEGylated lipids and formulations thereof, and the biological activity assays. These specific examples are for further detailed illustration of the present invention, not limiting the scope of the present invention. Wherein, in the examples of the preparation of non-linear PEGylated lipids, the structures of end-products were characterized by NMR, and the molecular weight was determined by GPC or MALDI-TOF.

### 2.1. Preparation of Non-Linear PEGylated Lipids

### Example 1: Non-Linear PEGylated Lipids E1-1 and E1-2

### Example 1.1: Preparation of E1-1

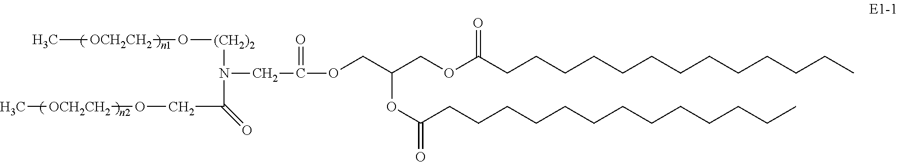

E1-1 corresponds to the general formula (1); wherein, X is —CH<, R₁ and R₂ are both tridecyl groups, B₁ is a methylene group, B₂ is a linking bond, L₁ and L₂ are both ester groups (—OC(═O)—), L_{d} is —CH₂C(═O)OCH₂—, y=2, T is a methyl group, and the two Lₓ are —(CH₂)₂— and —CH₂C(═O)—, respectively. The designed total molecular weight is approximately 2.7 kDa, wherein the molecular weight of each PEG chain is approximately 1 kDa, corresponding to n₁≈n₂≈22.

The preparation method is as follows:

Step a: Under argon atmosphere, glycerol derivative containing a TBS-protected hydroxyl group (S1-1, 0.41 g, 2.0 mmol), myristic acid (S1-2, 1.14 g, 5.0 mmol), and 4-(dimethylamino)pyridine (DMAP, 0.12 g, 1.0 mmol) were dissolved in dichloromethane (15 mL), and then the solution of dicyclohexylcarbodiimide (DCC, 1.13 g, 5.5 mmol) in dichloromethane (15 mL) was added dropwise in an ice bath. Upon completion of the dropwise addition, the reaction temperature was raised to room temperature, and the reaction was stirred for 24 h. After the reaction was over, the precipitates were removed by filtration. The reaction solution was concentrated under reduced pressure, 10 mL tetrahydrofuran (THF) was added, and then 10 mL of the 1M solution of tetra-n-butylammonium fluoride (TBAF) in tetrahydrofuran was added. The reaction was continued overnight to remove the TBS protection. Concentration and purification by column chromatography were carried out to obtain S1-3 (1.67 g).

Step b: Under argon atmosphere, the branched polyethylene glycol carboxylic acid derivative S1-4 (2.20 g, 1.0 mmol, Mₙ≈2.2 kDa, n₁≈n₂≈22, PDI=1.01), the low-molecular-weight lipid compound S1-3 (1.54 g, 3.0 mmol), and DMAP (24 mg, 0.2 mmol) were dissolved in dichloromethane (20 mL), and then the solution of DCC (0.23 g, 1.1 mmol) in dichloromethane (4 mL) was added dropwise in an ice bath. Upon completion of the dropwise addition, the reaction temperature was raised to room temperature, and the reaction was stirred for 24 h. After the reaction was over, the precipitates were removed by filtration.

After drying with anhydrous sodium sulfate, filtration, concentration, and purification by column chromatography, the PEGylated lipid E1-1 (1.05 g) was obtained. The main data of the ¹H-NMR spectrum of E1-1 are as follows: ¹H NMR (400 MHz, CDCl₃) δ: 5.32-5.22 (m, 1H, —CH<), 4.29-4.15 (m, 2H, —C(═O)OCH₂CH<; 2H, >CHCH₂OC(═O)—; 2H, —OCH₂C(═O)N<), 3.98 (s, 2H, >NCH₂C(═O)O—), 3.72-3.41 (m, PEG; 4H, —OCH₂CH₂N<), 3.36 (s, 6H, —OCH₃), 2.30 (t, 4H, —OC(═O)CH₂CH₂—), 1.69-1.00 (m, 40H, —CH₂CH₂CH₂—; 4H, —CH₂CH₃), 0.85 (t, 6H, —CH₂CH₃). The molecular weight determined by GPC was approximately 2.7 kDa, PDI=1.01.

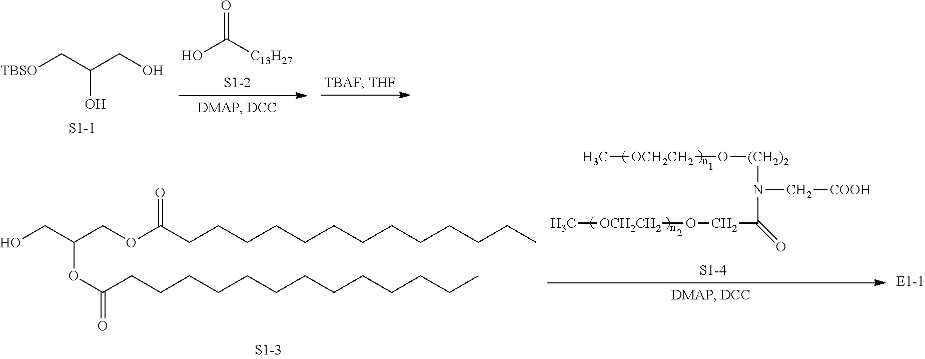

### Example 1.2: Preparation of E1-2

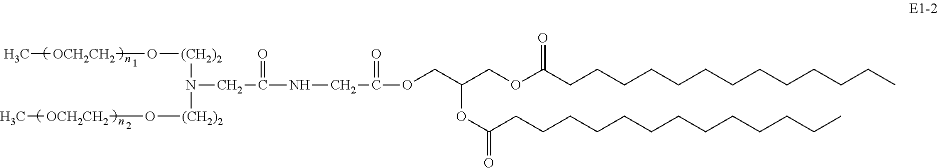

E1-2 corresponds to the general formula (1); wherein, X is —CH<, R₁ and R₂ are both tridecyl groups, B₁ is a methylene group, B₂ is a linking bond, L₁ and L₂ are both ester groups (—OC(═O)—), L_{d} is —CH₂C(═O)NHCH₂C(═O)OCH₂—, y=2, T is a methyl group, and the two Lₓ are both —(CH₂)₂—. The designed total molecular weight is approximately 2.7 kDa, wherein the molecular weight of each PEG chain is approximately 1 kDa, corresponding to n₁≈n₂≈22.

The preparation method is as follows:

Under argon atmosphere, the branched polyethylene glycol carboxylic acid derivative S1-5 (4.40 g, 2.0 mmol, Mₙ≈2.2 kDa, n₁≈n₂≈22, PDI=1.02, prepared following the methods in CN108530637B), the low-molecular-weight lipid compound S1-3 (3.08 g, 6.0 mmol), and DMAP (49 mg, 0.4 mmol) were dissolved in dichloromethane (50 mL), and then the solution of DCC (0.45 g, 2.2 mmol) in dichloromethane (8 mL) was added dropwise in an ice bath. Upon completion of the dropwise addition, the reaction temperature was raised to room temperature, and the reaction was stirred for 24 h. After the reaction was over, the precipitates were removed by filtration. After concentration and purification by column chromatography, the PEGylated lipid E1-2 (1.91 g) was obtained. The main data of the ¹H-NMR spectrum of E1-2 are as follows: ¹H NMR (400 MHz, CDCl₃) δ: 5.32-5.22 (m, 1H, —CH<), 4.29-4.15 (m, 2H, —C(═O)OCH₂CH<; 2H, >CHCH₂OC(═O)—), 4.11-4.04 (m, 2H, —C(═O)NHCH₂—), 3.75-3.43 (m, PEG; 4H, —OCH₂CH₂N<), 3.35 (s, 6H, —OCH₃), 3.27 (s, 2H, >NCH₂C(═O)NH—), 2.80 (t, 4H, —OCH₂CH₂N<), 2.30 (t, 4H, —OC(═O)CH₂CH₂—), 1.69-1.07 (m, 40H, —CH₂CH₂CH₂—; 4H, —CH₂CH₃), 0.88 (t, 6H, —CH₂CH₃). The molecular weight determined by GPC was approximately 2.7 kDa, PDI=1.02.

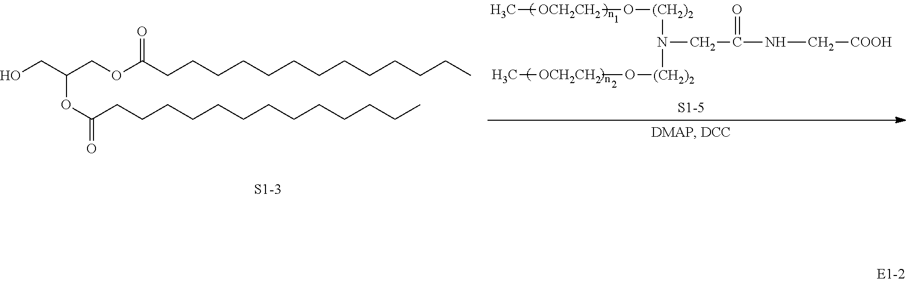

### Example 2: Non-Linear PEGylated Lipids E2-1-E2-4

### Example 2.1: Preparation of E2-1

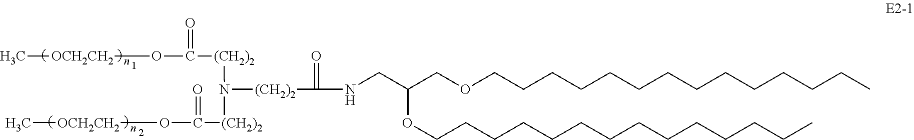

E2-1 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —(CH₂)₂C(═O)NHCH₂—, y=2, T is a methyl group, and the two Lₓ are both —C(═O)(CH₂)₂—. The designed total molecular weight is approximately 2.7 kDa, wherein the molecular weight of each PEG chain is approximately 1 kDa, corresponding to n₁˜n₂˜22.

The preparation method is as follows:

Step a: Under anhydrous and oxygen-free conditions, 300 mL tetrahydrofuran and excess diphenylmethyl potassium (DPMK, 24.72 g, 120.0 mmol) were added into a flask, followed by adding the sulfonate derivative of tetradecanol (C₁₄H₂₈-OTs, S2-1, 21.25 g, 57.6 mmol) and the 3-amino-1,2-propanediol containing a Boc-protected amino group (S2-2, 4.58 g, 24.0 mmol). After 12 hours of reaction at 30° C., the reactor was opened, the reaction solution was washed and concentrated, and the Boc protection was removed by the TFA/DCM mixed solution (1:1 v/v). After washing with purified water and extracting with dichloromethane, the extract obtained was dried over anhydrous sodium sulfate, filtered, concentrated, and purified by column chromatography to obtain the intermediate S2-3 (9.32 g) containing a free amino group.

Step b: The β-alanine derivative containing a Boc-protected amino group (S2-4, 2.84 g, 15.0 mmol) was dissolved in 80 mL anhydrous chloromethane, added with NHS (2.59 g, 22.5 mmol), and then added with DCC (4.64 g, 22.5 mmol). Into 120 mL solution of S2-3 (8.71 g, 18.0 mmol) in dichloromethane, DMAP (0.37 g, 3.0 mmol) was added. Said two solutions were mixed and then reacted while stirring at room temperature for 24 hours. The reaction solution was filtered to remove the precipitates, and then concentrated under reduced pressure. The TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the intermediate S2-5 (7.41 g) containing a free amino group.

Step c: First alkylation reaction: The ester bond-containing methoxypolyethylene glycol sulfonate derivative S2-6 (1.44 g, 1.2 mmol, Mₙ≈1.2 kDa, n₁≈22, PDI=1.01) was dissolved in dichloromethane, added with the low-molecular-weight lipid intermediate S2-5 (6.66 g, 12.0 mmol), and then reacted for 24 hours at room temperature. After the reaction was over, the reaction solution was concentrated and then precipitated twice in diethyl ether to remove impurities, therefore obtaining the intermediate S2-7 (1.61 g).

Step d: Second alkylation reaction: S2-7 (1.28 g, 0.8 mmol, Mₙ≈1.6 kDa, n₁≈22, PDI=1.02) and S2-6 (1.44 g, 1.2 mmol) were dissolved in dichloromethane and reacted for 24 hours at room temperature. After the reaction was over, the reaction solution was concentrated, added with a phosphate buffer of pH=7.0, stirred for 16 h, and purified by column chromatography to obtain the PEGylated lipid E2-1 (1.10 g). The main data of the ¹H-NMR spectrum of E2-1 are as follows: ¹H NMR (400 MHz, CDCl₃) δ: 4.23-4.06 (m, 4H, —OCH₂CH₂OC(═O)—), 3.80-3.28 (m, PEG; 4H, —OCH₂CH₂OC(═O)—; 2H, —C(═O)NHCH₂CH<; 1H, —C(═O)NHCH₂CH<; 2H, >CHCH₂O(CH₂)₂—; 4H, —OCH₂CH₂CH₂—; 6H, —OCH₃), 2.78-2.35 (m, 8H, —C(═O)CH₂CH₂N<; 4H, >NCH₂CH₂C(═O)NH—), 1.53-1.11 (m, 44H, —CH₂CH₂CH₂—; 4H, —CH₂CH₃), 0.88 (t, 6H, —CH₂CH₃). The molecular weight determined by GPC was approximately 2.7 kDa, PDI=1.02.

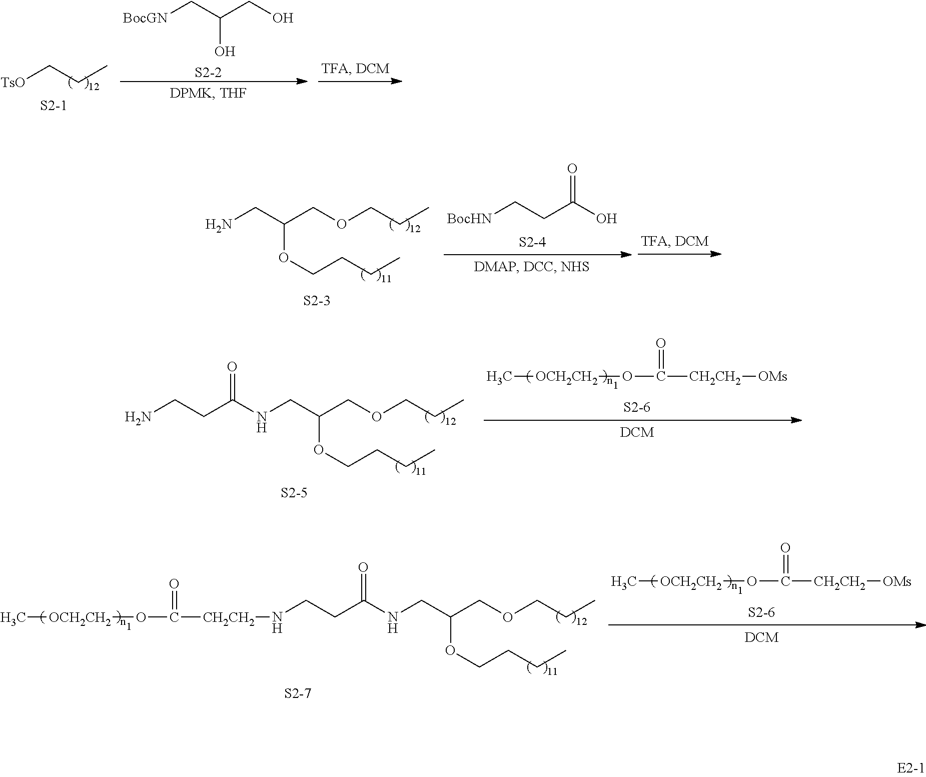

### Example 2.2: Preparation of E2-2

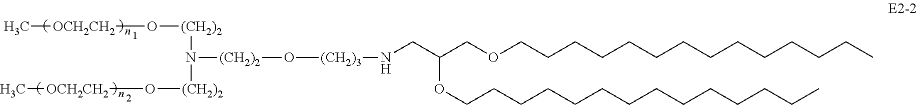

E2-2 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —(CH₂)₃O(CH₂)₃NHCH₂—, y=2, T is a methyl group, and the two Lₓ are both —(CH₂)₂—. The designed total molecular weight is approximately 2.7 kDa, wherein the molecular weight of each PEG chain is approximately 1 kDa, corresponding to n₁˜n₂˜22.

The preparation method is as follows:

Under nitrogen protection, the branched polyethylene glycol aldehyde derivative S2-8 (2.20 g, 1.0 mmol, Mₙ≈2.2 kDa, n₁≈n₂≈22, PDI=1.02, prepared following the methods in CN108530637B) was dissolved in dry tetrahydrofuran (20 mL), and then the compound S2-3 (1.45 g, 3.0 mmol), sodium triacetoxyborohydride (NaBH(OAc)₃, 0.32 g, 1.5 mmol), and glacial acetic acid (0.06 g, 1.0 mmol) were successively added. After stirring at 20° C. for 16 hours, the reaction was quenched with saturated sodium bicarbonate solution. 20 mL dichloromethane was added for extraction, then the organic phase was separated. The aqueous phase was further extracted with 10 mL dichloromethane. The organic phases were combined, dried over anhydrous sodium sulfate, filtered, concentrated, and purified by column chromatography to obtain the PEGylated lipid E2-2 (0.76 g). The main data of the ¹H-NMR spectrum of E2-2 are as follows: ¹H NMR (400 MHz, CDCl₃) δ: 4.05-3.42 (m, PEG; 4H, —OCH₂CH₂N<; 2H, >NCH₂CH₂O—; 2H, —OCH₂CH₂CH₂NH—; 1H, —NHCH₂CH<; 2H, >CHCH₂O—; 4H, —OCH₂CH₂CH₂CH₂—), 3.36 (s, 6H, —OCH₃), 2.83-2.45 (m, 4H, —OCH₂CH₂N<; 2H, >NCH₂CH₂O—; 4H, —CH₂NHCH₂—), 1.79-1.66 (m, 2H, —OCH₂CH₂CH₂NH—), 1.53-1.13 (m, 44H, —CH₂CH₂CH₂—; 4H, —CH₂CH₃), 0.89 (t, 6H, —CH₂CH₃). The molecular weight determined by GPC was approximately 2.7 kDa, PDI=1.02.

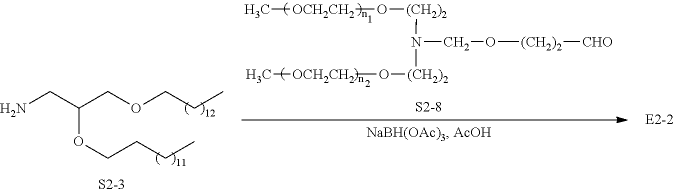

### Example 2.3: Preparation of E2-3

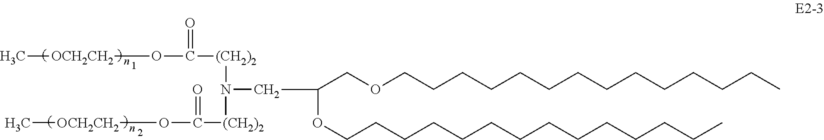

E2-3 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂—, y=2, T is a methyl group, and the two Lₓ are both —C(═O)(CH₂)₂—. The designed total molecular weight is approximately 2.6 kDa, wherein the molecular weight of each PEG chain is approximately 1 kDa, corresponding to n₁≈n₂≈22.

The preparation method is as follows:

Referring to the reaction conditions and feeding ratios of the Step c and Step d in Example 2.1, two alkylation reactions were carried out using S2-3 (5.81 g, 12.0 mmol) and the ester bond-containing methoxypolyethylene glycol sulfonate derivative S2-6 (1.44 g, 1.2 mmol per feed), and then the PEGylated lipid E2-3 (1.02 g) was obtained after purification by column chromatography. The main data of the ¹H-NMR spectrum of E2-3 are as follows: ¹H NMR (400 MHz, CDCl₃) δ: 4.24-4.10 (m, 4H, —OCH₂CH₂—OC(═O)—), 3.74-3.34 (m, PEG; 4H, —OCH₂CH₂OC(═O)—; 1H, >NCH₂CH<; 2H, >CHCH₂O—; 4H, —OCH₂CH₂CH₂—; 6H, —OCH₃), 2.79-2.35 (m, 8H, —C(═O)CH₂CH₂N<; 2H, >NCH₂CH<), 1.54-0.99 (m, 44H, —CH₂CH₂CH₂—; 4H, —CH₂CH₃), 0.85 (t, 6H, —CH₂CH₃). The molecular weight determined by GPC was approximately 2.6 kDa, PDI=1.01.

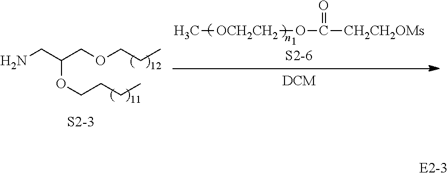

### Example 2.4: Preparation of E2-4 and E2-5

Following the preparation method of E2-3 in Example 2.3, using the corresponding starting materials, the polydisperse two-arm PEGylated lipid E2-4 with 2 kDa as the molecular weight of each PEG arm (n₁≈n₂≈45) and the monodisperse two-arm PEGylated lipid E2-5 with 500 Da as the molecular weight of each PEG arm (CH₃(OCH₂CH₂)₁₁—, n₁=n₂=11) were prepared. The structures of E2-4 and E2-5 were determined by ¹H-NMR, both of which have the same structural formula as that of E2-3, wherein the difference lies in the values of n₁ and n₂ only. The molecular weight of E2-4 determined by GPC was approximately 4.8 kDa, PDI=1.02; The molecular weight of E2-5 determined by MALDI-TOF MS was 1624 Da.

### Example 3: Non-Linear PEGylated Lipid E3-1

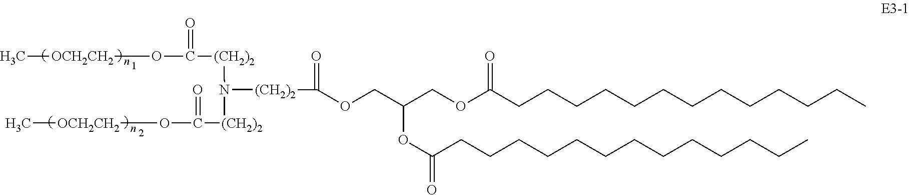

E3-1 corresponds to the general formula (1); wherein, X is —CH<, R₁ and R₂ are both tridecyl groups, B₁ is a methylene group, B₂ is a linking bond, L₁ and L₂ are both ester groups (—OC(═O)—), L_{d} is —(CH₂)₂C(═O)OCH₂—, y=2, T is a methyl group, and the two Lₓ are both —C(═O)(CH₂)₂—. The designed total molecular weight is approximately 2.7 kDa, wherein the molecular weight of each PEG chain is approximately 1 kDa, corresponding to n₁˜n₂˜22.

The preparation method is as follows:

Step a: Under argon atmosphere, S2-4 (1.89 g, 10.0 mmol), S1-3 (6.16 g, 12.0 mmol), and DMAP (0.24 g, 2.0 mmol) were dissolved in dichloromethane (80 mL), and then the solution of DCC (2.27 g, 11.0 mmol) in dichloromethane (30 mL) was added dropwise in an ice bath.

Upon completion of the dropwise addition, the reaction temperature was raised to room temperature, and the reaction was stirred for 24 h. After the reaction was over, the precipitates were removed by filtration. The reaction solution was concentrated under reduced pressure, and then the TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the intermediate S3-1 (4.86 g) containing a free amino group.

Step b: Referring to the reaction conditions and feeding ratios of the Step c and Step d in Example 2.1, two alkylation reactions were carried out using S3-1 (3.50 g, 6.0 mmol) and the ester bond-containing methoxypolyethylene glycol sulfonate derivative S2-6 (0.72 g, 0.6 mmol per feed), and then the PEGylated lipid E3-1 (0.56 g) was obtained after purification by column chromatography. The main data of the ¹H-NMR spectrum of E3-1 are as follows: ¹H NMR (400 MHz, CDCl₃) δ: 5.32-5.22 (m, 1H, —CH<), 4.29-4.15 (m, 2H, —C(═O)OCH₂CH<; 2H, >CHCH₂OC(═O)—; 4H, —OCH₂CH₂—OC(═O)—), 3.72-3.38 (m, PEG; 4H, —OCH₂CH₂—OC(═O)—), 3.36 (s, 6H, —OCH₃), 2.79-2.35 (m, 8H, —C(═O)CH₂CH₂N<; 4H, >NCH₂CH₂C(═O)O—), 2.28 (t, 4H, —OC(═O)CH₂CH₂CH₂—), 1.69-1.03 (m, 40H, —CH₂CH₂CH₂—; 4H, —CH₂CH₃), 0.86 (t, 6H, —CH₂CH₃). The molecular weight determined by GPC was approximately 2.7 kDa, PDI=1.02.

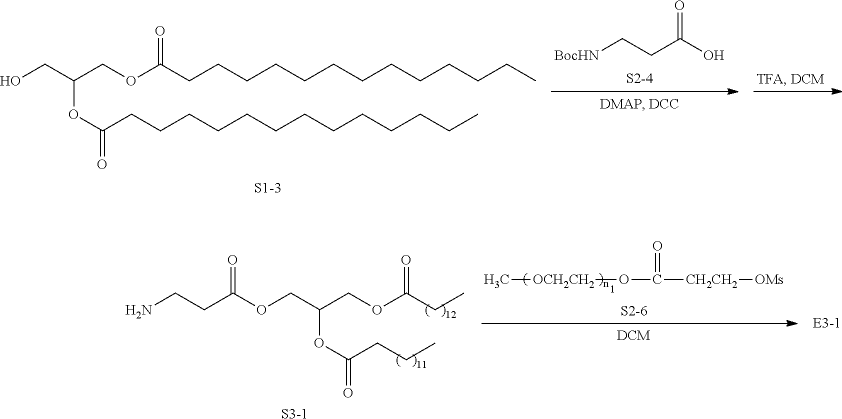

### Example 4: Non-Linear PEGylated Lipids E4-1-E4-3

### Example 4.1: Preparation of E4-1

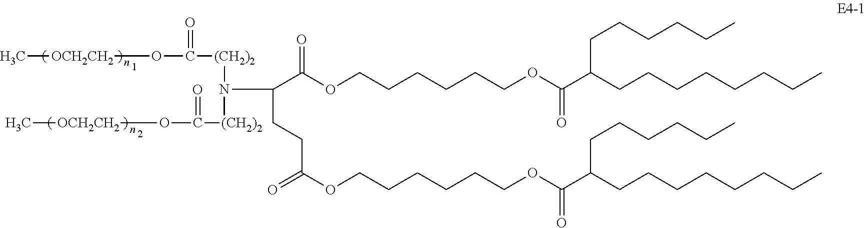

E4-1 corresponds to the general formula (1); wherein, X is —CH<, R₁ and R₂ are both

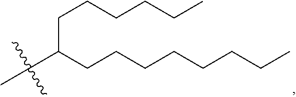

B₁ is a linking bond, B₂ is an ethylene group, L₁ and L₂ are both —C(═O)O(CH₂)₆OC(═O)—, L_{d} is a linking bond, y=2, T is a methyl group, and the two Lₓ are both —C(═O)(CH₂)₂—. The molecular weight of each PEG chain is approximately 1 kDa, corresponding to n₁≈n₂≈22.

The preparation method is as follows:

Step a: Under argon atmosphere, 2-hexyldecanoic acid (S4-1, 3.07 g, 12.0 mmol), 1,6-hexanediol (S4-2, 1.70 g, 14.4 mmol), and DMAP (0.29 g, 2.4 mmol) were dissolved in dichloromethane (50 mL), and then the solution of DCC (2.72 g, 13.2 mmol) in dichloromethane (40 mL) was added dropwise in an ice bath. Upon completion of the dropwise addition, the reaction temperature was raised to room temperature, and the reaction was stirred for 24 h. After the reaction was over, the precipitates were removed by filtration. Concentration and purification by column chromatography were carried out to obtain S4-3 (3.56 g).

Step b: Under argon atmosphere, S4-3 (3.43 g, 9.6 mmol), glutamic acid derivative containing a Boc-protected amino group (S4-4, 0.99 g, 4.0 mmol), and DMAP (0.20 g, 1.6 mmol) were dissolved in dichloromethane (50 mL), and then the solution of DCC (1.81 g, 8.8 mmol) in dichloromethane (30 mL) was added dropwise in an ice bath. Upon completion of the dropwise addition, the reaction temperature was raised to room temperature, and the reaction was stirred for 24 h. After the reaction was over, the precipitates were removed by filtration. The reaction solution was concentrated under reduced pressure, and then the TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the intermediate S4-5 (2.57 g) containing a free amino group.

Step c: Referring to the reaction conditions and feeding ratios of the Step c and Step d in Example 2.1, two alkylation reactions were carried out using S4-5 (2.47 g, 3.0 mmol) and the ester bond-containing methoxypolyethylene glycol sulfonate derivative S2-6 (0.36 g, 0.3 mmol per feed), and then the PEGylated lipid E4-1 (0.28 g) was obtained after purification by column chromatography. The main data of the ¹H-NMR spectrum of E4-1 are as follows: ¹H NMR (400 MHz, CDCl₃) δ: 4.27-4.04 (m, 4H, —OCH₂CH₂—OC(═O)—; 8H, —C(═O)OCH₂(CH₂)₄CH₂OC(═O)—), 3.74-3.41 (m, PEG; 4H, —OCH₂CH₂—OC(═O)—), 3.39 (t, 1H, >NCH<), 3.35 (s, 6H, —OCH₃), 2.80-2.35 (m, 8H, —C(═O)CH₂CH₂N<), 2.33-2.22 (m, 2H, —OC(═O)CH<; 2H, >CHCH₂CH₂C(═O)O—), 2.07-1.74 (m, 2H, >CHCH₂CH₂C(═O)O—), 1.70-1.11 (m, 48H, —CH₂CH₂CH₂—; 8H, —OC(═O)CHCH₂—; 8H, —CH₂CH₃), 0.84 (t, 12H, —CH₂CH₃). The molecular weight determined by GPC was approximately 3.0 kDa, PDI=1.02.

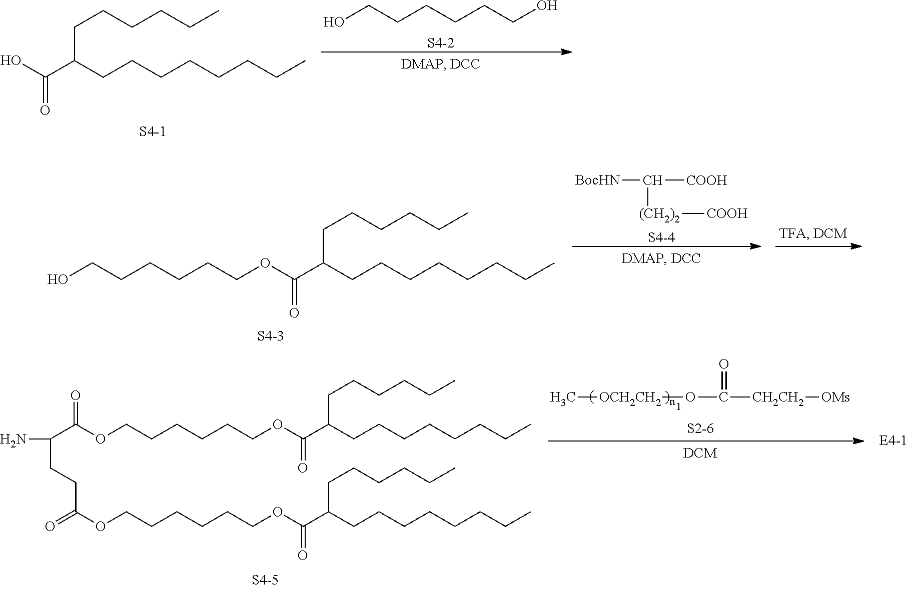

### Example 4.2: Preparation of E4-2

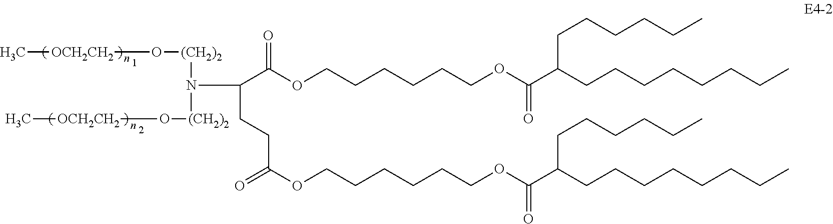

E4-2 corresponds to the general formula (1); wherein, X is —CH<, R₁ and R₂ are both

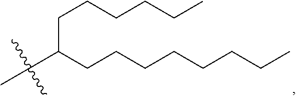

B₁ is a linking bond, B₂ is an ethylene group, L₁ and L₂ are both —C(═O)O(CH₂)₆OC(═O)—, L_{d} is a linking bond, y=2, T is a methyl group, and the two Lₓ are both —(CH₂)₂—. The designed total molecular weight is approximately 2.8 kDa, wherein the molecular weight of each PEG chain is approximately 1 kDa, corresponding to n₁≈n₂≈22.

The preparation method is as follows:

Step a: Under argon atmosphere, compound S4-6 (1.76 g, 7.2 mmol, prepared via the reaction between 1,6-hexanediol and 1-octanoic acid, following the specific steps in Example 4.1), glutamic acid derivative containing a Boc-protected amino group (S4-4, 1.48 g, 6.0 mmol), and DMAP (0.15 g, 1.2 mmol) were dissolved in dichloromethane (25 mL), and then the solution of DCC (1.36 g, 6.6 mmol) in dichloromethane (20 mL) was added dropwise in an ice bath. Upon completion of the dropwise addition, the reaction temperature was raised to room temperature, and the reaction was stirred for 24 h. After the reaction was over, the precipitates were removed by filtration. Concentration and purification by column chromatography were carried out to obtain the intermediate S4-7 (2.45 g).

Step b: Under argon atmosphere, S4-7 (1.90 g, 4.0 mmol), S4-3 (1.71 g, 4.8 mmol), and DMAP (0.10 g, 0.8 mmol) were dissolved in dichloromethane (30 mL), and then the solution of DCC (0.91 g, 4.4 mmol) in dichloromethane (15 mL) was added dropwise in an ice bath. Upon completion of the dropwise addition, the reaction temperature was raised to room temperature, and the reaction was stirred for 24 h. After the reaction was over, the precipitates were removed by filtration. The reaction solution was concentrated under reduced pressure, and then the TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the intermediate S4-8 (2.30 g) containing a free amino group.

Step c: Referring to the reaction conditions and feeding ratios of the Step c and Step d in Example 2.1, two alkylation reactions were carried out using S4-8 (2.14 g, 3.0 mmol) and the methoxypolyethylene glycol sulfonate derivative S4-9 (0.33 g, 0.3 mmol per feed; M&˜1.1 kDa, n₁˜22, PDI=1.01), and then the PEGylated lipid E4-2 (0.24 g) was obtained after purification by column chromatography. The main data of the ¹H-NMR spectrum of E4-2 are as follows: ¹H NMR (400 MHz, CDCl₃) δ: 4.08 (t, 8H, —C(═O)OCH₂(CH₂)₄CH₂OC(═O)—), 3.72-3.44 (m, PEG; 4H, —OCH₂CH₂N<), 3.40 (t, 1H, >NCH<), 3.37 (s, 6H, —OCH₃), 2.91-2.79 (m, 4H, —OCH₂CH₂N<), 2.39-2.20 (m, 1H, —OC(═O)CH<; 2H, >CHCH₂CH₂C(═O)O—; 2H, —OC(═O)CH₂(CH₂)SCH₃), 2.07-1.74 (m, 2H, >CHCH₂CH₂C(═O)O—), 1.68-1.08 (m, 40H, —CH₂CH₂CH₂—; 4H, —OC(═O)CHCH₂—; 6H, —CH₂CH₃), 0.85 (t, 12H, —CH₂CH₃). The molecular weight determined by GPC was approximately 2.8 kDa, PDI=1.02.

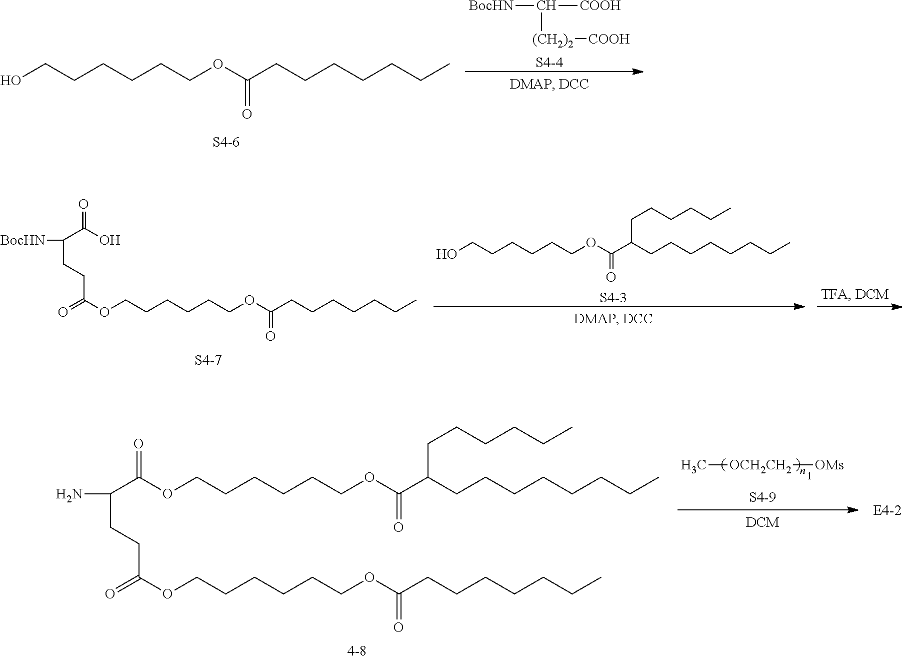

### Example 4.3: Preparation of E4-3

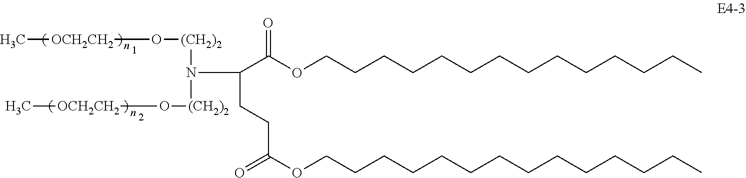

E4-3 corresponds to the general formula (1); wherein, X is —CH<, R₁ and R₂ are both tetradecyl groups, B₁ is a linking bond, B₂ is an ethylene group, L₁ and L₂ are both ester groups (—C(═O)O—), L_{d} is a linking bond, y=2, T is a methyl group, and the two Lₓ are both —(CH₂)₂—. The designed total molecular weight is approximately 2.6 kDa, wherein the molecular weight of each PEG chain is approximately 1 kDa, corresponding to n₁˜n₂˜22.

The preparation method is as follows:

Step a: Under argon atmosphere, 1-tetradecyl alcohol (S4-10, 2.05 g, 9.6 mmol), glutamic acid derivative containing a Boc-protected amino group (S4-4, 0.99 g, 4.0 mmol), and DMAP (0.20 g, 1.6 mmol) were dissolved in dichloromethane (30 mL), and then the solution of DCC (1.81 g, 8.8 mmol) in dichloromethane (30 mL) was added dropwise in an ice bath. Upon completion of the dropwise addition, the reaction temperature was raised to room temperature, and the reaction was stirred for 24 h. After the reaction was over, the precipitates were removed by filtration. The reaction solution was concentrated under reduced pressure, and then the TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the intermediate S4-11 (1.53 g) containing a free amino group.

Step b: Referring to the reaction conditions and feeding ratios of the Step c and Step d in Example 2.1, two alkylation reactions were carried out using S4-11 (1.62 g, 3.0 mmol) and the methoxypolyethylene glycol sulfonate derivative S4-9 (0.33 g, 0.3 mmol per feed), and then the PEGylated lipid E4-3 (0.19 g) was obtained after purification by column chromatography. The main data of the ¹H-NMR spectrum of E4-3 are as follows: ¹H NMR (400 MHz, CDCl₃) δ: 4.08 (t, 4H, —C(═O)OCH₂—), 3.72-3.42 (m, PEG; 4H, —OCH₂CH₂N—), 3.40 (t, 1H, >NCH<), 3.34 (s, 6H, —OCH₃), 2.93-2.80 (m, 4H, —OCH₂CH₂N<), 2.38-2.26 (m, 2H, —CH₂C(═O)O—), 2.07-1.74 (m, 2H, >CHCH₂CH₂C(═O)O—), 1.71-1.03 (m, 44H, —CH₂CH₂CH₂—; 4H, —CH₂CH₃), 0.88 (t, 12H, —CH₂CH₃). The molecular weight determined by GPC was approximately 2.6 kDa, PDI=1.02.

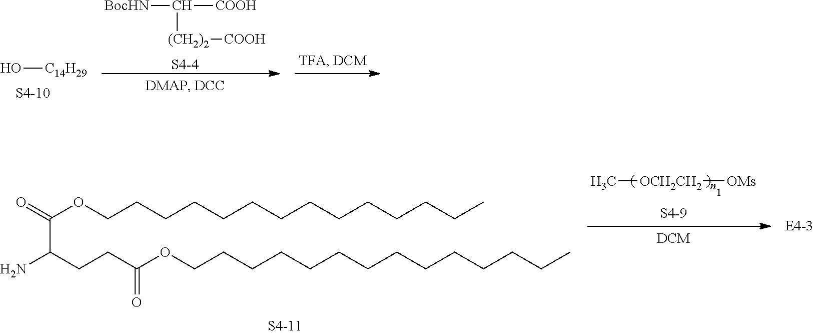

### Example 5: Non-Linear PEGylated Lipid E5-1

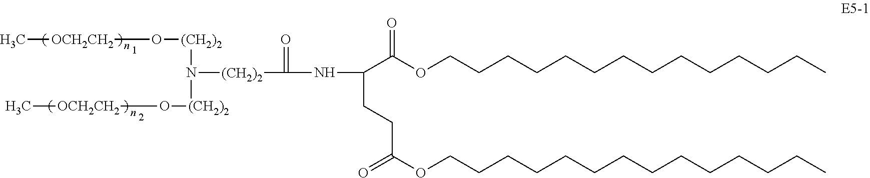

E5-1 corresponds to the general formula (1); wherein, X is —CH<, R₁ and R₂ are both tetradecyl groups, B₁ is a linking bond, B₂ is an ethylene group, L₁ and L₂ are both ester groups (—C(═O)O—), L_{d} is —(CH₂)₂C(═O)NH—, y=2, T is a methyl group, and the two Lₓ are both —(CH₂)₂—. The designed total molecular weight is approximately 2.7 kDa, wherein the molecular weight of each PEG chain is approximately 1 kDa, corresponding to n₁≈n₂≈22.

The preparation method is as follows:

Step a: The β-alanine derivative containing a Boc-protected amino group (S2-4, 2.84 g, 15.0 mmol) was dissolved in 60 mL anhydrous chloromethane, added with NHS (2.59 g, 22.5 mmol), and then added with DCC (4.64 g, 22.5 mmol). Into 140 mL solution of S4-11 (9.72 g, 18.0 mmol) in dichloromethane, DMAP (0.37 g, 3.0 mmol) was added. Said two solutions were mixed and then reacted while stirring at room temperature for 24 hours. The reaction solution was filtered to remove the precipitates, and then concentrated under reduced pressure. The TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the intermediate S5-1 (7.64 g) containing a free amino group.

Step b: Referring to the reaction conditions and feeding ratios of the Step c and Step d in Example 2.1, two alkylation reactions were carried out using S5-1 (7.33 g, 12.0 mmol) and the methoxypolyethylene glycol sulfonate derivative S4-9 (1.32 g, 1.2 mmol per feed), and then the PEGylated lipid E5-1 (0.71 g) was obtained after purification by column chromatography. The main data of the ¹H-NMR spectrum of E5-1 are as follows: ¹H NMR (400 MHz, CDCl₃) δ: 4.34-4.14 (m, 1H, —CH<), 4.09 (t, 4H, —C(═O)OCH₂—), 3.72-3.42 (m, PEG; 4H, —OCH₂CH₂N<), 3.36 (s, 6H, —OCH₃), 2.80-2.34 (m, 4H, —OCH₂CH₂N<; 4H, >N(CH₂)₂C(═O)NH—; 2H, Glu-γ-CH₂—), 2.12-1.76 (m, 2H, Glu-β-CH₂—), 1.71-1.08 (m, 44H, —CH₂CH₂CH₂—; 4H, —CH₂CH₃), 0.86 (t, 12H, —CH₂CH₃). The molecular weight determined by GPC was approximately 2.7 kDa, PDI=1.01.

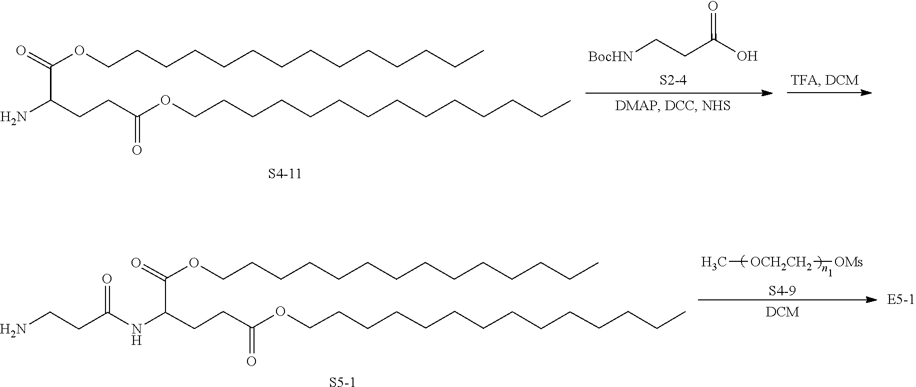

### Example 6: Non-Linear PEGylated Lipids E6-1 and E6-2

### Example 6.1: Preparation of E6-1

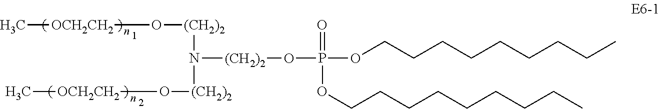

E6-1 corresponds to the general formula (1); wherein, X is

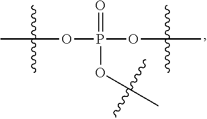

R₁ and R₂ are both nonyl groups, B₁ and B₂ are both linking bonds, L₁ and L₂ are both linking bonds, L_{d} is —(CH₂)₂—, y=2, T is a methyl group, and the two Lₓ are both —(CH₂)₂—. The molecular weight of each PEG chain is approximately 500 Da, corresponding to n₁=n₂=11.

The preparation method is as follows:

Step a: 1-nonanol (S6-1, 2.05 g, 18.0 mmol) and pyridine (0.97 mL, 12.0 mmol) were dissolved in 30 mL diethyl ether. The solution was placed in an ice bath and added with phosphorus trichloride (PCl₃, 0.52 mL, 6.0 mmol) in 1 hour. After the feeding, the reaction solution was slowly raised to room temperature. After 16 hours of reaction with stirring, the reaction solution was filtered. The precipitates were washed with 15 mL*2 diethyl ether. The filtrates were combined, dried over anhydrous sodium sulfate, filtered, concentrated under reduced pressure, and purified by column chromatography to obtain the intermediate S6-2 (1.90 g).

Step b: Under nitrogen protection, 20 mL solution of S6-2 (1.34 g, 4.0 mmol) in anhydrous acetonitrile was mixed with 5 mL solution of trichloroisocyanuric acid (TCICA, 0.31 g, 1.3 mmol) in anhydrous acetonitrile, and then reacted for 10 min at room temperature while stirring. White precipitates were formed. The stirring was continued for 2 hours, and then the precipitates were removed by filtration. The filtrate was concentrated under reduced pressure and purified by column chromatography to obtain the low-molecular weight chlorinated phospholipid compound intermediate S6-3 (1.35 g).

Step c: Under argon atmosphere, the monodisperse branched polyethylene glycol derivative S6-4 (2.29 g, 1.0 mmol, Mₙ=1146 Da, n₁=n₂=11, prepared following the methods in CN1243779C), DMAP (24 mg, 0.2 mmol), and pyridine (87 μL, 1.1 mmol) were dissolved in dichloromethane (15 mL). Compound S6-3 (1.11 g, 3.0 mmol) was added to the solution in an ice bath, and then the reaction temperature was raised to room temperature and the reaction was continued for 10 h while stirring. After the reaction was over, the reaction was quenched with water. The reaction solution was dried over anhydrous sodium sulfate, filtered, concentrated, and purified by column chromatography to obtain the PEGylated lipid E6-1 (0.77 g). The main data of the ¹H-NMR spectrum of E6-1 are as follows: ¹H NMR (400 MHz, CDCl₃) δ: 4.29-4.13 (m, 2H, >NCH₂CH₂O(P═O)—), 4.06-3.90 (m, 4H, —(P═O)OCH₂CH₂CH₂—), 3.72-3.43 (m, PEG; 4H, —CH₂OCH₂CH₂N<), 3.36 (s, 6H, —OCH₃), 2.86-2.71 (m, 4H, —CH₂OCH₂CH₂N<; 2H, >NCH₂CH₂O(P═O)—), 1.66-1.04 (m, 24H, —CH₂CH₂CH₂—; 4H, —CH₂CH₃), 0.86 (t, 12H, —CH₂CH₃). The molecular weight of E6-1 determined by MALDI-TOF MS was 1478 Da.

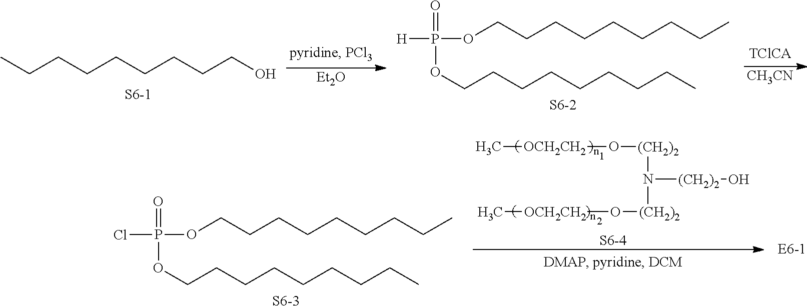

### Example 6.2: Preparation of E6-2

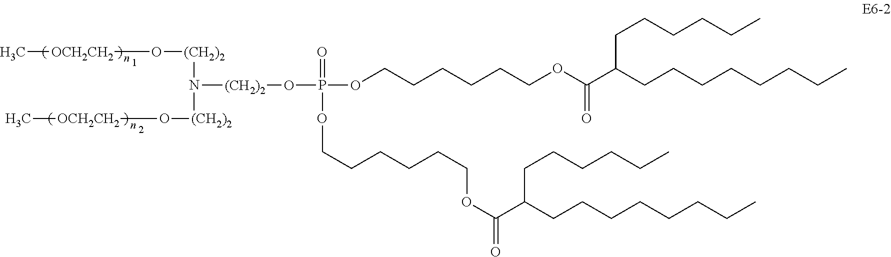

E6-2 corresponds to the general formula (1); wherein, X is

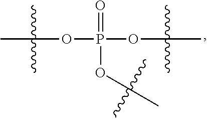

R₁ and R₂ are both

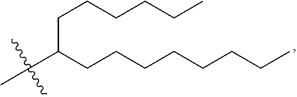 [SMILES: *C(CCCCCC)CCCCCCCC]

B₁ and B₂ are both hexylene groups, L₁ and L₂ are both ester groups (—OC(═O)—), L_{d} is —(CH₂)₂—, y=2, T is a methyl group, and the two Lₓ are both —(CH₂)₂—. The designed total molecular weight is approximately 2.9 kDa, wherein the molecular weight of each PEG chain is approximately 1 kDa, corresponding to n₁≈n₂≈22.

The preparation method is as follows:

Step a: S4-3 (6.43 g, 18.0 mmol) and pyridine (0.97 mL, 12.0 mmol) were dissolved in 80 mL diethyl ether. The solution was placed in an ice bath and added with phosphorus trichloride (PCl₃, 0.52 mL, 6.0 mmol) in 1 hour. After the feeding, the reaction solution was slowly raised to room temperature. After 16 hours of reaction with stirring, the reaction solution was filtered.

The precipitates were washed with 40 mL*2 diethyl ether. The filtrates were combined, dried over anhydrous sodium sulfate, filtered, concentrated under reduced pressure, and purified by column chromatography to obtain the intermediate S6-5 (4.40 g).

Step b: Under nitrogen protection, 50 mL solution of S6-5 (3.04 g, 4.0 mmol) in anhydrous acetonitrile was mixed with 5 mL solution of TCICA (0.31 g, 1.3 mmol) in anhydrous acetonitrile, and then reacted for 10 min at room temperature while stirring. White precipitates were formed. The stirring was continued for 2 hours, and then the precipitates were removed by filtration. The filtrate was concentrated under reduced pressure and purified by column chromatography to obtain the low-molecular weight chlorinated phospholipid compound intermediate S6-6 (2.86 g).

Step c: Under argon atmosphere, the polydisperse branched polyethylene glycol derivative S6-7 (2.10 g, 1.0 mmol, Mₙ≈2.1 kDa, n₁≈n₂≈22, PDI=1.01, having the same structure as S6-4, but with different values for n₁ and n₂), DMAP (24 mg, 0.2 mmol), and pyridine (87 μL, 1.1 mmol) were dissolved in dichloromethane (30 mL). Compound S6-6 (2.38 g, 3.0 mmol) was added to the solution in an ice bath, and then the reaction temperature was raised to room temperature and the reaction was continued for 10 h while stirring. After the reaction was over, the reaction was quenched with water. The reaction solution was dried over anhydrous sodium sulfate, filtered, concentrated, and purified by column chromatography to obtain the PEGylated lipid E6-2 (0.90 g). The main data of the ¹H-NMR spectrum of E6-2 are as follows: ¹H NMR (400 MHz, CDCl₃) δ: 4.30-4.12 (m, 2H, >NCH₂CH₂O(P═O)—), 4.09 (t, 4H, —CH₂OC(═O)—), 4.05-3.94 (m, 4H, —(P═O)OCH₂CH₂CH₂—), 3.72-3.42 (m, PEG; 4H, —CH₂OCH₂CH₂N<), 3.35 (s, 6H, —OCH₃), 2.84-2.70 (m, 4H, —CH₂OCH₂CH₂N<; 2H, >NCH₂CH₂O(P═O)—), 2.38-2.22 (m, 2H, —OC(═O)CH<), 1.70-1.00 (m, 48H, —CH₂CH₂CH₂—; 8H, —OC(═O)CHCH₂—; 8H, —CH₂CH₃), 0.85 (t, 12H, —CH₂CH₃). The molecular weight determined by GPC was approximately 2.9 kDa, PDI=1.01.

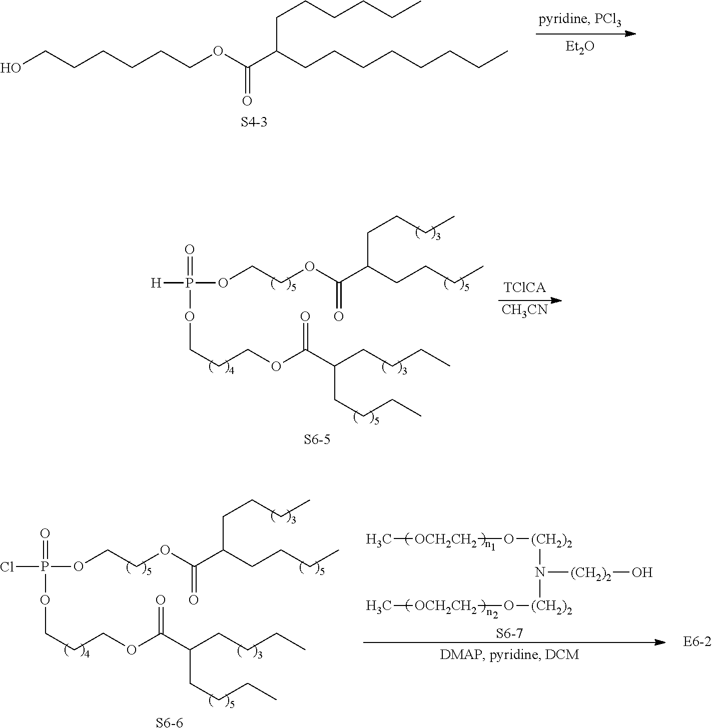

### Example 7: Non-Linear PEGylated Lipid E7-1

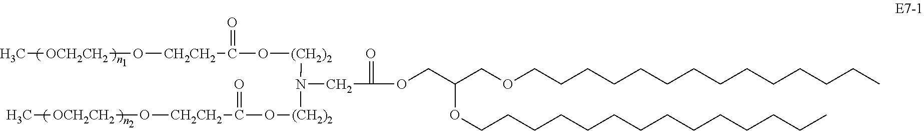

E7-1 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂C(═O)OCH₂—, y=2, T is a methyl group, and the two Lₓ are both —CH₂CH₂C(═O)OCH₂CH₂—. The molecular weight of each PEG chain is approximately 2 kDa, corresponding to n₁≈n₂≈45.

The preparation method is as follows:

Step a: Under argon atmosphere, compound S7-1 containing a free hydroxyl group (1.16 g, 2.4 mmol, prepared via the reaction between S2-1 and S1-1 and the subsequent removal of protecting groups, referring to the specific steps in Example 2.1), N,N-dihydroxyethylglycine derivative (S7-2, 0.78 g, 2.0 mmol), and DMAP (49 mg, 0.4 mmol) were dissolved in dichloromethane (20 mL), and then the solution of DCC (0.45 g, 2.2 mmol) in dichloromethane (5 mL) was added dropwise in an ice bath. Upon completion of the dropwise addition, the reaction temperature was raised to room temperature, and the reaction was stirred for 24 h. After the reaction was over, the precipitates were removed by filtration. The reaction solution was concentrated under reduced pressure, 10 mL tetrahydrofuran (THF) was added, and then 10 mL of the 1M solution of tetra-n-butylammonium fluoride (TBAF) in tetrahydrofuran was added. The reaction was conducted overnight to remove the TBS protection. After the reaction was over, the reaction solution was concentrated and extracted. The organic phases were combined, and then dried over anhydrous sodium sulfate, filtered, concentrated, and purified by column chromatography to obtain the intermediate S7-3 (1.11 g).

Step b: Under argon atmosphere, methoxypolyethylene glycol propionic acid S7-4 (4.20 g, 2.0 mmol, Mₙ≈2.1 kDa, n₁≈45, PDI=1.01), low-molecular-weight lipid compound S7-3 (0.50 g, 0.8 mmol), and DMAP (49 mg, 0.4 mmol) were dissolved in dichloromethane (25 mL), and then the solution of DCC (0.45 g, 2.2 mmol) in dichloromethane (5 mL) was added dropwise in an ice bath. Upon completion of the dropwise addition, the reaction temperature was raised to room temperature, and the reaction was stirred for 24 h. After the reaction was over, the precipitates were removed by filtration. The filtrate was dried over anhydrous sodium sulfate, concentrated, and purified by column chromatography to obtain the PEGylated lipid E7-1 (1.05 g). The main data of the ¹H-NMR spectrum of E7-1 are as follows: ¹H NMR (400 MHz, CDCl₃) δ:4.33-4.12 (m, 2H, —C(═O)OCH₂CH<; 4H, —C(═O)OCH₂CH₂N<), 3.78-3.40 (m, PEG; 4H, —OCH₂CH₂C(═O)O—; 1H, —C(═O)OCH₂CH<; 4H, >CHCH₂OCH₂CH₂—; 2H, >CHOCH₂CH₂—), 3.38 (s, 6H, —OCH₃), 3.32 (s, 2H, >NCH₂C(═O)O—), 3.05-2.93 (m, 4H, —C(═O)OCH₂CH₂N<), 2.66-2.50 (m, 4H, —OCH₂CH₂C(═O)O—), 1.53-0.98 (m, 44H, —CH₂CH₂CH₂—; 4H, —CH₂CH₃), 0.82 (t, 6H, —CH₂CH₃). The molecular weight determined by GPC was approximately 4.7 kDa, PDI=1.02.

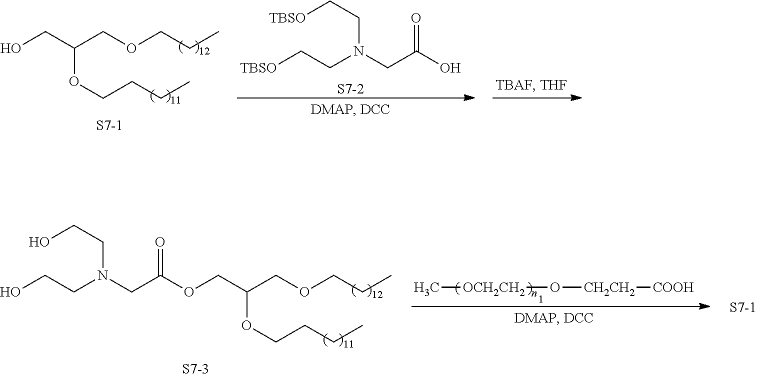

### Example 8: Non-Linear PEGylated Lipids E8-1 and E8-2

### Example 8.1: Preparation of E8-1

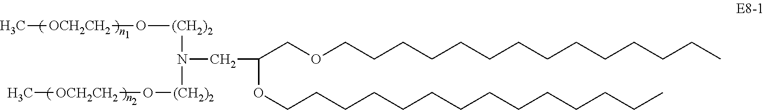

E8-1 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂—, y=2, T is a methyl group, and the two Lₓ are both —(CH₂)₂—. The designed total molecular weight is approximately 2.6 kDa, wherein the molecular weight of each PEG chain is approximately 1 kDa, corresponding to n₁≈n₂≈22.

### Preparation Method 1

Step a: S2-3 (2.42 g, 5.0 mmol), S8-1 (3.64 g, 11.0 mmol, S8-1 is an ethylene glycol derivative containing a TBS-protected hydroxyl group while the other hydroxyl group is replaced by the OTs substituent), and potassium carbonate (K₂CO₃, 2.42 g, 17.5 mmol) were successively dissolved in 50 mL acetonitrile (MeCN) and then stirred at 60° C. for 20 hours. After the reaction was over, the reaction solution was concentrated under reduced pressure, and then the TBAF/THF solution was used to remove the TBS protection. After the reaction was over, the reaction solution was concentrated and extracted. The organic phases were combined, and then dried over anhydrous sodium sulfate, filtered, concentrated, and purified by column chromatography to obtain the intermediate S8-2 (1.46 g).

Step b: Into a sealed reactor under anhydrous and oxygen-free conditions, 50 mL tetrahydrofuran was added, followed by adding diphenylmethyl potassium (DPMK, 2.06 g, 10.0 mmol) and S8-2 (1.14 g, 2.0 mmol). After thorough mixing, methoxypolyethylene glycol sulfonate derivative S8-3 (7.20 g, 6.0 mmol, Mₙ≈1.2 kDa, n₁≈22, PDI=1.01) was added. After 12 hours of reaction at 30° C., the reactor was opened. The reaction solution was concentrated and washed, and then the crude product was purified by column chromatography to obtain the PEGylated lipid E8-1 (1.56 g). The main data of the ¹H-NMR spectrum of E8-1 are as follows:

¹H NMR (400 MHz, CDCl₃) δ: 3.75-3.35 (m, PEG; 4H, —OCH₂CH₂N<; 3H, >CHCH₂O—; 4H, —OCH₂CH₂CH₂—; 6H, —OCH₃), 2.83-2.70 (m, 4H, —OCH₂CH₂N<), 2.54-2.48 (m, 2H, >NCH₂CH<), 1.52-1.01 (m, 44H, —CH₂CH₂CH₂—; 4H, —CH₂CH₃), 0.87 (t, 6H, —CH₂CH₃). The molecular weight determined by GPC was approximately 2.6 kDa, PDI=1.01.

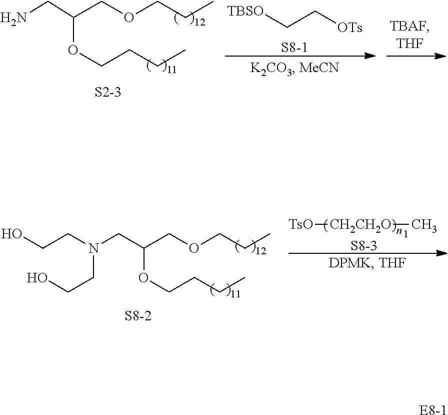

### Preparation Method 2

Step a: Into a sealed reactor under anhydrous and oxygen-free conditions, 250 mL tetrahydrofuran, small-molecule initiator S8-2 (1.43 g, 2.0 mmol), and diphenylmethyl potassium were successively added, and then a calculated amount of ethylene oxide (5.7 mL) was added. The temperature was gradually raised to 60° C., and the reaction was carried out for 48 hours.

Step b: Excess diphenylmethyl potassium (8.24 g, 40.0 mmol) and excess capping reagent iodomethane (14.20 g, 100.0 mmol) were added successively, and the reaction was conducted at 30° C. for 12 hours. After the reaction was over, the reactor was opened, the solvent was concentrated, and the precipitation was carried out in diethyl ether at 0° C. After filtration and drying, the crude product was purified by column chromatography to obtain the PEGylated lipid E8-1 (5.46 g). The structure of E8-1 was determined by ¹H NMR. The molecular weight of the E8-1 obtained through the preparation method 2 determined by GPC was approximately 2.6 kDa, PDI=1.02.

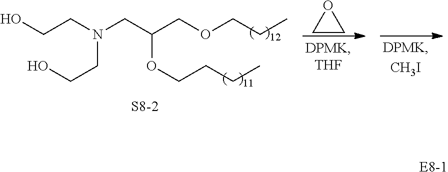

### Example 8.2: Preparation of E8-2

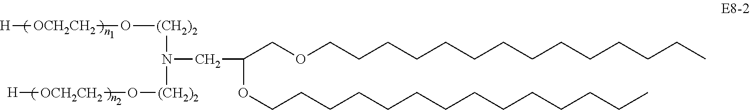

E8-2 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂—, y=2, T is a hydrogen atom, and the two Lₓ are both —(CH₂)₂—. The designed total molecular weight is approximately 2.5 kDa, wherein the molecular weight of each PEG chain is approximately 1 kDa, corresponding to n₁≈n₂≈22.

### Preparation Method 1

Into a sealed reactor under anhydrous and oxygen-free conditions, 50 mL tetrahydrofuran was added, followed by adding DPMK (2.06 g, 10.0 mmol) and S8-2 (1.14 g, 2.0 mmol). After thorough mixing, polyethylene glycol sulfonate derivative containing a TBS-protected hydroxyl group S8-5 (7.80 g, 6.0 mmol, Mₙ≈1.3 kDa, n₁≈22, PDI=1.01) was added. After 12 hours of reaction at 30° C., the reactor was opened. The reaction solution was concentrated and washed, and then the crude product was purified by column chromatography to obtain the PEGylated lipid E8-2 (1.72 g). The main data of the ¹H-NMR spectrum of E8-2 are as follows: ¹H NMR (400 MHz, CDCl₃) δ: 3.74-3.35 (m, PEG; 4H, —OCH₂CH₂N<; 3H, >CHCH₂O—; 4H, —OCH₂CH₂CH₂—), 2.85-2.70 (m, 4H, —OCH₂CH₂N<), 2.55-2.48 (m, 2H, >NCH₂CH<), 1.52-1.00 (m, 44H, —CH₂CH₂CH₂—; 4H, —CH₂CH₃), 0.85 (t, 6H, —CH₂CH₃). The molecular weight of E8-2 determined by GPC was approximately 2.5 kDa, PDI=1.02.

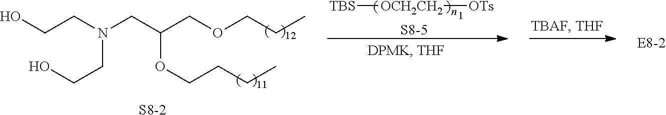

### Preparation Method 2

Referring to the preparation method 2 in Example 8.1, using the same starting materials and molar equivalents, using methanol as the capping reagent, the PEGylated lipid E8-2 (5.06 g) was obtained. The structure of E8-2 was determined by ¹H NMR. The molecular weight of the E8-2 obtained through the preparation method 2 determined by GPC was approximately 2.5 kDa, n₁≈n₂≈22, PDI=1.02.

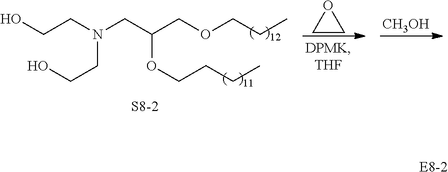

### Example 9: Non-Linear PEGylated Lipids E9-1 and E9-2

### Example 9.1: Preparation of E9-1

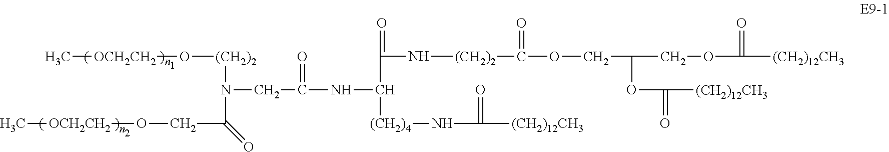

E9-1 corresponds to the general formula (1); wherein, X is —CH<, R₁ is

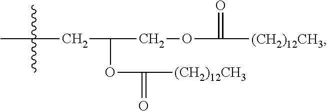

R₂ is a tridecyl group, B₁ is a linking bond, B₂ is a butylene group, L₁ is —C(═O)NH—(CH₂)₂—C(═O)O—, L₂ is an amide group (—NHC(═O)—), L_{d} is —CH₂C(═O)NH—, y=2, T is a methyl group, and the two Lₓ are —(CH₂)₂— and —CH₂C(═O)—, respectively. The designed total molecular weight is approximately 3.1 kDa, wherein the molecular weight of each PEG chain is approximately 1 kDa, corresponding to n₁≈n₂≈22.

The preparation method is as follows:

Step a: S1-2 (2.28 g, 10.0 mmol) was dissolved in 40 mL anhydrous chloromethane, added with NHS (1.73 g, 15.0 mmol), and then added with DCC (3.09 g, 15.0 mmol). Into 50 mL solution of the hetero-bifunctional, branched polyethylene glycol derivative S9-1 (7.92 g, 3.3 mmol, Mₙ≈2.4 kDa, n₁≈n₂≈22, PDI=1.02, prepared following the methods in CN108530617B, wherein the carboxyl group is protected by tBu) in dichloromethane, DMAP (0.24 g, 2.0 mmol) was added. Said two solutions were mixed and then reacted while stirring at room temperature for 24 hours. The reaction solution was filtered to remove the precipitates, and then concentrated under reduced pressure. The TFA/DCM mixed solution (1:1 v/v) was used to remove the tBu protection. After washing with purified water and extracting with dichloromethane, the extract obtained was dried over anhydrous sodium sulfate, filtered, concentrated, and purified by column chromatography to obtain the intermediate S9-2 (3.78 g).

Step b: Under argon atmosphere, S9-2 (2.60 g, 1.0 mmol, Mₙ≈2.6 kDa, n₁≈n₂˜22, PDI=1.02), S1-3 (1.54 g, 3.0 mmol), and DMAP (24 mg, 0.2 mmol) were dissolved in dichloromethane (20 mL), and then the solution of DCC (0.23 g, 1.1 mmol) in dichloromethane (5 mL) was added dropwise in an ice bath. Upon completion of the dropwise addition, the reaction temperature was raised to room temperature, and the reaction was stirred for 24 h. After the reaction was over, the precipitates were removed by filtration. The filtrate was dried over anhydrous sodium sulfate, concentrated, and purified by column chromatography to obtain the PEGylated lipid E9-1 (1.27 g). The main data of the ¹H-NMR spectrum of E9-1 are as follows: ¹H NMR (400 MHz, CDCl₃) δ: 5.32-5.21 (m, 1H, >CHCH₂OC(═O)—), 4.32-4.10 (m, 2H, —C(═O)OCH₂CH<; 2H, >CHCH₂OC(═O)—; 1H, —C(═O)NHCH<; 2H, —OCH₂C(═O)N<), 3.72-3.40 (m, PEG; 4H, —OCH₂CH₂N<; 2H, >NCH₂C(═O)NH—), 3.33 (s, 6H, —OCH₃), 3.26-3.14 (m, 2H, —CH₂CH₂NHC(═O)CH₂—), 2.68-2.47 (m, 4H, —C(═O)NH(CH₂)₂C(═O)O—), 2.30 (t, 4H, —OC(═O)CH₂CH₂CH₂—), 2.20-2.10 (m, 2H, —NHC(═O)CH₂CH₂—), 1.75-1.14 (m, 64H, —CH₂CH₂CH₂—; 2H, —CHCH₂CH₂—; 6H, —CH₂CH₃), 0.86 (t, 9H, —CH₂CH₃). The molecular weight determined by GPC was approximately 3.1 kDa, PDI=1.02.

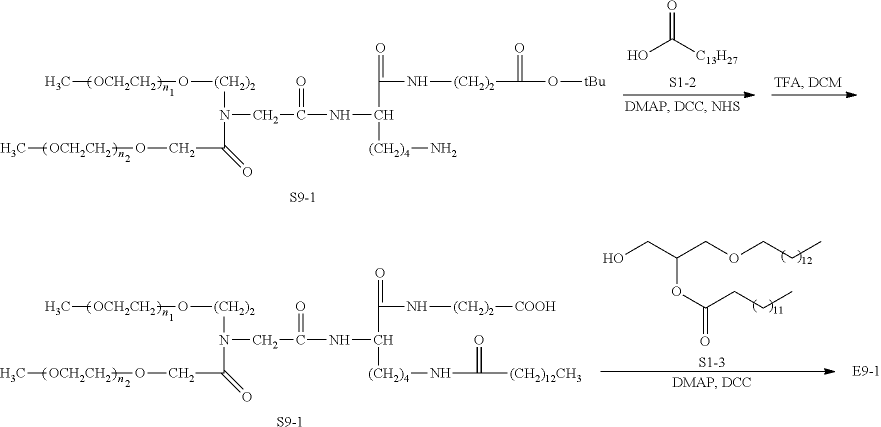

### Example 9.2: Preparation of E9-2

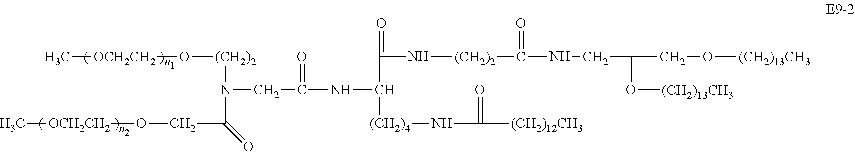

E9-2 corresponds to the general formula (1); wherein, X is —CH<, R₁ is

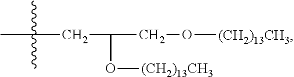

R₂ is a tridecyl group, B₁ is a linking bond, B₂ is an butylene group, L₁ is —C(═O)NH—(CH₂)₂—C(═O)NH—, L₂ is an amide group (—NHC(═O)—), L_{d} is —CH₂C(═O)NH—, y=2, two x are both 1, T is a methyl group, and the two Lₓ are —(CH₂)₂— and —CH₂C(═O)—, respectively. The designed total molecular weight is approximately 3.1 kDa, wherein the molecular weight of each PEG chain is approximately 1 kDa, corresponding to n₁≈n₂≈22.

The preparation method is as follows:

Under argon atmosphere, S9-2 (2.60 g, 1.0 mmol, Mₙ≈2.6 kDa, n₁≈n₂≈22, PDI=1.02), S2-3 (1.45 g, 3.0 mmol), and DMAP (24 mg, 0.2 mmol) were dissolved in dichloromethane (20 mL), and then the solution of DCC (0.23 g, 1.1 mmol) in dichloromethane (5 mL) was added dropwise in an ice bath. Upon completion of the dropwise addition, the reaction temperature was raised to room temperature, and the reaction was stirred for 24 h. After the reaction was over, the precipitates were removed by filtration. The filtrate was dried over anhydrous sodium sulfate, concentrated, and purified by column chromatography to obtain the PEGylated lipid E9-2 (1.12 g). The main data of the ¹H-NMR spectrum of E9-2 are as follows: ¹H NMR (400 MHz, CDCl₃) δ: 4.25-4.12 (m, 1H, —C(═O)NHCH<; 2H, —OCH₂C(═O)N<), 3.72-3.31 (m, PEG; 4H, —OCH₂CH₂N<; 2H, >NCH₂C(═O)NH—; 3H, >CHCH₂O—; 4H, —OCH₂CH₂CH₂—; 6H, —OCH₃), 3.25-3.15 (m, 2H, —CH₂CH₂NHC(═O)CH₂—), 3.07-2.94 (m, 2H, —C(═O)NHCH₂CH<), 2.68-2.47 (m, 4H, —C(═O)NH(CH₂)₂C(═O)O—), 2.21-2.10 (m, 2H, —NHC(═O)CH₂CH₂—), 1.75-1.15 (m, 68H, —CH₂CH₂CH₂—; 2H, —CHCH₂CH₂—; 6H, —CH₂CH₃), 0.88 (t, 9H, —CH₂CH₃). The molecular weight determined by GPC was approximately 3.1 kDa, PDI=1.02.

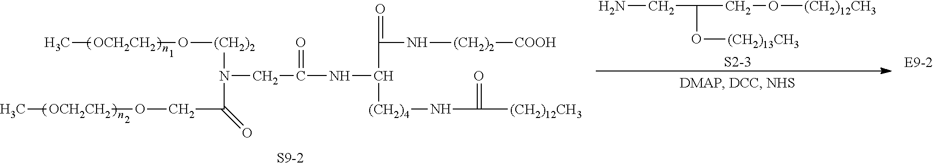

### Example 10: Non-Linear PEGylated Lipid Coupled with Folic Acid, E10-1

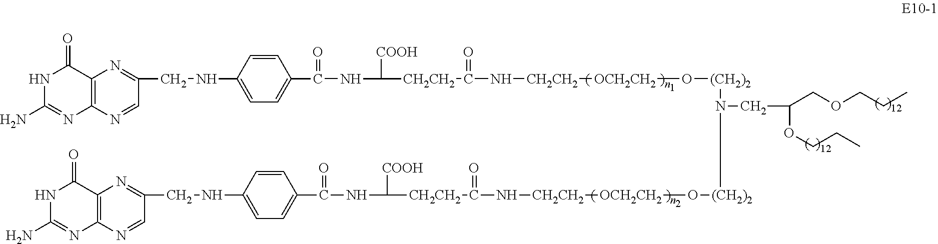

E10-1 corresponds to the general formula (1); wherein, X is —CH<, R₁ and R₂ are both tetradecyl groups, B₁ is a methylene group, B₂ is a linking bond, L₁ and L₂ are both ether group (—O—), L_{d} is —CH₂—, y=2, two x are both 1, T is R₀₁-L₀₁-, wherein L₀₁ is —NHCH₂CH₂—, R₀₁ is a residue of folic acid

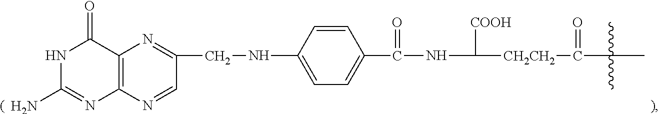

and the two Lₓ are both —(CH₂)₂—. The designed total molecular weight is approximately 3.4 kDa, wherein the molecular weight of each PEG chain is approximately 1 kDa, corresponding to n₁≈n₂≈22.

The preparation method is as follows:

Step a: Referring to the reaction conditions and feeding ratios of the Step c and Step d in Example 2.1, two alkylation reactions were carried out using S2-3 (8.71 g, 18.0 mmol) and the polyethylene glycol sulfonate derivative S10-1 containing a Boc-protected amino group (2.16 g, 1.8 mmol per feed, Mₙ≈1.2 kDa, n₁≈22, PDI=1.01), followed by using TFA/DCM to remove the Boc protecting group. After purification by column chromatography, the non-linear PEGylated lipid S10-2 containing two terminal amino groups (1.14 g) was obtained.

Step b: Folic acid (S10-3, 0.44 g, 1.0 mmol) was dissolved in 8 mL anhydrous dichloromethane, added with NHS (0.14 g, 1.2 mmol), and then added with DCC (0.25 g, 1.2 mmol). Into 5 mL solution of S10-2 (1.00 g, 0.4 mmol, Mₙ≈2.5 kDa, n₁≈n₂≈22, PDI=1.02) in dichloromethane, DMAP (24 mg, 0.2 mmol) was added. Said two solutions were mixed and then reacted while stirring at room temperature for 24 hours. The reaction solution was filtered to remove the precipitates, and then precipitated in anhydrous diethyl ether at 0° C. After filtration, concentration, and purification by column chromatography, the PEGylated lipid E10-1 (0.42 g) was obtained. The main data of the ¹H-NMR spectrum of E10-1 are as follows: ¹H NMR (400 MHz, CDCl₃) δ: 8.71 (s, 2H, pterinC7), 7.66-7.64 (d, 4H, Ph), 6.67 (d, 4H, Ph), 4.49-4.48 (d, 4H, pterinC6-CH₂NH—), 4.32-4.20 (m, 2H, —C(═O)NHCH(COOH)CH₂—), 3.75-3.35 (m, PEG; 4H, —OCH₂CH₂N<; 3H, >CHCH₂O—; 4H, —OCH₂CH₂CH₂—; 8H, —C(═O)NH(CH₂)₂₀—), 2.84-2.71 (m, 4H, —OCH₂CH₂N<), 2.54-2.46 (m, 2H, >NCH₂CH<), 2.36-2.25 (m, 4H, —CH(COOH)CH₂CH₂C(═O)NH—), 2.07-1.93 (m, 4H, —CH(COOH)CH₂CH₂C(═O)NH—), 1.53-1.01 (m, 44H, —CH₂CH₂CH₂—; 4H, —CH₂CH₃), 0.85 (t, 6H, —CH₂CH₃). The molecular weight determined by GPC was approximately 3.4 kDa, PDI=1.02.

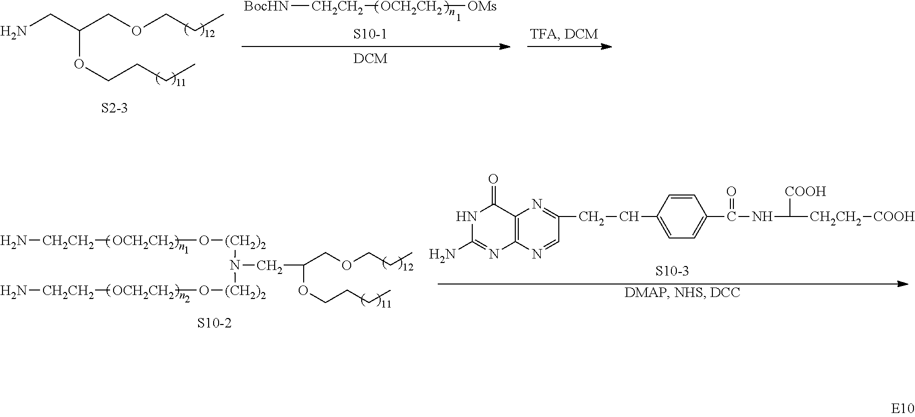

### Example 11: Non-Linear PEGylated Lipid E11-1

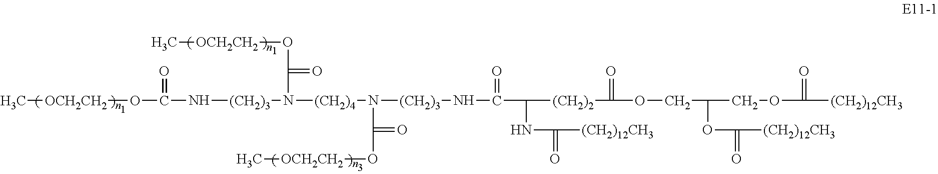

E11-1 corresponds to the general formula (1); wherein, X is —CH<, R₁ is

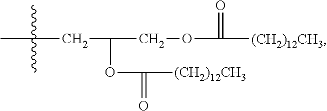

R₂ is a tridecyl group, B₁ is an ethylene group, B₂ is a linking bond, L₁ is an ester group (—C(═O)O—), L₂ is an amide group (—NHC(═O)—), L_{d} is —(CH₂)₃NHC(═O)—, N_{core} is

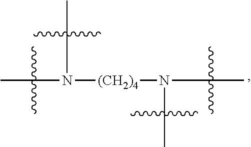

y=3, T is a methyl group, and the three Lₓ are —C(═O)NH(CH₂)₃—, —C(═O)—, and —C(═O)—, respectively. The designed total molecular weight is approximately 4.1 kDa, wherein the molecular weight of each PEG chain is approximately 1 kDa, corresponding to n₁≈n₂≈n₃≈22.

The preparation method is as follows:

Referring to the reaction conditions and feeding ratios of the Step a and Step b in Example 9.1, the branched three-arm polyethylene glycol amino carboxylic acid derivative (11.55 g, 3.3 mmol, Mₙ≈3.5 kDa, n₁≈n₂≈n₃≈22, PDI=1.02, prepared following the methods in CN108530617B, wherein the carboxyl group is protected by tBu) reacted with S1-2 and S1-3, successively. The three-arm PEGylated lipid E11-1 (1.46 g) was obtained after purification by column chromatography. The main data of the ¹H-NMR spectrum of E11-1 are as follows: ¹H NMR (400 MHz, CDCl₃) δ: 5.32-5.21 (m, 1H, —CH₂CHCH₂—), 4.30-4.14 (m, 4H, —CH₂CHCH₂—; 6H, —OCH₂CH₂OC(═O)—), 3.71-3.00 (m, PEG; 6H, —OCH₂CH₂OC(═O)—; 4H, —OC(═O)NHCH₂CH₂CH₂N<; 4H, >NCH₂CH₂CH₂NHC(═O)—; 4H, >NCH₂(CH₂)₂CH₂N<; 9H, —OCH₃), 2.46-2.32 (m, 2H, >CHCH₂CH₂C(═O)O—), 2.27 (t, 4H, —OC(═O)CH₂CH₂CH₂—), 2.18-2.09 (m, 2H, —NHC(═O)CH₂CH₂—; 2H, >CHCH₂CH₂C(═O)O—), 1.95-1.09 (m, 68H, —CH₂CH₂CH₂—; 6H, —CH₂CH₃), 0.87 (t, 9H, —CH₂CH₃). The molecular weight determined by GPC was approximately 4.1 kDa, PDI=1.02.

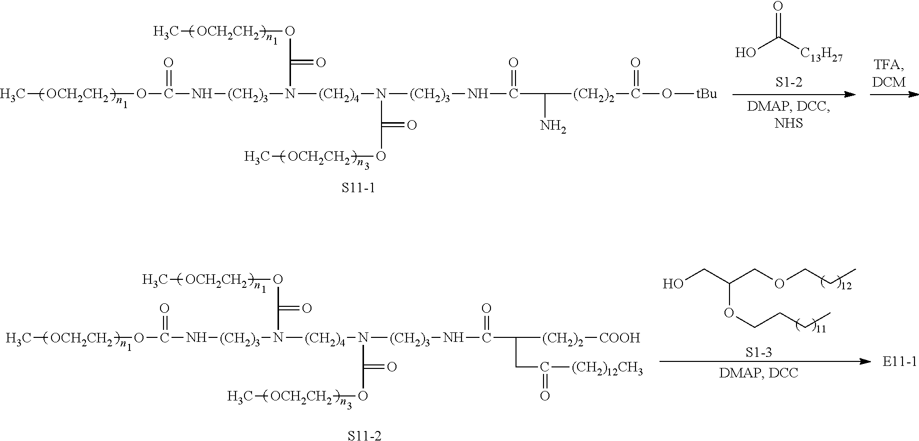

### Example 12: Non-Linear PEGylated Lipid E12-1

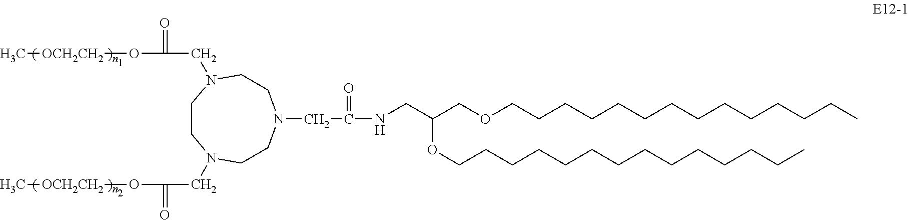

E12-1 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂C(═O)NHCH₂—, N_{core} is

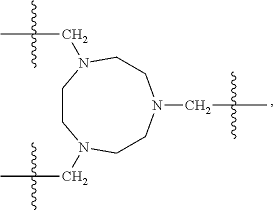

y=2, T is a methyl group, and the two Lₓ are both —C(═O)CH₂—. The designed total molecular weight is approximately 2.7 kDa, wherein the molecular weight of each PEG chain is approximately 1 kDa, corresponding to n₁≈n₂≈22.

The preparation method is as follows:

Step a: The small-molecule compound NOTA-bis(t-Bu ester) (S12-1, 1.25 g, 3.0 mmol) was dissolved in 20 mL anhydrous dichloromethane, added with NHS (0.52 g, 4.5 mmol), and then added with DCC (0.93 g, 4.5 mmol). Into 30 mL solution of S2-3 (1.74 g, 3.6 mmol) in dichloromethane, DMAP (73 mg, 0.6 mmol) was added. Said two solutions were mixed and then reacted while stirring at room temperature for 24 hours. The reaction solution was filtered to remove the precipitates, and then concentrated under reduced pressure. The TFA/DCM mixed solution (1:1 v/v) was used to remove the tBu protection. After washing with purified water and extracting with dichloromethane, the extract obtained was dried over anhydrous sodium sulfate, filtered, concentrated, and purified by column chromatography to obtain the intermediate S12-2 (1.85 g).

Step b: Under argon atmosphere, S12-2 (1.54 g, 2.0 mmol), methoxypolyethylene glycol S12-3 (6.00 g, 6.0 mmol, Mₙ≈1.0 kDa, n₁≈n₂≈22, PDI=1.01), and DMAP (0.10 g, 0.8 mmol) were dissolved in dichloromethane (40 mL), and then the solution of DCC (0.91 g, 4.4 mmol) in dichloromethane (15 mL) was added dropwise in an ice bath. Upon completion of the dropwise addition, the reaction temperature was raised to room temperature, and the reaction was stirred for 24 h. After the reaction was over, the precipitates were removed by filtration. The filtrate was concentrated under reduced pressure, and the TFA/DCM mixed solution (1:1 v/v) was used to remove the tBu protection. After washing with purified water and extracting with dichloromethane, the extract obtained was dried over anhydrous sodium sulfate, filtered, concentrated, and purified by column chromatography to obtain the PEGylated lipid E12-1 (1.78 g). The main data of the ¹H-NMR spectrum of E12-1 are as follows: ¹H NMR (400 MHz, CDCl₃) δ: 4.19-4.07 (m, 4H, —OCH₂CH₂OC(═O)—), 3.72-3.31 (m, PEG; 4H, —OCH₂CH₂OC(═O)—; 3H, >CHCH₂O—; 4H, —OCH₂CH₂CH₂—; 6H, —OCH₃), 3.24 (s, 4H, —OC(═O)CH₂N<), 3.21 (s, 2H, >NCH₂C(═O)NH—), 3.06-2.92 (m, 2H, —C(═O)NHCH₂CH<), 2.82-2.72 (m, 12H, >N(CH₂)₂N<), 1.55-1.11 (m, 44H, —CH₂CH₂CH₂—; 4H, —CH₂CH₃), 0.88 (t, 6H, —CH₂CH₃). The molecular weight determined by GPC was approximately 2.7 kDa, PDI=1.01.

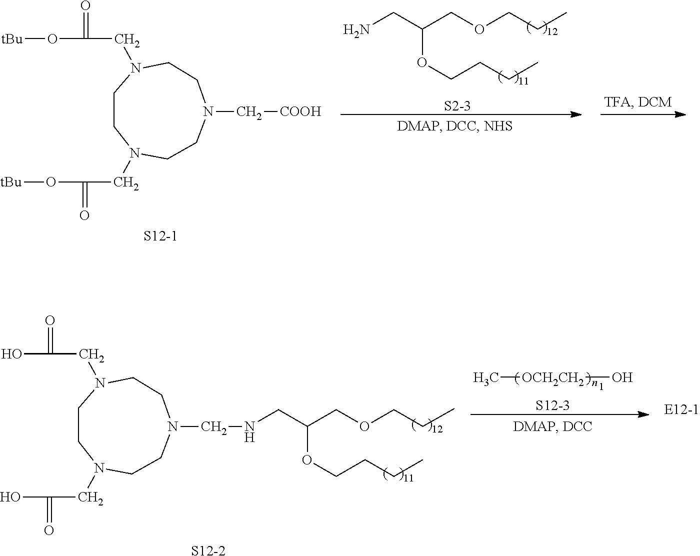

### Example 13: Non-Linear PEGylated Lipid E13-1

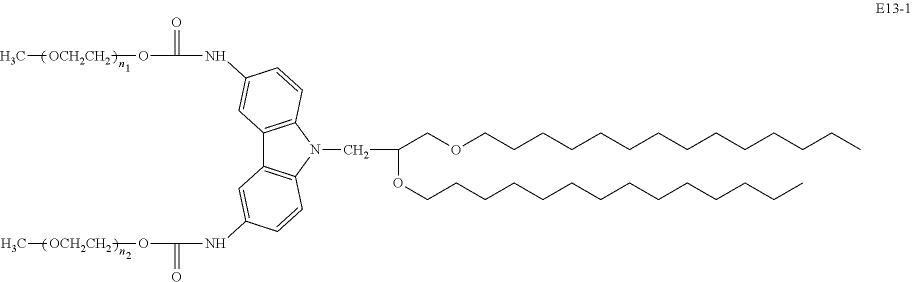

E13-1 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂—, N_{core} is

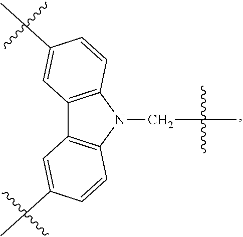

y=2, T is a methyl group, and the two Lₓ are both —C(═O)NH—. The designed total molecular weight is approximately 2.7 kDa, wherein the molecular weight of each PEG chain is approximately 1 kDa, corresponding to n₁≈n₂≈22.

The preparation method is as follows:

Step a: S7-1 (4.85 g, 10.0 mmol) was azeotroped with toluene (100 mL) at 140° C. to remove water. After distilling off 30 mL of solvent, the reaction was cooled to room temperature. Triethylamine (TEA, 2.02 g, 20.0 mmol) and methanesulfonyl chloride (MsCl, 2.05 g, 18.0 mmol) were added, and the reaction was stirred overnight at room temperature. After the reaction was over, the reaction solution was poured into water (100 mL), and then extracted with EtOAc twice (50 mL*2). The aqueous phase was retained and further extracted twice with dichloromethane (50 mL*2), and the organic phases obtained were then combined, dried, filtered, concentrated, and purified by column chromatography to obtain the compound S13-1 (4.84 g).

Step b: The carbazole derivative containing two protected amino groups S13-2 (1.99 g, 5.0 mmol) was dissolved in 40 mL dried THF, slowly added with NaH (60%, 2.00 g, 50.0 mmol) in an ice bath, and reacted for 1 hour in an ice bath. After the reaction was over, S13-1 (3.38 g, 6.0 mmol) was added, and the reaction was continued for 1 hour while stirring in an ice bath, after which the reaction solution was slowly returned to room temperature and the reaction was continued overnight. After the reaction was over, the reaction was placed in an ice bath, followed by adding water to quench the reaction. After stirring for 30 minutes, the solvent was removed via rotary evaporation, and then water (40 mL) was added and mixed while stirring. Then, extraction was performed twice using EtOAc (40 mL*2). The aqueous phase was retained and then extracted twice with dichloromethane (40 mL*2). The dichloromethane organic phases were combined and washed with saturated solution of sodium chloride (20 mL*4), and then dried over anhydrous sodium sulfate, filtered, concentrated, and purified by column chromatography to obtain the diamine small-molecule intermediate S13-3 (2.91 g).

Step c: The methoxypolyethylene glycol succinimide carbonate mPEG-SC (S13-4, 2.88 g, 2.4 mmol, Mₙ≈1.2 kDa, n₁≈22, PDI=1.01) was dissolved in 20 mL anhydrous dichloromethane, added with DMAP (61 mg, 0.5 mmol), and mixed while stirring. The solution of diamine small-molecule intermediate S13-3 (0.66 g, 1.0 mmol) in anhydrous dichloromethane was slowly added dropwise under the condition of an ice bath, mixed, and reacted at 25° C. for 16 h. After the reaction was over, the reaction solution was washed with sodium bicarbonate solution, 3M sodium hydroxide solution, and saturated brine. The organic phase was concentrated and purified by column chromatography to obtain the PEGylated lipid E13-1 (1.30 g). The main data of the ¹H-NMR spectrum of E13-1 are as follows: ¹H NMR (400 MHz, CDCl₃) δ: 8.21 (s, 2H, carbazole), 7.49-7.41 (m, 4H, carbazole), 4.30-4.11 (m, 4H, —OCH₂CH₂OC(═O)—; 2H, >NCH₂CH<), 3.75-3.33 (m, PEG; 4H, —OCH₂CH₂OC(═O)—; 3H, >CHCH₂O—; 4H, —OCH₂CH₂CH₂—; 6H, —OCH₃), 1.53-0.97 (m, 44H, —CH₂CH₂CH₂—; 4H, —CH₂CH₃), 0.83 (t, 6H, —CH₂CH₃). The molecular weight determined by GPC was approximately 2.7 kDa, PDI=1.02.

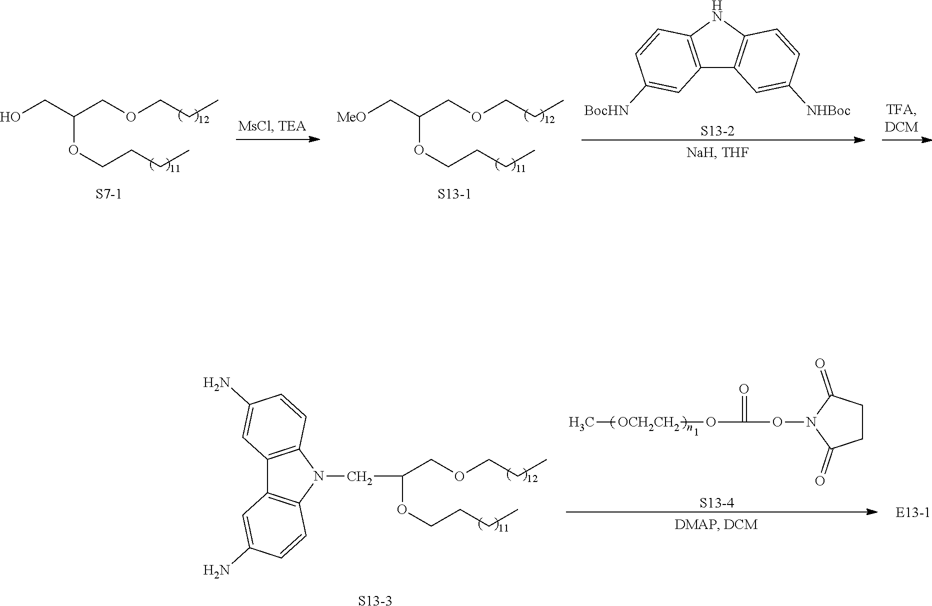

### Example 14: Non-Linear PEGylated Lipids E14-1 and E14-2

### Example 14.1: Preparation of E14-1

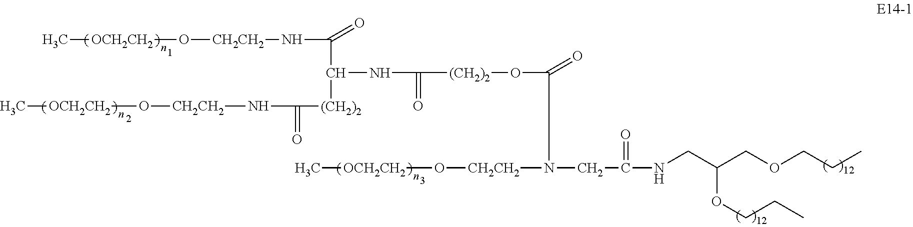

E14-1 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂C(═O)NHCH₂—, y=2, two XPEG contain 1 and 2 RPEG, respectively; T is a methyl group, one of XPEG has a non-linear structure and contains the

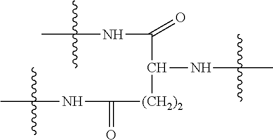

trivalent branching core, and the two Lₓ are —C(═O)(CH₂)₂OC(═O)— and —CH₂CH₂—, respectively. The designed total molecular weight is approximately 3.7 kDa, wherein the molecular weight of each PEG chain is approximately 1 kDa, corresponding to n₁≈n₂≈n₃≈22.

The preparation method is as follows:

Step a: The glutamic acid derivative containing a Boc-protected amino group (S4-4, 2.96 g, 12.0 mmol) was dissolved in 100 mL anhydrous dichloromethane, added with NHS (4.14 g, 36.0 mmol), and then added with DCC (7.42 g, 36.0 mmol). Into 200 mL solution of methoxypolyethylene glycol amine S14-1 (28.8 g, 28.8 mmol, Mₙ≈1.0 kDa, n₁≈22, PDI=1.01) in dichloromethane, DMAP (0.59 g, 4.8 mmol) was added. Said two solutions were mixed and then reacted while stirring at room temperature for 24 hours. The reaction solution was filtered to remove the precipitates, and then concentrated under reduced pressure. The TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the non-linear two-arm methoxypolyethylene glycol amine derivative S14-2 (9.07 g).

Step b: 3-Hydroxypropionic acid containing a TBS-protected hydroxyl group (S14-3, 2.45 g, 12.0 mmol) was dissolved in 50 mL anhydrous dichloromethane, added with NHS (2.07 g, 18.0 mmol), and then added with DCC (3.71 g, 18.0 mmol). Into 50 mL solution of S14-2 (8.32 g, 4.0 mmol, Mₙ≈2.1 kDa, n₁≈n₂≈22, PDI=1.01) in dichloromethane, DMAP (0.29 g, 2.4 mmol) was added. Said two solutions were mixed and then reacted while stirring at room temperature for 24 hours. The reaction solution was filtered to remove the precipitates, and then concentrated under reduced pressure. The TBAF/THF solution was used to remove the TBS protection. After the reaction was over, the reaction solution was concentrated and extracted. The organic phases were combined, and then dried over anhydrous sodium sulfate, filtered, concentrated, and purified by column chromatography to obtain the non-linear two-arm methoxypolyethylene glycol derivative S14-4 (4.70 g).

Step c: S14-4 (4.40 g, 2.0 mmol, Mₙ≈2.2 kDa, n₁≈n₂≈22, PDI=1.01) was dissolved in 30 mL anhydrous acetonitrile, added with TEA (0.33 mL, 2.4 mmol) and N,N′-disuccinimidyl carbonate (DSC, 0.61 g, 2.4 mmol), and then reacted at room temperature while stirring overnight. Then, the reaction solution was concentrated under reduced pressure. The residue was dissolved with 30 mL dichloromethane and then washed with saturated sodium bicarbonate solution (15 mL*3). The organic phase was dried over anhydrous sodium sulfate and filtered. The filtrate was added with the methoxypolyethylene glycol carboxylic acid derivative S14-5 (2.20 g, 2.0 mmol, Mₙ≈1.1 kDa, n₁≈22, PDI=1.01, S14-5 was prepared via an alkylation reaction between S4-9 and glycine (NH₂CH₂COOH), referring to the Step c in Example 2.1) and TEA (0.33 mL, 2.4 mmol). The reaction was conducted at room temperature for 2 hours, and then the reaction solution was concentrated and washed. The crude product was purified by column chromatography to obtain the non-linear three-arm methoxypolyethylene glycol carboxylic acid derivative S14-6 (3.14 g).

Step d: S14-6 (2.64 g, 0.8 mmol, Mₙ≈3.3 kDa, n₁≈n₂≈n₃≈22, PDI=1.02) was dissolved in 20 mL anhydrous dichloromethane, added with NHS (0.14 g, 1.2 mmol), and then added with DCC (0.25 g, 1.2 mmol). Into 15 mL solution of S2-3 (1.16 g, 2.4 mmol) in dichloromethane, DMAP (20 mg, 0.2 mmol) was added. Said two solutions were mixed and then reacted while stirring at room temperature for 24 hours. After the reaction was over, the precipitates were removed by filtration. The filtrate was dried over anhydrous sodium sulfate, filtered, concentrated, and purified by column chromatography to obtain the PEGylated lipid E14-1 (1.30 g). The main data of the ¹H-NMR spectrum of E14-1 are as follows: ¹H NMR (400 MHz, CDCl₃) δ: 4.28-4.12 (m, 1H, —NHC(═O)CH<; 2H, —C(═O)CH₂CH₂O—), 4.03 (s, 2H, >NCH₂C(═O)—), 3.73-3.33 (m, PEG; 3H, >CHCH₂O—; 4H, —OCH₂CH₂CH₂—; 8H, —OCH₂CH₂NHC(═O)—; 4H, —OCH₂CH₂N<; 9H, —OCH₃), 3.02-2.87 (m, 2H, —C(═O)NHCH₂CH<), 2.33 (t, 2H, —C(═O)CH₂CH₂O—), 2.05-1.91 (m, 4H, —NHC(═O)CH₂CH₂CH<), 1.55-1.01 (m, 44H, —CH₂CH₂CH₂—; 4H, —CH₂CH₃), 0.85 (t, 6H, —CH₂CH₃). The molecular weight determined by GPC was approximately 3.7 kDa, PDI=1.02.

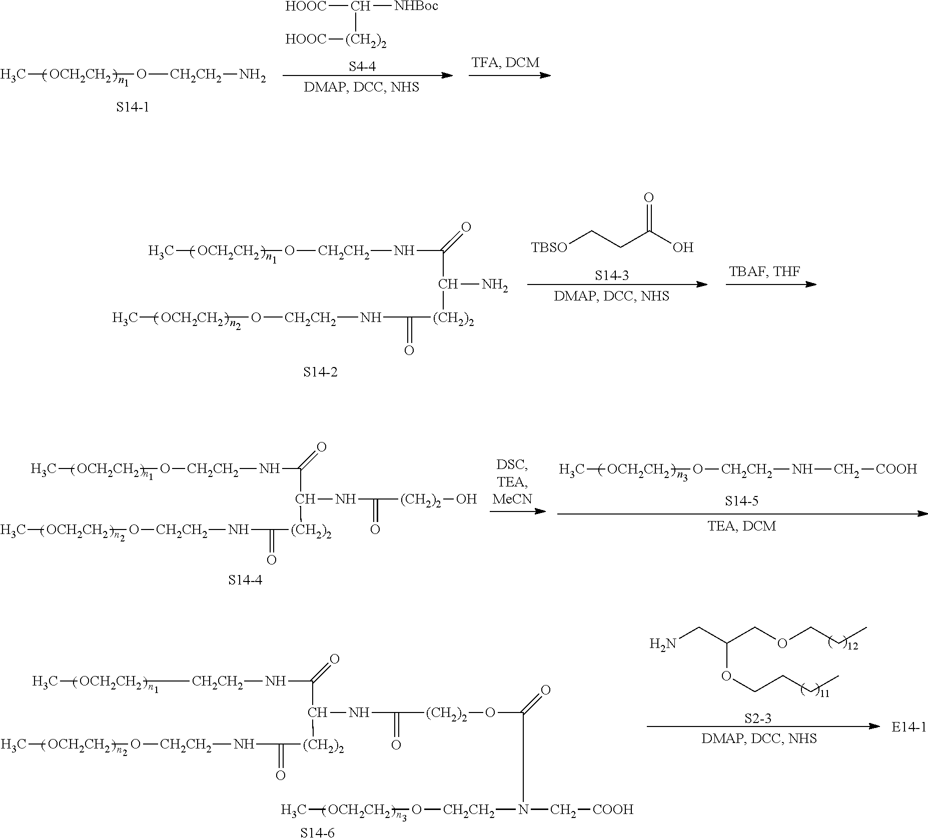

### Example 14.2: Preparation of E14-2

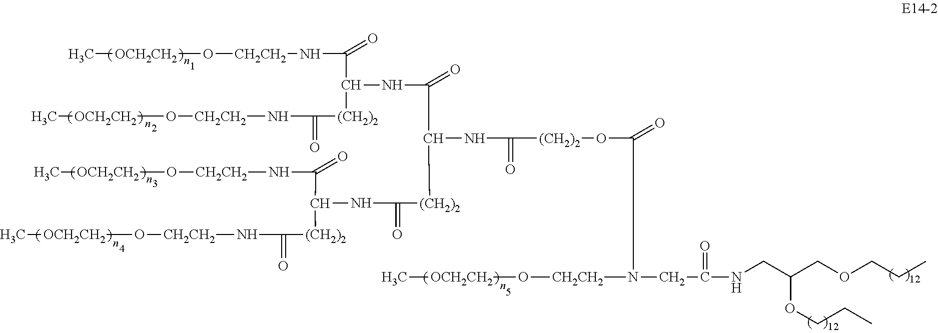

E14-2 corresponds to the general formula (1); wherein, X is —CH<, R₁ and R₂ are both tetradecyl groups, B₁ is a methylene group, B₂ is a linking bond, L₁ and L₂ are both ether groups (—O—), L_{d} is —CH₂C(═O)NHCH₂—, y=2, two XPEG contain 1 and 4 RPEG, respectively; T is a methyl group, one of XPEG has a non-linear structure and contains the

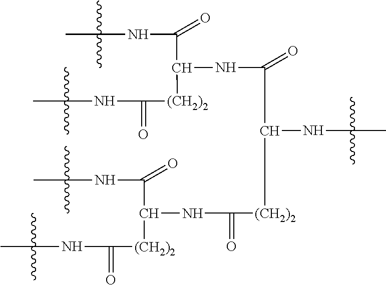

pentavalent branching core, and the two Lₓ are —C(═O)(CH₂)₂OC(═O)— and —CH₂CH₂—, respectively. The molecular weight of each PEG chain is approximately 1 kDa, corresponding to n₁≈n₂≈n₃≈n₄≈n₅≈22.

The preparation method is as follows:

Step a: The glutamic acid derivative containing a Boc-protected amino group (S4-4, 2.96 g, 12.0 mmol) was dissolved in 100 mL anhydrous dichloromethane, added with NHS (4.14 g, 36.0 mmol), and then added with DCC (7.42 g, 36.0 mmol). Into 200 mL solution of S14-2 (60.48 g, 28.8 mmol, Mₙ≈2.1 kDa, n₁≈n₂≈22, PDI=1.01) in dichloromethane, DMAP (0.59 g, 4.8 mmol) was added. Said two solutions were mixed and then reacted while stirring at room temperature for 24 hours. The reaction solution was filtered to remove the precipitates, and then concentrated under reduced pressure. The TFA/DCM mixed solution (1:1 v/v) was used to remove the Boc protection. After washing with purified water and extracting with dichloromethane, the extract obtained was dried over anhydrous sodium sulfate, filtered, concentrated, and purified by column chromatography to obtain the non-linear four-arm methoxypolyethylene glycol amine derivative S14-7 (19.45 g).

Step b: 3-Hydroxypropionic acid containing a TBS-protected hydroxyl group (S14-3, 2.45 g, 12.0 mmol) was dissolved in 50 mL anhydrous dichloromethane, added with NHS (2.07 g, 18.0 mmol), and then added with DCC (3.71 g, 18.0 mmol). Into 100 mL solution of S14-7 (17.03 g, 4.0 mmol, Mₙ≈4.3 kDa, n₁≈n₂≈n₃≈n₄≈22, PDI=1.01) in dichloromethane, DMAP (0.29 g, 2.4 mmol) was added. Said two solutions were mixed and then reacted while stirring at room temperature for 24 hours. The reaction solution was filtered to remove the precipitates, and then concentrated under reduced pressure. The TBAF/THF solution was used to remove the TBS protection. After the reaction was over, the reaction solution was concentrated and extracted. The organic phases were combined, and then dried over anhydrous sodium sulfate, filtered, concentrated, and purified by column chromatography to obtain the non-linear four-arm methoxypolyethylene glycol derivative S14-8 (9.44 g).

Step c: S14-8 (8.80 g, 2.0 mmol, Mₙ≈4.4 kDa, n₁≈n₂≈n₃≈n₄≈22, PDI=1.02) was dissolved in 60 mL anhydrous acetonitrile, added with TEA (0.33 mL, 2.4 mmol) and DSC (0.61 g, 2.4 mmol), and then reacted at room temperature while stirring overnight. Then, the reaction solution was concentrated under reduced pressure. The residue was dissolved with 50 mL dichloromethane and then washed with saturated sodium bicarbonate solution (20 mL*3). The organic phase was dried over anhydrous sodium sulfate and filtered. The filtrate was added with the methoxypolyethylene glycol carboxylic acid derivative S14-5 (2.20 g, 2.0 mmol, Mₙ≈1.1 kDa, n₁≈22, PDI=1.01) and TEA (0.33 mL, 2.4 mmol). The reaction was conducted at room temperature for 2 hours, and then the reaction solution was concentrated and washed. The crude product was purified by column chromatography to obtain the non-linear three-arm methoxypolyethylene glycol carboxylic acid derivative S14-9 (4.62 g).

Step d: S14-9 (4.40 g, 0.8 mmol, Mₙ≈5.5 kDa, n₁≈n2≈n3≈n4≈n₅≈22, PDI=1.02) was dissolved in 30 mL anhydrous dichloromethane, added with NHS (0.14 g, 1.2 mmol), and then added with DCC (0.25 g, 1.2 mmol). Into 15 mL solution of S2-3 (1.16 g, 2.4 mmol) in dichloromethane, DMAP (20 mg, 0.2 mmol) was added. Said two solutions were mixed and then reacted while stirring at room temperature for 24 hours. After the reaction was over, the precipitates were removed by filtration. The filtrate was dried over anhydrous sodium sulfate, filtered, concentrated, and purified by column chromatography to obtain the PEGylated lipid E14-2 (1.20 g). The main data of the ¹H-NMR spectrum of E14-2 are as follows: ¹H NMR (400 MHz, CDCl₃) δ: 4.30-4.11 (m, 3H, —NHC(═O)CH<; 2H, —NHC(═O)CH₂CH₂O—), 4.03 (s, 2H, >NCH₂C(═O)NH—), 3.78-3.33 (m, PEG; 3H, >CHCH₂O—; 4H, —OCH₂CH₂CH₂—; 16H, —OCH₂CH₂NHC(═O)—; 4H, —OCH₂CH₂N<; 15H, —OCH₃), 3.09-2.91 (m, 2H, —C(═O)NHCH₂CH<), 2.33 (t, 2H, —NHC(═O)CH₂CH₂OC(═O)—), 2.07-1.90 (m, 12H, —NHC(═O)CH₂CH₂CH<), 1.56-1.07 (m, 44H, —CH₂CH₂CH₂—; 4H, —CH₂CH₃), 0.88 (t, 6H, —CH₂CH₃). The molecular weight determined by GPC was approximately 6.0 kDa, PDI=1.02.

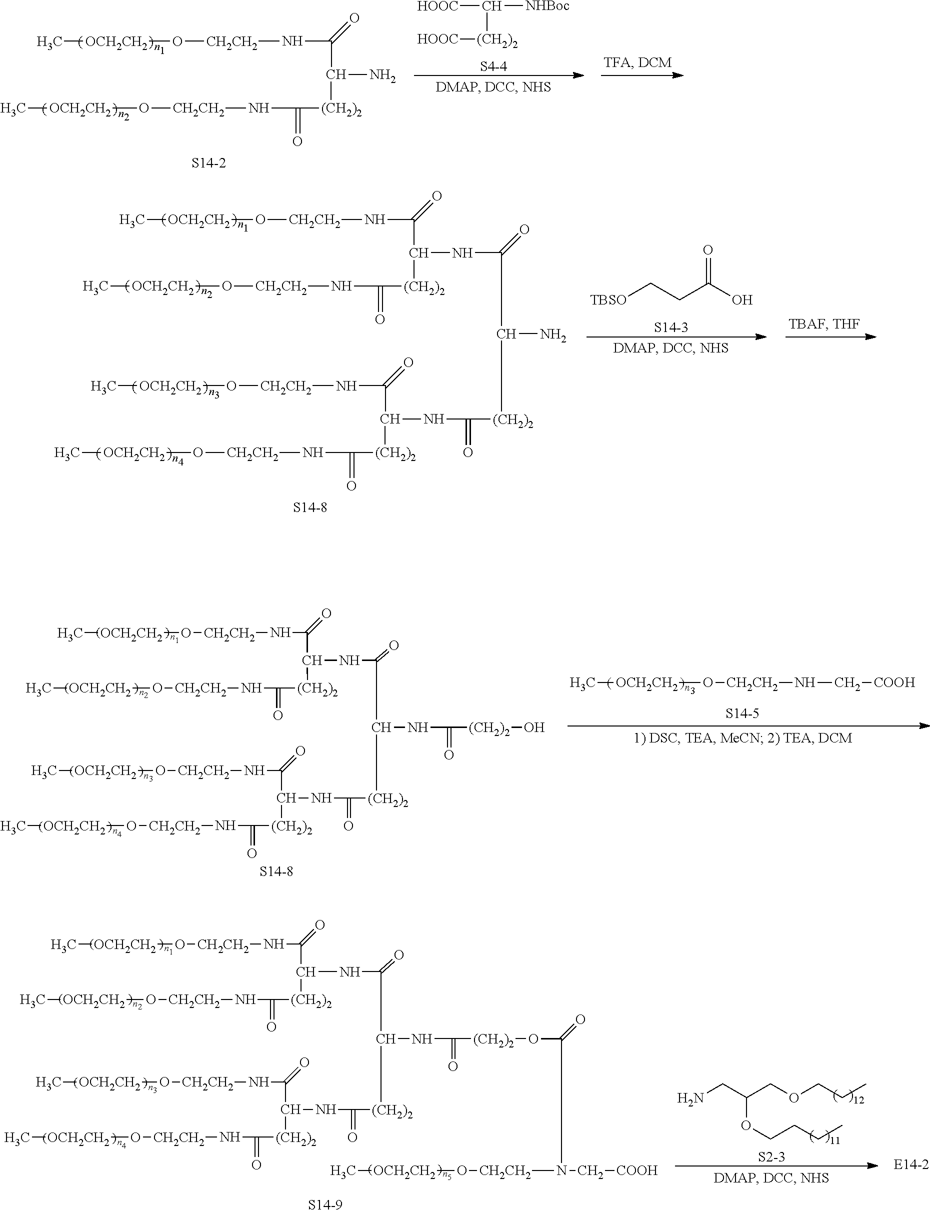

### 2.2. Lipid Pharmaceutical Compositions

### Example 15: Preparation of LNP-mRNA Pharmaceutical Compositions

In this embodiment, LNP-mRNA pharmaceutical compositions containing Fluc-mRNA (LNP/Fluc-mRNA) were prepared; wherein, the phospholipids contained were all DSPC, the steroid lipids contained were all cholesterol, the cationic lipids contained were all CL-1, but the PEGylated lipids contained were different; CL-1 was obtained by referring to the preparation method disclosed by CN113402405A, and its structure is as follows:

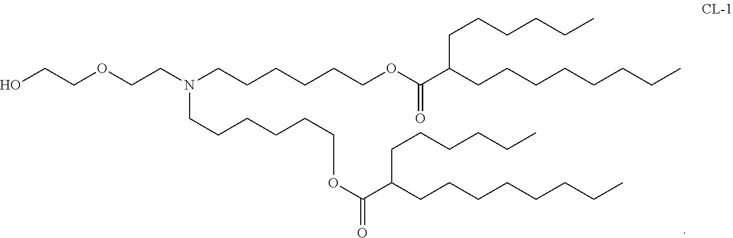 [SMILES: CCCCCCCCC(CCCCCC)C(=O)OCCCCCCN(CCCCCCOC(=O)C(CCCCCC)CCCCCCCC)CCOCCO]

The preparation method of LNP/Fluc-mRNA is as follows:

A certain amount of stock solutions of CL-1, DSPC, cholesterol and PEGylated lipid were pipetted to be dissolved in ethanol, wherein the molar ratio of CL-1, DSPC, cholesterol and PEGylated lipid was 50:10:38:1.5, therefore obtaining the ethanol phase solution (specific lipid formulation of each group is illustrated in Table 1 of Example 16; the PEGylated lipid of the control group L-0 is PEG2k-DMG (abbreviated as DMG); the PEGylated lipids of the experimental groups are the non-linear PEGylated lipids of the present invention). The Fluc-mRNA was added into a 10-50 mM citrate buffer (pH=4) to obtain the aqueous phase solution. The ethanol phase solution and the aqueous phase solution were mixed (1:3 v/v) to prepare the LNP/Fluc-mRNA. The DPBS ultrafiltration was performed multiple times to wash and remove ethanol and free molecules. Finally, the solution was passed through a 0.2 m sterile filter for further use.

Following the above steps, the molar ratio of lipids was adjusted to CL-1:DSPC:cholesterol:PEGylated lipid=50:10:38:0.75, with the PEGylated lipid being E1-1. All other conditions remained unchanged to prepare the LNP/Fluc-mRNA (L1-1-half).

Following the above steps, the molar ratio of lipids was adjusted to CL-1:DSPC:cholesterol:PEGylated lipid=48:9:42:1.5, with the PEGylated lipid being E1-1. All other conditions remained unchanged to prepare the LNP/Fluc-mRNA (L1-1-d).

### Example 16: Biological Assays for Lipid Pharmaceutical Compositions

### (1) Determination of Nanoparticle Size and Nucleic Acid Complexation Ability

Determination of nucleic acid complexation ability: The gel electrophoresis experiment was carried out to examine the nucleic acid complexation ability of LNP/Fluc-mRNA. 0.8 g agarose was weighed and dissolved in 40 mL solution of TAE. The solution was heated in a microwave until the granules of agarose were completely dissolved, and then cooled. 5 L nucleic acid dye (GelGreen) was added in the cooled agarose gel, and then the gel was added into the gel slot and dried with natural air. The mixed solution of LNP/Fluc-mRNA and 2 L loading buffer was added into the agarose gel well, and the electrophoresis voltage was set to 90 V. The electrophoresis was carried out at room temperature for 10 min. The result showed that free Fluc-mRNA hardly existed in both the experimental groups and the control group, indicating that the lipid composition containing the PEGylated lipid of the present invention has a good ability to complex with nucleic acids.

Determination of encapsulation efficiency: The LNP/Fluc-mRNA was ultracentrifuged (4° C., 60000 rpm, 1 h) with an ultracentrifuge. The concentration of unencapsulated Fluc-mRNA in the supernatant was determined by a nucleic acid quantifier. The encapsulation efficiency of LNP for Fluc-mRNA was calculated. The results are summarized in Table 1, showing that the LNPs of the present invention have higher encapsulation efficiency for nucleic acid drugs; wherein, the encapsulation efficiency of the experimental groups containing non-linear two-arm, three-arm, or five-arm PEGylated lipids with a molecular weight of approximately 1 kDa or 2 kDa per arm are all above 91%, and meanwhile the encapsulation efficiency of the experimental groups containing non-linear PEGylated lipids with a molecular weight of approximately 0.5 kDa per arm (L2-5, L6-1) also exceeds 80%. Particularly, the molar ratio of PEGylated lipid in L1-1-half is only half of that in L1-1 or the control group L-0; however, the encapsulation efficiency of L1-1-half is very close to that of L-0, wherein the former is 91.9% and the latter is 92.3%, indicating that even in a circumstance where the non-linear PEGylated lipid of the present invention is used in an appropriately reduced amount, the encapsulation efficiency of LNP for Fluc-mRNA is not significantly affected.

Determination of particle size: According to the literature (Hassett et al., J. Controlled Release 2021, 335, 237-246), an LNP formulation loaded with nucleic acid drugs can exhibit better pharmaceutical efficacy when its particle size is in the range of 60-150 nm. In this embodiment, the particle size of LNP/Fluc-mRNA was determined by dynamic light scattering (DLS). The measured sizes of LNP/Fluc-mRNA were relatively uniform, and the PDI values were all smaller than 0.3. The results showed that the particle sizes of LNP/Fluc-mRNA prepared with the non-linear two-arm, three-arm, or five-arm PEGylated lipids of the present invention with a molecular weight of approximately 1 kDa or 2 kDa per arm were ranging from approximately 59 nm to approximately 90 nm; when the two-arm non-linear PEGylated lipid was used, the particle size of the corresponding LNP/Fluc-mRNA was slightly larger than 100 nm; all of these particle sizes were within the range of the particle size that can achieve better pharmaceutical efficacy.

TABLE 1
Determination of Particle Size and Encapsulation Efficiency

| LNP | L-0 | L1-1 | L1-2 | L2-1 |
|---|---|---|---|---|
| PEGylated Lipid | PEG2k-DMG | E1-1 | E1-2 | E2-1 |
| Encapsulation | 92.3 | 93.0 | 93.1 | 89.1 |
| Efficiency/% | | | | |
| Particle Size/nm | 97 | 83 | 77 | 88 |
| LNP | L2-2 | L2-3 | L2-4 | L2-5 |
| PEGylated Lipid | E2-2 | E2-3 | E2-4 | E2-5 |
| Encapsulation | 93.1 | 98.2 | 92.7 | 81.2 |
| Efficiency/% | | | | |
| Particle Size/nm | 84 | 71 | 101 | 59 |
| LNP | L3-1 | L4-1 | L4-2 | L4-3 |
| PEGylated Lipid | E3-1 | E4-1 | E4-2 | E4-3 |
| Encapsulation | 95.2 | 91.4 | 92.3 | 93.4 |
| Efficiency/% | | | | |
| Particle Size/nm | 72 | 85 | 72 | 69 |
| LNP | L5-1 | L6-1 | L6-2 | L7-1 |
| PEGylated Lipid | E5-1 | E6-1 | E6-2 | E7-1 |
| Encapsulation | 92.4 | 90.9 | 97.1 | 93.5 |
| Efficiency/% | | | | |
| Particle Size/nm | 89 | 61 | 74 | 106 |
| LNP | L8-1 | L8-2 | L9-1 | L9-2 |
| PEGylated Lipid | E8-1 | E8-2 | E9-1 | E9-2 |
| Encapsulation | 95.0 | 97.2 | 98.0 | 98.2 |
| Efficiency/% | | | | |
| Particle Size/nm | 69 | 76 | 70 | 73 |
| LNP | L10-1 | L11-1 | L12-1 | L13-1 |
| PEGylated Lipid | E10-1 | E11-1 | E12-1 | E13-1 |
| Encapsulation | 99.6 | 98.6 | 97.3 | 92.6 |
| Efficiency/% | | | | |
| Particle Size/nm | 68 | 84 | 80 | 83 |
| LNP | L14-1 | L14-2 | L1-1-half | L1-1-d |
| PEGylated Lipid | E14-1 | E14-2 | E1-1 | E1-1 |
| Encapsulation | 97.5 | 95.7 | 91.9 | 94.1 |
| Efficiency/% | | | | |
| Particle Size/nm | 85 | 87 | 81 | 84 |

### (2) Evaluation of Stability in Serum

Cationic lipids play an important role in the LNP for delivering nucleic acid drugs. During the delivery process in the systemic circulation, there exist quantities of negatively charged serum proteins that are easily adsorbed to positively charged cationic lipid nanoparticles to form large-sized aggregates which can be cleared by the mononuclear phagocytic system, therefore resulting in a lower genetic transfection efficiency of LNP. The non-linear PEGylated lipid of the present invention can impart the “stealth effect” to LNP.

The aforementioned LNP/Fluc-mRNA (1 control group L-0 and 27 experimental groups L1-1-L1-1-d) were respectively added to the culture media containing 10% fetal bovine serum (FBS), stirred at 37° C., and sampled at a regular interval to determine the change of particle size of LNP/Fluc-mRNA to analyze the stability of nucleic acid pharmaceutical formulation in serum. The results showed that, in 7 days, the particle sizes of the experimental groups L2-5 and L6-1, characterized by a molecular weight of 0.5 kDa per arm for polyethylene glycol components, exhibited relatively large changes (11% and 15%, respectively), while all the rest experimental groups and the control group had the changes in particle size of less than 8%; particularly, the experimental groups characterized by a molecular weight of 2 kDa per arm for polyethylene glycol components experienced almost no change in particle size (approximately 1%); particularly, the change of particle size of L1-1-half was approximately 5% which was not significantly different from those of L-0 and L-1-1 (approximately 4% and 3%, respectively), while the amount of PEGylated lipid used in L1-1-half was only half of that of L-0 or L1-1. These results indicate that the LNP-nucleic acid pharmaceutical compositions prepared with the non-linear two-arm or multi-arm PEGylated lipids of the present invention, with a molecular weight of approximately 1 kDa per arm of polyethylene glycol, have good stability in serum; when the molecular weight of PEGylated lipid is close to one another (e.g., DMQ E1-1 of the present invention), the non-linear structure with shorter PEG chains can achieve better stability of LNP pharmaceutical composition in serum, compared with the linear structure with a longer PEG chain; even when used in a smaller amount, the non-linear PEGylated lipids can still achieve relatively good stability of LNP pharmaceutical composition in serum.

(3) Evaluation of cytotoxicity

The cells used were HeLa cells, and the method used was to measure cell viability by CCK-8 kit.

The commercial transfection reagent Lipofectamine 2000 (L2K) was used to prepare the L2K/Fluc-mRNA complex according to the method in Example 15.

HeLa cells were inoculated onto a 96-well plate at 6,000 cells/well, 100 L per well, and then divided into the control group (blank control group), the L2K/Fluc-mRNA group (positive control group) and the LNP/Fluc-mRNA groups (experimental groups L1-1-L14-2), and then incubated at 37° C., 5% CO₂. After 24 hours of cell incubation, into the blank control group was added 10 μL PBS solution, into the positive control group and the experimental groups were added 3.3 g/mL L2K/Fluc-mRNA (10 μL) and 3.3 g/mL LNP/Fluc-mRNA (10 L), respectively, and the incubation was continued at 37° C., 5% CO₂. After another 24 hours of incubation, the 96-well plate was retrieved while protected from light. The culture medium was aspirated, and then a diluted solution of CCK-8 was added at a volume of 120 μL per well. Then, the incubation was continued for 1-4 hours at 37° C., 5% CO₂.

The 96-well plate was retrieved, and a microplate reader was used to measure the absorbance of each well at a wavelength of 450 nm.

The results of three repeated assays were averaged, and showed that the cell viability of the positive control group L2K/Fluc-mRNA was 92%, those of the experimental groups were all above 87%, and particularly, those of L1-1, L1-2, L3-1, L4-1, L4-2, L8-2 and L9-1 were all above 98%.

These results indicate that, compared with the L2K/Fluc-mRNA which contains the commercial transfection reagent Lipofectamine 2000, the LNP/Fluc-mRNA of the present invention also has no obvious cytotoxicity toward HeLa cells.

### (4) Evaluation of Transfection Efficiency

In order to examine the transfection efficiency of mRNA at cell level for each group of LNP/Fluc-mRNA composition prepared in Example 15, assays were performed on the basis of luciferase bioluminescence. The formulation of LNP/Fluc-mRNA composition was dissolved in the culture medium to the required dose, and HeLa cells, as a cell model, were inoculated at a density of 6,000 cells/well. The cell suspension was inoculated at 100 μL/well onto a 96-well black frame plate with clear wells. After the inoculation, the cells were incubated for 24 h in a cell culture incubator. Then, a dose of 0.2 ug mRNA per well was given, while the blank control group was added with free Fluc-mRNA of the corresponding dose. After 24 hours of transfection, the old culture medium was removed and replaced with a new medium containing the D-fluorescein sodium substrate (1.5 mg/mL). After 5 minutes of incubation, bioluminescence was detected using a microplate reader. Stronger fluorescence meant more Fluc-mRNA were transported into the cytoplasm and translated into the corresponding fluorescent protein. The results are shown in Table 2, wherein, the relative value of fluorescence intensity is the ratio of the fluorescence intensity of each group to that of the blank control group. The results indicate that the LNP/Fluc-mRNA compositions prepared in the present invention all have excellent transfection efficiency in vitro, i.e., the LNPs of the control group and the experimental groups are all effective carriers for delivery, and most of the experimental groups featuring a degradable linking group in the PEGylated lipid have higher transfection efficiency than the control group. Particularly, the LNP/Fluc-mRNA compositions L1-1, L2-1, L2-3, L4-1, and L4-2 of the present invention all have significantly higher transfection efficiency than L-0 which employs DMG as the PEGylated lipid.

TABLE 2
Results of Cell Transfection Assays

| | Relative | | Relative | | Relative |
|---|---|---|---|---|---|
| | Value of | | Value of | | Value of |
| | Fluorescence | | Fluorescence | | Fluorescence |
| No. | Intensity | No. | Intensity | No. | Intensity |
| Blank | 1 | L1-1 | 9.8 | L4-1 | 10.5 |
| L-0 | 8.0 | L1-2 | 9.0 | L4-2 | 10.8 |
| | | L2-1 | 10.8 | L4-3 | 9.3 |
| | | L2-2 | 9.3 | L5-1 | 8.3 |
| | | L2-3 | 10.0 | L6-1 | 8.8 |
| | | L2-4 | 8.8 | L6-2 | 8.0 |
| | | L2-5 | 9.5 | L7-1 | 8.5 |
| | | L3-1 | 8.0 | L9-1 | 7.5 |
| | | L8-1 | 7.3 | L9-2 | 8.3 |
| | | L8-2 | 8.0 | | |

### (5) Evaluation of Targeting Ability

This embodiment examines the targeting ability of the LNP containing E10-1 which is modified by folic acid at the polyethylene glycol ends, and compares it with E2-3, a structure most similar in structure except for the ends of PEG

### I Preparation of the Formulation of Non-Linear PEGylated Lipid Nanoparticle/Oxaliplatin Composition:

Step a: DSPC (4 g) and cholesterol (667 mg) were dissolved in 60 mL dichloromethane, added with 20 mL aqueous solution of 80 mg oxaliplatin, stirred for 30 min, and ultrasonicated for 20 min. The organic solvent was removed by rotary evaporation at 40° C. After the gel was collapsed, components including E2-3 (0.50 g) and 50 mL aqueous solution of poloxamer F68 (400 mg) were added respectively. The evaporation was continued for 30 min, followed by high-pressure homogenization at 400 bar for 5 min. The volume was adjusted to 100 mL with water, and then the tangential flow ultrafiltration was used to isolate oxaliplatin. A tangential-flow membrane pack with a molecular weight cut-off of 10 kDa was used for the ultrafiltration. The ultrafiltration was performed repeatedly for 3 times, using water as the displacement liquid. The ultrafiltration centrifuge tube was used to measure the encapsulation efficiency, and the high performance liquid chromatography was used to measure the final concentration of drug. The encapsulation efficiency was 95.33%. The particle size was 124 nm. The concentration of drug in the LNP suspension was adjusted to 500 g/mL with water, therefore obtaining the formulation of non-linear PEGylated lipid nanoparticle/oxaliplatin composition, LP-1.

Step b: Using the same preparation method as that of the abovementioned experimental group, E10-1 (0.7 g) was used to replace E2-3 (0.5 g) in the preparation to obtain the formulation of non-linear PEGylated lipid nanoparticle/oxaliplatin composition, LP-2.

II. The HCT-116 human colon cancer cell line was recovered and cultured, and the HCT-116 human colon cancer tumor-bearing nude mouse model was established. The nude mice were inoculated and grew naturally, and the tumor volume was measured with a vernier caliper.

After the tumor grew to 50-75 mm³, the mice were randomly divided into 4 groups on the basis of the tumor volume:
(1) Blank control group: 6 mice; the tumor tissue was retrieved after one administration via the tail vein with 5% dextrose injection;
(2) Control group 1: one administration with 10 mg/kg oxaliplatin injection; 12 mice;
(3) Control group 2: one administration with 10 mg/kg LP-1 formulation; 12 mice;
(4) Experimental group: one administration with 10 mg/kg LP-2 formulation; 12 mice;
wherein, the control group 1, the control group 2 and the experimental group were administered via the tail vein and sacrificed at different time points of 0.05, 0.5, 1, 1.5, 2, 2.5, 3, 3.5, 4, 6, 8, and 48 h. The tumor masses of nude mice were placed in a freezer for storage at −20° C., and then the HPLC was used to detect the concentration of drug. The results showed that, compared with the oxaliplatin injection, LP-1 did not exhibit a higher targeting ability; the targeting ability of LP-2 was significantly enhanced, and the corresponding peak concentration in plasma was significantly increased (by 77%), indicating that the functional modification with targeting groups at the polyethylene glycol ends of a PEGylated lipid in the present invention can improve the targeting efficiency of LNP.

Those described above are only embodiments of the present invention, not limiting the patent scope of the present invention. Any transformation of equivalent structure or equivalent route based on the specification content of the present invention, directly or indirectly applied in other related arts, should be included in the protected patent scope of the present invention in the same way.

For those skilled in the art, without deviating from the purpose and the scope of the present invention, and without unnecessary experimentation, the present invention can be implemented in a wider range with equivalent parameters, concentrations, and conditions. While the present invention has provided particular examples, it should be understood that the present invention can be further modified. In conclusion, in accordance with the principles of the present invention, the present application is intended to cover any alterations, uses, or improvements of the present invention, including changes deviating from the scope disclosed in this application but made using conventional techniques known in the art.

1.-32. (canceled)

33. A PEGylated lipid of the general formula (1): 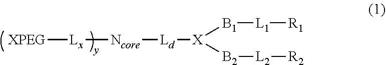
wherein, X is —CRₐ< or 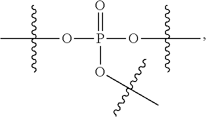 [SMILES: *OP(=O)(O*)O*]
and Rₐ is H or a C₁₋₁₂ alkyl group;
B₁ and B₂ are each independently a linking bond or a C₁₋₂₀ alkylene group;
L₁ and L₂ are each independently a linking bond or a divalent linking group;
R₁ and R₂ are each independently a C₁₋₅₀ aliphatic hydrocarbon group or a C₁₋₅₀ residue of aliphatic hydrocarbon derivative, containing 0 to 10 heteroatoms; the heteroatom is B, O, N, Si, P or S;
L_{d} is a linking bond or a divalent linking group;
N_{core} is a multivalent group having a valence of y+1, and contains a trivalent nitrogen-atom branching core connected to L_{d};
y is 2,3,4,5, 6, 7, 8 or 9, or y>10;
y instances of Lₓ are each independently a linking bond or a divalent linking group;
XPEG is a polyethylene glycol component; each of y instances of XPEG independently contains one, two, three or four RPEG; RPEG is a single-chain polyethylene glycol component containing at least 4 EO units, and the EO unit is —CH₂CH₂O— or —OCH₂CH₂—; RPEG in the same XPEG have the same terminal group T; T is a hydrogen atom, an alkyl group or R₀₁-L₀₁-, wherein L₀₁ is a linking bond or a divalent linking group, and R₀₁ is a functional group that can interact with bio-related substances;
the alkyl group, alkylene group, aliphatic hydrocarbon group, and residue of aliphatic hydrocarbon derivative are each independently substituted or unsubstituted;
the PEGylated lipid is monodisperse or polydisperse;
or a salt, tautomer, stereoisomer or solvate thereof.

34. The PEGylated lipid of claim 33, wherein L₁ and L₂ correspond to any of the following cases:
Case (1): one of L₁ and L₂ is a linking bond, and the other is a divalent linking group;
Case (2): both L₁ and L₂ are linking bonds;
Case (3): both L₁ and L₂ are divalent linking groups, and L₁ and L₂ have the same or different structures;
in any of Case (1), Case (2) and Case (3), the divalent linking group is selected from the group consisting of —CH₂—, —O—, —S—, —C(═O)—, —NR_{c}—, and combinations thereof; wherein, Rₑ is, at each occurrence, independently a hydrogen atom or a C₁₋₅ alkyl group;
specifically, L₁ and L₂ are each independently selected from the group consisting of a linking bond, —O—, —NHC(═O)—, —C(═O)NH—, —OC(═O)—, —C(═O)O—, —OC(═O)O—, —C(═O)—, —C(═O)O—(CH₂)ₓ—OC(═O)—, —C(═O)O—(CH₂)ₓ—C(═O)O—, —OC(═O)—(CH₂)ₓ—OC(═O)—, —OC(═O)—(CH₂)ₓ—C(═O)O—, —C(═O)NH—(CH₂)ₓ—C(═O)O—, —C(═O)NH—(CH₂)ₓ—OC(═O)—, —NHC(═O)—(CH₂)ₓ—C(═O)O—, —NHC(═O)—(CH₂)ₓ—OC(═O)—, —C(═O)NH—(CH₂)ₓ—C(═O)NH—, —C(═O)NH—(CH₂)ₓ—NHC(═O)—, —NHC(═O)—(CH₂)ₓ—C(═O)NH—, and —NHC(═O)—(CH₂)ₓ—NHC(═O)—;
wherein x is an integer in the range of 2 to 8.

35. The PEGylated lipid of claim 33, wherein B₁ and B₂ correspond to any of the following cases:
Case (1): one of B₁ and B₂ is a linking bond, and the other is a C₁₋₂₀ alkylene group;
Case (2): both B₁ and B₂ are linking bonds;
Case (3): both B₁ and B₂ are C₁₋₂₀ alkylene groups, and B₁ and B₂ have the same or different structures;
the C₁₋₂₀ alkylene group has 0 to 4 hydrogen atoms replaced by 0 to 4 R_{q}; R_{q} is, at each occurrence, independently selected from the group consisting of —(CH₂)_{tq}C[(CH₂)_{tq}H]₃, —(CH₂)_{tq}O(CH₂)_{tq}H, —(CH₂)_{tq}S(CH₂)_{tq}H, and —(CH₂)_{tq}N[(CH₂)_{tq}H]₂, wherein tq is, at each occurrence, independently an integer in the range of 0 to 4.

36. The PEGylated lipid of claim 33, wherein R₁ and R₂ are each independently selected from the group consisting of R_{L}, R_{B}, and Rᵣ; R₁ and R₂ each independently contains 0 to 10 Rₘ substituents; Rₘ is, at each occurrence, independently a linear, branched or ring-containing C₁₋₈ hydrocarbon group;
R₁ and R₂ each independently contains 0 to 4 carbon-carbon double bonds and/or 0 to 4 carbon-carbon triple bonds;
R_{L} is selected from the group consisting of the following structures and cis-/trans-isomers thereof: 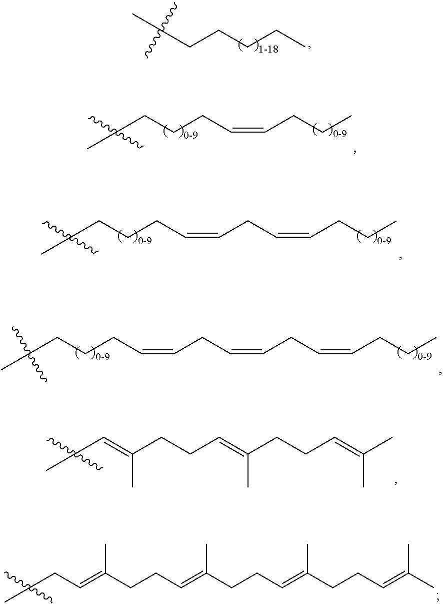
wherein, the structure of R_{B} is 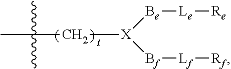
wherein X is CH or N; the t of R_{B} is an integer in the range of 0 to 5; Bₑ and B_{f} are each independently a linking bond or a C₁₋₁₀ alkylene group; Lₑ and L_{f} are each independently a linking bond, —O—, —OC(═O)—, —C(═O)O—, —NHC(═O)—, or —C(═O)NH—; Rₑ and R_{f} are each independently a C₁₋₁₂ alkyl group;
wherein, Rᵣ is a ring-containing C₄₋₃₀ alkyl group or C₄₋₃₀ heteroalkyl group.

37. The PEGylated lipid of claim 33, wherein each of y instances of XPEG independently has a linear or non-linear structure; the linear structure contains one RPEG; the non-linear structure contains two, three, or four RPEG and also contains a trivalent, tetravalent or pentavalent branching core, wherein the branching core is selected from the group consisting of the following structures: 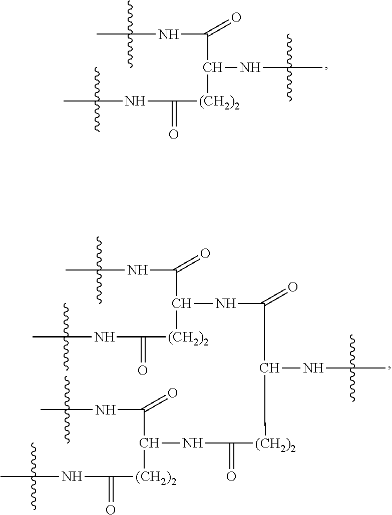
wherein the right end is connected to Lₓ.

38. The PEGylated lipid of claim 33, wherein the structure of RPEG is 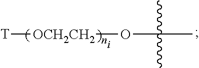
wherein, nᵢ is the degree of polymerization of the polyethylene glycol chain, being an integer in the range of 4 to 250, wherein the polyethylene glycol chain is polydisperse or monodisperse; the i in nᵢ is an integer selected from 1 to m, and m is equal to the total number of RPEG in the PEGylated lipid.

39. The PEGylated lipid of claim 38, wherein the nᵢ of quantity m are each independently an integer in the range of 4 to 100.

40. The PEGylated lipid of claim 38, wherein the number average molecular weight of RPEG is selected from 0.5 kDa to 20 kDa.

41. The PEGylated lipid of claim 33, the structure of which is represented by the general formula (2) or (3): 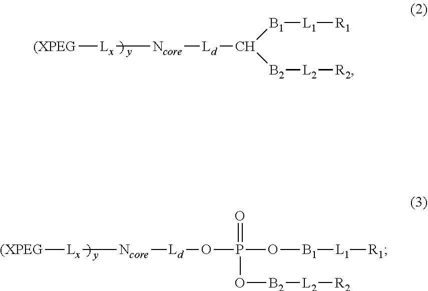
wherein, y is 2 or 3;
when y is 2, N_{core} is selected from the group consisting of 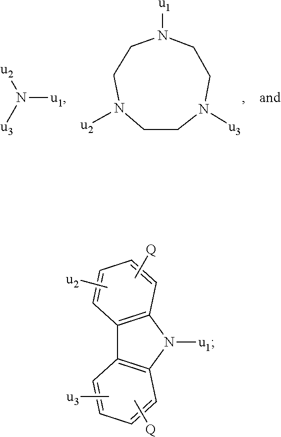
when y is 3, N_{core} is 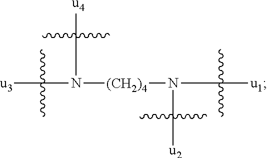
wherein, u₁, u₂, u₃, and u₄ are each independently a linking bond connected to L_{d} or Lₓ, and any two of u₁, u₂, u₃, and u₄ are not simultaneously connected to the same L_{d} or Lₓ;
wherein, Q is an electron-changing group, the number of which is 0, 1 or greater than 1; when the number of Q is greater than 1, any two Q have the same or different structures;
wherein, T is a methyl group.

42. The PEGylated lipid of claim 41, wherein T is R₀₁-L₀₁-; wherein, L₀₁ is a linking bond, or selected from the group consisting of —CH₂—, —O—, —S—, —C(═O)—, —NH—, and combinations thereof;
L₀₁ is specifically selected from the group consisting of a linking bond, —(CH₂)ᵣ—, —NH(CH₂)ₜ—, —NH(CH₂)ₜC(═O)NH(CH₂)ₜ, —O(CH₂)ₜ—, —NH(CH₂)ₜC(═O)O(CH₂)ₜ, —OC(═O)(CH₂)ᵣ—, —OC(═O)O(CH₂)ₜ, —OC(═O)(CH₂)ₜC(═O)—, and —(CH₂)ₜC(═O)NH(CH₂)ₜ; the left end of L₀₁ is connected to R₀₁; the t of L₀₁ is an integer in the range of 1 to 4.

43. The PEGylated lipid of claim 42, wherein R₀₁ is a reactive group selected from the group consisting of a hydroxyl group, a thiol group, an active ester group, an active carbonate group, a sulfonate group, an amino group, a maleimide group, a succinimide group, a carboxyl group, an acyl chloride group, an aldehyde group, an azido group, a cyano group, an alkenyl group, an alkynyl group, an epoxyalkyl group, a rhodamine group, a folate residue, a biotin residue, a monosaccharide group, and a polysaccharide group, or a modified form thereof; the modified form is selected from the group consisting of the precursors of reactive groups, the active forms to which reactive groups are precursors, the active forms in which reactive groups are substituted, and the inactive forms in which reactive groups are protected; wherein, the precursors of reactive groups refer to the structures which can be transformed into the reactive groups through at least one process among oxidation, reduction, hydration, dehydration, electronic rearrangement, structural rearrangement, salt complexation and decomplexation, ionization, protonation, and deprotonation;
R₀₁ is specifically selected from the group consisting of the following structures:

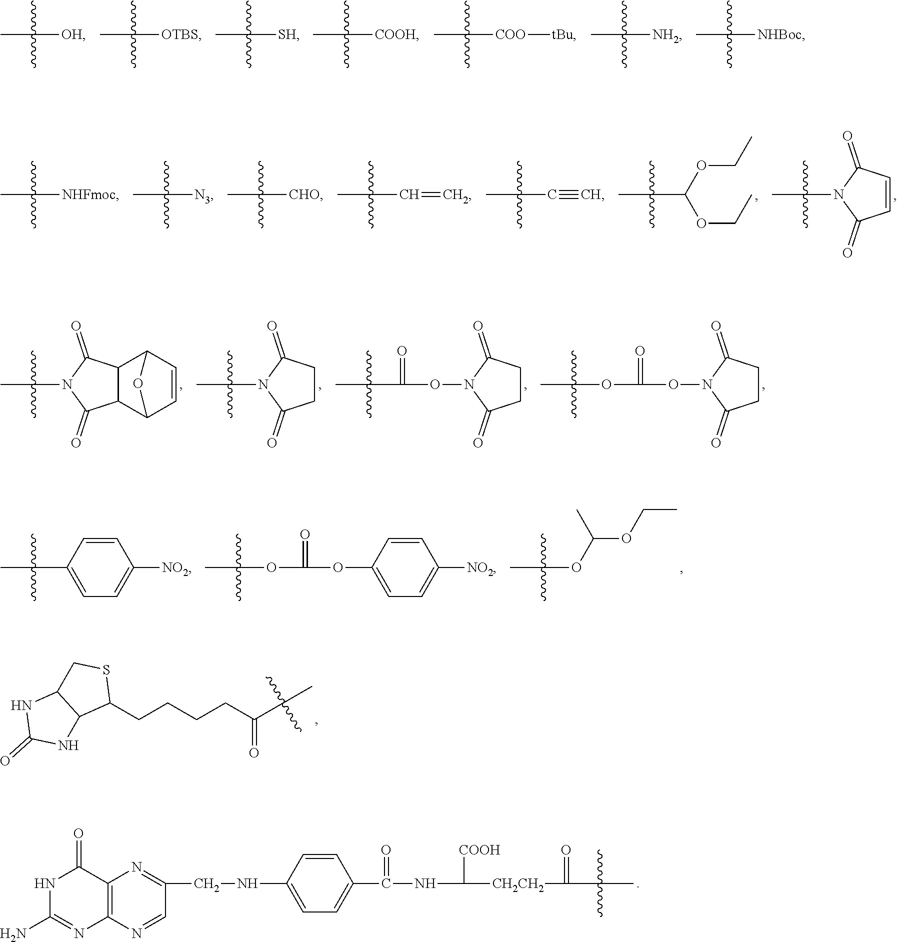

44. The PEGylated lipid of claim 41, wherein y instances of Lₓ are each independently selected from the group consisting of a linking bond, a hydrocarbylene group, a heteroatom-containing divalent linking group, and combinations thereof;
the heteroatom-containing divalent linking group is specifically —O—, —S—, —S—S—, —C(═O)—, —C(═O)O—, —OC(═O)—, —OC(═O)O—, —NH—, —C(═O)NH—, —NHC(═O)—, —OC(═O)NH—, or —NHC(═O)O—;
the hydrocarbylene group is specifically a C₁₋₅ alkylene group;
specifically, each Lₓ is independently selected from the group consisting of —(CH₂)₂—, —C(═O)—, —CH₂C(═O)—, —C(═O)CH₂—, —C(═O)(CH₂)₂—, —CH₂CH₂C(═O)OCH₂CH₂—, —C(═O)NH—, —C(═O)NH(CH₂)₃—, and —C(═O)(CH₂)₂OC(═O)—, wherein the right end of Lₓ is connected to N_{core}.

45. The PEGylated lipid of claim 41, wherein L_{d} is selected from the group consisting of a linking bond, —CH₂—, —(CH₂)₂—, —CH₂C(═O)O—, —CH₂C(═O)OCH₂—, —(CH₂)₂C(═O)O—, —(CH₂)₂C(═O)OCH₂—, —(CH₂)₃OC(═O)—, —CH₂C(═O)NH—, —CH₂C(═O)NHCH₂—, —(CH₂)₂C(═O)NH—, —(CH₂)₂C(═O)NHCH₂—, —(CH₂)₃NHC(═O)—, —CH₂C(═O)NHCH₂C(═O)OCH₂—, and —(CH₂)₃O(CH₂)₃NHCH₂—.

46. The PEGylated lipid of claim 41, the structure of which is selected from the group consisting of the following structures:

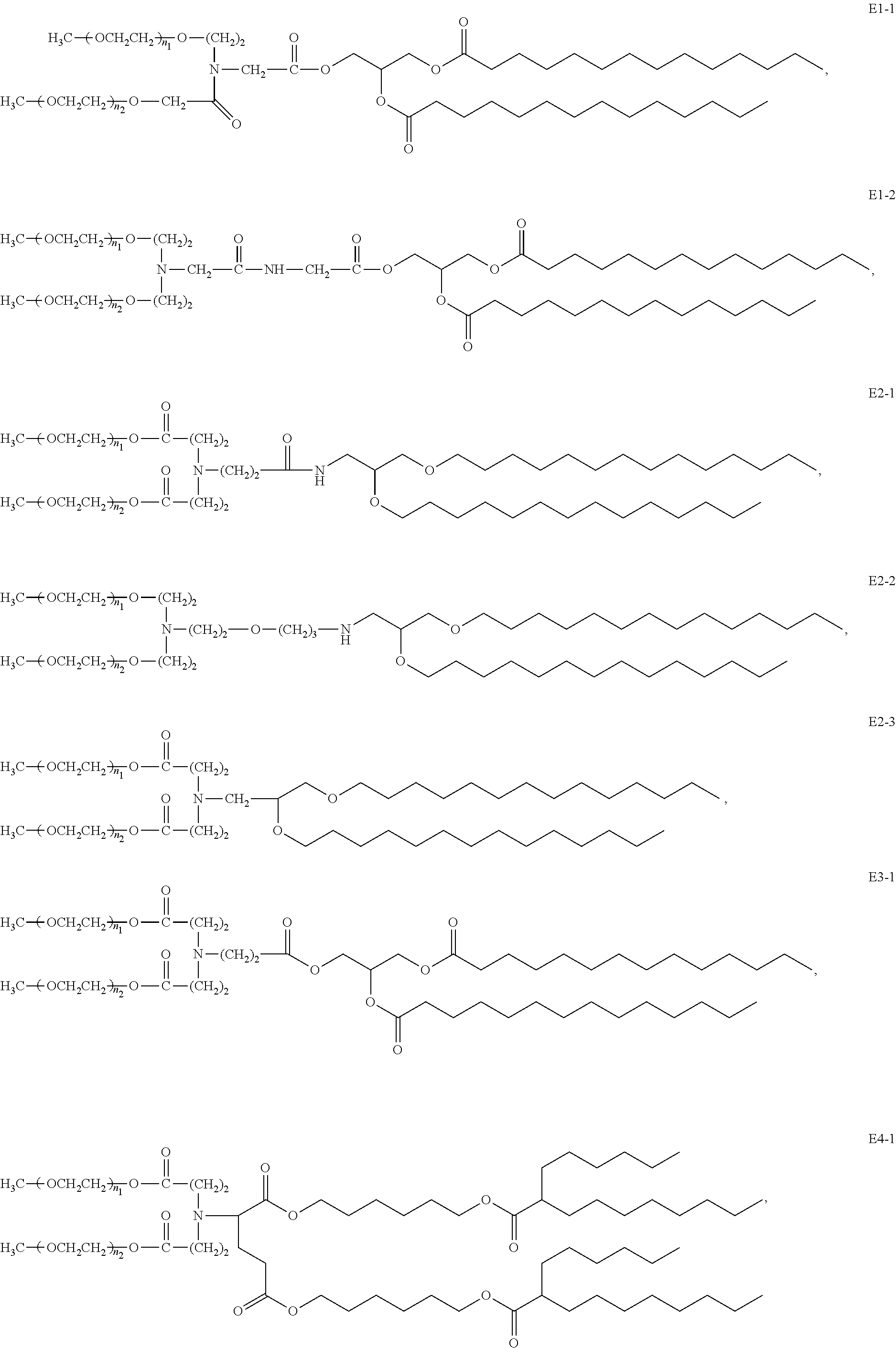

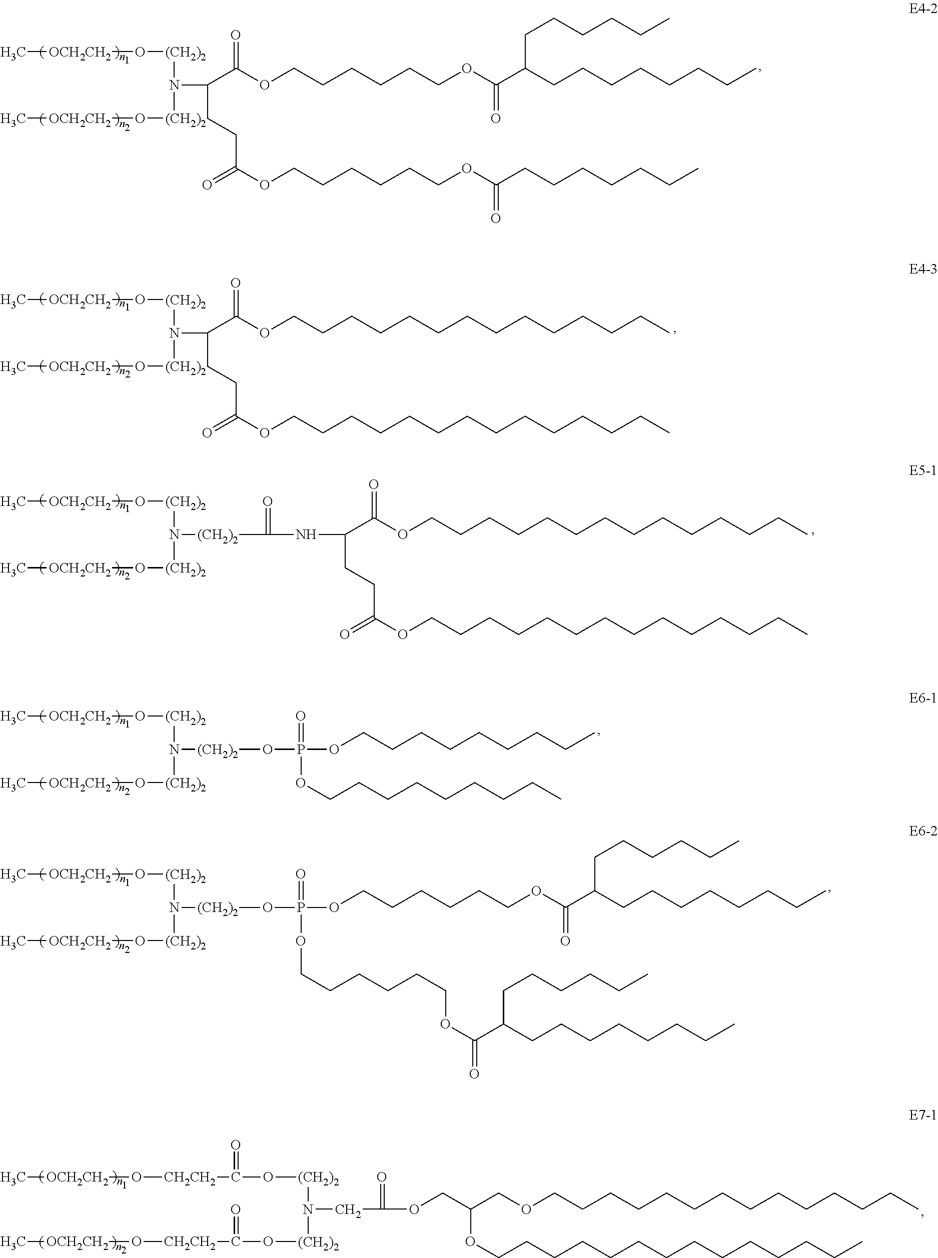

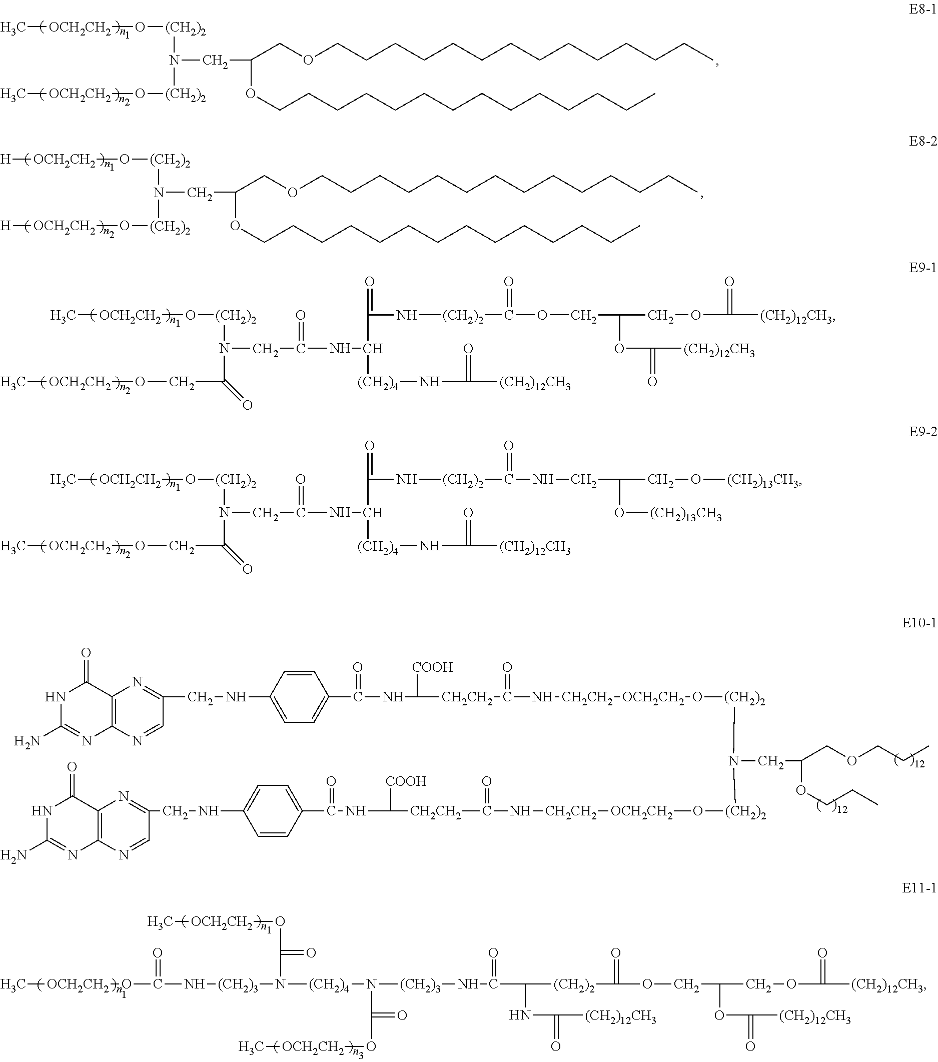

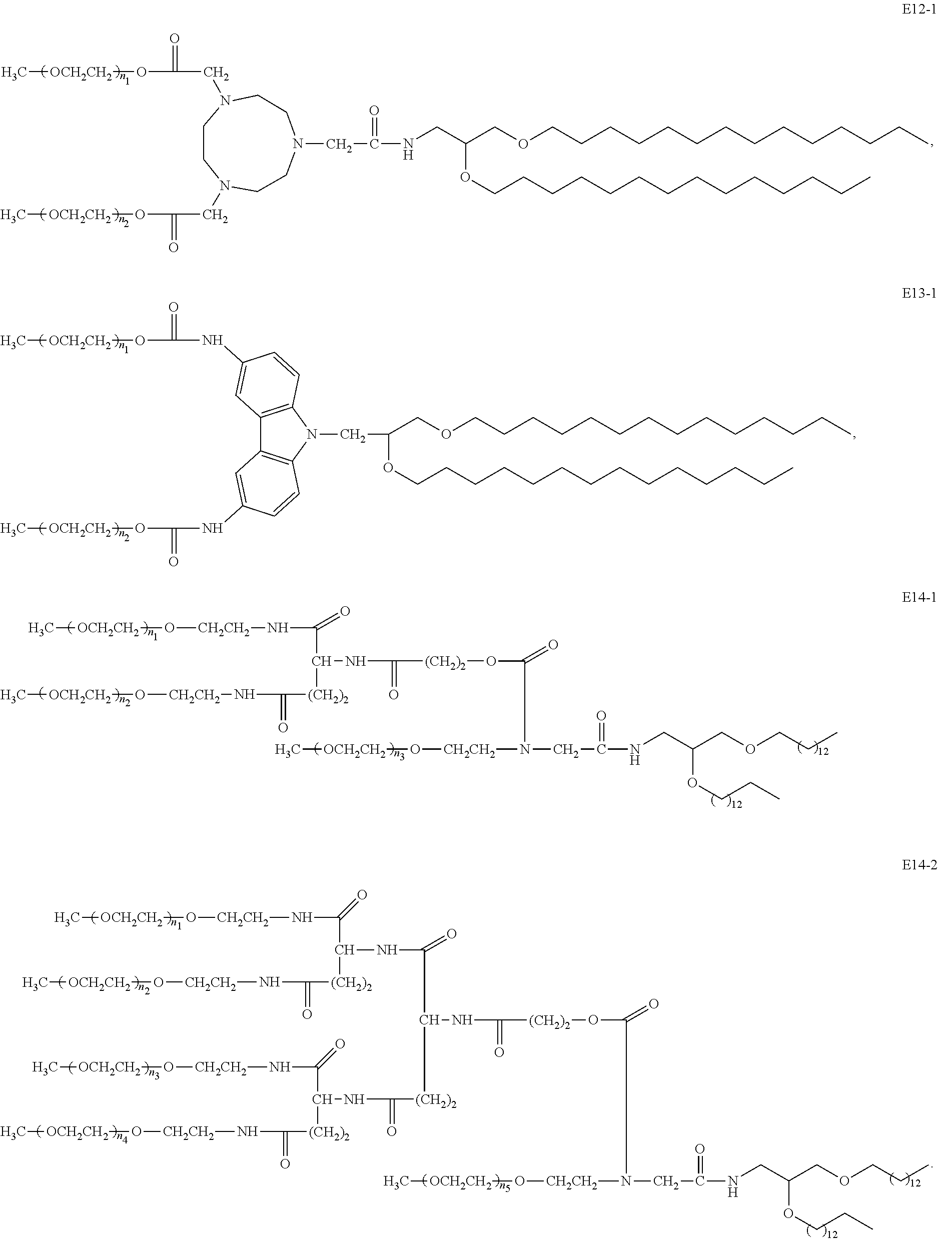

47. A lipid composition containing a PEGylated lipid of claim 33.

48. The lipid composition of claim 47, which contains another one or more types of lipids selected from the group consisting of phospholipid, steroid lipid, and cationic lipid, corresponding to any of the following cases:
Case (1): the lipid composition also contains a phospholipid;
Case (2): the lipid composition also contains a steroid lipid;
Case (3): the lipid composition also contains a cationic lipid;
Case (4): the lipid composition also contains a phospholipid and a steroid lipid;
Case (5): the lipid composition also contains a phospholipid and a cationic lipid;
Case (6): the lipid composition also contains a steroid lipid and a cationic lipid;
Case (7): the lipid composition also contains a phospholipid, a steroid lipid, and a cationic lipid.

49. The lipid composition of claim 48, wherein the phospholipid is selected from the group consisting of 1,2-dilinoleoyl-sn-glycero-3-phosphocholine, 1,2-dimyristoyl-sn-glycero-phosphocholine, 1,2-dioleoyl-sn-glycero-3-phosphocholine, 1,2-dipalmitoyl-sn-glycero-3-phosphocholine, 1,2-distearoyl-sn-glycero-3-phosphocholine, 1,2-diundecanoyl-sn-glycero-phosphocholine, 1-palmitoyl-2-oleoyl-sn-glycero-3-phosphocholine, 1,2-di-O-octadecenyl-sn-glycero-3-phosphocholine, 1-oleoyl-2-cholesterylhemisuccinoyl-sn-glycero-3-phosphocholine, 1-hexadecyl-sn-glycero-3-phosphocholine, 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine, 1,2-diphytanoyl-sn-glycero-3-phosphoethanolamine, 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt, dioleoylphosphatidylserine, dipalmitoylphosphatidylglycerol, palmitoyloleoylphosphatidylethanolamine, distearoylphosphatidylethanolamine, dipalmitoylphosphatidylethanolamine, dimyristoylphosphoethanolamine, 1-stearoyl-2-oleoyl-phosphatidyethanolamine, 1-stearoyl-2-oleoyl-phosphatidylcholine, sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine, and compositions thereof.

50. The lipid composition of claim 48, wherein the steroid lipid is selected from the group consisting of cholesterol, coprostanol, sitosterol, ergosterol, campesterol, stigmasterol, brassicasterol tomatidine, ursolic acid, α-tocopherol, and compositions thereof.

51. The lipid composition of claim 48, wherein the cationic lipid is selected from the group consisting of N,N-dioleyl-N,N-dimethylammonium chloride, N,N-distearyl-N,N-dimethylammonium bromide, N-[1-(2,3-dioleoyloxy)propyl]-N,N,N-trimethylammonium chloride, N-[1-(2,3-dioleyloxy)propyl]-N,N,N-trimethylammonium chloride, N,N-dimethyl-2,3-dioleyloxy-1-(dimethylamino)propane, 3-(didodecylamino)-N1,N1,4-tridodecyl-1-piperazineethanamine, N1-[2-(didodecylamino)ethyl]-N1,N4,N4-tridodecyl-1,4-piperazinediethanamine, 14,25-ditridecyl-15,18,21,24-tetraaza-octatriacontane, 1,2-dilinoleyloxy-N,N-dimethylaminopropane, 2,2-dilinoleyl-4-dimethylaminomethyl-[1,3]-dioxolane, heptatriaconta-6,9,28,31-tetraen-19-yl 4-(dimethylamino)butanoate, 2,2-dilinoleyl-4-(2-dimethylaminoethyl)-[1,3]-dioxolane, ((4-hydroxybutyl)azanediyl)bis(hexane-6,1-diyl)bis(2-hexyldecanoate), heptadecan-9-yl 8-((2-hydroxyethyl)(6-oxo-6-(undecyloxy)hexyl)amino)octanoate, 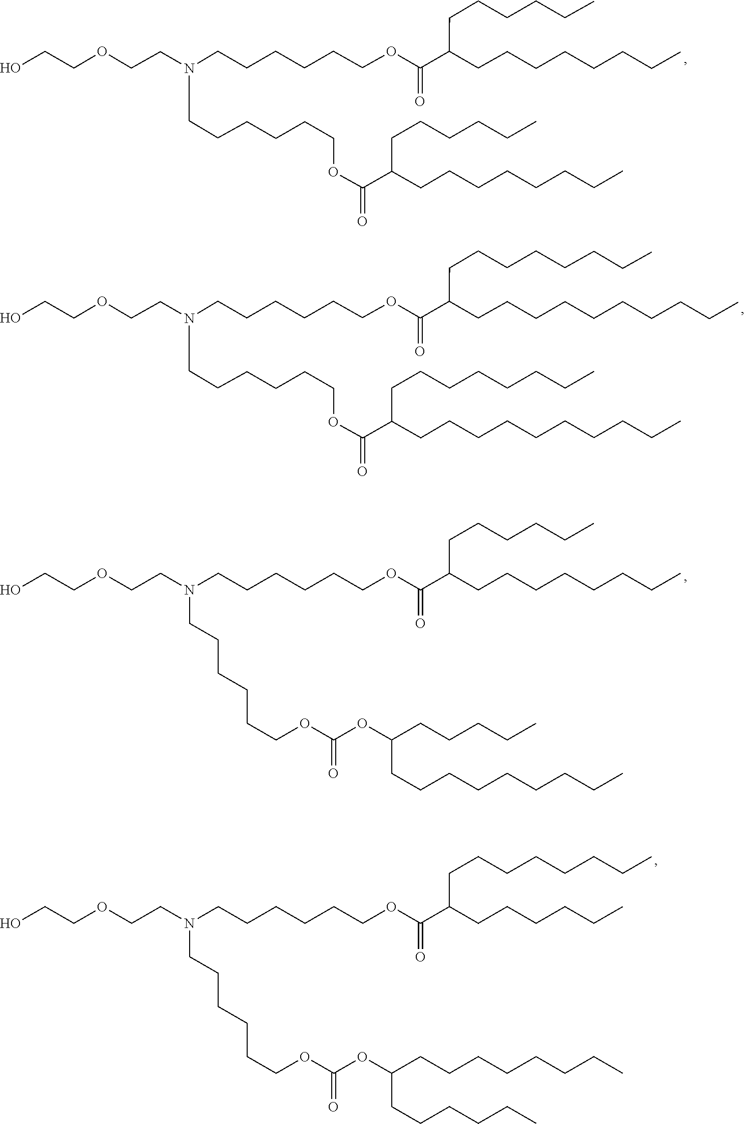 [SMILES: CCCCCCCCC(CCCCCC)C(=O)OCCCCCCN(CCCCCCOC(=O)C(CCCCCC)CCCCCCCC)CCOCCO ; CCCCCCCCCCC(CCCCCCCC)C(=O)OCCCCCCN(CCCCCCOC(=O)C(CCCCCCCC)CCCCCCCCCC)CCOCCO ; CCCCCCCCCC(CCCCC)OC(=O)OCCCCCCN(CCCCCCOC(=O)C(CCCCCC)CCCCCCCC)CCOCCO ; CCCCCCCCC(CCCCCC)OC(=O)OCCCCCCN(CCCCCCOC(=O)C(CCCCCC)CCCCCCCC)CCOCCO] 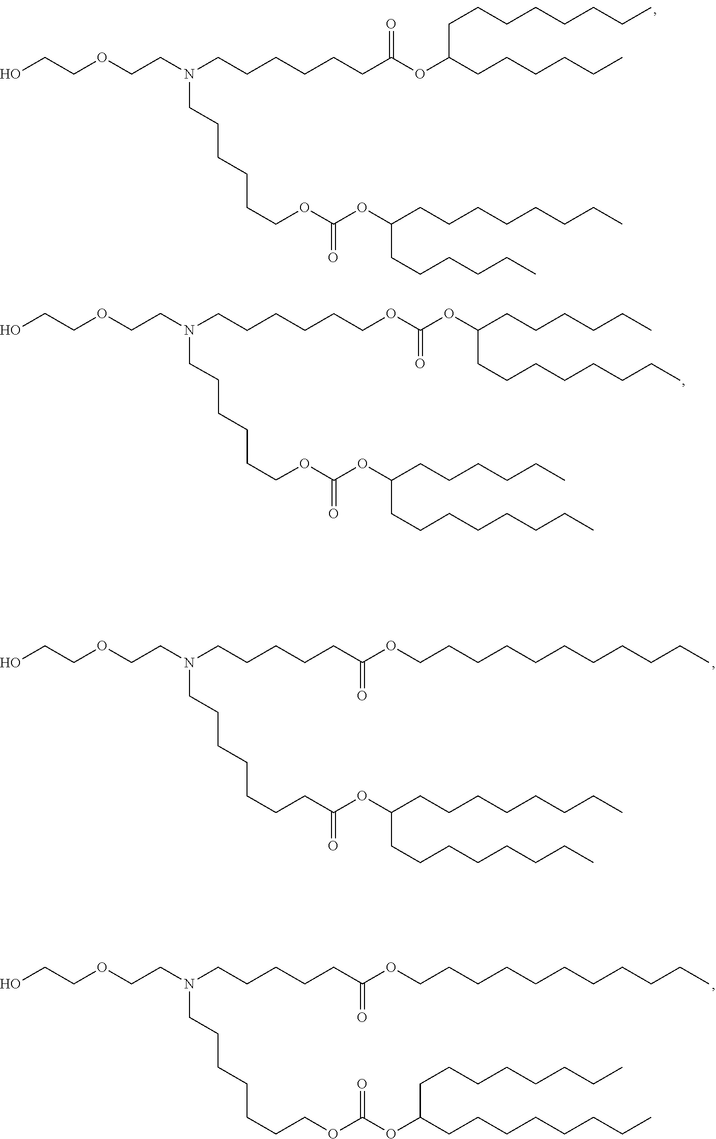 [SMILES: CCCCCCCCC(CCCCCC)OC(=O)CCCCCCN(CCCCCCOC(=O)OC(CCCCCC)CCCCCCCC)CCOCCO ; CCCCCCCCC(CCCCCC)OC(=O)OCCCCCCN(CCCCCCOC(=O)OC(CCCCCC)CCCCCCCC)CCOCCO ; CCCCCCCCCCCOC(=O)CCCCCN(CCCCCCCC(=O)OC(CCCCCCCC)CCCCCCCC)CCOCCO ; CCCCCCCCCCCOC(=O)CCCCCN(CCCCCCCOC(=O)OC(CCCCCCCC)CCCCCCCC)CCOCCO] 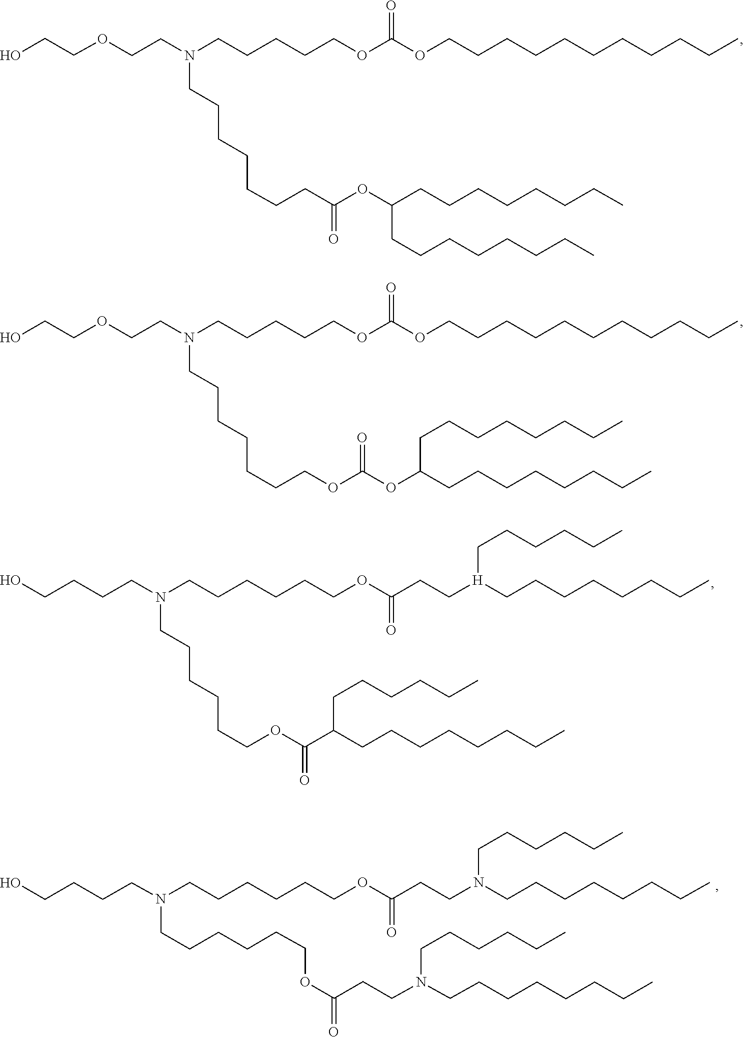 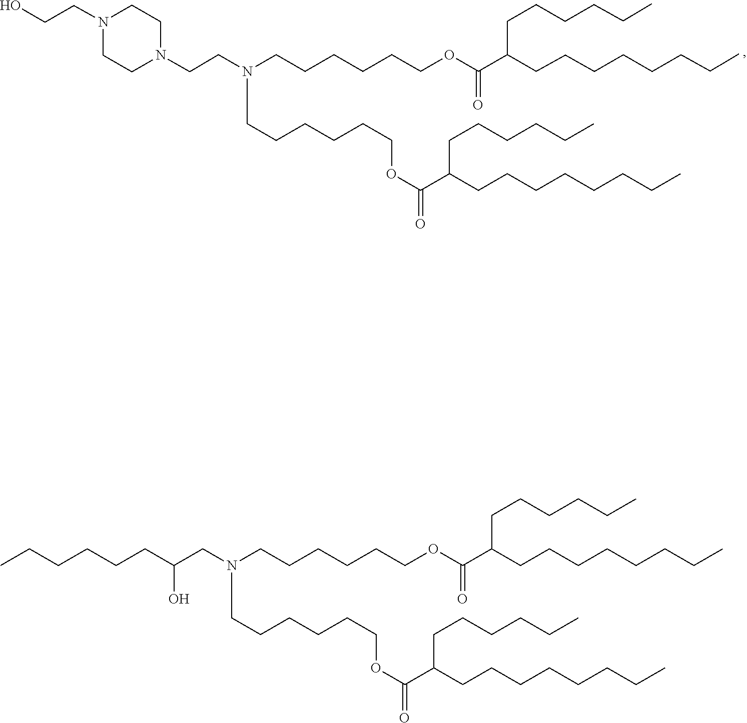
and compositions thereof.

52. The lipid composition of claim 48, wherein,
the molar percentage of PEGylated lipid in total lipids is 0.5 to 5%;
the molar percentage of cationic lipid in total lipids is 30 to 65%;
the molar percentage of phospholipid in total lipids is 7.5 to 13%;
the molar percentage of steroid lipid in total lipids is 35 to 50%.

53. A lipid pharmaceutical composition containing a lipid composition of claim 47 and a drug selected from the group consisting of nucleic acid drug, genetic vaccine, anti-neoplastic drug, small molecule drug, peptide drug, and protein drug.

54. The lipid pharmaceutical composition of claim 53, wherein the drug is a nucleic acid drug selected from the group consisting of DNA, RNA, antisense nucleic acid, plasmid, interfering nucleic acid, aptamer, antagomir, and ribozyme, wherein the RNA is selected from the group consisting of mRNA, saRNA, circRNA, miRNA, and siRNA.

55. The lipid pharmaceutical composition of claim 53, which is used as a medicine selected from the group consisting of drugs for treating cancer, anti-infective agents, antibiotic agents, antiviral agents, antifungal agents, and vaccines.

56. The lipid pharmaceutical composition of claim 53, which is an LNP-pharmaceutical composition, an LPP-pharmaceutical composition, or a PNP-pharmaceutical composition.
